(19) United States
(12) Patent Application Publication
(10) Pub. No.: US 2022/0089745 A1
(43) Pub. Date: Mar. 24, 2022

(54) ANTI-GLYCOPROTEIN IIb/IIIa ANTIBODIES

(71) Applicant: Biogen MA Inc., Cambridge, MA (US)

(72) Inventors: Bradley Pearse, Somerville, MA (US); Joe Salas, Wayland, MA (US); Robert Peters, Needham, MA (US); Christilyn Graff, Cambridge, MA (US)

(73) Assignee: Biogen MA Inc., Cambridge, MA (US)

(21) Appl. No.: 17/353,402

(22) Filed: Jun. 21, 2021

Related U.S. Application Data
(63) Continuation of application No. 16/721,162, filed on Dec. 19, 2019, now abandoned.
Application No. 16/721,162 is a continuation of application No. 15/521,683, filed on Apr. 25, 2017, now abandoned.
(60) Provisional application No. 62/073,348, filed on Oct. 31, 2014.

(51) Int. Cl. C07K 16/28; C07K 14/745; C12N 5/078; C12N 9/64; G01N 33/569

(52) U.S. Cl. CPC C07K 16/2848; C07K 2319/35; C12N 5/0644; C12N 9/6437; G01N 33/56966; C07K 2317/92; C07K 2319/33; C07K 2319/00; C07K 2317/33; C07K 2317/34; C07K 2317/522; C07K 2317/55; C07K 2317/56; C07K 2317/565; C07K 2317/622; C07K 2317/76; C07K 2319/30; C07K 14/745

(57) ABSTRACT

Antibodies and antigen-binding antibody fragments that bind to GPIIb/IIIa and chimeric polypeptides comprising these binding molecules are disclosed. Some of these antibodies and antigen-binding antibody fragments preferentially bind GPIIb/IIIa on activated platelets while others do not show a preference for binding GPIIb/IIIa on resting versus activated platelets. Some of these antibodies and antibody fragments do not inhibit the interaction of GPIIb/IIIa with fibrinogen, while some others do. The disclosed antibodies do not induce platelet activation. Some of these antibodies and antigen-binding antibody fragments are useful in targeting therapeutic agents such as dotting factors to platelets while others are useful in reducing platelet aggregation and/or thrombus formation.

### CROSS-REFERENCE TO RELATED APPLICATIONS

This application claims the benefit of priority of U.S. Provisional Application No. 62/073,348, filed Oct. 31, 2014, the contents of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Oct. 27, 2015, is named 13751-0224WO1_SL.txt and is 321,548 bytes in size.

### FIELD

This invention relates generally to antibodies or antigen-binding fragments thereof that bind to glycoprotein IIb/IIIa, chimeric polypeptides comprising same, and uses thereof.

### BACKGROUND

Glycoprotein IIb/IIIa (GPIIb/IIIa, also known as integrin α_{IIb}β₃) is an integrin complex that is expressed specifically and at high levels on the surface of platelets. This complex serves as a receptor for ligands such as fibrinogen and von Willebrand factor and plays an important role in regulating platelet f unction (e.g., platelet activation). The GPIIb/IIIa integrin complex is formed by the calcium-dependent association of GPIIb and GPIIIa, a required step in normal platelet aggregation and endothelial adherence. Platelet activation leads to a conformational change in GPIIb/IIIa receptors that induces binding to fibrinogen.

The GPIIb/IIIa receptor is a target of several drugs such as GPIIb/IIIa inhibitors (e.g., abciximab, eptifibatide, tirofiban). Such inhibitors work by reducing or preventing platelet aggregation and thrombus formation. They are useful to treat acute coronary syndromes without percutaneous coronary intervention. GPIIb/IIIa inhibitors are also used for treating patients who have unstable angina, certain types of heart attacks, and in combination with angioplasty with or without stent placement. The drugs are generally given in combination with heparin or aspirin (blood-thinning agents) to prevent clotting before and during invasive heart procedures.

In addition, agents that target GPIIb/IIIa receptors can be used to enhance rather than prevent or inhibit clotting. For example, agents that bind or target GPIIb/IIIa receptors but do not inhibit its interaction with fibrinogen can be used to target clotting factors to platelets to enhance clotting in a subject in need of such treatment. Clotting factors have been administered to patients to improve hemostasis for some time. The advent of recombinant DNA technology has significantly improved treatment for patients with clotting disorders, allowing for the development of safe and consistent protein therapeutics. For example, recombinant activated factor VII has become widely used for the treatment of major bleeding, such as that which occurs in patients having hemophilia A or B, deficiency of coagulation Factors XI or VII, defective platelet function, thrombocytopenia, or von Willebrand's disease. Although such recombinant molecules are effective, there is a need for improved versions which localize the therapeutic agent to sites of coagulation, have improved pharmacokinetic properties, improved manufacturability, reduced thrombogenicity, or enhanced activity, or more than one of these characteristics.

Accordingly, there is an unmet medical need for better treatment and prevention options for patients with coagulation disorders (e.g., hemophilia patients with inhibitors in which the activity of the FVIIa protein is increased). In addition, there is an unmet medical need for improved therapeutic agents that can be used in treating conditions that require inhibition or prevention of clotting. Furthermore agents that are effective in transporting a therapeutic agent to platelets are desired.

### SUMMARY

The present disclosure features antibodies and antigen-binding fragments thereof that bind to GPIIb/IIIa. These antibodies can be grouped into at least three classes: one class (Class I) includes antibodies that preferentially bind GPIIb/IIIa on activated platelets compared to GPIIb/IIIa on resting platelets; a second class (Class II) does not show preferential binding for GPIIb/IIIa on activated platelets compared to GPIIb/IIIa on resting platelets and does not compete with fibrinogen for binding GPIIb/IIIa; and a third class (Class III) does not show preferential binding for GPIIb/IIIa on activated platelets compared to GPIIb/IIIa on resting platelets and competes with fibrinogen for binding GPIIb/IIIa. All of these classes of antibodies do not activate platelets. Class I and Class II anti-GPIIb/IIIa antibodies and antigen-binding fragments thereof can be used, for example, to target or transport any agent of interest (e.g., a therapeutic molecule such as a clotting factor) to platelets. Specifically, Class I antibodies or antigen-binding fragments can be used as a delivery agent to activated platelets, whereas Class II antibodies or antigen-binding fragments can be used as a delivery agent to all platelets. For example, the Class I and Class II antibodies can be used as delivery agents for a clotting factor like Factor VII (FVII). The clotting factor FVIIa has low affinity for platelets, the site of action for clot formation. Thus, one approach to increase activity of a clotting factor like FVIIa is to target this clotting factor to platelet receptors via targeting moieties (e.g., Fab or scFv of a Class I or Class II anti-GPIIb/IIIa antibody), which can increase the affinity of FVIIa for platelets thereby boosting activity. Such chimeric molecules can include a heterologous moiety to improve the pharmacokinetic parameters of the molecules such as its half-life. Class III anti-GPIIb/IIIa antibodies and antigen-binding fragments thereof described herein can be used, for example, to reduce, inhibit or prevent clotting in a subject in need thereof. They are also useful to reduce preventing platelet aggregation and thrombus formation in a subject in need thereof. Chimeric molecules of Class III antibodies are antigen-binding fragment thereof can include a heterologous moiety to improve the pharmacokinetic parameters of the molecules such as its half-life. In addition to their use as targeting moieties, the anti-GPIIb/IIIa antibodies and antigen-binding fragments thereof of this disclosure can be used as diagnostics, for example, by conjugation to a detectable label, and also for isolating or separating platelets from a sample. Class I antibodies can be used to separate activated platelets from resting platelets or enrich for activated platelets. Class III antibodies can also be used as a diagnostic tool for evaluating fibrinogen blocking.

In one aspect, the disclosure features an antibody or antigen-binding fragment thereof that specifically binds to Glycoprotein IIb/IIIa (GPIIb/IIIa), wherein the antibody or antigen-binding fragment thereof preferentially binds to GPIIb/IIIa on activated platelets compared to resting platelets and does not activate platelets. In certain embodiments, the antibody or antigen-binding fragment thereof does not inhibit the association of fibrinogen with GPIIb/IIIa. In some embodiments, the antibody or antigen-binding fragment thereof comprises the complementarity determining regions (CDRs) of the heavy chain variable domain (VH) amino acid sequence set forth in SEQ ID NOs. 9, 29, 33, or 37, with zero to four mutations in one or more of the CDRs. In other embodiments, the antibody or antigen-binding fragment thereof comprises the complementarity determining regions of the VH amino acid sequence set forth in SEQ ID NOs. 9, 29, 33, or 37. In certain embodiments, the antibody or antigen-binding fragment thereof comprises an amino acid sequence that is at least 85% identical to the VH amino acid sequence set forth in SEQ ID NOs. 9, 29, 33, or 37. In some embodiments, the antibody or antigen-binding fragment thereof comprises the VH amino acid sequence set forth in SEQ ID NOs. 9, 29, 33, or 37. In further embodiments, the antibody or antigen-binding fragment thereof comprises the complementarity determining regions of the light chain variable domain (VL) amino acid sequence set forth in SEQ ID NOs. 11, 31, 35, or 39, with zero to four mutations in one or more of the CDRs. In certain embodiments, the antibody or antigen-binding fragment thereof comprises the light chain variable domain (VL) amino acid sequence set forth in SEQ ID NOs. 11, 31, 35, or 39.

In another aspect, the disclosure features an antibody or antigen-binding fragment thereof that specifically binds to Glycoprotein IIb/IIIa (GPIIb/IIIa), wherein the antibody or antigen-binding fragment thereof binds to GPIIb/IIIa on both activated platelets and resting platelets and does not activate platelets. In some embodiments, the antibody or antigen-binding fragment thereof does not inhibit the association of fibrinogen with GPIIb/IIIa. In certain embodiments, the antibody or antigen-binding fragment thereof binds to GPIIb/IIIa on activated platelets and resting platelets with the same or substantially the same binding affinity. In one embodiment, the antibody or antigen-binding fragment thereof comprises the complementarity determining regions of the VH amino acid sequence set forth in SEQ ID NOs. 5, 13, 17, 21, 25, 41, 45, or 49, with zero to four mutations in one or more of the CDRs. In another embodiment, the antibody or antigen-binding fragment thereof comprises the complementarity determining regions of the VH amino acid sequence set forth in SEQ ID NOs. 5, 13, 17, 21, 25, 41, 45, or 49. In a further embodiment, the antibody or antigen-binding fragment thereof comprises a VH amino acid sequence that is at least 85% identical to the amino acid sequence set forth in SEQ ID NOs. 5, 13, 17, 21, 25, 41, 45, or 49. In a certain embodiment, the antibody or antigen-binding fragment thereof comprises the VH amino acid sequence set forth in SEQ ID NOs. 5, 13, 17, 21, 25, 41, 45, or 49. In another embodiment, the antibody or antigen-binding fragment thereof comprises the complementarity determining regions of the VL amino acid sequence set forth in SEQ ID NOs. 7, 15, 19, 23, 27, 43, 47, or 51. In a certain embodiment, the antibody or antigen-binding fragment thereof comprises a VL amino acid sequence that is at least 85% identical to the amino acid sequence set forth in SEQ ID NOs. 7, 15, 19, 23, 27, 43, 47, or 51. In another embodiment, the antibody or antigen-binding fragment thereof comprises a VL amino acid sequence that is identical to the amino acid sequence set forth in SEQ ID NOs. 7, 15, 19, 23, 27, 43, 47, or 51.

In a third aspect, the disclosure features an antibody or antigen-binding fragment thereof that specifically binds to Glycoprotein IIb/IIIa (GPIIb/IIIa), wherein the antibody or antigen-binding fragment thereof binds to GPIIb/IIIa on both activated platelets and resting platelets, does not activate platelets, and inhibits the association of fibrinogen with GPIIb/IIIa. In certain embodiments, the antibody or antigen-binding fragment thereof binds to GPIIb/IIIa on activated platelets and resting platelets with the same or substantially the same binding affinity. In some embodiments, the antibody or antigen-binding fragment thereof comprises the complementarity determining regions of the VH amino acid sequence set forth in: SEQ ID NOs. 13 or 17. In some embodiments, the antibody or antigen-binding fragment thereof comprises the heavy chain variable domain (VH) amino acid sequence set forth in: SEQ ID NOs. 13 or 17. In certain embodiments, the antibody or antigen-binding fragment thereof comprises the complementarity determining regions of the VL amino acid sequence set forth in: SEQ ID NOs. 15 or 19. In certain embodiments, the antibody or antigen-binding fragment comprises VL amino acid sequence set forth in: SEQ ID NOs. 15 or 19.

In another aspect, the disclosure relates to an antibody or antigen-binding fragment thereof that specifically binds to GPIIb/IIIa, wherein the antibody or antigen-binding fragment thereof specifically binds to GPIIb/IIIa at the same epitope as an antibody comprising the VH and the VL amino acid sequences set forth in: SEQ ID NOs. 5 and 7; SEQ ID NOs. 9 and 11; SEQ ID NOs. 13 and 15; SEQ ID NOs. 17 and 19; SEQ ID NOs. 21 and 23; SEQ ID NOs. 25 and 27; SEQ ID NOs. 29 and 31; SEQ ID NOs. 33 and 35; SEQ ID NOs. 37 and 39; SEQ ID NOs. 41 and 43; SEQ ID NOs. 45 and 47; or SEQ ID NOs. 49 and 51.

In yet another aspect, the disclosure provides to an antibody or antigen-binding fragment thereof that specifically binds to GPIIb/IIIa, wherein the antibody or antigen-binding fragment thereof competitively inhibits or cross blocks GPIIb/IIIa binding by an antibody comprising the VH and the VL amino acid sequences set forth in: SEQ ID NOs. 5 and 7; SEQ ID NOs. 9 and 11; SEQ ID NOs. 13 and 15; SEQ ID NOs. 17 and 19; SEQ ID NOs. 21 and 23; SEQ ID NOs. 25 and 27; SEQ ID NOs. 29 and 31; SEQ ID NOs. 33 and 35; SEQ ID NOs. 37 and 39; SEQ ID NOs. 41 and 43; SEQ ID NOs. 45 and 47; or SEQ ID NOs. 49 and 51.

In a further aspect, the disclosure relates to an antibody or antigen-binding fragment thereof that specifically binds to GPIIb/IIIa, wherein the antibody or antigen-binding fragment thereof comprises at least three, at least four, or at least five CDRs of the VH and the VL amino acid sequences set forth in: SEQ ID NOs. 5 and 7; SEQ ID NOs. 9 and 11; SEQ ID NOs. 13 and 15; SEQ ID NOs. 17 and 19; SEQ ID NOs. 21 and 23; SEQ ID NOs. 25 and 27; SEQ ID NOs. 29 and 31; SEQ ID NOs. 33 and 35; SEQ ID NOs. 37 and 39; SEQ ID NOs. 41 and 43; SEQ ID NOs. 45 and 47; or SEQ ID NOs. 49 and 51.

In another aspect, the disclosure features an antibody or antigen-binding fragment thereof that specifically binds to GPIIb/IIIa, comprising:
(i) a variable heavy chain CDR-1 (VH-CDR1) sequence YTFTSYGIS (SEQ ID NO:53) or YTFTSYGIS (SEQ ID NO:53) with three, two, or one substitutions, a variable heavy chain CDR-2 (VH-CDR2) sequence (WISAYNGNTNYAQKLQG (SEQ ID NO:54) or (WISAYNGNTNYAQKLQG (SEQ ID NO:54) with three, two, or one substitutions; and a variable heavy chain CDR-3 (VH-CDR3) sequence (ARDLEYYDSSGYAYGYFDL (SEQ ID NO:55) or ARDLEYYDSSGYAYGYFDL (SEQ ID NO:55) with three, two, or one substitutions;
(ii) a VH-CDR1 sequence GTFSSYAIS (SEQ ID NO:56) or GTFSSYAIS (SEQ ID NO:56) with three, two, or one substitutions, a VH-CDR2 sequence GIIPIFGTANYAQKFQG (SEQ ID NO:57) or GIIPIFGTANYAQKFQG (SEQ ID NO:57) with three, two, or one substitutions; and a VH-CDR3 sequence ARDTGYYGASLYFDY (SEQ ID NO:58) or ARDTGYYGASLYFDY (SEQ ID NO:58) with three, two, or one substitutions;
(iii) a VH-CDR1 sequence GTFSSYAIS (SEQ ID NO:56) or GTFSSYAIS (SEQ ID NO:56) with three, two, or one substitutions, a VH-CDR2 sequence (GIIPIFGTANYAQKFQG (SEQ ID NO:57) or GIIPIFGTANYAQKFQG (SEQ ID NO:57) with three, two, or one substitutions; and a VH-CDR3 sequence ARGPPSAYGDYVWDI (SEQ ID NO:59) or ARGPPSAYGDYVWDI (SEQ ID NO:59) with three, two, or one substitutions;
(iv) a VH-CDR1 sequence FTFSDHHMD (SEQ ID NO:60) or FTFSDHHMD (SEQ ID NO:60) with three, two, or one substitutions, a VH-CDR2 sequence RTRNKANSYTTEYAASVKG (SEQ ID NO:61) or RTRNKANSYTTEYAASVKG (SEQ ID NO:61) with three, two, or one substitutions; and a VH-CDR3 sequence ARGPPYYADLGMGV (SEQ ID NO:62) or ARGPPYYADLGMGV (SEQ ID NO:62) with three, two, or one substitutions;
(v) a VH-CDR1 sequence YTFTSYSMH (SEQ ID NO:63) or YTFTSYSMH (SEQ ID NO:63) with three, two, or one substitutions, a VH-CDR2 sequence IINPSGGSTSYAQKFQG (SEQ ID NO:64) or IINPSGGSTSYAQKFQG (SEQ ID NO:64) with three, two, or one substitutions; and a VH-CDR3 sequence ARSYDIGYFDL (SEQ ID NO:65) or ARSYDIGYFDL (SEQ ID NO:65) with three, two, or one substitutions;
(vi) a VH-CDR1 sequence (YTFTSYGIS (SEQ ID NO:53) or YTFTSYGIS (SEQ ID NO:53) with three, two, or one substitutions, a VH-CDR2 sequence WISAYNGNTNYAQKLQG (SEQ ID NO:54) or WISAYNGNTNYAQKLQG (SEQ ID NO:54) with three, two, or one substitutions; and a VH-CDR3 sequence ARGRPYDHYFDY (SEQ ID NO:66) or ARGRPYDHYFDY (SEQ ID NO:66) with three, two, or one substitutions;
(vii) a VH-CDR1 sequence GSISSSSYYWG (SEQ ID NO:67) or GSISSSSYYWG (SEQ ID NO:67) with three, two, or one substitutions, a VH-CDR2 sequence SIYYSGSTYYNPSLKS (SEQ ID NO:68) or SIYYSGSTYYNPSLKS (SEQ ID NO:68) with three, two, or one substitutions; and a VH-CDR3 sequence ARDFYSSVYGMDV (SEQ ID NO:69) or ARDFYSSVYGMDV (SEQ ID NO:69) with three, two, or one substitutions;
(viii) a VH-CDR1 sequence YTFTSYGIS (SEQ ID NO:53) or YTFTSYGIS (SEQ ID NO:53) with three, two, or one substitutions, a VH-CDR2 sequence WISAYNGNTNYAQKLQG (SEQ ID NO:54) or WISAYNGNTNYAQKLQG (SEQ ID NO:54) with three, two, or one substitutions; and a VH-CDR3 sequence ARDGLGSSPWSAFDI (SEQ ID NO:70) or ARDGLGSSPWSAFDI (SEQ ID NO:70) with three, two, or one substitutions;
(ix) a VH-CDR1 sequence YTFTSYYMH (SEQ ID NO:71) or YTFTSYYMH (SEQ ID NO:71) with three, two, or one substitutions, a VH-CDR2 sequence VINPSGGSTSYAQKFQG (SEQ ID NO:72) or VINPSGGSTSYAQKFQG (SEQ ID NO:72) with three, two, or one substitutions; and a VH-CDR3 sequence ARLMSGSSGS (SEQ ID NO:73) or ARLMSGSSGS (SEQ ID NO:73) with three, two, or one substitutions;
(x) a VH-CDR1 sequence YTFTGYYMH (SEQ ID NO:74) or YTFTGYYMH (SEQ ID NO:74) with three, two, or one substitutions, a VH-CDR2 sequence SINPNSGGTNYAQKFQG (SEQ ID NO:75) or SINPNSGGTNYAQKFQG (SEQ ID NO:75) with three, two, or one substitutions; and a VH-CDR3 sequence ARDSSWKHDY (SEQ ID NO:76) or ARDSSWKHDY (SEQ ID NO:76) with three, two, or one substitutions;
(xi) a VH-CDR1 sequence YSISSGYYWG (SEQ ID NO:77) or YSISSGYYWG (SEQ ID NO:77) with three, two, or one substitutions, a VH-CDR2 sequence SIYHSGSTNYNPSLKS (SEQ ID NO:78) or SIYHSGSTNYNPSLKS (SEQ ID NO:78) with three, two, or one substitutions; and a VH-CDR3 sequence ARSPRWRSTYANWFNP (SEQ ID NO:79) or ARSPRWRSTYANWFNP (SEQ ID NO:79) with three, two, or one substitutions, or
(xii) a VH-CDR1 sequence YSISSGYYWA (SEQ ID NO:80) or YSISSGYYWA (SEQ ID NO:80) with three, two, or one substitutions, a VH-CDR2 sequence SIYHSGSTYYNPSLKS (SEQ ID NO:81) or SIYHSGSTYYNPSLKS (SEQ ID NO:81) with three, two, or one substitutions; and a VH-CDR3 sequence AREHSSSGQWNV (SEQ ID NO: 82) or AREHSSSGQWNV (SEQ ID NO: 82) with three, two, or one substitutions.
   In certain embodiments, the anti-GPIIb/IIIa antibody further includes:
(i) a variable light chain CDR-1 (VL-CDR1) sequence RSSQSLLHSNGYNYLD (SEQ ID NO:83) or RSSQSLLHSNGYNYLD (SEQ ID NO:83) with three, two, or one substitutions, a variable light chain CDR-2 (VL-CDR2) sequence LGSNRAS (SEQ ID NO:84) or LGSNRAS (SEQ ID NO:84) with three, two, or one substitutions; and a variable light chain CDR-3 (VL-CDR3) sequence MQALRLPRT (SEQ ID NO:85) or MQALRLPRT (SEQ ID NO:85) with three, two, or one substitutions;
(ii) a variable light chain CDR-1 (VL-CDR1) sequence RASQSVSSYLA (SEQ ID NO:86) or RASQSVSSYLA (SEQ ID NO:86) with three, two, or one substitutions, a variable light chain CDR-2 (VL-CDR2) sequence DASNRAT (SEQ ID NO:87) or DASNRAT (SEQ ID NO:87) with three, two, or one substitutions; and a variable light chain CDR-3 (VL-CDR3) sequence QQRSALPRT (SEQ ID NO:88) or QQRSALPRT (SEQ ID NO:88) with three, two, or one substitutions;
(iii) a variable light chain CDR-1 (VL-CDR1) sequence RASQSVSSYLA (SEQ ID NO:86) or RASQSVSSYLA (SEQ ID NO:86) with three, two, or one substitutions, a variable light chain CDR-2 (VL-CDR2) sequence DSSNRAT (SEQ ID NO:89) or DSSNRAT (SEQ ID NO:89) with three, two, or one substitutions; and a variable light chain CDR-3 (VL-CDR3) sequence QQRSHLPPT (SEQ ID NO:90) or QQRSHLPPT (SEQ ID NO:90) with three, two, or one substitutions;
(iv) a variable light chain CDR-1 (VL-CDR1) sequence RASQSVSSNLA (SEQ ID NO:91) or RASQSVSSNLA (SEQ ID NO:91) with three, two, or one substitutions, a variable light chain CDR-2 (VL-CDR2) sequence GASTRAT (SEQ ID NO:92) or GASTRAT (SEQ ID NO:92) with three, two, or one substitutions; and a variable light chain CDR-3 (VL-CDR3) sequence QQFNLYPYT (SEQ ID NO:93) or QQFNLYPYT (SEQ ID NO:93) with three, two, or one substitutions;
(v) a variable light chain CDR-1 (VL-CDR1) sequence RASQSVSSYLA (SEQ ID NO:86) or RASQSVSSYLA (SEQ ID NO:86) with three, two, or one substitutions, a variable light chain CDR-2 (VL-CDR2) sequence DASKRAT (SEQ ID NO:94) or DASKRAT (SEQ ID NO:94) with three, two, or one substitutions; and a variable light chain CDR-3 (VL-CDR3) sequence QQDSFLPFT (SEQ ID NO:95) or QQDSFLPFT (SEQ ID NO:95) with three, two, or one substitutions;
(vi) a variable light chain CDR-1 (VL-CDR1) sequence RASQSVSSYLA (SEQ ID NO:86) or RASQSVSSYLA (SEQ ID NO:86) with three, two, or one substitutions, a variable light chain CDR-2 (VL-CDR2) sequence DASNRAT (SEQ ID NO:87) or DASNRAT (SEQ ID NO:87) with three, two, or one substitutions; and a variable light chain CDR-3 (VL-CDR3) sequence QQAYNYPFT (SEQ ID NO:96) or QQAYNYPFT (SEQ ID NO:96) with three, two, or one substitutions;
(vii) a variable light chain CDR-1 (VL-CDR1) sequence RASQSISSFLN (SEQ ID NO:97) or RASQSISSFLN (SEQ ID NO:97) with three, two, or one substitutions, a variable light chain CDR-2 (VL-CDR2) sequence AASSLQS (SEQ ID NO:98) or AASSLQS (SEQ ID NO:98) with three, two, or one substitutions; and a variable light chain CDR-3 (VL-CDR3) sequence QQSYVHPLT (SEQ ID NO:99) or QQSYVHPLT (SEQ ID NO:99) with three, two, or one substitutions;
(viii) a variable light chain CDR-1 (VL-CDR1) sequence RSSQSLLHSNGYNYLD (SEQ ID NO:100) or RSSQSLLHSNGYNYLD (SEQ ID NO:100) with three, two, or one substitutions, a variable light chain CDR-2 (VL-CDR2) sequence LGSNRAS (SEQ ID NO:101) or LGSNRAS (SEQ ID NO:101) with three, two, or one substitutions; and a variable light chain CDR-3 (VL-CDR3) sequence MQARRSPLT (SEQ ID NO:102) or MQARRSPLT (SEQ ID NO:102) with three, two, or one substitutions;
(ix) a variable light chain CDR-1 (VL-CDR1) sequence RASQSVSSSYLA (SEQ ID NO:103) or RASQSVSSSYLA (SEQ ID NO:103) with three, two, or one substitutions, a variable light chain CDR-2 (VL-CDR2) sequence GASSRAT (SEQ ID NO:104) or GASSRAT (SEQ ID NO:104) with three, two, or one substitutions; and a variable light chain CDR-3 (VL-CDR3) sequence QQYGGFPLT (SEQ ID NO:105) or QQYGGFPLT (SEQ ID NO:105) with three, two, or one substitutions;
(x) a variable light chain CDR-1 (VL-CDR1) sequence RASQSVSSYLA (SEQ ID NO:86) or RASQSVSSYLA (SEQ ID NO:86) with three, two, or one substitutions, a variable light chain CDR-2 (VL-CDR2) sequence DASNRAT (SEQ ID NO:87) or DASNRAT (SEQ ID NO:87) with three, two, or one substitutions; and a variable light chain CDR-3 (VL-CDR3) sequence QQYSFYPLT (SEQ ID NO:106) or QQYSFYPLT (SEQ ID NO:106) with three, two, or one substitutions;
(xi) a variable light chain CDR-1 (VL-CDR1) sequence RASQGISSWLA (SEQ ID NO:107) or RASQGISSWLA (SEQ ID NO:107) with three, two, or one substitutions, a variable light chain CDR-2 (VL-CDR2) sequence GASSLQS (SEQ ID NO:108) or GASSLQS (SEQ ID NO:108) with three, two, or one substitutions; and a variable light chain CDR-3 (VL-CDR3) sequence QQAAPFPLT (SEQ ID NO:109) or QQAAPFPLT (SEQ ID NO:109) with three, two, or one substitutions; or
(xii) a variable light chain CDR-1 (VL-CDR1) sequence RASQSVSSYLA (SEQ ID NO:86) or RASQSVSSYLA (SEQ ID NO:86) with three, two, or one substitutions, a variable light chain CDR-2 (VL-CDR2) sequence DASNRAT (SEQ ID NO:87) or DASNRAT (SEQ ID NO:87) with three, two, or one substitutions; and a variable light chain CDR-3 (VL-CDR3) sequence QQRSFYFT (SEQ ID NO:110) or QQRSFYFT (SEQ ID NO:110) with three, two, or one substitutions.

In certain embodiments of all of the above aspects of the anti-GPIIb/IIIa antibody or antigen-binding fragment thereof, the VH CDR1 comprises or consists of an amino acid sequence set forth in SEQ ID NOs.:111 or 112; the VH CDR2 comprises or consists of an amino acid sequence set forth in SEQ ID NOs.: 113 or 114; and VH CDR3 comprises or consists of the amino acid sequence of the VH CDR3 of any one of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. In other embodiments of all of the above aspects of the anti-GPIIb/IIIa antibody or antigen-binding fragment thereof, the VH CDR1 comprises or consists of an amino acid sequence set forth in SEQ ID NOs.:115 or 116; the VH CDR2 comprises or consists of an amino acid sequence set forth in SEQ ID NO: 117; and VH CDR3 comprises or consists of the amino acid sequence of the VH CDR3 of any one of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. In yet other embodiments of all of the above aspects of the anti-GPIIb/IIIa antibody or antigen-binding fragment thereof, the VL CDR1 comprises or consists of an amino acid sequence set forth in SEQ ID NO:118; the VL CDR2 comprises or consists of an amino acid sequence set forth in SEQ ID NO:119; and VH CDR3 comprises or consists of the amino acid sequence set forth in SEQ ID NO:120. In a specific embodiment, the anti-GPIIb/IIIa antibody or antigen-binding fragment thereof contains a VH-CDR1, VH-CDR2, VH-CDR3, VL-CDR1, VL-CDR2, and VL-CDR3, wherein
(i) the VH-CDR1 sequence comprises YTFTSYGIS (SEQ ID NO:53), the VH-CDR2 sequence comprises WISAYNGNTNYAQKLQG (SEQ ID NO:54), the VH-CDR3 sequence comprises ARDLEYYDSSGYAYGYFDL (SEQ ID NO:55), the VL-CDR1 sequence comprises RSSQSLLHSNGYNYLD (SEQ ID NO:83), the VL-CDR2 sequence comprises LGSNRAS (SEQ ID NO:84), and the VL-CDR3 sequence comprises MQALRLPRT (SEQ ID NO:85);
(ii) the VH-CDR1 sequence comprises GTFSSYAIS (SEQ ID NO:56), the VH-CDR2 sequence comprises GIIPIFGTANYAQKFQG (SEQ ID NO:57), the VH-CDR3 sequence comprises ARDTGYYGASLYFDY (SEQ ID NO:58), the VL-CDR1 sequence comprises RASQSVSSYLA (SEQ ID NO:86), the VL-CDR2 sequence comprises DASNRAT (SEQ ID NO:87), and the VL-CDR3 sequence comprises QQRSALPRT (SEQ ID NO:88);
(iii) the VH-CDR1 sequence comprises GTFSSYAIS (SEQ ID NO:56), the VH-CDR2 sequence comprises GIIPIFGTANYAQKFQG (SEQ ID NO:57), the VH-CDR3 sequence comprises ARGPPSAYGDYVWDI (SEQ ID NO:59), the VL-CDR1 sequence comprises RASQSVSSYLA (SEQ ID NO:86), the VL-CDR2 sequence comprises DSSNRAT (SEQ ID NO:89), and the VL-CDR3 sequence comprises QQRSHLPPT (SEQ ID NO:90);
(iv) the VH-CDR1 sequence comprises FTFSDHHMD (SEQ ID NO:60), the VH-CDR2 sequence comprises RTRNKANSYTTEYAASVKG (SEQ ID NO:61), the VH-CDR3 sequence comprises ARGPPYYADLGMGV (SEQ ID NO:62), the VL-CDR1 sequence comprises RASQSVSSNLA (SEQ ID NO:91), the VL-CDR2 sequence comprises GASTRAT (SEQ ID NO:92), and the VL-CDR3 sequence comprises QQFNLYPYT (SEQ ID NO:93);
(v) the VH-CDR1 sequence comprises YTFTSYSMH (SEQ ID NO:63), the VH-CDR2 sequence comprises IINPSGGSTSYAQKFQG (SEQ ID NO:64), the VH-CDR3 sequence comprises ARSYDIGYFDL (SEQ ID NO:65), the VL-CDR1 sequence comprises RASQSVSSYLA (SEQ ID NO:86), the VL-CDR2 sequence comprises DASKRAT (SEQ ID NO:94), and the VL-CDR3 sequence comprises QQDSFLPFT (SEQ ID NO:95);
(vi) the VH-CDR1 sequence comprises YTFTSYGIS (SEQ ID NO:53), the VH-CDR2 sequence comprises WISAYNGNTNYAQKLQG (SEQ ID NO:54), the VH-CDR3 sequence comprises ARGRPYDHYFDY (SEQ ID NO:66), the VL-CDR1 sequence comprises RASQSVSSYLA (SEQ ID NO:86), the VL-CDR2 sequence comprises DASNRAT (SEQ ID NO:87), and the VL-CDR3 sequence comprises QQAYNYPFT (SEQ ID NO:96);
(vii) the VH-CDR1 sequence comprises GSISSSSYYWG (SEQ ID NO:67), the VH-CDR2 sequence comprises SIYYSGSTYYNPSLKS (SEQ ID NO:68), the VH-CDR3 sequence comprises ARDFYSSVYGMDV (SEQ ID NO:69), the VL-CDR1 sequence comprises RASQSISSFLN (SEQ ID NO:97), the VL-CDR2 sequence comprises AASSLQS (SEQ ID NO:98), and the VL-CDR3 sequence comprises QQSYVHPLT (SEQ ID NO:99);
(viii) the VH-CDR1 sequence comprises YTFTSYGIS (SEQ ID NO:53), the VH-CDR2 sequence comprises WISAYNGNTNYAQKLQG (SEQ ID NO:54), the VH-CDR3 sequence comprises ARDGLGSSPWSAFDI (SEQ ID NO:70), the VL-CDR1 sequence comprises RSSQSLLHSNGYNYLD (SEQ ID NO:100), the VL-CDR2 sequence comprises LGSNRAS (SEQ ID NO:101), and the VL-CDR3 sequence comprises MQARRSPLT (SEQ ID NO:102);
(ix) the VH-CDR1 sequence comprises YTFTSYYMH (SEQ ID NO:71), the VH-CDR2 sequence comprises VINPSGGSTSYAQKFQG (SEQ ID NO:72), the VH-CDR3 sequence comprises ARLMSGSSGS (SEQ ID NO:73), the VL-CDR1 sequence comprises RASQSVSSSYLA (SEQ ID NO:103), the VL-CDR2 sequence comprises GASSRAT (SEQ ID NO:104), and the VL-CDR3 sequence comprises QQYGGFPLT (SEQ ID NO:105);
(x) the VH-CDR1 sequence comprises YTFTGYYMH (SEQ ID NO:74), the VH-CDR2 sequence comprises SINPNSGGTNYAQKFQG (SEQ ID NO:75), the VH-CDR3 sequence comprises ARDSSWKHDY (SEQ ID NO:76), the VL-CDR1 sequence comprises RASQSVSSYLA (SEQ ID NO:86), the VL-CDR2 sequence comprises DASNRAT (SEQ ID NO:87), and the VL-CDR3 sequence comprises QQYSFYPLT (SEQ ID NO:106);
(xi) the VH-CDR1 sequence comprises YSISSGYYWG (SEQ ID NO:77), the VH-CDR2 sequence comprises SIYHSGSTNYNPSLKS (SEQ ID NO:78), the VH-CDR3 sequence comprises ARSPRWRSTYANWFNP (SEQ ID NO:79), the VL-CDR1 sequence comprises RASQGISSWLA (SEQ ID NO:107), the VL-CDR2 sequence comprises GASSLQS (SEQ ID NO:108), and the VL-CDR3 sequence comprises QQAAPFPLT (SEQ ID NO:109); or
(xii) the VH-CDR1 sequence comprises YSISSGYYWA (SEQ ID NO:80), the VH-CDR2 sequence comprises SIYHSGSTYYNPSLKS (SEQ ID NO:81), the VH-CDR3 sequence comprises AREHSSSGQWNV (SEQ ID NO: 82), the VL-CDR1 sequence comprises RASQSVSSYLA (SEQ ID NO:86), the VL-CDR2 sequence comprises DASNRAT (SEQ ID NO:87), and the VL-CDR3 sequence comprises QQRSFYFT (SEQ ID NO:110).

In another aspect, the disclosure provides an antibody or antigen-binding fragment thereof that specifically binds to GPIIb/IIIa, comprising a VH comprising an amino acid sequence that is at least 80%, at least 85%, at least 90%, at least 95%, at least 97%, or 100% identical to any one of SEQ ID NOS: 5, 9, 13, 17, 21, 25, 29, 33, 37, 41, 45, or 49. In some embodiments, the antibody or antigen-binding fragment thereof further includes a VL comprising an amino acid sequence that is at least 80%, at least 85%, at least 90%, at least 95%, at least 97%, or 100% identical to any one of SEQ ID NOS: 7, 11, 15, 19, 23, 27, 31, 35, 39, 43, 47, or 51. In certain embodiments of this aspect, the VH CDR1 comprises or consists of an amino acid sequence set forth in SEQ ID NOs.:111 or 112; the VH CDR2 comprises or consists of an amino acid sequence set forth in SEQ ID NOs.: 113 or 114; and VH CDR3 comprises or consists of the amino acid sequence of the VH CDR3 of any one of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. In certain embodiments of this aspect, the VH CDR1 comprises or consists of an amino acid sequence set forth in SEQ ID NOs.:115 or 116; the VH CDR2 comprises or consists of an amino acid sequence set forth in SEQ ID NO:117; and VH CDR3 comprises or consists of the amino acid sequence of the VH CDR3 of any one of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. In certain embodiments of this aspect, the VL CDR1 comprises or consists of an amino acid sequence set forth in SEQ ID NO:118; the VL CDR2 comprises or consists of an amino acid sequence set forth in SEQ ID NO:119; and VH CDR3 comprises or consists of the amino acid sequence set forth in SEQ ID NO:120.

In yet another aspect, the disclosure relates to an antibody or antigen-binding fragment thereof that specifically binds to GPIIb/IIIa, comprising
(i) a VH comprising an amino acid sequence that is at least 80%, at least 85%, at least 90%, at least 95%, at least 97%, or 100% identical to SEQ ID NO:5 and a VL comprising an amino acid sequence that is at least 80%, at least 85%, at least 90%, at least 95%, at least 97%, or 100% identical to of SEQ ID NO:7;
(ii) a VH comprising an amino acid sequence that is at least 80%, at least 85%, at least 90%, at least 95%, at least 97%, or 100% identical to SEQ ID NO:9 and a VL comprising an amino acid sequence that is at least 80%, at least 85%, at least 90%, at least 95%, at least 97%, or 100% identical to SEQ ID NO:11;
(iii) a VH comprising an amino acid sequence that is at least 80%, at least 85%, at least 90%, at least 95%, at least 97%, or 100% identical to SEQ ID NO:13 and a VL comprising an amino acid sequence that is at least 80%, at least 85%, at least 90%, at least 95%, at least 97%, or 100% identical to SEQ ID NO:15;
(iv) a VH comprising an amino acid sequence that is at least 80%, at least 85%, at least 90%, at least 95%, at least 97%, or 100% identical to SEQ ID NO:17 and a VL comprising an amino acid sequence that is at least 80%, at least 85%, at least 90%, at least 95%, at least 97%, or 100% identical to SEQ ID NO:19;
(v) a VH comprising an amino acid sequence that is at least 80%, at least 85%, at least 90%, at least 95%, at least 97%, or 100% identical to SEQ ID NO:21 and a VL comprising an amino acid sequence that is at least 80%, at least 85%, at least 90%, at least 95%, at least 97%, or 100% identical to SEQ ID NO:23;
(vi) a VH comprising an amino acid sequence that is at least 80%, at least 85%, at least 90%, at least 95%, at least 97%, or 100% identical to SEQ ID NO:25 and a VL comprising an amino acid sequence that is at least 80%, at least 85%, at least 90%, at least 95%, at least 97%, or 100% identical to SEQ ID NO:27;
(vii) a VH comprising an amino acid sequence that is at least 80%, at least 85%, at least 90%, at least 95%, at least 97%, or 100% identical to SEQ ID NO:29 and a VL comprising an amino acid sequence that is at least 80%, at least 85%, at least 90%, at least 95%, at least 97%, or 100% identical to SEQ ID NO:31;
(viii) a VH comprising an amino acid sequence that is at least 80%, at least 85%, at least 90%, at least 95%, at least 97%, or 100% identical to SEQ ID NO:33 and a VL comprising an amino acid sequence that is at least 80%, at least 85%, at least 90%, at least 95%, at least 97%, or 100% identical to SEQ ID NO:35;
(ix) a VH comprising an amino acid sequence that is at least 80%, at least 85%, at least 90%, at least 95%, at least 97%, or 100% identical to SEQ ID NO:37 and a VL comprising an amino acid sequence that is at least 80%, at least 85%, at least 90%, at least 95%, at least 97%, or 100% identical to SEQ ID NO:39;
(x) a VH comprising an amino acid sequence that is at least 80%, at least 85%, at least 90%, at least 95%, at least 97%, or 100% identical to SEQ ID NO:41 and a VL comprising an amino acid sequence that is at least 80%, at least 85%, at least 90%, at least 95%, at least 97%, or 100% identical to SEQ ID NOS:43;
(xi) a VH comprising an amino acid sequence that is at least 80%, at least 85%, at least 90%, at least 95%, at least 97%, or 100% identical to SEQ ID NO:45 and a VL comprising an amino acid sequence that is at least 80%, at least 85%, at least 90%, at least 95%, at least 97%, or 100% identical to SEQ ID NO:47; or (xii) a VH comprising an amino acid sequence that is at least 80%, at least 85%, at least 90%, at least 95%, at least 97%, or 100% identical to SEQ ID NO:49 and a VL comprising an amino acid sequence that is at least 80%, at least 85%, at least 90%, at least 95%, at least 97%, or 100% identical to SEQ ID NO:51.
   In certain embodiments, the antibody or antigen-binding fragment thereof comprises a VH and a VL comprising the amino acid sequence set forth in: SEQ ID NOs. 5 and 7; SEQ ID NOs. 9 and 11; SEQ ID NOs. 13 and 15; SEQ ID NOs. 17 and 19; SEQ ID NOs. 21 and 23; SEQ ID NOs. 25 and 27; SEQ ID NOs. 29 and 31; SEQ ID NOs. 33 and 35; SEQ ID NOs. 37 and 39; SEQ ID NOs. 41 and 43; SEQ ID NOs. 45 and 47; or SEQ ID NOs. 49 and 51. In some embodiments, the antibody or antigen-binding fragment thereof comprises a VH-CDR1, VH-CDR2, and VH-CDR3, wherein
(i) the VH-CDR1 sequence comprises YTFTSYGIS (SEQ ID NO:53), the VH-CDR2 sequence comprises WISAYNGNTNYAQKLQG (SEQ ID NO:54), and the VH-CDR3 sequence comprises ARDLEYYDSSGYAYGYFDL (SEQ ID NO:55);
(ii) the VH-CDR1 sequence comprises GTFSSYAIS (SEQ ID NO:56), the VH-CDR2 sequence comprises GIIPIFGTANYAQKFQG (SEQ ID NO:57), and the VH-CDR3 sequence comprises ARDTGYYGASLYFDY (SEQ ID NO:58);
(iii) the VH-CDR1 sequence comprises GTFSSYAIS (SEQ ID NO:56), the VH-CDR2 sequence comprises GIIPIFGTANYAQKFQG (SEQ ID NO:57), and the VH-CDR3 sequence comprises ARGPPSAYGDYVWDI (SEQ ID NO:59);
(iv) the VH-CDR1 sequence comprises FTFSDHHMD (SEQ ID NO:60), the VH-CDR2 sequence comprises RTRNKANSYTTEYAASVKG (SEQ ID NO:61), and the VH-CDR3 sequence comprises ARGPPYYADLGMGV (SEQ ID NO:62);
(v) the VH-CDR1 sequence comprises YTFTSYSMH (SEQ ID NO:63), the VH-CDR2 sequence comprises IINPSGGSTSYAQKFQG (SEQ ID NO:64), and the VH-CDR3 sequence comprises ARSYDIGYFDL (SEQ ID NO:65);
(vi) the VH-CDR1 sequence comprises YTFTSYGIS (SEQ ID NO:53), the VH-CDR2 sequence comprises WISAYNGNTNYAQKLQG (SEQ ID NO:54), and the VH-CDR3 sequence comprises ARGRPYDHYFDY (SEQ ID NO:66);
(vii) the VH-CDR1 sequence comprises GSISSSSYYWG (SEQ ID NO:67), the VH-CDR2 sequence comprises SIYYSGSTYYNPSLKS (SEQ ID NO:68), and the VH-CDR3 sequence comprises ARDFYSSVYGMDV (SEQ ID NO:69);
(viii) the VH-CDR1 sequence comprises YTFTSYGIS (SEQ ID NO:53), the VH-CDR2 sequence comprises WISAYNGNTNYAQKLQG (SEQ ID NO:54), and the VH-CDR3 sequence comprises ARDGLGSSPWSAFDI (SEQ ID NO:70);
(ix) the VH-CDR1 sequence comprises YTFTSYYMH (SEQ ID NO:71), the VH-CDR2 sequence comprises VINPSGGSTSYAQKFQG (SEQ ID NO:72), and the VH-CDR3 sequence comprises ARLMSGSSGS (SEQ ID NO:73);
(x) the VH-CDR1 sequence comprises YTFTGYYMH (SEQ ID NO:74), the VH-CDR2 sequence comprises SINPNSGGTNYAQKFQG (SEQ ID NO:75), and the VH-CDR3 sequence comprises ARDSSWKHDY (SEQ ID NO:76);
(xi) the VH-CDR1 sequence comprises YSISSGYYWG (SEQ ID NO:77), the VH-CDR2 sequence comprises SIYHSGSTNYNPSLKS (SEQ ID NO:78), and the VH-CDR3 sequence comprises ARSPRWRSTYANWFNP (SEQ ID NO:79); or
(xii) the VH-CDR1 sequence comprises YSISSGYYWA (SEQ ID NO:80), the VH-CDR2 sequence comprises SIYHSGSTYYNPSLKS (SEQ ID NO:81), and the VH-CDR3 sequence comprises AREHSSSGQWNV (SEQ ID NO: 82).
   In some embodiments, the anti-GPIIb/IIIa antibody or antigen-binding fragment thereof comprises a VL-CDR1, VL-CDR2, and VL-CDR3, wherein
(i) the VL-CDR1 sequence comprises RSSQSLLHSNGYNYLD (SEQ ID NO:83), the VL-CDR2 sequence comprises LGSNRAS (SEQ ID NO:84), and the VL-CDR3 sequence comprises MQALRLPRT (SEQ ID NO:85);
(ii) the VL-CDR1 sequence comprises RASQSVSSYLA (SEQ ID NO:86), the VL-CDR2 sequence comprises DASNRAT (SEQ ID NO:87), and the VL-CDR3 sequence comprises QQRSALPRT (SEQ ID NO:88);
(iii) the VL-CDR1 sequence comprises RASQSVSSYLA (SEQ ID NO:86), the VL-CDR2 sequence comprises DSSNRAT (SEQ ID NO:89), and the VL-CDR3 sequence comprises QQRSHLPPT (SEQ ID NO:90);
(iv) the VL-CDR1 sequence comprises RASQSVSSNLA (SEQ ID NO:91), the VL-CDR2 sequence comprises GASTRAT (SEQ ID NO:92), and the VL-CDR3 sequence comprises QQFNLYPYT (SEQ ID NO:93);
(v) the VL-CDR1 sequence comprises RASQSVSSYLA (SEQ ID NO:86), the VL-CDR2 sequence comprises DASKRAT (SEQ ID NO:94), and the VL-CDR3 sequence comprises QQDSFLPFT (SEQ ID NO:95);
(vi) the VL-CDR1 sequence comprises RASQSVSSYLA (SEQ ID NO:86), the VL-CDR2 sequence comprises DASNRAT (SEQ ID NO:87), and the VL-CDR3 sequence comprises QQAYNYPFT (SEQ ID NO:96)
(vii) the VL-CDR1 sequence comprises RASQSISSFLN (SEQ ID NO:97), the VL-CDR2 sequence comprises AASSLQS (SEQ ID NO:98), and the VL-CDR3 sequence comprises QQSYVHPLT (SEQ ID NO:99);
(viii) the VL-CDR1 sequence comprises RSSQSLLHSNGYNYLD (SEQ ID NO:100), the VL-CDR2 sequence comprises LGSNRAS (SEQ ID NO:101), and the VL-CDR3 sequence comprises MQARRSPLT (SEQ ID NO:102);
(ix) the VL-CDR1 sequence comprises RASQSVSSSYLA (SEQ ID NO:103), the VL-CDR2 sequence comprises GASSRAT (SEQ ID NO:104), and the VL-CDR3 sequence comprises QQYGGFPLT (SEQ ID NO:105);
(x) the VL-CDR1 sequence comprises RASQSVSSYLA (SEQ ID NO:86), the VL-CDR2 sequence comprises DASNRAT (SEQ ID NO:87), and the VL-CDR3 sequence comprises QQYSFYPLT (SEQ ID NO:106);
(xi) the VL-CDR1 sequence comprises RASQGISSWLA (SEQ ID NO:107), the VL-CDR2 sequence comprises GASSLQS (SEQ ID NO:108), and the VL-CDR3 sequence comprises QQAAPFPLT (SEQ ID NO:109); or
(xii) the VL-CDR1 sequence comprises RASQSVSSYLA (SEQ ID NO:86), the VL-CDR2 sequence comprises DASNRAT (SEQ ID NO:87), and the VL-CDR3 sequence comprises QQRSFYFT (SEQ ID NO:110).

In certain embodiments of all of the above aspects, the antibody or antigen binding fragment thereof is a whole antibody, a Fab, a Fab′, a F(ab)2, an scFv, an sc(Fv)2, or a diabody. In a specific embodiment, the antibody or antigen binding fragment thereof is a Fab. In certain embodiments of all of the above aspects, the antibody or antigen binding fragment thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM. In certain embodiments of all of the above aspects, the antibody or antigen binding fragment thereof binds to GPIIb/IIIa (SEQ ID NOS.:1, 3) but not to alpha v beta 3 (SEQ ID NOs.:245, 3). In other embodiments of all of the above aspects, the antibody or antigen binding fragment thereof binds to both GPIIb/IIIa (SEQ ID NOS.:1, 3) and alpha v beta 3 (SEQ ID NOs.:245, 3).

In a different aspect, the disclosure features a chimeric molecule comprising an antibody or antigen-binding fragment thereof disclosed herein and a heterologous moiety. In some embodiments, the heterologous moiety comprises a clotting factor. In some embodiments, the clotting factor is FVII, FIX, or FX. In other embodiments, the clotting factor is FVII zymogen, activatable FVII, activated FVII (FVIIa), FX zymogen, activatable FX, or activated FX (FXa). In certain embodiments, the clotting factor comprises a single polypeptide chain or two polypeptide chains. In certain embodiments, the chimeric molecule further comprises a linker. In some embodiments, the linker is a peptide linker. The peptide linker can comprises at least two, at least three, at least four, at least five, at least 10, at least 15, at least 20, at least 30, at least 40, at least 50, at least 60, at least 70, at least 80, at least 90, or at least 100 amino acids. In a particular embodiment, the peptide linker comprises a peptide having the formula [(Gly)ₓ-Ser_{y}]_{z} where x is from 1 to 4, y is 0 or 1, and z is from 1 to 50 (e.g., 1, 2, 3, 4, 5, 6, 7, 8, 9, 10, 12, 15, 20, 25, 30, 35, 40, 45, 50) (SEQ ID NO: 156). In some embodiments, the chimeric molecule comprises a second heterologous moiety. In a particular embodiment, the second heterologous moiety comprises a half-life extending moiety. In some instances, the half-life extending moiety is a low-complexity polypeptide. In other embodiments, the half-life extending moiety is albumin, albumin binding polypeptide or fatty acid, an Fc region, transferrin, PAS, the C-terminal peptide (CTP) of the β subunit of human chorionic gonadotropin, polyethylene glycol (PEG), hydroxyethyl starch (HES), albumin-binding small molecules, vWF, or a clearance receptor or a fragment thereof which blocks binding of the chimeric molecule to a clearance receptor.

In one aspect, the disclosure features a chimeric molecule a Class I or Class II antibody or antigen-binding fragment thereof disclosed herein, a Factor VII molecule (e.g., recombinant Factor VII (e.g., rFVIIa)) including a heavy chain and a light chain, and a half-life extending moiety. In some embodiments, the antibody or antigen-binding fragment thereof is an Fab. In other embodiments, the antibody or antigen-binding fragment thereof is an scFv. In certain embodiments, the heavy chain of the Factor VII is linked to the half-life extending moiety and the half-life extending moiety is linked to the antibody or antigen-binding fragment thereof. In some embodiments, the Factor VII is linked to the half-life extending moiety via a first peptide linker and the half-life extending moiety is linked to the antibody or antigen-binding fragment thereof via a second peptide linker. In a particular embodiment, the heavy chain of the recombinant Factor VIIa is linked to the half-life extending moiety via a first peptide linker and the half-life extending moiety is linked to the light chain of the antibody or antigen-binding fragment thereof via a second peptide linker. In certain embodiments, the light chain of the antibody in the chimeric molecule (e.g., a Fab light chain) is associated with its counterpart heavy chain (e.g., a Fab heavy chain). The light chain of the Factor VII is associated with the heavy chain of the Factor VII in these chimeric molecules. In certain embodiments, the first and second peptide linkers comprise a peptide having the formula [(Gly)ₓ-Ser_{y}]_{z} where x is from 1 to 4, y is 0 or 1, and z is from 1 to 6 (SEQ ID NO: 249).

In one aspect, the application provides a chimeric polypeptide comprising an amino acid sequence that is at least 80%, at least 85%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO:246. The heavy chain component of the Fab in the chimeric polypeptide (e.g., the polypeptide having the sequence of SEQ ID NO:246) can associate with the light chain component of the Fab set forth in SEQ ID NO:247. Thus, this disclosure features a composition comprising a first polypeptide comprising an amino acid sequence that is at least 80%, at least 85%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO:246 and a second polypeptide comprising an amino acid sequence that is at least 80%, at least 85%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence set forth in SEQ ID NO:247. In a specific embodiment, this disclosure features a first polypeptide comprising the amino acid sequence set forth in SEQ ID NO:246 and a second polypeptide comprising the amino acid sequence set forth in SEQ ID NO:247. In addition, the above-mentioned chimeric polypeptide can be modified so as to replace the VH of BIIB-4-309 with the VH of any one of BIIB-4-147, BIIB-4-156, BIIB-4-174, BIIB-4-175, BIIB-4-204, BIIB-4-209, BIIB-4-224, BIIB-4-311, BIIB-4-317, BIIB-4-318, or BIIB-4-319. Thus, this application features a chimeric polypeptide comprising SEQ ID NO:246 except that the VH of the heavy chain component of the Fab is a VH from any one of BIIB-4-147, BIIB-4-156, BIIB-4-174, BIIB-4-175, BIIB-4-204, BIIB-4-209, BIIB-4-224, BIIB-4-311, BIIB-4-317, BIIB-4-318, or BIIB-4-319. In specific embodiments, the VH of BIIB-4-309 in the chimeric polypeptide is replaced with the VH of any one of BIIB-4-147, BIIB-4-174, BIIB-4-175, BIIB-4-224, BIIB-4-311, or BIIB-4-318. The above-mentioned chimeric polypeptides can be modified so as to remove one or both linkers (i.e., SEQ ID NOs: 197 and 172), or replace one or both the linkers with other linkers (e.g., those described herein). If the VH of the chimeric polypeptide is replaced, then the VL of the counterpart light chain component of the Fab (SEQ ID NO:247) is replaced with a VL that pairs with the VH in the chimeric polypeptide. In certain embodiments, the chimeric polypeptide and the light chain component of the Fab shows specificity for the active conformation of GPIIb/IIIa compared to the inactive conformation of GPIIb/IIIa.

In another aspect, the disclosure features a pharmaceutical composition comprising an antibody or antigen-binding fragment thereof or a chimeric molecule disclosed herein, and a pharmaceutically acceptable carrier.

In a different aspect, the disclosure relates to a method of reducing the frequency or degree of a bleeding episode in a subject in need thereof, comprising administering to the subject an effective amount of an antibody or antigen-binding fragment thereof (a Class I or Class II antibody or antigen-binding fragment thereof), a chimeric molecule (comprising a Class I or Class II antibody or antigen-binding fragment thereof), or a pharmaceutical composition disclosed herein. In some embodiments, the subject has developed or has a tendency to develop an inhibitor against Factor VIII (“FVIII”), Factor IX (“FIX”), or both. In certain embodiments, the inhibitor against FVIII or FIX is a neutralizing antibody against FVIII, FIX, or both. In some embodiments, the bleeding episode is the result of hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, or any combinations thereof. In some embodiments, the subject is human.

In another aspect, the Class I or Class II antibodies or antigen-binding fragments thereof, and chimeric molecules based on Class I or Class II antibodies described herein can be used to treat, prevent, or ameliorate bleeding episodes and in the pen-operative management of patients with congenital hemophilia A and B with inhibitors, acquired hemophilia, congenital FVII deficiency, and Glanzmann's thrombasthenia. In certain aspects embodiments, these agents can be used to treat, prevent, or ameliorate hemophilia A and B, or trauma in a subject in need thereof.

In another aspect, the disclosure provides a method of treating a blood coagulation disorder in a subject in need thereof, comprising administering to the subject an effective amount of an antibody or antigen-binding fragment thereof (a Class I or Class II antibody or antigen-binding fragment thereof), a chimeric molecule (comprising a Class I or Class II antibody or antigen-binding fragment thereof), or a pharmaceutical composition disclosed herein. In certain embodiments, the blood coagulation disorder is hemophilia A or hemophilia B. In some embodiments, the subject is human.

In another aspect, the disclosure provides a method of reducing, inhibiting, or preventing platelet aggregation and/or platelet thrombus formation in a subject in need thereof. The method comprises administering to the subject an effective amount of an antibody or antigen-binding fragment thereof (a Class III antibody or antigen-binding fragment thereof), a chimeric molecule (comprising a Class III antibody or antigen-binding fragment thereof), or a pharmaceutical composition disclosed herein. In certain embodiments, the subject has or is at risk of developing intracoronary atherothrombosis. In some embodiments, the subject is human.

In yet another aspect, the disclosure provides a method of treating a subject having or at risk of developing unstable angina. The method involves administering to the subject an effective amount of an antibody or antigen-binding fragment thereof (a Class III antibody or antigen-binding fragment thereof), a chimeric molecule (comprising a Class III antibody or antigen-binding fragment thereof), or a pharmaceutical composition disclosed herein. In some embodiments, the subject is human.

In a further aspect, the disclosure provides a method of treating a human subject undergoing high-risk percutaneous transluminal coronary angioplasty (PTCA). The method involves administering to the subject an effective amount of an antibody or antigen-binding fragment thereof (a Class III antibody or antigen-binding fragment thereof), a chimeric molecule (comprising a Class III antibody or antigen-binding fragment thereof), or a pharmaceutical composition disclosed herein.

In a different aspect, the disclosure features a method of detecting platelets. The method involves contacting a sample (e.g., human blood preparation) with an antibody or antigen-binding fragment thereof disclosed herein and detecting cells in the sample to which the antibody or antigen-binding fragment thereof binds.

In one aspect, the disclosure provides a method for enriching platelets. The method involves contacting a sample (e.g., human blood preparation) with an antibody or antigen-binding fragment thereof disclosed herein and enriching cells to which the antibody or antigen-binding fragment thereof are bound as compared to those cells in the sample that are not bound by the antibody or antigen-binding fragment thereof.

In another aspect, the disclosure features a method for enriching for activated platelets in a sample. The method comprises contacting a sample with a Class I antibody or antigen-binding fragment thereof disclosed herein and enriching cells to which the Class I antibody or antigen-binding fragment thereof are bound as compared to those cells in the sample that are not bound by the antibody or antigen-binding fragment thereof.

In a different aspect, the disclosure relates to the use of Class III antibodies or antigen-binding fragments thereof as diagnostic tools for evaluating fibrinogen blocking. The method involves, e.g., contacting a sample with a Class III antibody or antigen-binding fragment thereof disclosed herein in complex with a detectable label and identifying cells to which the Class III antibody or antigen-binding fragment thereof are bound as a sample that is capable of binding to fibrinogen when compared to those cells in the sample that are not bound by the antibody or antigen-binding fragment thereof.

In another aspect, the disclosure features an isolated nucleic acid comprising a nucleotide sequence that is at least 80% at least 85%, at least 86%, at least 87%, at least 88%, at least 89% at least 90%, at least 95%, at least 96%, at least 97%, at least 98%, or 100% identical to a nucleotide sequence selected from the group consisting of SEQ ID NOs: 6, 8, 10, 12, 14, 16, 18, 20, 22, 24, 26, 28, 30, 32, 34, 36, 38, 40, 42, 44, 46, 48, 50, and 52.

In another aspect, the disclosure features an isolated nucleic acid comprising a nucleotide sequence that encodes a polypeptide comprising an amino acid sequence that is at least 75%, at least 80% at least 85%, at least 86%, at least 87%, at least 88%, at least 89% at least 90%, at least 95%, at least 96%, at least 97%, at least 98%, or 100% identical to an amino acid sequence selected from the group consisting of SEQ ID NOs: 5, 7, 9, 11, 13, 15, 17, 19, 21, 23, 25, 27, 29, 31, 33, 35, 37, 39, 41, 43, 45, 47, 49, and 51.

In one aspect, the disclosure relates to an isolated polypeptide encoded by the nucleic acids of this disclosure. In another aspect, the disclosure relates to a recombinant vector comprising the nucleic acids of this disclosure. In yet another aspect, the disclosure provides a host cell comprising the recombinant vectors of this disclosure.

In a different aspect, the disclosure relates to a method of preparing an antibody or antigen-binding fragment thereof. The method comprises culturing a host cell comprising recombinant vectors comprising the nucleic acid sequences set forth in SEQ ID NOs: 6 and 8; SEQ ID NOs: 10 and 12; SEQ ID NOs: 14 and 16; SEQ ID NOs: 18 and 20; SEQ ID NOs: 22 and 24; SEQ ID NOs: 26 and 32; SEQ ID NOs: 34 and 36; SEQ ID NOs: 38 and 40; SEQ ID NOs: 42 and 44; SEQ ID NOs: 46 and 48; or SEQ ID NOs: 50 and 52, under conditions appropriate for expression and production of the antibody or antigen-binding fragment thereof. In some embodiments, the method further involves isolating the antibody or antigen-binding fragment thereof. In specific embodiments, the host cell is a 293 cell, a CHO cell or a DG44i cell.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A is a schematic representation of the inactive/bent conformation of the GPIIb/IIIa integrin compared with the active/extended conformation.

FIG. 1B depicts the protein constructs utilized in the selection and screening of antibodies to glycoprotein IIb/IIIa (GPIIb/IIIa). The top schematic shows the ectodomain of the am integrin (GPIIb) chain with or without a mutation at L959C. The bottom schematic shows the ectodomain of the β₃ integrin (GPIIIa) chain with or without a mutation at P688C. These mutations are reported to trap GPIIb/IIIa in an inactive conformation (Zhu et al., Mol Cell, 32(6):849-61 (2008)).

FIG. 1C depicts the strategy of antibody selection and screening campaigns to identify antibodies that are capable of recognizing the active/extended conformation of GPIIb/IIIa preferentially over the inactive/bent conformation.

FIG. 2 depicts the selection and screening strategy utilized in identifying the desired antibodies.

FIG. 3 is a CLUSTAL format multiple sequence alignment by MAFFT (v7.205) of the VH segments of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. Degree of amino acid conservation is indicating above the alignment (“*”=identical; “:”=strongly conserved; “.”=poorly conserved), as well as the bars below the alignment. The VH CDRs are underlined. The sequence before VH-CDR1 is framework region (FR) 1; the sequence after VH-CDR1 and before VH-CDR2 is FR2; the sequence after VH-CDR2 and before VH-CDR3 is FR3; and the sequence after VH-CDR3 is FR4.

FIG. 4 is a CLUSTAL format multiple sequence alignment by MAFFT (v7.205) of the VL segments of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. Degree of amino acid conservation is indicating above the alignment (“*”=identical; “:”=strongly conserved; “.”=poorly conserved), as well as the bars below the alignment. The VL CDRs are underlined. The sequence before VL-CDR1 is framework region (FR) 1; the sequence after VL-CDR1 and before VL-CDR2 is FR2; the sequence after VL-CDR2 and before VL-CDR3 is FR3; and the sequence after VL-CDR3 is FR4.

FIG. 5 is a table listing the amino acid sequences of the CDRs found in the VH and VL domains of the twelve antibodies described herein as well as their germline families. Sequences are assigned below (from left to right):

Antibody BIIB_4_147 discloses SEQ ID NOS 53-55 and 83-85;

Antibody BIIB_4_156 discloses SEQ ID NOS 56-58 and 86-88;

Antibody BIIB_4_174 discloses SEQ ID NOS 56-57, 59, 86 and 89-90;

Antibody BIIB_4_175 discloses SEQ ID NOS 60-62 and 91-93;

Antibody BIIB_4_204 discloses SEQ ID NOS 63-65, 86 and 94-95;

Antibody BIIB_4_209 discloses SEQ ID NOS 53-54, 66, 86-87 and 96;

Antibody BIIB_4_224 discloses SEQ ID NOS 67-69 and 97-99;

Antibody BIIB_4_309 discloses SEQ ID NOS 53-54, 70, 83-84 and 102;

Antibody BIIB_4_311 discloses SEQ ID NOS 71-73 and 103-105;

Antibody BIIB_4_317 discloses SEQ ID NOS 74-76, 86-87 and 106;

Antibody BIIB_4_318 discloses SEQ ID NOS 77-79 and 107-109; and

Antibody BIIB_4_319 discloses SEQ ID NOS 80-82, 86-87 and 110.

FIGS. 6A-F show the measurement of binding by BioLayer Interferometry (BLI) of Fab to sensor-associated GPIIb/IIIa (heterodimer formed by association of amino acid sequences encoded by SEQ ID NOs.: 1 and 3), as a function of time. FIG. 6A depicts BIIB-4-147. FIG. 6B depicts BIIB-4-174. FIG. 6C depicts BIIB-4-318. FIG. 6D depicts BIIB-4-175. FIG. 6E depicts BIIB-4-204. FIG. 6F depicts BIIB-4-311.

FIGS. 7A-D show the measurement of binding by BLI of Fab to sensor-associated GPIIb/IIIa (heterodimer formed by association of amino acid sequences encoded by SEQ ID NOs.: 1 and 3) or mutant GPIIb/IIIa (heterodimer formed by association of amino acid sequences encoded by SEQ ID NOs.: 2 and 4), as a function of time. FIG. 7A depicts BIIB-4-156. FIG. 7B depicts BIIB-4-224. FIG. 7C depicts BIIB-4-309. FIG. 7D depicts BIIB-4-311.

FIG. 8A provides the germline families and the CDRs of the antibodies that were determined to bind preferentially to GPIIb/IIIa (heterodimer formed by association of amino acid sequences encoded by SEQ ID NOs.: 1 and 3). Sequences are assigned below (from left to right):

Antibody BIIB_4_156 discloses SEQ ID NOS 56-58 and 86-88;

Antibody BIIB_4_224 discloses SEQ ID NOS 67-69 and 97-99;

Antibody BIIB_4_309 discloses SEQ ID NOS 53-54, 70, 83-84 and 102; and

Antibody BIIB_4_311 discloses SEQ ID NOS 71-73 and 103-105

FIG. 8B provides the germline families and the CDRs of the antibodies that were demonstrated to have no binding preference for active vs. inactive GPIIb/IIIa (i.e., they bind similarly to both). Sequences are assigned below (from left to right):

Antibody BIIB_4_147 discloses SEQ ID NOS 53-55 and 83-85;

Antibody BIIB_4_174 discloses SEQ ID NOS 56-57, 59, 86 and 89-90;

Antibody BIIB_4_175 discloses SEQ ID NOS 60-62 and 91-93;

Antibody BIIB_4_204 discloses SEQ ID NOS 63-65, 86 and 94-95;

Antibody BIIB_4_209 discloses SEQ ID NOS 53-54, 66, 86-87 and 96;

Antibody BIIB_4_317 discloses SEQ ID NOS 74-76, 86-87 and 106;

Antibody BIIB_4_318 discloses SEQ ID NOS 77-79 and 107-109; and

Antibody BIIB_4_319 discloses SEQ ID NOS 80-82, 86-87 and 110.

FIGS. 9A-D depict SPR traces for the association of conformation-selective Fabs with GPIIb/IIIa (heterodimer formed by association of amino acid sequences encoded by SEQ ID NOs.: 1 and 3) or mutant GPIIb/IIIa (heterodimer formed by association of amino acid sequences encoded by SEQ ID NOs.: 2 and 4), as a function of time. FIG. 9A depicts BIIB-4-224. FIG. 9B depicts BIIB-4-309. FIG. 9C depicts BIIB-4-311. FIG. 9D depicts BIIB-4-156.

FIG. 10 is a table listing the monovalent affinities measured for the binding of the identified antibodies to the GPIIb/IIIa ectodomain.

FIG. 11 is a representative example of 94 antibodies screened for propensity to self-associate by self-interaction nanoparticle spectroscopy. A threshold value of 540 nm as the max wavelength is set, with antibodies falling below threshold not highlighted and antibodies falling above threshold highlighted. A negative control with previously demonstrated good biophysical behavior and a positive control with previously demonstrated poor biophysical behavior are used as comparators.

FIG. 12A depicts binding of Fab of BIIB_4-224 to activated or resting platelets measured by flow cytometry. Plots are mean fluorescence intensity (MFI), a measurement of the amount of bound antibody to the surface of platelets, as a function of antibody concentration.

FIG. 12B depicts binding of Fab of BIIB_4-156 to activated or resting platelets measured by flow cytometry. Plots are mean fluorescence intensity (MFI).

FIG. 12C depicts binding of Fab of BIIB_4-309 to activated or resting platelets measured by flow cytometry. Plots are mean fluorescence intensity (MFI).

FIG. 12D is a table listing the antibodies that showed (and those that did not show) preferential binding to activated platelets.

FIG. 13 is a bar graph showing the measurement of platelet activation by flow cytometry. Buffer or Fabs were added to resting platelets and the binding of PAC-1 is compared to that of stimulated platelets, to assess the capability of GPIIb/IIIa antibody binding to indirectly activate platelets. Plots are mean fluorescence intensity (MFI), a measurement of the amount of bound antibody (PAC-1) to the surface of platelets, as a function of buffer alone, Fab addition, or a positive control of activated platelets.

FIG. 14A is a bar graph of a representative example of a fibrinogen competition assay performed by flow cytometry. Fab of BIIB-4-156 was added at 0, 0.5, or 5 μg/ml to activated platelets. Binding of fluorescently labeled fibrinogen was then detected. MFI on the y-axis indicates the amount of fibrinogen bound to platelets in the presence of either BIIB-4-156 or a previously identified competitor antibody.

FIG. 14B is a table identifying antibodies that were capable of or not capable of inhibiting fibrinogen binding to platelets.

FIG. 15A is a table listing the germline family and amino acid sequences of the CDRs of the antibodies that inhibit fibrinogen association with GPIIb/IIIa. Sequences are assigned below (from left to right):

Antibody BIIB_4_174 discloses SEQ ID NOS 56-57, 59, 86 and 89-90; and

Antibody BIIB_4_175 discloses SEQ ID NOS 60-62 and 91-93.

FIG. 15B is a table listing the germline family and amino acid sequences of the CDRs of the antibodies that do not inhibit fibrinogen association with GPIIb/IIIa. Sequences are assigned below (from left to right):

Antibody BIIB_4_147 discloses SEQ ID NOS 53-55 and 83-85;

Antibody BIIB_4_156 discloses SEQ ID NOS 56-58 and 86-88;

Antibody BIIB_4_204 discloses SEQ ID NOS 63-65, 86 and 94-95;

Antibody BIIB_4_209 discloses SEQ ID NOS 53-54, 66, 86-87 and 96;

Antibody BIIB_4_224 discloses SEQ ID NOS 67-69 and 97-99;

Antibody BIIB_4_309 discloses SEQ ID NOS 53-54, 70, 83-84 and 102;

Antibody BIIB_4_311 discloses SEQ ID NOS 71-73 and 103-105;

Antibody BIIB_4_317 discloses SEQ ID NOS 74-76, 86-87 and 106;

Antibody BIIB_4_318 discloses SEQ ID NOS 77-79 and 107-109; and

Antibody BIIB_4_319 discloses SEQ ID NOS 80-82, 86-87 and 110.

FIG. 16 is a graphical depiction of ROTEM assay results in human blood comparing BIIB-4-147_rFVIIa (a platelet-targeted chimeric protein comprising an anti-GPIIb/IIIa Fab (BBB-4-147) and recombinant FVIIa) compared to recombinant FVIIa alone.

FIG. 17 shows the measurement of binding by BLI of the indicated Fab followed by the second indicated Fab to sensor-associated GPIIb/IIIa (heterodimer formed by association of amino acid sequences encoded by SEQ ID NOs.: 1 and 3), as a function of time. The table depicts the cross-blocking assignments based on epitope binning observations.

FIG. 18A-F show possible configurations for chimeric molecules comprising the heavy and light chains of a clotting factor (e.g., a FVII), an Fab or scFv targeting moiety (e.g., derived from or based on the GPIIb/IIIa-specific antibodies described herein), a heterologous moiety (e.g., a half-life extending moiety), and at least one optional linker. FIG. 18A depicts exemplary chimeric molecule 1. FIG. 18B depicts exemplary chimeric molecule 2. FIG. 18C depicts exemplary chimeric molecule 3. FIG. 18D depicts exemplary chimeric molecule 4. FIG. 18E depicts exemplary chimeric molecule 5. FIG. 18F depicts exemplary chimeric molecule 6.

FIG. 19 shows possible configuration for chimeric molecules comprising one or two heterologous moieties (H1 and/or H2) and scFv moieties derived from or based on the GPIIb/IIIa-specific antibodies described herein. It is to be understood that an Fab derived from the anti-GPIIb/IIIa antibodies can be used instead of the scFv in these chimeric molecules.

FIG. 20A-D shows the measurement of binding by BLI of the indicated yeast purified Fab to sensor-associated GPIIb/IIIa (SEQ ID NO:1 and 3) or integrin alpha V beta III (SEQ ID NO:245 and 3), as a function of time. FIG. 20A depicts BIIB-4-147. FIG. 20B depicts BIIB-4-156. FIG. 20C depicts BIIB-4-174. FIG. 20D depicts BBB-4-319.

FIG. 20E is a table listing the apparent integrin binding specificity, as assessed by BLI in the monovalent format, of the indicated yeast purified Fab.

FIG. 21 shows the results of SPR studies using BIIB_4_309-FVIIa and the active and inactive forms of GPIIb/IIIa. These data demonstrate that the specificity of Fab BIIB_4_309 for the active conformation of GPIIb/IIIa is maintained when fused to FVIIa.

### DETAILED DESCRIPTION

This disclosure features antibodies and antigen-binding fragments that specifically bind GPIIb/IIIa, an integrin that is expressed at high levels on platelets. Upon activation, the GPIIb/IIIa receptors change from a bent low ligand affinity conformation to an extended high ligand affinity conformation. Activated GPIIb/IIIa receptor binds fibrinogen and modulates platelet aggregation. Anti-GPIIb/IIIa antibodies with different properties are described herein. A first class of the anti-GPIIb/IIIa antibodies and antigen-binding fragments thereof are capable of preferentially targeting the active compared to the non-active form of the GPIIb/IIIa receptor. A second class of the anti-GPIIb/IIIa antibodies and antigen-binding fragments thereof are capable of binding to both the active and the non-active form of the GPIIb/IIIa receptor with the same or similar affinity. A subset of the antibodies and antigen-binding fragments of this second class, represent a third class, in that unlike members of the second class, they can compete with fibrinogen for binding GPIIb/IIIa. All three classes of the anti-GPIIb/IIIa antibodies and antigen-binding fragments derived from these antibodies do not activate platelets and do not disrupt platelet function. The antibodies described herein can be used, for example, to target agents (e.g., therapeutic agents such as clotting factors or other molecules capable of having a pharmacological effect in platelets) to the platelet surface: the first class of antibodies and antigen-binding fragments to activated platelets; and the second class to all platelets. In addition to their use as platelet-targeting moieties, the antibodies and antigen-binding fragments thereof described herein can be used for diagnostics, for example, by conjugation to a detectable label, and also used for isolating and separating platelets from a sample, and enriching for activated platelets. Some of the antibodies described herein (e.g., antibodies of the third class) can be used to reduce or prevent platelet aggregation and thrombus formation as well as diagnostic tools for evaluating fibrinogen blocking.

This disclosure also provides chimeric molecules comprising the anti-GPIIb/IIIa antibodies and antigen-binding fragments thereof disclosed herein. Such chimeric molecules can include the antibodies or antigen-binding fragments thereof and one or more (e.g., one, two, three, four) heterologous moieties. For example, the chimeric molecules can comprise a heterologous moiety comprising a therapeutic molecule (e.g., a procoagulant molecule such as a clotting factor), and optionally a second heterologous moiety comprising, for example, a pharmacokinetic (PK) enhancing moiety (i.e., a molecule which can improve various pharmacokinetic properties, e.g., half-life). The heterologous moieties can optionally be connected by linkers (e.g., peptide linkers). In addition the targeting moiety of the chimeric molecule (e.g., an Fab or scFv of an anti-GPIIb/IIIa antibody described herein) can optionally be connected to the heterologous moiety or moieties via linkers (e.g., a peptide linker). Exemplary anti-GPIIb/IIIa antibodies and antigen-binding fragments thereof, as well as exemplary constructs (chimeric molecules) comprising such antibodies and antigen-binding fragments thereof (e.g., scFv or Fab) are illustrated in the instant description and figures. See, e.g., the chimeric molecules having the structures set forth in FIGS. 18 and 19.

The disclosure also provides polynucleotides encoding the antibodies and antigen-binding fragments thereof as well as the chimeric molecule constructs described herein.

In addition, this disclosure relates to methods of using some of the anti-GPIIb/IIIa antibodies and antigen-binding fragments thereof in the treatment of coagulation deficiencies such as hemophilia well as coagulation deficiencies other than hemophilia characterized by an impaired thrombin generation and life-threatening bleeding.

Furthermore, this disclosure relates to methods of using certain of the anti-GPIIb/IIIa antibodies and antigen-binding fragments thereof described in the reducing or preventing platelet aggregation and thrombus formation in a subject in need thereof.

In order to provide a clear understanding of the specification and claims, the following definitions are provided below.

### A. Definitions

It is understood that wherever embodiments are described herein with the language “comprising,” otherwise analogous embodiments described in terms of “consisting of” and/or “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “antibody” means an immunoglobulin molecule that recognizes and specifically binds to a target, such as a protein (e.g., the GPIIb/IIIa receptor, a subunit thereof, or the receptor complex), polypeptide, peptide, carbohydrate, polynucleotide, lipid, or combinations of the foregoing through at least one antigen recognition site within the variable region of the immunoglobulin molecule. A typical antibody comprises at least two heavy (HC) chains and two light (LC) chains interconnected by disulfide bonds. Each heavy chain is comprised of a “heavy chain variable region” or “heavy chain variable domain” (abbreviated herein as VH) and a heavy chain constant region. The heavy chain constant region is comprised of three domains, CH1, CH2, and CH3. Each light chain is comprised of a “light chain variable region” or “light chain variable domain” (abbreviated herein as VL) and a light chain constant region. The light chain constant region is comprised of one domain, Cl. The VH and VL regions can be further subdivided into regions of hypervariablity, termed Complementarity Determining Regions (CDR), interspersed with regions that are more conserved, termed framework regions (FRs). Each VH and VL region is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. As used herein, the term “antibody” encompasses intact polyclonal antibodies, intact monoclonal antibodies, antibody fragments (such as Fab, Fab′, F(ab′)2, Fd, Facb, and Fv fragments), single chain Fv (scFv), minibodies (e.g., sc(Fv)2, diabody), multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. Thus, the term “antibody” includes whole antibodies and any antigen-binding fragment or single chains thereof. Antibodies can be naked or conjugated to other molecules such as toxins, radioisotopes, small molecule drugs, polypeptides, etc.

The term “antigen binding fragment” refers to a portion of an intact antibody and refers to the antigenic determining variable regions of an intact antibody. It is known in the art that the antigen binding function of an antibody can be performed by fragments of a full-length antibody. Examples of antigen-binding antibody fragments include, but are not limited to Fab, Fab′, F(ab′)2, Facb, Fd, and Fv fragments, linear antibodies, single chain antibodies, and multispecific antibodies formed from antibody fragments. In some instances, antibody fragments may be prepared by proteolytic digestion of intact or whole antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments can be enzymatically or chemically produced by fragmentation of an intact antibody, recombinantly produced from a gene encoding the partial antibody sequence, or it can be wholly or partially synthetically produced. The term “F(ab′)2” refers to an antibody fragment that is essentially equivalent to a fragment obtained by digestion of an immunoglobulin (typically IgG) with the enzyme pepsin at pH 4.0-4.5. Such fragments can be enzymatically or chemically produced by fragmentation of an intact antibody, recombinantly produced from a gene encoding the partial antibody sequence, or it can be wholly or partially synthetically produced. The term “Fv” refers to an antibody fragment that consists of one NH and one N domain held together by noncovalent interactions.

As used herein the term “scFv” or “scFv molecule” includes binding molecules which consist of one light chain variable domain (VL) or a portion thereof, and one heavy chain variable domain (VH) or a portion thereof, wherein each variable domain (or a portion thereof) is derived from the same or different antibodies. Single chain Fv molecules preferably comprise an scFv linker interposed between the VH domain and the VL domain. Exemplary scFv molecules are known in the art and are described, for example, in U.S. Pat. No. 5,892,019; Ho et al., Gene, 77:51 (1989); Bird et al., Science, 242:423 (1988); Pantoliano et al., Biochemistry, 30:10117 (1991); Milenic et al., Cancer Research, 51:6363 (1991); Takkinen et al., Protein Engineering, 4:837 (1991). The term “scFv linker” as used herein refers to a moiety interposed between the VL and VH domains of the scFv. The scFv linkers preferably maintain the scFv molecule in an antigen-binding conformation. In one embodiment, a scFv linker comprises or consists of an scFv linker peptide. In certain embodiments, an scFv linker peptide comprises or consists of a Gly-Ser peptide linker. In other embodiments, an scFv linker comprises a disulfide bond.

The terms “GPIIb/IIIa antibody,” “anti-GPIIb/IIIa antibody,” “anti-GPIIb/IIIa,” “antibody that binds to GPIIb/IIIa” and any grammatical variations thereof refer to an antibody that is capable of specifically binding to the GPIIb/IIIa receptor with sufficient affinity such that the antibody is useful as a therapeutic agent or diagnostic reagent in targeting GPIIb/IIIa. The extent of binding of an anti-GPIIb/IIIa antibody disclosed herein to an unrelated, non-GPIIb/IIIa protein is less than about 10% of the binding of the antibody to GPIIb/IIIa as measured, e.g., by a radioimmunoassay (RIA), BIACORE™ (using recombinant GPIIb/IIIa as the analyte and antibody as the ligand, or vice versa), or other binding assays known in the art. In certain embodiments, an antibody that binds to GPIIb/IIIa has a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM. The anti-GPIIb/IIIa antibody can comprise a VH and VL domain. Examples of anti-GPIIb/IIIa antibodies include an antibody selected from BIIB-4-147 (VH: SEQ ID NO:5; VL: SEQ ID NO:7), BIIB-4-156 (VH: SEQ ID NO:9; VL: SEQ ID NO:11), BIIB-4-174 (VH: SEQ ID NO:13; VL: SEQ ID NO:15), BIIB-4-175 (VH: SEQ ID NO:17; VL: SEQ ID NO:19), BIIB-4-204 (VH: SEQ ID NO:21; VL: SEQ ID NO:23), BIIB-4-209 (VH: SEQ ID NO:25; VL: SEQ ID NO:27), BIIB-4-224 (VH: SEQ ID NO:29; VL: SEQ ID NO:31), BIIB-4-309 (VH: SEQ ID NO:33; VL: SEQ ID NO:35), BIIB-4-311 (VH: SEQ ID NO:37; VL: SEQ ID NO:39), BIIB-4-317 (VH: SEQ ID NO:41; VL: SEQ ID NO:43), BIIB-4-318 (VH: SEQ ID NO:45; VL: SEQ ID NO:47), and BIIB-4-319 (VH: SEQ ID NO:49; VL: SEQ ID NO:51).

As used herein, the term “epitope” designates a specific amino acid sequence, modified amino acid sequence, or protein secondary or tertiary structure which is specifically recognized by an antibody. The terms “specifically recognizing,” “specifically recognizes,” and any grammatical variants mean that the antibody or antigen-binding molecule thereof is capable of specifically interacting with and/or binding to at least two, at least three, or at least four amino acids of an epitope, e.g., a GPIIb/IIIa epitope. Such binding can be exemplified by the specificity of a “lock-and-key-principle.” Thus, specific motifs in the amino acid sequence of the antigen-binding domain the GPIIb/IIIa antibody or antigen-binding molecule thereof and the epitope bind to each other as a result of their primary, secondary or tertiary structure as well as the result of secondary modifications of the structure.

A platelet is said to be “resting” when it does not express one or more markers of platelet activation such as P-selectin (CD62p) and/or PAC-1. In certain instances, a resting platelet expresses the CD41 marker. A platelet is said to be “activated” when it expresses one or more markers of platelet activation such as P-selectin (CD62p) and/or PAC-1.

The term “% identical” between two polypeptide (or polynucleotide) sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2 seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org (ClustalX is a version of the ClustalW2 program ported to the Windows environment). Another suitable program is MUSCLE, available from www.drive5.com/muscle. ClustalW2 and MUSCLE are alternatively available, e.g., from the EBI.

As used herein, the term “targeting moiety” refers to a moiety capable of interacting with a target molecule (e.g., the GPIIb/IIIa receptor, or a molecule comprising the α and/or β subunits of the GPIIb/IIIa receptor). Targeting moieties having limited cross-reactivity are generally preferred. In certain embodiments, suitable targeting moieties include, for example, any member of a specific binding pair, antibodies, monoclonal antibodies, or derivatives or analogs thereof, including without limitation: Fv fragments, single chain Fv (scFv) fragments, Fab fragments, F(ab′)2 fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((scFv) fragments), diabodies, tribodies or tetrabodies, which typically are covalently linked or otherwise stabilized (i.e., leucine zipper or helix stabilized) scFv fragments; and other targeting moieties include for example, aptamers, receptors, ligands, and fusion proteins.

The terms “linked” or “fused” refers to linkage via a peptide bonds (e.g., genetic fusion), chemical conjugation, or other means known in the art. For example, one way in which molecules or moieties can be linked employs peptide linkers that link the molecules or moieties via peptide bonds.

The term “associated with” refers to a covalent or non-covalent bond formed between a first amino acid chain and a second amino acid chain. In one embodiment, the term “associated with” means a covalent, non-peptide bond or a non-covalent bond. In another embodiment, the term “associated with” refers to a covalent, non-peptide bond or a non-covalent bond that is not chemically crosslinked. In another embodiment, it means a covalent bond except a peptide bond. In some embodiments this association is indicated by a colon, i.e., (:). For example, when representing the structure of the clotting factor, “CFH:CFL” refers to a dimer comprising a heavy chain of a clotting factor (CFH) disulfide bonded to a light chain of a clotting factor (CFL) in a N-terminus to C-terminus orientation.

The term “moiety” refers to a component part or constituent of a chimeric molecule of the present disclosure.

The term “heterologous moiety” refers to a moiety genetically fused, conjugated, and/or otherwise associated to a targeting molecule (e.g., GPIIb/IIIa antibody or antigen-binding molecule thereof).

The term “therapeutic agent” refers to any biological or chemical agent used in the treatment of a disease or disorder. Therapeutic agents include any suitable biologically active chemical compounds, biologically derived components such as cells, peptides, antibodies, and polynucleotides, and radiochemical therapeutic agents such as radioisotopes. In some embodiments, the therapeutic agent comprises a clotting factor.

The term “stability” refers to an art-recognized measure of the maintenance of one or more physical properties of the chimeric molecule in response to an environmental condition (e.g., an elevated or lowered temperature). In certain embodiments, the physical property can be the maintenance of the covalent structure of the chimeric molecule (e.g., the absence of proteolytic cleavage, unwanted oxidation or deamidation). In other embodiments, the physical property can also be the presence of the chimeric molecule in a properly folded state (e.g., the absence of soluble or insoluble aggregates or precipitates). In one embodiment, the stability of the chimeric molecule is measured by assaying a biophysical property of the chimeric molecule, for example thermal stability, pH unfolding profile, stable removal of glycosylation, solubility, biochemical function (e.g., ability to bind to a protein, receptor or ligand), etc., and/or combinations thereof. In another embodiment, biochemical function is demonstrated by the binding affinity of the interaction. In one embodiment, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

The term “clotting factor” refers to molecules, or analogs thereof, naturally occurring or recombinantly produced which prevent or decrease the duration of a bleeding episode in a subject. In other words, it means molecules having pro-clotting activity, i.e., are responsible for the conversion of fibrinogen into a mesh of insoluble fibrin causing the blood to coagulate or clot. The term “clotting factor,” as used herein encompasses clotting factors (e.g., vWF, FV, FVa, FVII, FVIIa, FVIII, FVIIIa, FIX, FIXa, FX, FXa, FXI, FXIa, FXII, FXIIa, FXIII, or FXIIIa), fragments, variants, analogs, or derivatives thereof, naturally occurring, recombinantly produced, or synthetically produced which prevent or decrease the duration of a bleeding episode in a subject.

The term “activatable clotting factor” refers to a clotting factor in an inactive form (e.g., in its zymogen form) that is capable of being converted to an active form.

As used herein, a “zymogen-like” protein or polypeptide refers to a protein that has been activated by proteolytic cleavage, but still exhibits properties that are associated with a zymogen, such as, for example, low or no activity, or a conformation that resembles the conformation of the zymogen form of the protein. For example, when it is not bound to tissue factor, the two-chain activated form of FVII is a zymogen-like protein; it retains a conformation similar to the uncleaved FVII zymogen, and, thus, exhibits very low activity. Upon binding to tissue factor, the two-chain activated form of FVII undergoes conformational change and acquires its full activity as a coagulation factor.

As used herein, the term “half-life extending moiety” refers to a heterologous moiety which increases the in vivo half-life of a protein, for example, a chimeric molecule. The term “half-life” refers to a biological half-life of a particular protein or polypeptide (e.g., a clotting factor or a chimeric molecule disclosed herein) in vivo. Half-life can be represented by the time required for half the quantity administered to a subject to be cleared from the circulation and/or other tissues in the animal. When a clearance curve of a given polypeptide or chimeric molecule of the invention is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, procoagulant compounds of the invention are monophasic, and thus do not have an alpha phase, but just the single beta phase. In certain embodiments, the term half-life as used herein refers to the half-life of the procoagulant compound in the β-phase. The typical β-phase half-life of a human antibody in humans is 21 days. In vivo half-life of a chimeric molecule can be determined by any method known to those of skill in the art. In certain embodiments, the half-life extending moiety can comprise an attachment site for a non-polypeptide moiety (e.g., PEG).B. GPIIb/IIIa

The terms “GPIIb/IIIa” and “GPIIb/IIIa receptor” refer to glycoprotein IIb/IIIa (also known as integrin aIIbβ3), an integrin complex found on platelets. Integrins are composed of two chains, an α subunit and a β subunit, which are held together by noncovalent bonds in a calcium dependent manner. GPIIb constitutes the a subunit, which comprises divalent cation binding domains, whereas GPIIIa is a pro typical β subunit (β3). On each circulating platelet, there are about 35,000 to 100,000 GPIIb/IIIa complexes: most are distributed on the platelet surface, while a smaller pool is found in an internal reserve. The GPIIb/IIIa complex does not interact with its plasma ligands until platelets have been activated by exogenous agonists such as ADP or thrombin. When this occurs, an inside-out signal is generated that results in a conformational change in the extracellular portion of the complex that renders the molecule capable of binding fibrinogen and other ligands. The amino acid sequences of the two chains of this platelet receptor can be found in Uniprot entries P05106 (ITB3_HUMAN; GPIIIa: CD61; integrin beta-3; integrin β3) and P08514 (ITA2B_HUMAN; GPIIb; CD41; integrin alpha-2b; integrin αII) as published in Universal Protein Resource (Uniprot) database release 2013_05 (May 1, 2013), which are incorporated by reference in their entireties.

### GPIIb:

The amino acid sequence of the human GPIIb protein is shown below:

(SEQ ID NO: 1)
MARALCPLQALWLLEWVLLLLGPCAAPPAWALNLD
PVQLTFYAGPNGSQFGFSLDFHKDSHGRVAIVVGA
PRTLGPSQEETGGVFLCPWRAEGGQCPSLLFDLRD
ETRNVGSQTLQTFKARQGLGASVVSWSDVIVACAP
WQHWNVLEKTEEAEKTPVGSCFLAQPESGRRAEYS
PCRGNTLSRIYVENDFSWDKRYCEAGFSSVVTQAG
ELVLGAPGGYYFLGLLAQAPVADIFSSYRPGILLW
HVSSQSLSFDSSNPEYFDGYWGYSVAVGEFDGDLN
TTEYVVGAPTWSWTLGAVEILDSYYQRLHRLRGEQ
MASYFGHSVAVTDVNGDGRHDLLVGAPLYMESRAD
RKLAEVGRVYLFLQPRGPHALGAPSLLLTGTQLYG
RFGSAIAPLGDLDRDGYNDIAVAAPYGGPSGRGQV
LVFLGQSEGLRSRPSQVLDSPFPTGSAFGFSLRGA
VDIDDNGYPDLIVGAYGANQVAVYRAQPVVKASVQ
LLVQDSLNPAVKSCVLPQTKTPVSCFNIQMCVGAT
GHNIPQKLSLNAELQLDRQKPRQGRRVLLLGSQQA
GTTLNLDLGGKHSPICHTTMAFLRDEADFRDKLSP
IVLSLNVSLPPTEAGMAPAVVLHGDTHVQEQTRIV
LDCGEDDVCVPQLQLTASVTGSPLLVGADNVLELQ
MDAANEGEGAYEAELAVHLPQGAHYMRALSNVEGF
ERLICNQKKENETRVVLCELGNPMKKNAQIGIAML
VSVGNLEEAGESVSFQLQIRSKNSQNPNSKIVLLD
VPVRAEAQVELRGNSFPASLVVAAEEGEREQNSLD
SWGPKVEHTYELHNNGPGTVNGLHLSIHLPGQSQP
SDLLYILDIQPQGGLQCFPQPPVNPLKVDWGLPIP
SPSPIHPAHHKRDRRQIFLPEPEQPSRLQDPVLVS
CDSAPCTVVQCDLQEMARGQRAMVTVLAFLWLPSL
YQRPLDQFVLQSHAWFNVSSLPYAVPPLSLPRGEA
QVWTQLLRALEERA

The amino acid sequence of a mutated human GPIIb protein that has an L959C mutation (highlighted, boldened, and underlined), is shown below:

(SEQ ID NO: 2)
MARALCPLQALWLLEWVLLLLGPCAAPPAWALNLDPVQLTFYAGPNGSQFGFSLDFHKDSHGRVAIVVGAPRTLG
PSQEETGGVFLCPWRAEGGQCPSLLFDLRDETRNVGSQTLQTFKARQGLGASVVSWSDVIVACAPWQHWNVLEKT
EEAEKTPVGSCFLAQPESGRRAEYSPCRGNTLSRIYVENDFSWDKRYCEAGFSSVVTQAGELVLGAPGGYYFLGL
LAQAPVADIFSSYRPGILLWHVSSQSLSFDSSNPEYFDGYWGYSVAVGEFDGDLNTTEYVVGAPTWSWTLGAVEI
LDSYYQRLHRLRGEQMASYFGHSVAVTDVNGDGRHDLLVGAPLYMESRADRKLAEVGRVYLFLQPRGPHALGAPS
LLLTGTQLYGRFGSAIAPLGDLDRDGYNDIAVAAPYGGPSGRGQVLVFLGQSEGLRSRPSQVLDSPFPTGSAFGF
SLRGAVDIDDNGYPDLIVGAYGANQVAVYRAQPVVKASVQLLVQDSLNPAVKSCVLPQTKTPVSCFNIQMCVGAT
GHNIPQKLSLNAELQLDRQKPRQGRRVLLLGSQQAGTTLNLDLGGKHSPICHTTMAFLRDEADFRDKLSPIVLSL
NVSLPPTEAGMAPAVVLHGDTHVQEQTRIVLDCGEDDVCVPQLQLTASVTGSPLLVGADNVLELQMDAANEGEGA
YEAELAVHLPQGAHYMRALSNVEGFERLICNQKKENETRVVLCELGNPMKKNAQIGIAMLVSVGNLEEAGESVSF
QLQIRSKNSQNPNSKIVLLDVPVRAEAQVELRGNSFPASLVVAAEEGEREQNSLDSWGPKVEHTYELHNNGPGTV
NGLHLSIHLPGQSQPSDLLYILDIQPQGGLQCFPQPPVNPLKVDWGLPIPSPSPIHPAHHKRDRRQIFLPEPEQP
SRLQDPVLVSCDSAPCTVVQCDLQEMARGQRAMVTVLAFLWLPSLYQRPLDQFVLQSHAWFNVSSLPYAVPPLSL

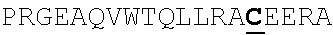

### GPIIIa:

The amino acid sequence of the human GPIIIa protein is shown below:

(SEQ ID NO: 3)
MRARPRPRPLWATVLALGALAGVGVGGPNICTTRGV
SSCQQCLAVSPMCAWCSDEALPLGSPRCDLKENLL
KDNCAPESIEFPVSEARVLEDRPLSDKGSGDSSQV
TQVSPQRIALRLRPDDSKNFSIQVRQVEDYPVDIY
YLMDLSYSMKDDLWSIQNLGTKLATQMRKLTSNLR
IGFGAFVDKPVSPYMYISPPEALENPCYDMKTTCL
PMFGYKHVLTLTDQVTRFNEEVKKQSVSRNRDAPE
GGFDAIMQATVCDEKIGWRNDASHLLVFTTDAKTH
IALDGRLAGIVQPNDGQCHVGSDNHYSASTTMDYP
SLGLMTEKLSQKNINLIFAVTENVVNLYQNYSELI
PGTTVGVLSMDSSNVLQLIVDAYGKIRSKVELEVR
DLPEELSLSFNATCLNNEVIPGLKSCMGLKIGDTV
SFSIEAKVRGCPQEKEKSFTIKPVGFKDSLIVQVT
FDCDCACQAQAEPNSHRCNNGNGTFECGVCRCGPG
WLGSQCECSEEDYRPSQQDECSPREGQPVCSQRGE
CLCGQCVCHSSDFGKITGKYCECDDFSCVRYKGEM
CSGHGQCSCGDCLCDSDWTGYYCNCTTRTDTCMSS
NGLLCSGRGKCECGSCVCIQPGSYGDTCEKCPTCP
DACTFKKECVECKKFDRGALHDENTCNRYCRDEIE
SVKELKDTGKDAVNCTYKNEDDCVVRFQYYEDSSG
KSILYVVEEPECPKG

The amino acid sequence of a mutated human GPIIIa protein that has a P688C mutation (highlighted, boldened, and underlined) is shown below:

(SEQ ID NO: 4)
MRARPRPRPLWATVLALGALAGVGVGGPNICTTRGVSSCQQCLAVSPMCAWCSDEALPLGSPRCDLKENLLKDNC
APESIEFPVSEARVLEDRPLSDKGSGDSSQVTQVSPQRIALRLRPDDSKNFSIQVRQVEDYPVDIYYLMDLSYSM
KDDLWSIQNLGTKLATQMRKLTSNLRIGFGAFVDKPVSPYMYISPPEALENPCYDMKTTCLPMFGYKHVLTLTDQ
VTRFNEEVKKQSVSRNRDAPEGGFDAIMQATVCDEKIGWRNDASHLLVFTTDAKTHIALDGRLAGIVQPNDGQCH
VGSDNHYSASTTMDYPSLGLMTEKLSQKNINLIFAVTENVVNLYQNYSELIPGTTVGVLSMDSSNVLQLIVDAYG
KIRSKVELEVRDLPEELSLSFNATCLNNEVIPGLKSCMGLKIGDTVSFSIEAKVRGCPQEKEKSFTIKPVGFKDS
LIVQVTFDCDCACQAQAEPNSHRCNNGNGTFECGVCRCGPGWLGSQCECSEEDYRPSQQDECSPREGQPVCSQRG
ECLCGQCVCHSSDFGKITGKYCECDDFSCVRYKGEMCSGHGQCSCGDCLCDSDWTGYYCNCTTRTDTCMSSNGLL
CSGRGKCECGSCVCIQPGSYGDTCEKCPTCPDACTFKKECVECKKFDRGALHDENTCNRYCRDEIESVKELKDTG

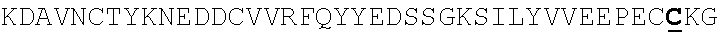

### C. Anti-GPIIb/IIIa Antibodies

This disclosure provides antibodies and antigen-binding fragments thereof that specifically bind to GPIIb/IIIa. In certain embodiments, these antibodies and antigen-binding fragments thereof are fully human antibodies or antigen-binding fragments thereof. In certain embodiments, these antibodies and antigen-binding fragments thereof bind the GPIIb/IIIa receptors located on the surface of platelets. In other embodiments, these antibodies and antigen-binding fragments thereof bind the GPIIb/IIIa found within the platelets. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM.

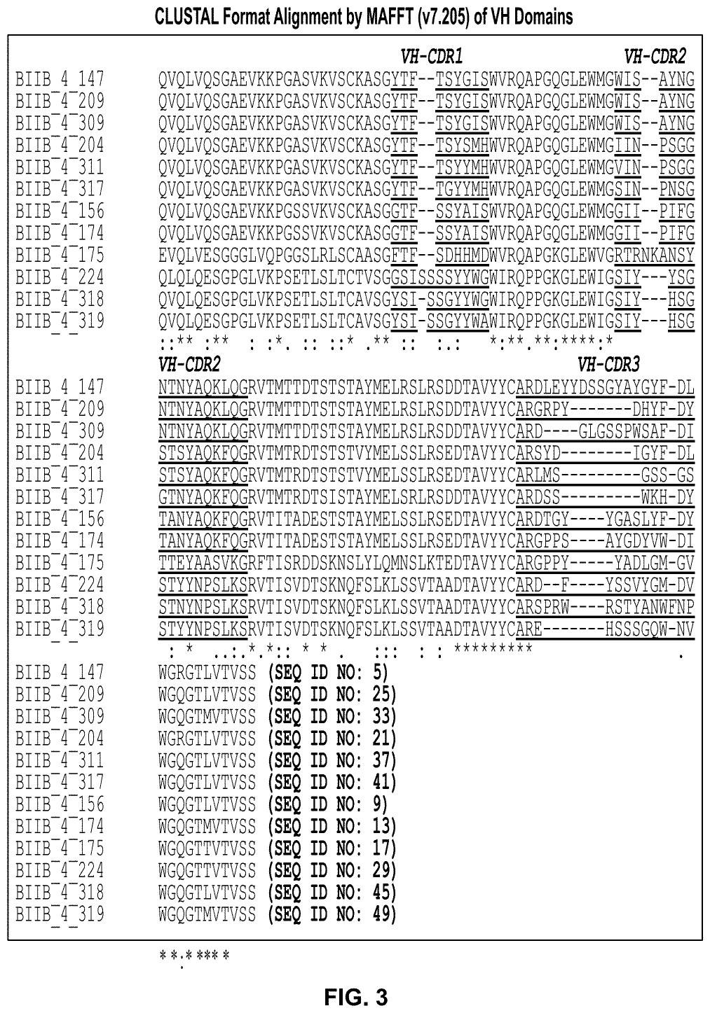

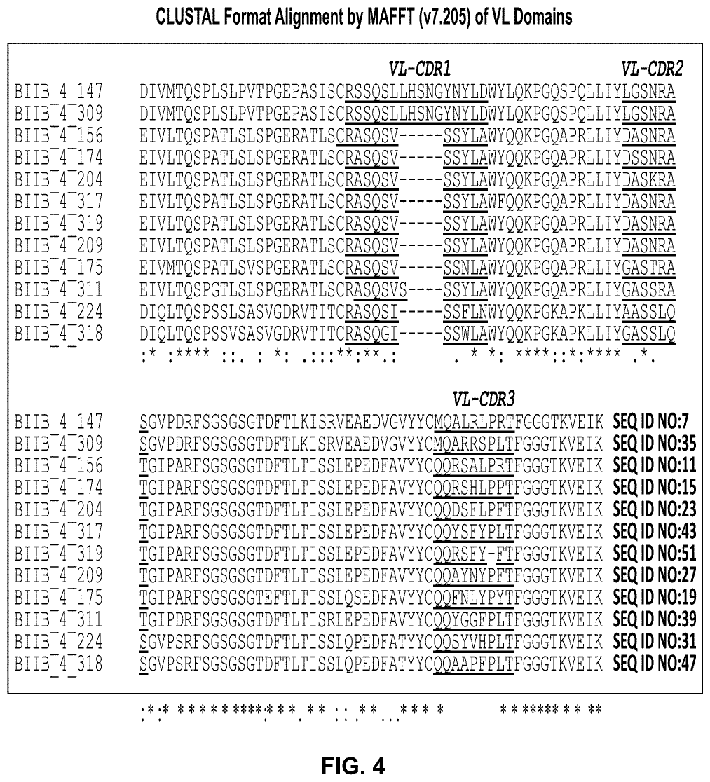

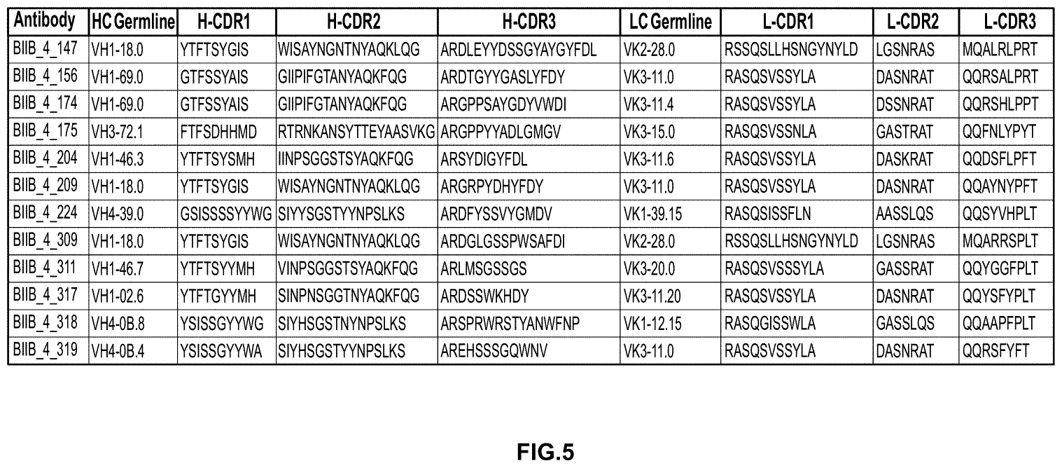

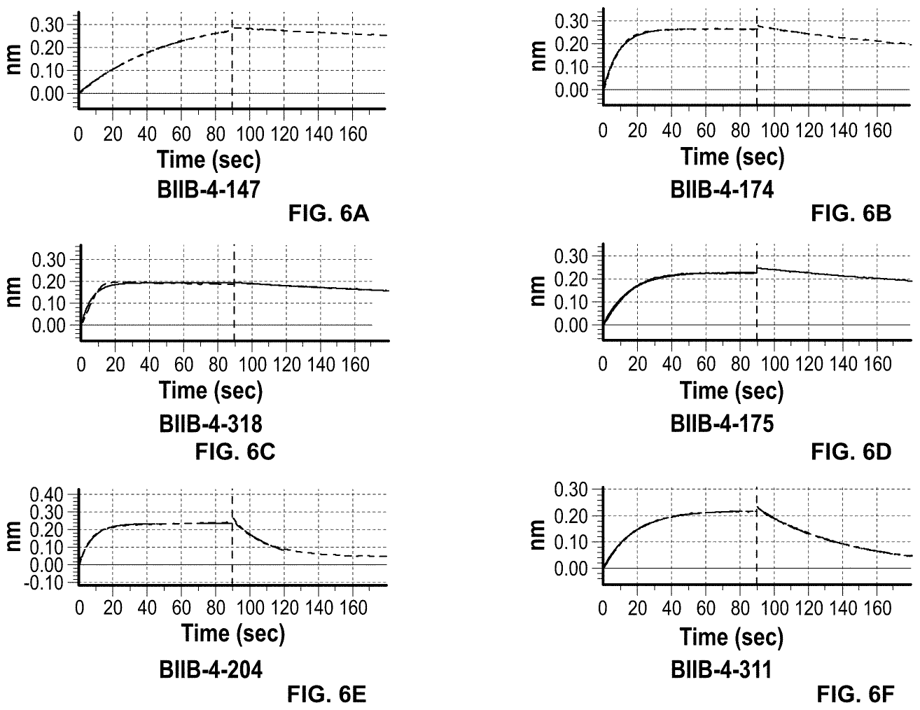

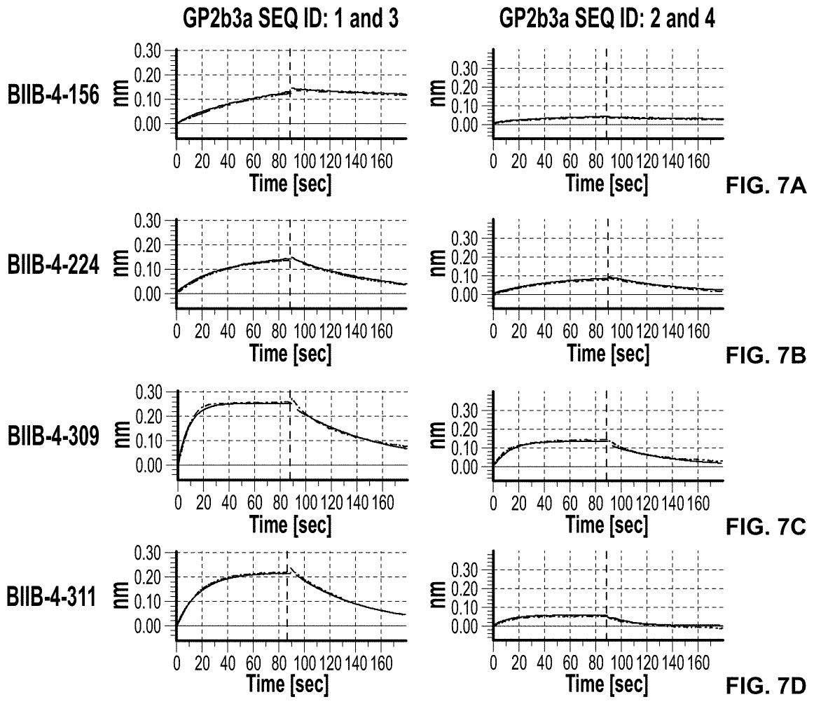

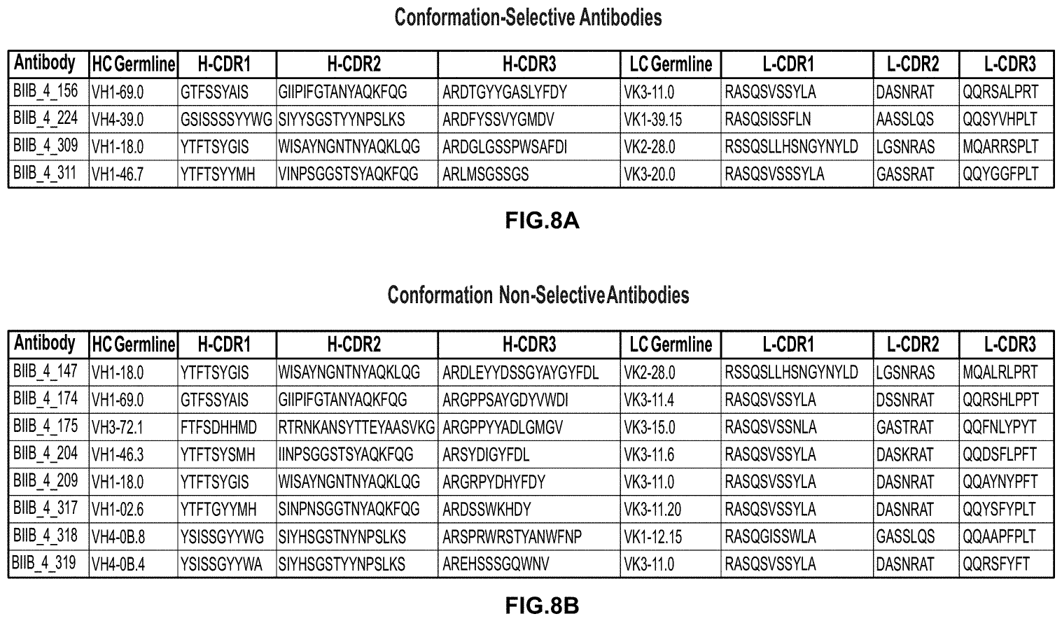

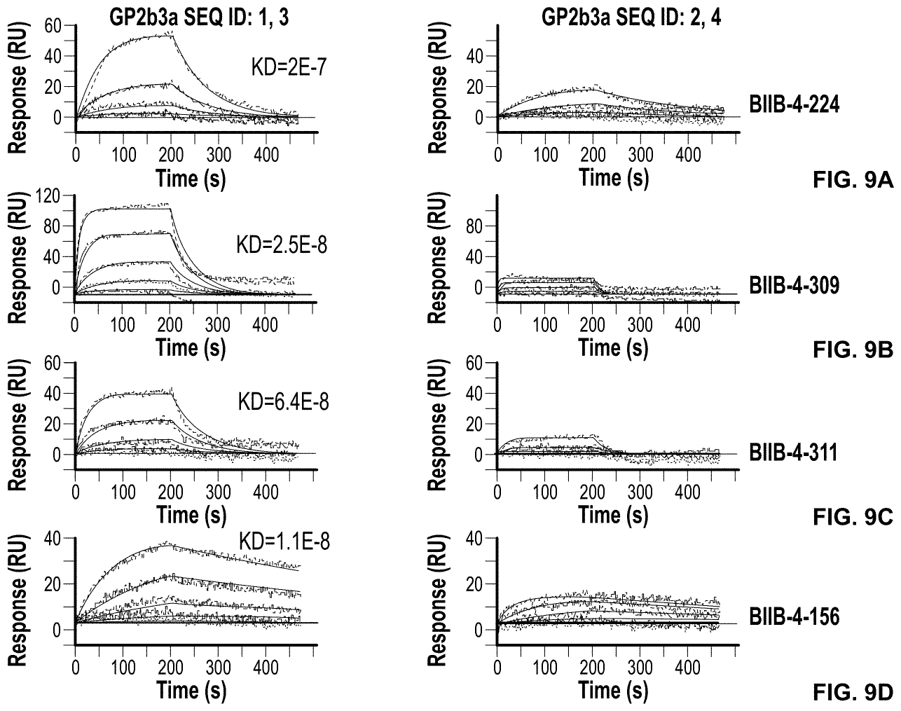

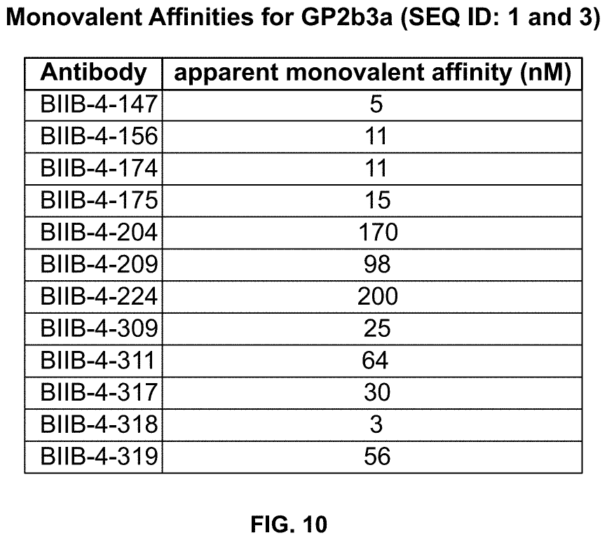

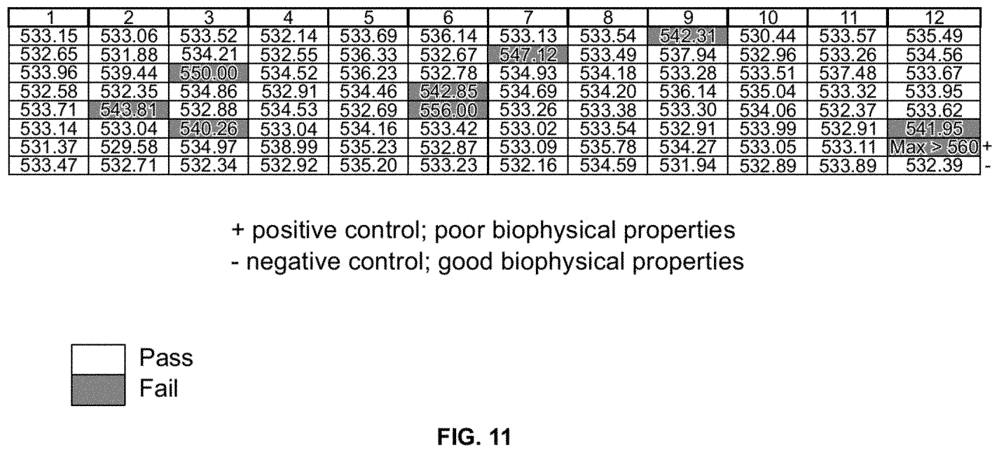

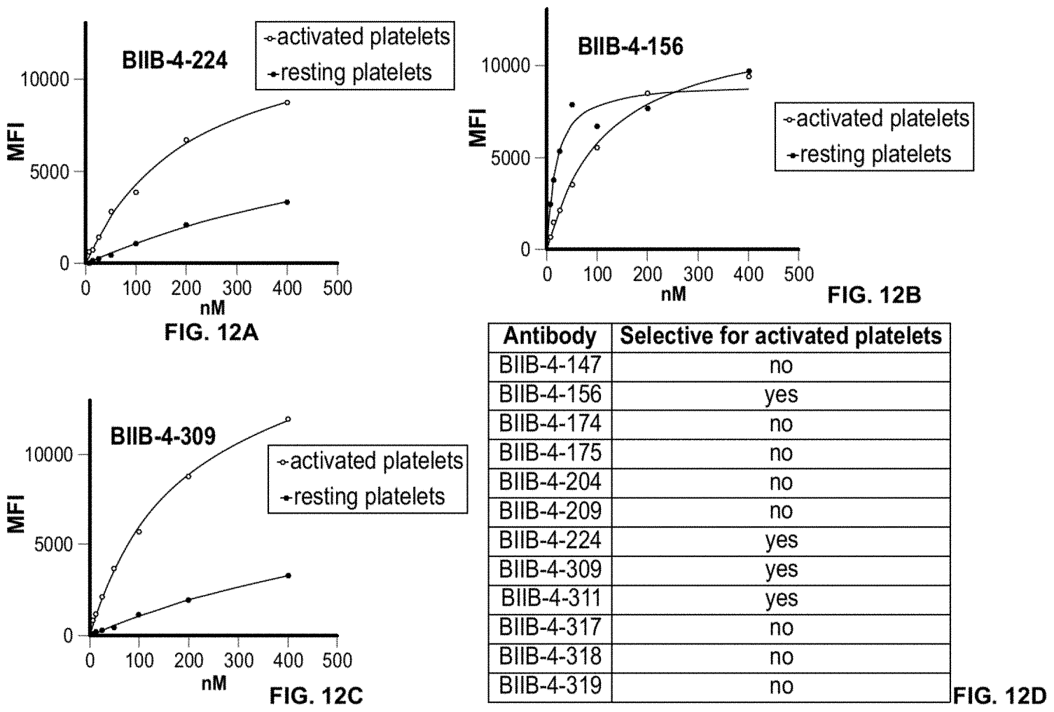

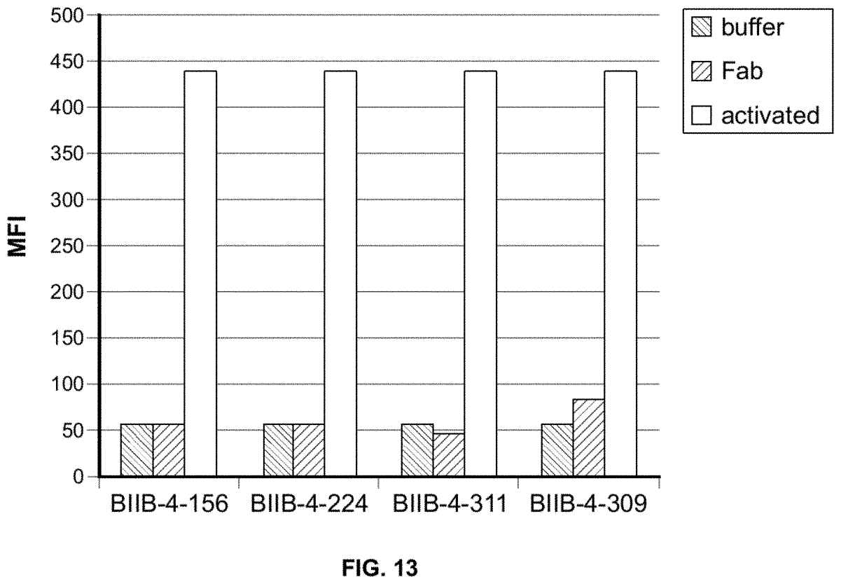

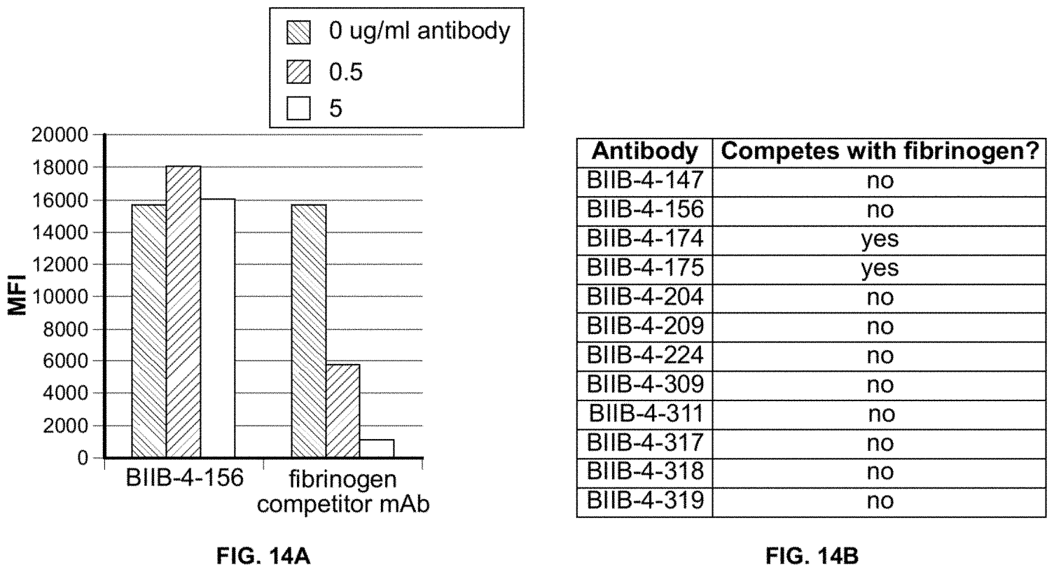

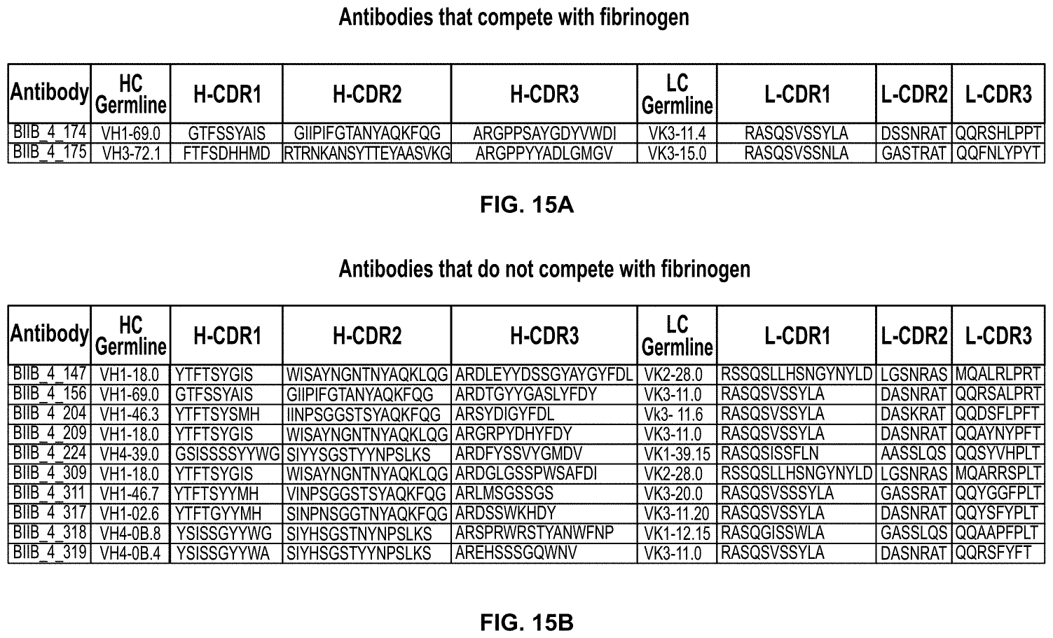

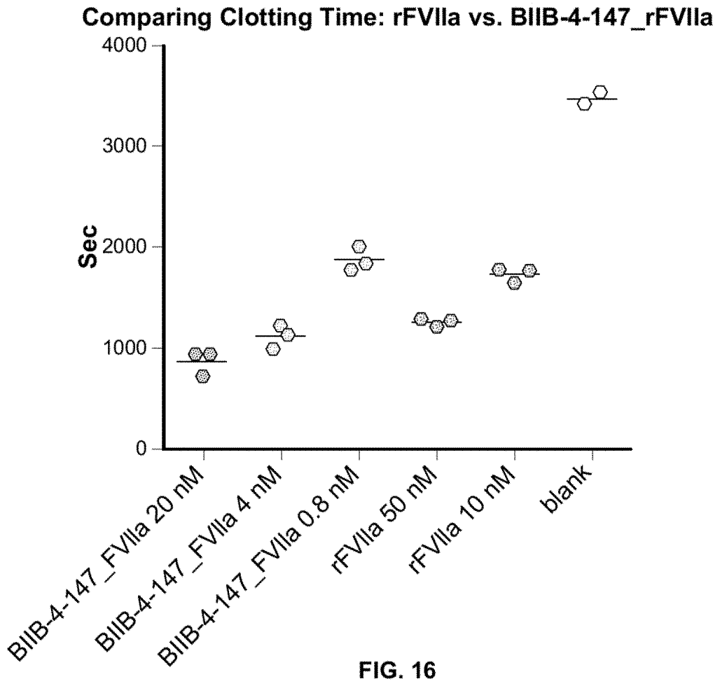

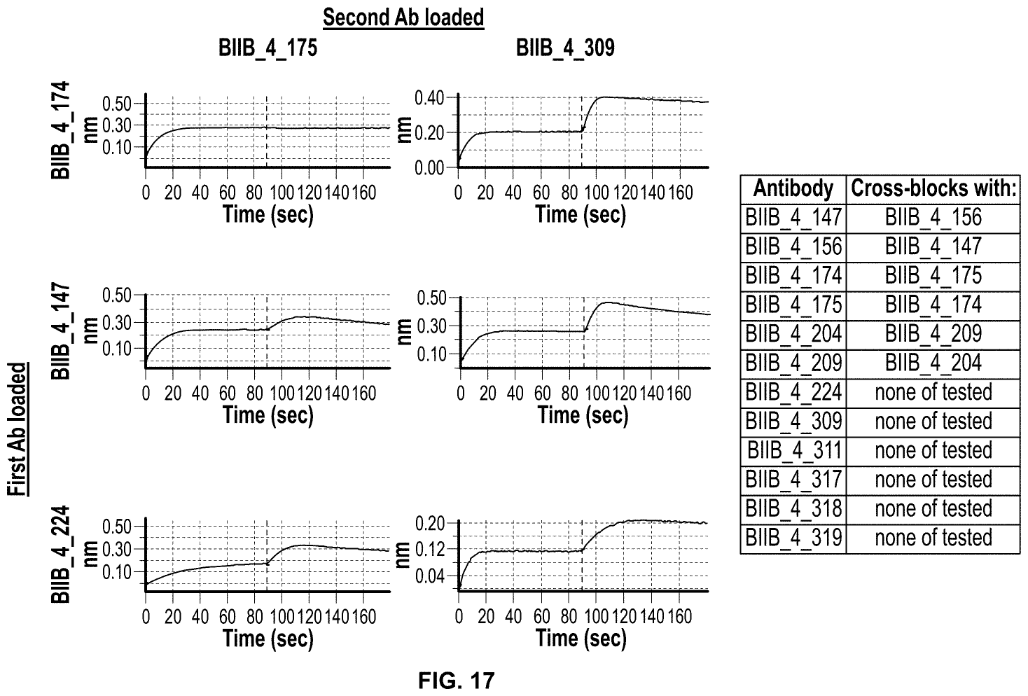

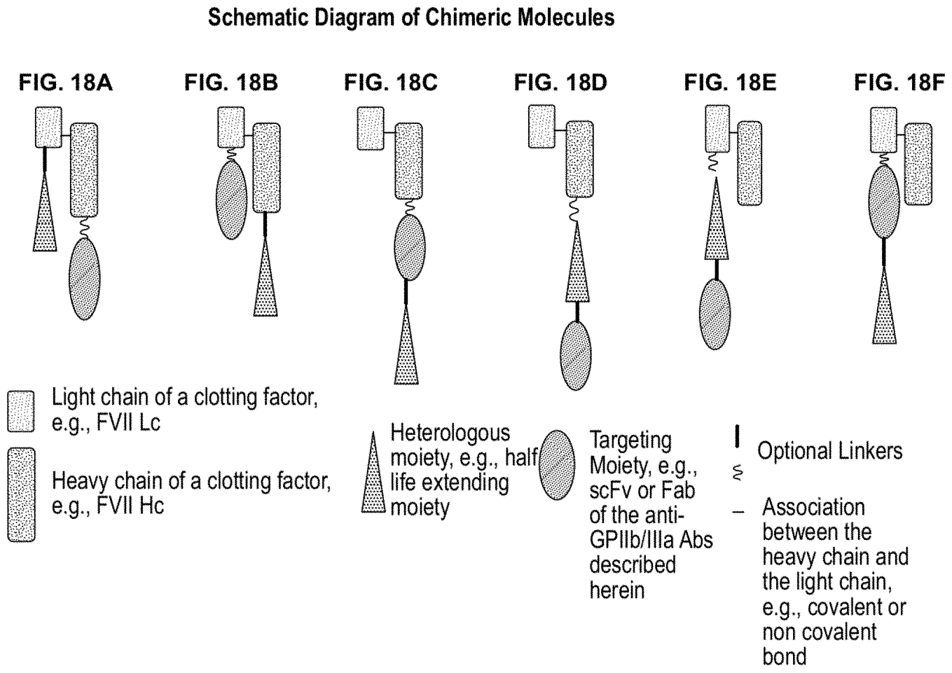

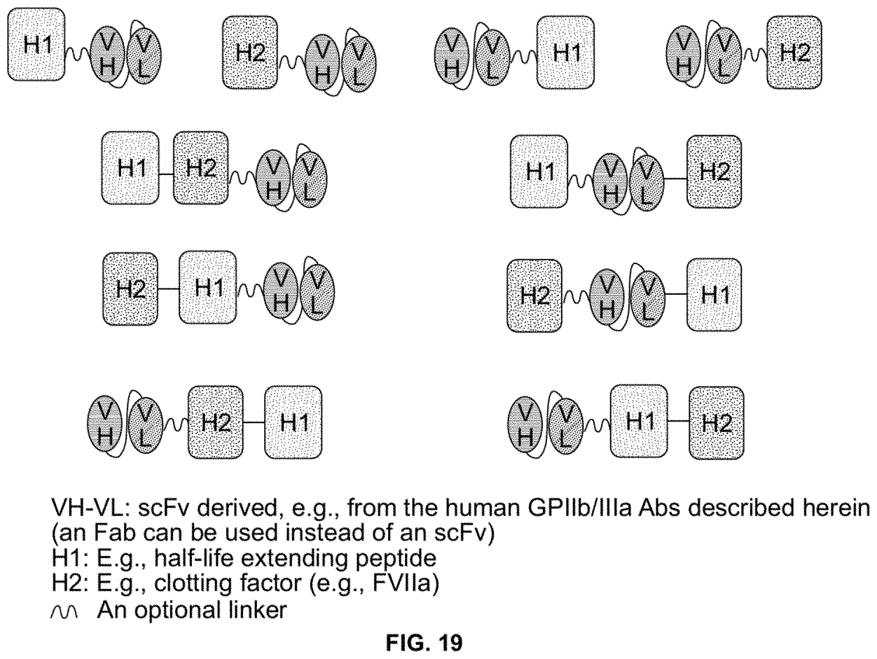

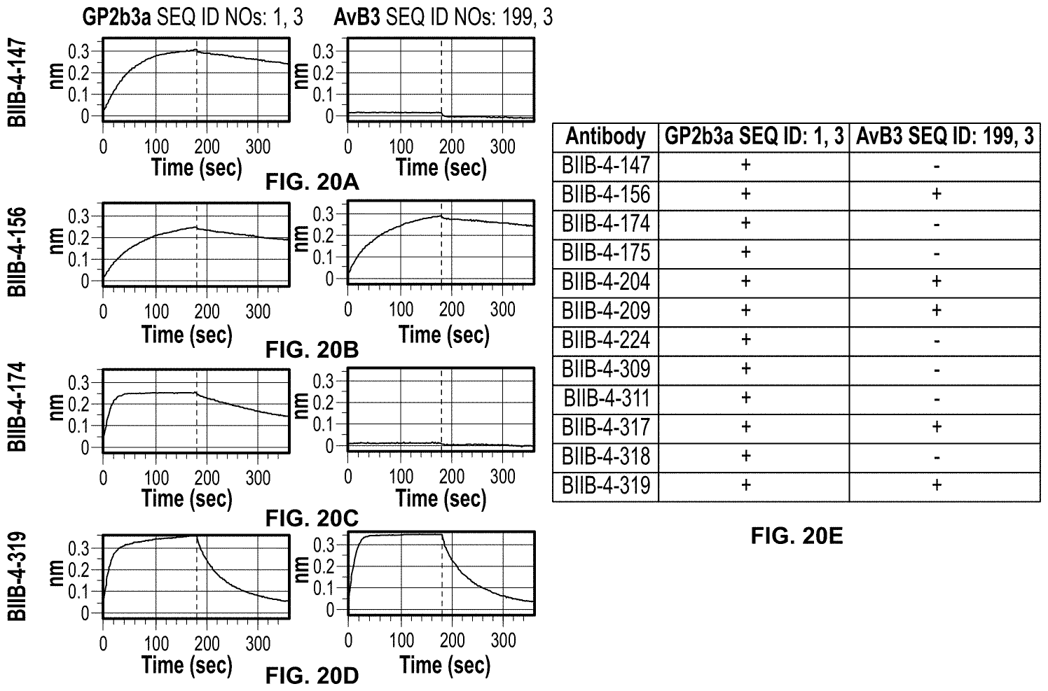

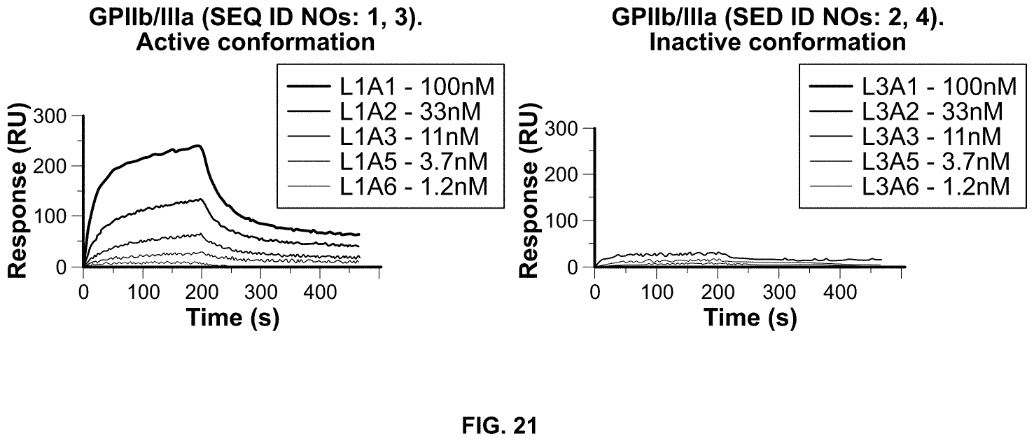

Example 1 of the application discloses twelve fully human anti-GPIIb/IIIa antibodies. The complementarity determining regions (CDRs) of these antibodies are provided in TABLE 1 below. This disclosure encompasses anti-GPIIb/IIIa antibodies or antigen binding fragments comprising or consisting of at least two, at least three, at least four, at least five or the six CDRs of each of the antibodies listed in Table 1. In addition, this disclosure encompasses anti-GPIIb/IIIa antibodies or antigen binding fragments comprising or consisting of the CDRs disclosed in Table 1 with at least seven, at least six, at least five, at least four, at least three, at least two, or one substitutions, deletions, and/or insertions in one, two, three, four, five or all six CDRs. Identifying amino acids for substitution(s), deletion(s), and/or insertion(s) in a CDR of an anti-GPIIb/IIIa antibody or antigen-binding fragment thereof can be done by aligning the amino acid sequences of the CDRs (especially closely related CDR sequences) and identify the variant amino acid sequences (see, e.g., FIGS. 3 and 4). The locations where variations occur especially in closely related sequences are the sites suitable for making amino acid substitution(s), deletion(s), and/or insertion(s). For example, if the VH-CDR1 sequence is from BIIB_4_147, i.e., YTFTSYGIS (SEQ ID NO: 53), by comparing that sequence with closely related VH-CDR1 sequences in FIG. 3, one could, e.g., make an amino acid substitution of Gin SEQ ID NO:53 to S, Y, A, or H by looking at the other residues occupying that position in other VH-CDR1 sequences. Similarly one could, e.g., make an amino acid substitution of I in SEQ ID NO:53 to M or W by looking at the other residues occupying that position in other VH-CDR1 sequences. In addition, one could, e.g., make an amino acid substitution of the C-terminal “S” in SEQ ID NO:53 to H or D, and the N-terminal Y to G. The anti-GPIIb/IIIa antibodies can include the CDRs described herein in the context of any suitable heavy and light chain human acceptor framework. In some instances, the heavy chain framework is from VH1-18.0, VH1-69.0, VH3-72.1, VH1-46.3, VH4-39.0, VH1-46.7, VH1-02.6, VH4-0B.4, or VH4-0B.8. In some instances, the light chain framework is from VK2-28.0, VK3-11.0, VK3-11.4, VK3-15.0, VK3-11.6, VK1-39.15, VK3-20.0, VK3-11.20, or VK1-12.15.

TABLE 1
VH and VL CDR Sequences of Exemplary Antibodies

| Antibody | Germlines | Sequence |
|---|---|---|
| BIIB_4_147 | HC: VH1-18.0; | VH-CDR1: |
| | LC: VK2-28.0 | YTFTSYGIS |
| | | (SEQ ID NO: 53) |
| | | VH-CDR2: |
| | | WISAYNGNTNYAQKLQG |
| | | (SEQ ID NO: 54) |
| | | VH-CDR3: |
| | | ARDLEYYDSSGYAYGYFDL |
| | | (SEQ ID NO: 55) |
| | | VL-CDR1: |
| | | RSSQSLLHSNGYNYLD |
| | | (SEQ ID NO: 83) |
| | | VL-CDR2: |
| | | LGSNRAS |
| | | (SEQ ID NO: 84) |
| | | VL-CDR3: |
| | | MQALRLPRT |
| | | (SEQ ID NO: 85) |
| BIIB_4_156 | HC: VH 1-69.0; | VH-CDR1: |
| | LC: VK3-11.0 | GTFSSYAIS |
| | | (SEQ ID NO: 56) |
| | | VH-CDR2: |
| | | GIIPIFGTANYAQKFQG |
| | | (SEQ ID NO: 57) |
| | | VH-CDR3: |
| | | ARDTGYYGASLYFDY |
| | | (SEQ ID NO: 58) |
| | | VL-CDR1: |
| | | RASQSVSSYLA |
| | | (SEQ ID NO: 86) |
| | | VL-CDR2: |
| | | DASNRAT |
| | | (SEQ ID NO: 87) |
| | | VL-CDR3: |
| | | QQRSALPRT |
| | | (SEQ ID NO: 88) |
| BIIB_4_174 | HC: VH 1-69.0: | VH-CDR1: |
| | LC: VK3-11.4 | GTFSSYAIS |
| | | (SEQ ID NO: 56) |
| | | VH-CDR2: |
| | | GIIPIFGTANYAQKFQG |
| | | (SEQ ID NO: 57) |
| | | VH-CDR3: |
| | | ARGPPSAYGDYVWDI |
| | | (SEQ ID NO: 59) |
| | | VL-CDR1: |
| | | RASQSVSSYLA |
| | | (SEQ ID NO: 86) |
| | | VL-CDR2: |
| | | DSSNRAT |
| | | (SEQ ID NO: 89) |
| | | VL-CDR3: |
| | | QQRSHLPPT |
| | | (SEQ ID NO: 90) |
| BIIB_4_175 | HC: VH3-72.1; | VH-CDR1: |
| | LC: VK3-15.0 | FTFSDHHMD |
| | | (SEQ ID NO: 60) |
| | | VH-CDR2: |
| | | RTRNKANSYTTEYAASVKG |
| | | (SEQ ID NO: 61) |
| | | VH-CDR3: |
| | | ARGPPYYADLGMGV |
| | | (SEQ ID NO: 62) |
| | | VL-CDR1: |
| | | RASQSVSSNLA |
| | | (SEQ ID NO: 91) |
| | | VL-CDR2: |
| | | GASTRAT |
| | | (SEQ ID NO: 92) |
| | | VL-CDR3: |
| | | QQFNLYPYT |
| | | (SEQ ID NO: 93) |
| BI1B_4_204 | HC: VH1-46.3; | VH-CDR1: |
| | LC: VK3-11.6 | YTFTSYSMH |
| | | (SEQ ID NO: 63) |
| | | VH-CDR2: |
| | | IINPSGGSTSYAQKFQG |
| | | (SEQ ID NO: 64) |
| | | VH-CDR3: |
| | | ARSYDIGYFDL |
| | | (SEQ ID NO: 65) |
| | | VL-CDR1: |
| | | RASQSVSSYLA |
| | | (SEQ ID NO: 86) |
| | | VL-CDR2: |
| | | DASKRAT |
| | | (SEQ ID NO: 94) |
| | | VL-CDR3: |
| | | QQDSFLPFT |
| | | (SEQ ID NO: 95) |
| BIIB_4_209 | HC: VH1-18.0; | VH-CDR1: |
| | LC: VK3-11.0 | YTFTSYGIS |
| | | (SEQ ID NO: 53) |
| | | VH-CDR2: |
| | | WISAYNGNTNYAQKLQG |
| | | (SEQ ID NO: 54) |
| | | VH-CDR3: |
| | | ARGRPYDHYFDY |
| | | (SEQ ID NO: 66) |
| | | VL-CDR1: |
| | | RASQSVSSYLA |
| | | (SEQ ID NO: 86) |
| | | VL-CDR2: |
| | | DASNRAT |
| | | (SEQ ID NO: 87) |
| | | VL-CDR3: |
| | | QQAYNYPFT |
| | | (SEQ ID NO: 96) |
| BI1B_4_224 | HC: VH4-39.0; | VH-CDR1: |
| | LC: VK1-39.15 | GSISSSSYYWG |
| | | (SEQ ID NO: 67) |
| | | VH-CDR2: |
| | | SIYYSGSTYYNPSLKS |
| | | (SEQ ID NO: 68) |
| | | VH-CDR3: |
| | | ARDFYSSVYGMDV |
| | | (SEQ ID NO: 69) |
| | | VL-CDR1: |
| | | RASQSISSFLN |
| | | (SEQ ID NO: 97) |
| | | VL-CDR2: |
| | | AASSLQS |
| | | (SEQ ID NO: 98) |
| | | VL-CDR3: |
| | | QQSYVHPLT |
| | | (SEQ ID NO: 99) |
| BIIB_4_309 | HC: VH 1-18.0; | VH-CDR1: |
| | LC: VK2-28.0 | YTFTSYGIS |
| | | (SEQ ID NO: 53) |
| | | VH-CDR2: |
| | | WISAYNGNTNYAQKLQG |
| | | (SEQ ID NO: 54) |
| | | VH-CDR3: |
| | | ARDGLGSSPWSAFDI |
| | | (SEQ ID NO: 70) |
| | | VL-CDR1: |
| | | RSSQSLLHSNGYNYLD |
| | | (SEQ ID NO: 100) |
| | | VL-CDR2: |
| | | LGSNRAS |
| | | (SEQ ID NO: 101) |
| | | VL-CDR3: |
| | | MQARRSPLT |
| | | (SEQ ID NO: 102) |
| BIIB_4_311 | HC: VH1-46.7; | VH-CDR1: |
| | LC: VK3-20.0 | YTFTSYYMH |
| | | (SEQ ID NO: 71) |
| | | VH-CDR2: |
| | | VINPSGGSTSYAQKFQG |
| | | (SEQ ID NO: 72) |
| | | VH-CDR3: |
| | | ARLMSGSSGS |
| | | (SEQ ID NO: 73) |
| | | VL-CDR1: |
| | | RASQSVSSSYLA |
| | | (SEQ ID NO: 103) |
| | | VL-CDR2: |
| | | GASSRAT |
| | | (SEQ ID NO: 104) |
| | | VL-CDR3: |
| | | QQYGGFPLT |
| | | (SEQ ID NO: 105) |
| BIIB_4_317 | HC: VH 1-02.6; | VH-CDR1: |
| | LC: VK3-11.20 | YTFTGYYMH |
| | | (SEQ ID NO: 74) |
| | | VH-CDR2: |
| | | SINPNSGGTNYAQKFQG |
| | | (SEQ ID NO: 75) |
| | | VH-CDR3: |
| | | ARDSSWKHDY |
| | | (SEQ ID NO: 76) |
| | | VL-CDR1: |
| | | RASQSVSSYLA |
| | | (SEQ ID NO: 86) |
| | | VL-CDR2: |
| | | DASNRAT |
| | | (SEQ ID NO: 87) |
| | | VL-CDR3: |
| | | QQYSFYPLT |
| | | (SEQ ID NO: 106) |
| BIIB_4_318 | HC: VH4-0B.8; | VH-CDR1: |
| | LC: VK1-12.15 | YSISSGYYWG |
| | | (SEQ ID NO: 77) |
| | | VH-CDR2: |
| | | SIYHSGSTNYNPSLKS |
| | | (SEQ ID NO: 78) |
| | | VH-CDR3: |
| | | ARSPRVVRSTYANWFNP |
| | | (SEQ ID NO: 79) |
| | | VL-CDR1: |
| | | RASQGISSWLA |
| | | (SEQ ID NO: 107) |
| | | VL-CDR2: |
| | | GASSLQS |
| | | (SEQ ID NO: 108) |
| | | VL-CDR3: |
| | | QQAAPFPLT |
| | | (SEQ ID NO: 109) |
| BIIB_4_319 | HC: VH4-0B.4; | VH-CDR1: |
| | LC: VK3-11.0 | YSISSGYYWA |
| | | (SEQ ID NO: 80) |
| | | VH-CDR2: |
| | | SIYHSGSTYYNPSLKS |
| | | (SEQ ID NO: 81) |
| | | VH-CDR3: |
| | | AREHSSSGQWNV |
| | | (SEQ ID NO: 82) |
| | | VL-CDR1: |
| | | RASQSVSSYLA |
| | | (SEQ ID NO: 86) |
| | | VL-CDR2: |
| | | DASNRAT |
| | | (SEQ ID NO: 87) |
| | | VL-CDR3: |
| | | QQRSFYFT |
| | | (SEQ ID NO: 110) |

HC = heavy chain; LC = light chain.

Although Table 1 discloses the CDRs according to Kabat (Kabat et al., Sequences of Proteins of Immunological Interest, 5th Ed. Public Health Service, National Institutes of Health, Bethesda, Md. (1991)), the antibodies of this disclosure can comprise CDRs of an anti-GPIIb/IIIa antibody disclosed herein according to any CDR definition (e.g., Kabat, Chothia, enhanced Chothia, contact, IMGT, AbM). The CDRs of an antibody according to the different CDR definitions can be determined, e.g., by using the AbYsis database (www.bioinforg.uk/abysis/sequence_input/key_annotation/key_annotation.cgi). According to the classical Kabat numbering, Kabat VH-CDR1 is at positions 31-35, VH-CDR2 is a positions 50-65, and VH-CDR3 is at positions 95-102; and, VL-CDR1, VL-CDR2, and VL-CDR3 are at positions 24-34, 50-56 and 89-97, respectively. According to the Chothia definition, VH-CDR1 is at positions 26-32 (Chothia numbering), VH-CDR2 is at positions 52-56, VH-CDR3 is at positions 95-102, VL-CDR1 is at positions 24-34, VL-CDR2 is at positions 50-56, and VL-CDR3 is at positions 89-97. According to the contact definition, VH-CDR1 is at positions 30-35 (Chothia numbering), VH-CDR2 is at positions 47-58, VH-CDR3 is at positions 93-101, VL-CDR1 is at positions 30-36, VL-CDR2 is at positions 46-55, and VL-CDR3 is at positions 89-96. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

The anti-GPIIb/IIIa antibodies and antigen binding fragments of this disclosure can be divided into at least the following three classes:

Class I: antibodies and antigen binding fragments that preferentially bind GPIIb/IIIa on activated platelets compared to GPIIb/IIIa on resting platelets and that do not activate the platelets. In some embodiments, they also do not compete with fibrinogen for binding GPIIb/IIIa. These antibodies can preferentially bind to the heterodimer formed by the amino acid sequences set forth in SEQ ID NOs.: 1 and 3 over the heterodimer formed by the amino acid sequences set forth in SEQ ID NOs.: 2 and 4. Examples include antibodies designated: BIIB-4-156, BIIB-4-224, BIIB-4-309, and BIIB-4-311 (see, FIG. 8).

Class II: antibodies and antigen binding fragments that are not selective with respect to binding GPIIb/IIIa on resting versus activated platelets, that do not activate the platelets, and that do not compete with fibrinogen for binding GPIIb/IIIa. These antibodies do not show a preference for binding to the heterodimer formed by the amino acid sequences set forth in SEQ ID NOs.: 1 and 3 over the heterodimer formed by the amino acid sequences set forth in SEQ ID NOs.: 2 and 4. Examples include antibodies designated: BIIB-4-147, BIIB-4-204, BIIB-4-209, BIIB-4-317, BIIB-4-318, and BIIB-4-319 (see, FIG. 8).

Class III: antibodies and antigen binding fragments that are not selective with respect to binding GPIIb/IIIa on resting versus activated platelets, that do not activate the platelets, and that do compete with fibrinogen for binding GPIIb/IIIa. These antibodies do not show a preference for binding to the heterodimer formed by the amino acid sequences set forth in SEQ ID NOs.: 1 and 3 over the heterodimer formed by the amino acid sequences set forth in SEQ ID NOs.: 2 and 4. Examples include antibodies designated: BIIB-4-174 and BIIB-4-175, (see, FIG. 15).

In one embodiment, the anti-GPIIb/IIIa antibodies or antigen-binding fragments thereof of this disclosure preferentially bind to GPIIb/IIIa on activated vs. resting platelets and do not activate platelets. The platelets can be from a human subject. In certain instances, these antibodies or antigen-binding fragments thereof do not inhibit the association of fibrinogen with GPIIb/IIIa. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM. In some embodiments, these anti-GPIIb/IIIa antibodies or antigen-binding fragments thereof include at least one, at least two or three of the VH-CDR1, VH-CDR2, and VH-CDR3 of any one of BIIB-4-156, BIIB-4-224, BIIB-4-309, or BIIB-4-311, wherein these CDRs have a total of six, five, four, three, two, one or no substitutions, insertions and/or deletions in one, two, or three CDRs. In other embodiments these anti-GPIIb/IIIa antibodies or antigen-binding fragments thereof include at least one, at least two or three of the VL-CDR1, VL-CDR2, and VL-CDR3 of any one of BIIB-4-156, BIIB-4-224, BIIB-4-309, or BIIB-4-311, wherein these CDRs have a total of six, five, four, three, two, one or no substitutions, insertions and/or deletions in one, two, or three CDRs. In certain embodiments, these anti-GPIIb/IIIa antibodies or antigen-binding fragments thereof comprise at least four, at least five, or all six CDRs of any one of BIIB-4-156, BIIB-4-224, BIIB-4-309, or BIIB-4-311. In some embodiments, these anti-GPIIb/IIIa antibodies or antigen-binding fragments thereof comprise a VH domain having at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identity to the VH domain of any one of BIIB-4-156, BIIB-4-224, BIIB-4-309, or BIIB-4-311. In other embodiments, these anti-GPIIb/IIIa antibodies or antigen-binding fragments thereof comprise a VL domain having at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identity to the VL domain of any one of BIIB-4-156, BIIB-4-224, BIIB-4-309, or BIIB-4-311.

In another embodiment, the anti-GPIIb/IIIa antibodies or antigen-binding fragments thereof of this disclosure bind to GPIIb/IIIa on both resting and activated platelets (i.e., there is no preferential binding of the antibody or fragment to GPIIb/IIIa on activated or resting platelets) and do not activate the platelets. The platelets can be from a human subject. In certain instances, the anti-GPIIb/IIIa antibodies or antigen-binding fragments thereof bind to GPIIb/IIIa on both resting and activated platelets with the same or similar affinity. In some cases, the anti-GPIIb/IIIa antibodies or antigen-binding fragments thereof do not inhibit the interaction of fibrinogen with GPIIb/IIIa. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM. In some embodiments, these anti-GPIIb/IIIa antibodies or antigen-binding fragments thereof include at least one, at least two or three of the VH-CDR1, VH-CDR2, and VH-CDR3 of any one of BIIB-4-147, BIIB-4-204, BIIB-4-209, BIIB-4-317, BIIB-4-318, or BIIB-4-319, wherein these CDRs have a total of six, five, four, three, two, one or no substitutions, insertions and/or deletions in one, two, or three CDRs. In other embodiments these anti-GPIIb/IIIa antibodies or antigen-binding fragments thereof comprise at least one, at least two or three of the VL-CDR1, VL-CDR2, and VL-CDR3 of any one of BIIB-4-147, BIIB-4-204, BIIB-4-209, BIIB-4-317, BIIB-4-318, or BIIB-4-319, wherein these CDRs have a total of six, five, four, three, two, one or no substitutions, insertions and/or deletions in one, two, or three CDRs. In certain embodiments, these anti-GPIIb/IIIa antibodies or antigen-binding fragments thereof comprise at least four, at least five, or all six CDRs of any one of BIIB-4-147, BIIB-4-204, BIIB-4-209, BIIB-4-317, BIIB-4-318, or BIIB-4-319. In some embodiments, these anti-GPIIb/IIIa antibodies or antigen-binding fragments thereof comprise a VH domain having at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identity to the VH domain of any one of BIIB-4-147, BIIB-4-204, BIIB-4-209, BIIB-4-317, BIIB-4-318, or BIIB-4-319. In certain instances, these anti-GPIIb/IIIa antibodies or antigen-binding fragments thereof comprise a VL domain having at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identity to the VL domain of any one of BIIB-4-147, BIIB-4-204, BIIB-4-209, BIIB-4-317, BIIB-4-318, or BIIB-4-319.

In another embodiment, the anti-GPIIb/IIIa antibodies or antigen-binding fragments thereof of this disclosure bind to GPIIb/IIIa on both resting and activated platelets (i.e., there is no preferential binding of the antibody or fragment to GPIIb/IIIa on activated or resting platelets), do not activate the platelets, and inhibit the interaction of fibrinogen with GPIIb/IIIa. The platelets can be from a human subject. In certain instances, the anti-GPIIb/IIIa antibodies or antigen-binding fragments thereof bind to GPIIb/IIIa on both resting and activated platelets with the same or similar affinity. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM. In some embodiments, these anti-GPIIb/IIIa antibodies or antigen-binding fragments thereof include at least one, at least two or three of the VH-CDR1, VH-CDR2, and VH-CDR3 of any one of BIIB-4-174 or BIIB-4-175, wherein these CDRs have a total of six, five, four, three, two, one or no substitutions, insertions and/or deletions in one, two, or three CDRs. In other embodiments these anti-GPIIb/IIIa antibodies or antigen-binding fragments thereof comprise at least one, at least two or three of the VL-CDR1, VL-CDR2, and VL-CDR3 of any one of BIIB-4-174 or BIIB-4-175, wherein these CDRs have a total of six, five, four, three, two, one or no substitutions, insertions, and/or deletions in one, two, or three CDRs. In certain embodiments, these anti-GPIIb/IIIa antibodies or antigen-binding fragments thereof comprise at least four, at least five, or all six CDRs of any one of BIIB-4-174 or BIIB-4-175. In some embodiments, these anti-GPIIb/IIIa antibodies or antigen-binding fragments thereof comprise a VH domain having at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identity to the VH domain of any one of BIIB-4-174 or BIIB-4-175. In certain instances, these anti-GPIIb/IIIa antibodies or antigen-binding fragments thereof comprise a VL domain having at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identity to the VL domain of any one of BIIB-4-174 or BIIB-4-175.

In another embodiment, the anti-GPIIb/IIIa antibodies or antigen-binding fragments thereof of this disclosure bind preferentially to a GPIIb/IIIa heterodimer formed by the amino acid sequences set forth in SEQ ID NOS.: 1 and 3, compared with the GPIIb/IIIa heterodimer formed by the amino acid sequences set forth in SEQ ID NOS.: 2 and 4. These antibodies or antigen-binding fragments do not activate platelets. In some embodiments the platelets are from a human subject. In certain instances, these antibodies or antigen-binding fragments do not inhibit fibrinogen binding to GPIIb/IIIa. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM. In some embodiments, these anti-GPIIb/IIIa antibodies or antigen-binding fragments thereof have the VH-CDR1, VH-CDR2, and VH-CDR3 of any one of BIIB-4-156, BIIB-4-224, BIIB-4-309, or BIIB-4-311, wherein these CDRs have a total of six, five, four, three, two, one or no substitutions, insertions and/or deletions in one, two, or three CDRs. In other embodiments these anti-GPIIb/IIIa antibodies or antigen-binding fragments thereof comprise VL-CDR1, VL-CDR2, and VL-CDR3 of any one of BIIB-4-156, BIIB-4-224, BIIB-4-309, or BIIB-4-311, wherein these CDRs have a total of six, five, four, three, two, one or no substitutions, insertions, and/or deletions in one, two, or three CDRs. In certain embodiments, these anti-GPIIb/IIIa antibodies or antigen-binding fragments thereof comprise at least four, at least five, or all six CDRs of any one of BIIB-4-156, BIIB-4-224, BIIB-4-309, or BIIB-4-311. In some embodiments, these anti-GPIIb/IIIa antibodies or antigen-binding fragments thereof comprise a VH domain having at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identity to the VH domain of any one of BIIB-4-156, BIIB-4-224, BIIB-4-309, or BIIB-4-311. In other embodiments, these anti-GPIIb/IIIa antibodies or antigen-binding fragments thereof comprise a VL domain having at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identity to the VL domain of any one of BIIB-4-156, BIIB-4-224, BIIB-4-309, or BIIB-4-311.

The antibody or antigen-binding molecules thereof that specifically bind to a GPIIb/IIIa epitope, can comprise or overlap with the GPIIb/IIIa binding epitope of an anti-GPIIb/IIIa antibody comprising at least three CDRs of the VH domain, at least four CDRs, at least five CDRs, all six CDRs, the VH domain, or the VH and VL domains of an antibody selected from BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. In some embodiments, the anti-GPIIb/IIIa antibody or antigen-binding molecules thereof specifically bind to a GPIIb/IIIa epitope, which is the same GPIIb/IIIa binding epitope of an anti-GPIIb/IIIa antibody comprising three CDRs of the VH domain, at least four CDRs, at least five CDRs, all six CDRs, the VH domain, or the VH and VL domains of an antibody selected from BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM.

The antibody or antigen-binding molecules thereof that specifically bind to a GPIIb/IIIa epitope, can competitively inhibit or cross block GPIIb/IIIa binding by an anti-GPIIb/IIIa antibody comprising at least three CDRs of the VH domain, at least four CDRs, at least five CDRs, all six CDRs, the VH domain, or the VH and VL domains of an antibody selected from BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM.

In certain embodiments, the antibody or antigen-binding molecule thereof which specifically binds to a GPIIb/IIIa epitope comprises:
(i) a variable heavy chain CDR-1 (VH-CDR1) sequence at least about 60%, 70%, 80%, 90%, 95%, or 100% identical to VH-CDR1 of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319;
(ii) a variable heavy chain CDR-2 (VH-CDR2) sequence at least about 60%, 70%, 80%, 90%, 95%, or 100% identical to VH-CDR2 of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319; and
(iii) a variable heavy chain CDR-3 (VH-CDR3) sequence at least about 60%, 70%, 80%, 90%, 95%, or 100% identical to VH-CDR3 of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319.
   In some instances, the above anti-GPIIb/IIIa antibodies or antigen-binding fragments further comprise at least one, at least two, or all three of the CDRs of the VL domain of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319.

In certain embodiments, the antibody or antigen-binding molecule thereof which specifically binds to a GPIIb/IIIa epitope comprises:
(i) a variable light chain CDR-1 (VL-CDR1) sequence at least about 60%, 70%, 80%, 90%, 95%, or 100% identical to VL-CDR1 of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319;
(ii) a variable light chain CDR-2 (VL-CDR2) sequence at least about 60%, 70%, 80%, 90%, 95%, or 100% identical to VL-CDR2 of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319; and
(iii) a variable light chain CDR-3 (VH-CDR3) sequence at least about 60%, 70%, 80%, 90%, 95%, or 100% identical to VL-CDR3 of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319.
   In some instances, the above anti-GPIIb/IIIa antibodies or antigen-binding fragments further comprise at least one, at least two, or all three of the CDRs of the VH domain of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319.

In certain embodiments, the anti-GPIIb/IIIa antibody or antigen-binding molecule thereof which specifically binds to a GPIIb/IIIa epitope comprises:
(i) a variable heavy chain CDR-1 (VH-CDR1) sequence at least about 60%, 70%, 80%, 90%, 95%, or 100% identical to VH-CDR1 of an antibody selected from BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319;
(ii) a variable heavy chain CDR-2 (VH-CDR2) sequence at least about 60%, 70%, 80%, 90%, 95%, or 100% identical to VH-CDR2 of an antibody selected from BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319;
(iii) a variable heavy chain CDR-3 (VH-CDR3) sequence at least about 60%, 70%, 80%, 90%, 95%, or 100% identical to VH-CDR3 of an antibody selected from BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319;
(iv) a variable light chain CDR-1 (VL-CDR1) sequence at least about 60%, 70%, 80%, 90%, 95%, or 100% identical to VL-CDR1 of an antibody selected from BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319;
(v) a variable light chain CDR-2 (VL-CDR2) sequence at least about 60%, 70%, 80%, 90%, 95%, or 100% identical to VL-CDR2 of an antibody selected from BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319, and/or
(vi) a variable light chain CDR-3 (VL-CDR3) sequence at least about 60, 70, 80, 90, or 95% identical to VL-CDR3 of an antibody selected from BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319.

In certain embodiments, the anti-GPIIb/IIIa antibody or antigen-binding molecule thereof which specifically binds to a GPIIb/IIIa epitope comprises:
(i) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-147 antibody;
(ii) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-156 antibody;
(iii) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-174 antibody;
(iv) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-175 antibody;
(v) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-204 antibody;
(vi) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-209 antibody;
(vii) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-224 antibody;
(viii) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-309 antibody;
(ix) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-311 antibody;
(x) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-317 antibody;
(xi) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-318 antibody; or
(xii) VH-CDR1, VH-CDR2, VH-CDR3, VL-CDR1, VL-CDR2, and VL-CDR3 sequences at least 60%, at least 65%, at least 70%, at least 75%, at least 80%, at least 85%, at least 86%, at least 87%, at least 88%, at least 89% at least 90%, at least 95%, at least 96%, at least 97%, at least 98%, or 100% identical to the VH-CDR1, VH-CDR2, VH-CDR3, VL-CDR1, VL-CDR2, and VL-CDR3 sequences of BIIB-4-319 antibody.
   In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM.

In certain embodiments, the anti-GPIIb/IIIa antibody or antigen-binding molecule thereof which specifically binds to a GPIIb/IIIa epitope comprises:
(i) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-147 antibody except for a total of six, five, four, three, two, or one amino acid substitutions, deletions and/or insertions in six, five, four, three, two, or one of these CDRs;
(ii) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-156 antibody except for a total of six, five, four, three, two, or one amino acid substitutions, deletions and/or insertions in six, five, four, three, two, or one of these CDRs;
(iii) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-174 antibody except for a total of six, five, four, three, two, or one amino acid substitutions, deletions and/or insertions in six, five, four, three, two, or one of these CDRs;
(iv) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-175 antibody except for a total of six, five, four, three, two, or one amino acid substitutions, deletions and/or insertions in six, five, four, three, two, or one of these CDRs;
(v) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-204 antibody except for a total of six, five, four, three, two, or one amino acid substitutions, deletions and/or insertions in six, five, four, three, two, or one of these CDRs;
(vi) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-209 antibody except for a total of six, five, four, three, two, or one amino acid substitutions, deletions and/or insertions in six, five, four, three, two, or one of these CDRs;
(vii) VH-CDR1, VH-CDR2, VH-CDR3, VL-CDR1, VL-CDR2, and VL-CDR3 that are identical to the VH-CDR1, VH-CDR2, VH-CDR3, VL-CDR1, VL-CDR2, and VL-CDR3 sequences of BIIB-4-224 antibody except for a total of six, five, four, three, two, or one amino acid substitutions, deletions and/or insertions in six, five, four, three, two, or one of these CDRs;
(viii) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-309 antibody except for a total of six, five, four, three, two, or one amino acid substitutions, deletions and/or insertions in six, five, four, three, two, or one of these CDRs;
(ix) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-311 antibody except for a total of six, five, four, three, two, or one amino acid substitutions, deletions and/or insertions in six, five, four, three, two, or one of these CDRs;
(x) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-317 antibody;
(xi) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-318 antibody except for a total of six, five, four, three, two, or one amino acid substitutions, deletions and/or insertions in six, five, four, three, two, or one of these CDRs; or
(xii) VH-CDR1, VH-CDR2, VH-CDR3, VL-CDR1, VL-CDR2, and VL-CDR3 sequences that are identical to the VH-CDR1, VH-CDR2, VH-CDR3, VL-CDR1, VL-CDR2, and VL-CDR3 sequences of BIIB-4-319 antibody except for a total of six, five, four, three, two, or one amino acid substitutions, deletions and/or insertions in six, five, four, three, two, or one of these CDRs.
   In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM.

In certain embodiments, the anti-GPIIb/IIIa antibody or antigen-binding fragment thereof which specifically binds to a GPIIb/IIIa epitope comprises:
(i) a VH-CDR1 comprising the consensus amino acid sequence X₁TFX₂X₃YX₄X₅X₆, wherein X₁ is Y or G; X₂ is T or S; X₃ is S or G; X₄ is G, A, S, or Y; X₅ is I, M, or H; and X₆ is S or H (SEQ ID NO:111); or X₁TFX₂X₃YX₄IS, wherein X₁ is Y or G; X₂ is T or S; X₃ is S or G; X₄ is G or A (SEQ ID NO: 112);
(ii) a VH-CDR2 comprising the consensus amino acid sequence X₁INPX₂X₃ GX₄TX₅YAQKFQG, wherein X₁ is I, V, or S; X₂ is S or N; X₃ is G or S; X₄ is S or G; X₅ or S or N (SEQ ID NO:113); or X₁INPSGGSTSYAQKFQG, wherein X₁ is I or V (SEQ ID NO:114); and
(iii) a VH-CDR3 comprising VH-CDR3 of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319.
   These antibodies do not activate platelets. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM.

In other embodiments, the anti-GPIIb/IIIa antibody or antigen-binding fragment thereof which specifically binds to GPIIb/IIIa comprises:
(i) a VH-CDR1 comprising the consensus amino acid sequence X₁SISSGYYWX₂, wherein X₁ is Y or G; and X₂ is G or A (SEQ ID NO:115); or X₁SISSX₂X₃YYWG, wherein X₁ is Y or G; X₂ is G or S; X₃ is S or absent (SEQ ID NO: 116);
(ii) a VH-CDR2 comprising the consensus amino acid sequence SIYHSGSTX₁YNPSLKS, wherein X₁ is N or Y (SEQ ID NO:117); and
(iii) a VH-CDR3 comprising VH-CDR3 of an antibody selected from the group consisting of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319.
   These antibodies do not activate platelets. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM.

In some instances of the above two embodiments, the anti-GPIIb/IIIa antibodies or antigen-binding fragments thereof which specifically binds to GPIIb/IIIa further comprises:

(i) a VL-CDR1 comprising the consensus amino acid sequence RASQX₁X₂SSX₃X₄LX₅, wherein X₁ is S or G; X₂ is V or I; X₃ is S or absent; X₄ is Y, N, F, or W; and X₅ is A or N(SEQ ID NO: 118); and/or
(ii) a VL-CDR2 comprising the consensus amino acid sequence X₁X₂SX₃RAX₄, wherein X₁ is D, G, or L; X₂ is A, S, or G; X₃ is N, T, S, or K; and X₄ is T or S (SEQ ID NO: 119); and/or

(iii) a VL-CDR3 comprising the consensus amino acid sequence X₁QX₂X₃X₄X₅PX₆T, wherein X₁ is Q or M; X₂ is A, S, D, Y, F, or R; X₃ is A, Y, S, L, R, G, or N; X₄ is P, V, F, R, G, L, N, A or H; X₅ is F, H, Y, L, or S; and X₆ is L, F, R, Y, or P (SEQ ID NO: 120).

These antibodies do not activate platelets. In certain embodiments, these antibodies and antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM.

The anti-GPIIb/IIIa antibody or antigen binding fragment can comprise a VH region comprising VH-CDR1, VH-CDR2, and VH-CDR3 domains, wherein:
(i) VH-CDR1, VH-CDR2, VH-CDR3 domains comprise or consist of amino acid sequences set forth in SEQ ID NOs.: 53, 54, and 55, respectively;
(ii) VH-CDR1, VH-CDR2, VH-CDR3 domains comprise or consist of amino acid sequences set forth in SEQ ID NOs.: 56, 57, and 58, respectively;
(iii) VH-CDR1, VH-CDR2, VH-CDR3 domains comprise or consist of amino acid sequences set forth in SEQ ID NOs.: 56, 57, and 59, respectively;
(iv) VH-CDR1, VH-CDR2, VH-CDR3 domains comprise or consist of amino acid sequences set forth in SEQ ID NOs.: 60, 61, and 62, respectively;
(v) VH-CDR1, VH-CDR2, VH-CDR3 domains comprise or consist of amino acid sequences set forth in SEQ ID NOs.: 63, 64, and 65, respectively;
(vi) VH-CDR1, VH-CDR2, VH-CDR3 domains comprise or consist of amino acid sequences set forth in SEQ ID NOs.: 53, 54, and 66, respectively;
(vii) VH-CDR1, VH-CDR2, VH-CDR3 domains comprise or consist of amino acid sequences set forth in SEQ ID NOs.: 67, 68, and 69, respectively;
(viii) VH-CDR1, VH-CDR2, VH-CDR3 domains comprise or consist of amino acid sequences set forth in SEQ ID NOs.: 53, 54, and 70, respectively;
(ix) VH-CDR1, VH-CDR2, VH-CDR3 domains comprise or consist of amino acid sequences set forth in SEQ ID NOs.: 71, 72, and 73, respectively;
(x) VH-CDR1, VH-CDR2, VH-CDR3 domains comprise or consist of amino acid sequences set forth in SEQ ID NOs.: 74, 75, and 76, respectively;
(xi) VH-CDR1, VH-CDR2, VH-CDR3 domains comprise or consist of amino acid sequences set forth in SEQ ID NOs.: 77, 78, and 79, respectively; or
(xii) VH-CDR1, VH-CDR2, VH-CDR3 domains comprise or consist of amino acid sequences set forth in SEQ ID NOs.: 80, 81, and 82, respectively.
   In certain embodiments, the anti-GPIIb/IIIa antibody or antigen binding fragment described above can further comprise a VL region comprising at least one, at least two, or all three of the VL-CDR1, VL-CDR2, and VL-CDR3 domains, wherein:
(i) VL-CDR1, VL-CDR2, and VL-CDR3 domains comprise or consist of amino acid sequences set forth in SEQ ID NOs.: 83, 84, and 85, respectively;
(ii) VL-CDR1, VL-CDR2, and VL-CDR3 domains comprise or consist of amino acid sequences set forth in SEQ ID NOs.: 86, 87, and 88, respectively;
(iii) VL-CDR1, VL-CDR2, and VL-CDR3 domains comprise or consist of amino acid sequences set forth in SEQ ID NOs.: 86, 89, and 90, respectively;
(iv) VL-CDR1, VL-CDR2, and VL-CDR3 domains comprise or consist of amino acid sequences set forth in SEQ ID NOs.: 91, 92, and 93, respectively;
(v) VL-CDR1, VL-CDR2, and VL-CDR3 domains comprise or consist of amino acid sequences set forth in SEQ ID NOs.: 86, 94, and 95, respectively;
(vi) VL-CDR1, VL-CDR2, and VL-CDR3 domains comprise or consist of amino acid sequences set forth in SEQ ID NOs.: 86, 87, and 96, respectively;
(vii) VL-CDR1, VL-CDR2, and VL-CDR3 domains comprise or consist of amino acid sequences set forth in SEQ ID NOs.: 97, 98, and 99, respectively;
(viii) VL-CDR1, VL-CDR2, and VL-CDR3 domains comprise or consist of amino acid sequences set forth in SEQ ID NOs.: 100, 101, and 102, respectively;
(ix) VL-CDR1, VL-CDR2, and VL-CDR3 domains comprise or consist of amino acid sequences set forth in SEQ ID NOs.: 103, 104, and 105, respectively;
(x) VL-CDR1, VL-CDR2, and VL-CDR3 domains comprise or consist of amino acid sequences set forth in SEQ ID NOs.: 86, 87, and 106, respectively;
(xi) VL-CDR1, VL-CDR2, and VL-CDR3 domains comprise or consist of amino acid sequences set forth in SEQ ID NOs.: 107, 108, and 109, respectively; or
(xii) VL-CDR1, VL-CDR2, and VL-CDR3 domains comprise or consist of amino acid sequences set forth in SEQ ID NOs.: 86, 87, and 110, respectively.

The anti-GPIIb/IIIa antibodies or antigen binding fragments of this disclosure can comprise, consist essentially of, or consist of a heavy chain variable domain (VH) comprising, consisting essentially of, or consisting of an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to an amino acid sequence set forth in any one of SEQ ID NOs.: 5, 9, 13, 17, 21, 25, 29, 33, 37, 41, 45, and 49.

The anti-GPIIb/IIIa antibodies or antigen binding fragments of this disclosure can comprise, consist essentially of, or consist of a light chain variable domain (VL) comprising, consisting essentially of, or consisting of an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to an amino acid sequence set forth in any one of SEQ ID NOs.: 7, 11, 15, 19, 23, 27, 31, 35, 39, 43, 47, and 51.

In certain embodiments, the anti-GPIIb/IIIa antibodies or antigen binding fragments of this disclosure can comprise, consist essentially of, or consist of a heavy chain variable domain (VH) comprising, consisting essentially of, or consisting of an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to an amino acid sequence set forth in any one of SEQ ID NOs.: 5, 9, 13, 17, 21, 25, 29, 33, 37, 41, 45, and 49, and further comprise, consist essentially of, or consist of a light chain variable domain (VL) comprising, consisting essentially of, or consisting of an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to an amino acid sequence set forth in any one of SEQ ID NOs.: 7, 11, 15, 19, 23, 27, 31, 35, 39, 43, 47, and 51.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 5 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 7. These anti-GPIIb/IIIa antibodies or antigen binding fragments can comprise a VH region comprising VH-CDR1, VH-CDR2, and VH-CDR3 domains from BIIB_4_147. In certain embodiments, these anti-GPIIb/IIIa antibodies or antigen binding fragments can comprise a VL region comprising VL-CDR1, VL-CDR2, and VL-CDR3 domains from BIIB_4_147. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 53, 54, and 55, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 83, 84, and 85, respectively.

In some embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 9 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 11. These anti-GPIIb/IIIa antibodies or antigen binding fragments can comprise a VH region comprising VH-CDR1, VH-CDR2, and VH-CDR3 domains from BIIB_4_156. In certain embodiments, these anti-GPIIb/IIIa antibodies or antigen binding fragments can comprise a VL region comprising VL-CDR1, VL-CDR2, and VL-CDR3 domains from BIIB_4_156. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 56, 57, and 58, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 86, 87, and 88, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 13 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 15. These anti-GPIIb/IIIa antibodies or antigen binding fragments can comprise a VH region comprising VH-CDR1, VH-CDR2, and VH-CDR3 domains from BIIB_4_174. In certain embodiments, these anti-GPIIb/IIIa antibodies or antigen binding fragments can comprise a VL region comprising VL-CDR1, VL-CDR2, and VL-CDR3 domains from BIIB_4_174. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 56, 57, and 59, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 86, 89, and 90, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 17 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 19. These anti-GPIIb/IIIa antibodies or antigen binding fragments can comprise a VH region comprising VH-CDR1, VH-CDR2, and VH-CDR3 domains from BIIB_4_175. In certain embodiments, these anti-GPIIb/IIIa antibodies or antigen binding fragments can comprise a VL region comprising VL-CDR1, VL-CDR2, and VL-CDR3 domains from BIIB_4_175. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 60, 61, and 62, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 91, 92, and 93, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 21 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 23. These anti-GPIIb/IIIa antibodies or antigen binding fragments can comprise a VH region comprising VH-CDR1, VH-CDR2, and VH-CDR3 domains from BIIB_4_204. In certain embodiments, these anti-GPIIb/IIIa antibodies or antigen binding fragments can comprise a VL region comprising VL-CDR1, VL-CDR2, and VL-CDR3 domains from BIIB_4_204. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 63, 64, and 65, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 86, 94, and 95, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 25 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 27. These anti-GPIIb/IIIa antibodies or antigen binding fragments can comprise a VH region comprising VH-CDR1, VH-CDR2, and VH-CDR3 domains from BIIB_4_209. In certain embodiments, these anti-GPIIb/IIIa antibodies or antigen binding fragments can comprise a VL region comprising VL-CDR1, VL-CDR2, and VL-CDR3 domains from BIIB_4_209. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 53, 54, and 66, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 86, 87, and 96, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 29 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 31. These anti-GPIIb/IIIa antibodies or antigen binding fragments can comprise a VH region comprising VH-CDR1, VH-CDR2, and VH-CDR3 domains from BIIB_4_224. In certain embodiments, these anti-GPIIb/IIIa antibodies or antigen binding fragments can comprise a VL region comprising VL-CDR1, VL-CDR2, and VL-CDR3 domains from BIIB_4_224. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 67, 68, and 69, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 97, 98, and 99, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 33 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 35. These anti-GPIIb/IIIa antibodies or antigen binding fragments can comprise a VH region comprising VH-CDR1, VH-CDR2, and VH-CDR3 domains from BIIB_4_309. In certain embodiments, these anti-GPIIb/IIIa antibodies or antigen binding fragments can comprise a VL region comprising VL-CDR1, VL-CDR2, and VL-CDR3 domains from BIIB_4_309. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 53, 54, and 70, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 100, 101, and 102, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 37 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 39. These anti-GPIIb/IIIa antibodies or antigen binding fragments can comprise a VH region comprising VH-CDR1, VH-CDR2, and VH-CDR3 domains from BIIB_4_311. In certain embodiments, these anti-GPIIb/IIIa antibodies or antigen binding fragments can comprise a VL region comprising VL-CDR1, VL-CDR2, and VL-CDR3 domains from BIIB_4_311. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 71, 72, and 73, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 103, 104, and 105, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 41 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 43. These anti-GPIIb/IIIa antibodies or antigen binding fragments can comprise a VH region comprising VH-CDR1, VH-CDR2, and VH-CDR3 domains from BIIB_4_317. In certain embodiments, these anti-GPIIb/IIIa antibodies or antigen binding fragments can comprise a VL region comprising VL-CDR1, VL-CDR2, and VL-CDR3 domains from BIIB_4_317. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 74, 75, and 76, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 86, 87, and 106, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 45 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 47. These anti-GPIIb/IIIa antibodies or antigen binding fragments can comprise a VH region comprising VH-CDR1, VH-CDR2, and VH-CDR3 domains from BIIB_4_318. In certain embodiments, these anti-GPIIb/IIIa antibodies or antigen binding fragments can comprise a VL region comprising VL-CDR1, VL-CDR2, and VL-CDR3 domains from BIIB_4_318. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 77, 78, and 79, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 107, 108, and 109, respectively.

In certain embodiments the anti-GPIIb/IIIa antibody or antigen-binding molecule thereof comprises a VH region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 49 and a VL region comprising an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to the amino acid sequence of SEQ ID NO: 51. These anti-GPIIb/IIIa antibodies or antigen binding fragments can comprise a VH region comprising VH-CDR1, VH-CDR2, and VH-CDR3 domains from BIIB_4_319. In certain embodiments, these anti-GPIIb/IIIa antibodies or antigen binding fragments can comprise a VL region comprising VL-CDR1, VL-CDR2, and VL-CDR3 domains from BIIB_4_319. These anti-GPIIb/IIIa antibodies or antigen binding fragments can comprise a VH region comprising VH-CDR1, VH-CDR2, and VH-CDR3 domains, wherein VH-CDR1, VH-CDR2, VH-CDR3 domains comprise or consist of amino acid sequences set forth in SEQ ID NOs.: 80, 81, and 82, respectively. These anti-GPIIb/IIIa antibodies or antigen binding fragments can further comprise a VL region comprising VL-CDR1, VL-CDR2, and VL-CDR3 domains, wherein the VL-CDR1, VL-CDR2, and VL-CDR3 domains comprise or consist of amino acid sequences set forth in SEQ ID NOs.: 86, 87, and 110, respectively.

In some embodiments, the above antibodies or antigen-binding fragments thereof do not activate platelets. In certain embodiments, these antibodies or antigen-binding fragments thereof bind to GPIIb/IIIa with a dissociation constant (KD) of ≤1 μM, ≤750 nM, ≤500 nM, ≤250 nM, ≤200 nM, ≤150 nM, ≤100 nM, ≤75 nM, ≤50 nM, ≤10 nM, ≤1 nM, ≤0.1 nM, ≤10 pM, ≤1 pM, or ≤0.1 pM.

In some embodiments, the above-described anti-GPIIb/IIIa antibodies can comprise a kappa light chain constant region. In other embodiments, these anti-GPIIb/IIIa antibodies can comprise a lambda light chain constant region. In one embodiment, the light chain constant region comprises the following amino acid sequence:

(SEQ ID NO: 121)
RTVA APSVFIFPPS DEQLKSGTAS WCLLNNFYP
REAKVQWKVD NALQSGNSQE SVTEQDSKDS
TYSLSSTLTL SKADYEKHKV YACEVTHQGL
SSPVTKSFNR GEC.

In other embodiments, the light chain constant region comprises an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, or at least 99% identical to SEQ ID NO:121.

The anti-GPIIb/IIIa antibodies or antigen-binding fragments thereof of this disclosure can also comprise a heavy chain constant region or a portion thereof (e.g. the CH1 domain). In certain embodiments the heavy chain constant region is from an IgG1 or IgG4 antibody. In one embodiment, the heavy chain constant region comprises the following amino acid sequence:

(SEQ ID NO: 122)
AS TKGPSVFPLA PSSKSTSGGT AALGCLVKDY
FPEPVTVSWN SGALTSGVHT FPAVLQSSGL
YSLSSVVTVP SSSLGTQTYI CNVNHKPSNT
KVDKKVEPKS C.

In other embodiments, the heavy chain constant region comprises an amino acid sequence that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, or at least 99% identical to SEQ ID NO:122.
In another embodiment, the heavy chain constant region comprises the following amino acid sequence:

(SEQ ID NO: 123)
ASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKY
GPPCPPCPAPEFLGGPSVFLFPPKPKDTLMISRTPEVTCVVVDVSQEDPEV
QFNWYVDGVEVHNAKTKPREEQFQSTYRVVSVLTVLHQDWLNGKEYKCKVS
NKGLPSSIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCS
VMHEALHNHYTQKSLSLSPG.

In certain embodiments, the anti-GPIIb/IIIa antibody has an isotype selected from the group consisting of IgG1, IgG2, IgG3, and IgG4. The heavy chain constant region can be a wild-type human Fc region, or a human Fc region that includes one or more amino acid substitutions. The antibodies can have mutations that stabilize the disulfide bond between the two heavy chains of an immunoglobulin, such as mutations in the hinge region of IgG4, as disclosed in the art (e.g., Angal et al., Mol. Immunol., 30:105-08 (1993)). See also, e.g., U.S. 2005/0037000. The heavy chain constant region can also have substitutions that modify the properties of the antibody (e.g., decrease one or more of: Fc receptor binding, antibody glycosylation, deamidation, binding to complement, or methionine oxidation). In some instances, the antibodies may have mutations such as those described in U.S. Pat. Nos. 5,624,821 and 5,648,260. In some embodiments, the antibody is modified to reduce or eliminate effector function. In some embodiments, the heavy chain constant region has one or more of the following mutations: S228P; N297Q; and T299A (numbering according to Kabat). The heavy chain constant region can be chimeric, e.g., the Fc region can comprise the CH1 and CH2 domains of an IgG antibody of the IgG4 isotype, and the CH3 domain from an IgG antibody of the IgG1isotype (see, e.g., U.S. Patent Appl. No. 2012/0100140A1 which is incorporated by reference in its entirety herein). In a specific embodiment, the anti-GPIIb/IIIa antibodies described herein have a chimeric constant region comprising the CH1 and CH2 domains of an IgG antibody of the IgG4 isotype, and the CH3 domain from an IgG antibody of the IgG1isotype and further contain the S228P and N297Q mutations (numbering according to Kabat).

Antigen-binding fragments of the anti-GPIIb/IIIa antibodies are also encompassed by this disclosure. In some embodiments, the anti-GPIIb/IIIa antibody or antigen-binding molecule thereof comprises or consists of (i) a single chain Fv (“scFv”); (ii) a diabody; (iii) an sc(Fv)2; (iv) a polypeptide chain of an antibody; (v) F(ab′)2; or (vi) F(ab). In one embodiment, the antigen-binding fragment is an Fab molecule. The fragment antigen-binding (Fab fragment) is a region on an antibody that binds to antigens. It is composed of one constant and one variable domain of each of the heavy and the light chain. These domains shape the paratope, i.e., the antigen-binding site. The enzyme papain can be used to cleave an immunoglobulin monomer into two Fab fragments and an Fc fragment. Recombinant methods can also be used to make an Fab molecule. In one embodiment, the antibody fragment that specifically binds GPIIb/IIIa is an Fab molecule comprising aVH and a VL domain that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or identical to the VH and VL domains of any one of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319. In certain embodiments, these Fab fragments further comprise a Fab heavy chain that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or identical to the amino acid sequence set forth in SEQ ID NO:122. In certain embodiments, these Fab fragments further comprise a Fab light chain that is at least 65% identical, at least 70% identical, at least 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or identical to the amino acid sequence set forth in SEQ ID NO:121. In another embodiment, the antigen-binding fragment is a single-chain fragment variable (scFv). An scFv is comprised of the variable regions of the heavy and light chains of an antibody. It is only half the size of the Fab fragment and yet retains the original specificity of the parent immunoglobulin. Methods of making an scFv are well known in the art (see, e.g., Ahmad et al., Clinical and Developmental Immunology, vol. 2012, Article ID 980250, 15 pages, 2012. doi:10.1155/2012/980250). The invention encompasses scFvs that are identical to, or that have at least 65% to at least 99% identity to, the VH and VL domains of any one of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, and BIIB-4-319.

In certain embodiments, the anti-GPIIb/IIIa antibody or antigen-binding fragment thereof can be a targeting moiety. These targeting moieties are useful in ferrying an agent of interest (e.g., a therapeutic agent, a coagulation factor, a small molecule drug) to platelets. In some embodiments, an anti-GPIIb/IIIa antibody or antigen-binding fragment thereof disclosed herein can target GPIIb/IIIa located on the surface of platelets. In certain embodiments, these antibodies or antigen-binding fragments thereof are or derived from Class I or Class II antibodies.

In certain embodiments, the anti-GPIIb/IIIa antibody or antigen-binding fragment thereof can be used to reduce platelet aggregation and/or thrombus formation. In certain embodiments, these antibodies or antigen-binding fragments thereof are or derived from Class III antibodies.

### D. Chimeric Molecules Comprising Anti-GPIIb/IIIa Antibodies

The present disclosure also provides “chimeric molecules” comprising, for example, at least one of the GPIIb/IIIa antibodies or antigen-binding fragments thereof disclosed herein that is linked and/or conjugated and/or otherwise associated with at least one heterologous moiety. In certain embodiments, the heterologous moiety is an agent that to be transported or delivered to a platelet or its local environment. Such an agent can be e.g., a therapeutic agent such as a clotting factor (e.g., FVII, rFVIIa).

A chimeric molecule disclosed herein encompasses any molecule comprising (i) a GPIIb/IIIa antibody or antigen-binding molecule thereof disclosed herein (e.g., an Fab or scFv derived from a GPIIb/IIIa antibody disclosed herein), and (ii) at least one (e.g., one two, three, four) heterologous moiety (e.g., a therapeutic moiety, a clotting factor, a half-life extending moiety) and optionally including one or more linkers. In some embodiments, a chimeric molecule is a chimeric protein, i.e., a chimeric molecule in which all its components (heterologous moieties and/or linkers) are polypeptides. Other chimeric molecules can comprise non-polypeptide heterologous moieties (e.g., PEG, lipids, carbohydrates, nucleic acids, small molecule therapeutic agents, radionuclides, fluorescent probes, etc.) and/or non-polypeptide linkers.

In some embodiments, a chimeric molecule comprises a first amino acid sequence derived from a first source, bonded, covalently or non-covalently, to a second amino acid sequence derived from a second source, wherein the first and second source are not the same. A first source and a second source that are not the same can include two different biological entities, or two different proteins from the same biological entity, or a biological entity and a non-biological entity. A chimeric molecule can include for example, a protein derived from at least two different biological sources. A biological source can include any non-synthetically produced nucleic acid or amino acid sequence (e.g., a genomic or cDNA sequence, a plasmid or viral vector, a native virion or a mutant or analog, as further described herein, of any of the above). A synthetic source can include a protein or nucleic acid sequence produced chemically and not by a biological system (e.g., solid phase synthesis of amino acid sequences). A chimeric molecule can also include a protein derived from at least 2 different synthetic sources or a protein derived from at least one biological source and at least one synthetic source. A chimeric molecule can also comprise a first amino acid sequence derived from a first source, covalently or non-covalently linked to a nucleic acid, derived from any source or a small organic or inorganic molecule derived from any source. The chimeric molecule can also comprise a linker molecule between the first and second amino acid sequence or between the first amino acid sequence and the nucleic acid, or between the first amino acid sequence and the small organic or inorganic molecule.

In some embodiments, the chimeric molecule has, for example, a formula: (i) Ab-(L)-H or (ii) H-(L)-Ab, wherein, H is a heterologous moiety; L is an optional linker; and, Ab is an anti-GPIIb/IIIa antibody or antigen-binding molecule thereof disclosed herein. One or more copies (e.g., one, two, three, four) of the same heterologous moiety may be included in the chimeric molecule.

In some embodiments, the chimeric molecule further comprises a second heterologous moiety. Accordingly, in some embodiments, the chimeric molecule has a formula selected from:

(i) H1-(L1)-Ab-(L2)-H2;

(ii) H2-(L2)-Ab-(L1)-H1;

(iii) H1-(L1)-H2-(L2)-Ab;

(iv) H2-(L2)-H1-(L1)-Ab;

(v) Ab-(L1)-H1-(L2)-H2; or,

(vi) Ab-(L2)-H2-(L1)-H1;

wherein, Ab is an anti-GPIIb/IIIa antibody or antigen-binding molecule thereof disclosed herein; H1 is a first heterologous moiety, H2 is a second heterologous moiety, L1 is a first optional linker, and L2 is a second optional linker. One or more copies (e.g., one, two, three, four) of the same heterologous moiety may be included in the chimeric molecule.

In some embodiments, the first heterologous moiety and the second heterologous moiety are the same. In other embodiments, the first heterologous moiety and the second heterologous moiety are different. In some embodiments, L1 and L2 are the same. In other embodiments, L1 and L2 are different.

The chimeric molecule formulas disclosed are oriented from N-terminus (left) to C-terminus (right). One skilled in the art would understand that the chimeric molecule formulas disclosed herein are non-limiting examples of chimeric molecules comprising the disclosed anti-GPIIb/IIIa antibodies or antigen-binding fragments thereof. For example, the formulas can comprise further sequences at their N-terminal or C-terminal ends, or inserted between elements of the formula. Accordingly, a chimeric molecule can comprise one, two, three, four, five, or more than five heterologous moieties. In some embodiments, the hyphen (-) in a formula indicates a peptide bond or one or more amino acids. Exemplary chimeric molecules are presented in FIGS. 18 and 19.

In some embodiments, a chimeric protein comprises a first polypeptide chain and a second polypeptide chain, which are associated with each other. In some embodiments, the first polypeptide chain comprises a light chain of a clotting factor (e.g., FVII) and a heterologous moiety (e.g., a half-life extending moiety), and the second polypeptide chain comprises a heavy chain of the clotting factor (e.g., FVII) and a GPIIb/IIIa antibody or antigen-binding molecule thereof disclosed herein. In other embodiments, the first polypeptide chain comprises a light chain of a clotting factor (e.g., FVII) and a GPIIb/IIIa antibody or antigen-binding molecule thereof disclosed herein, and the second polypeptide chain comprises a heavy chain of the clotting factor (e.g., FVII) and a heterologous moiety (e.g., a half-life extending moiety). In yet another embodiment, the first polypeptide chain comprises a light chain of a clotting factor (e.g., FVII) and the second polypeptide chain comprises a heavy chain of the clotting factor (e.g., FVII), a GPIIb/IIIa antibody or antigen-binding molecule thereof disclosed herein, and a heterologous moiety (e.g., a half-life extending moiety). In some embodiments, the first polypeptide chain comprises a light chain of a clotting factor (e.g., FVII) and the second polypeptide chain comprises a heavy chain of the clotting factor (e.g., FVII), a heterologous moiety (e.g., a half-life extending moiety), and a GPIIb/IIIa antibody or antigen-binding molecule thereof disclosed herein. In other embodiments, the first polypeptide chain comprises a light chain of a clotting factor (e.g., FVII), a heterologous moiety (e.g., a half-life extending moiety), and a GPIIb/IIIa antibody or antigen-binding molecule thereof disclosed herein, and the second polypeptide chain comprises a heavy chain of the clotting factor (e.g., FVII). In some embodiments, the first polypeptide chain comprises a light chain of a clotting factor (e.g., FVII), a GPIIb/IIIa antibody or antigen-binding molecule thereof disclosed herein, and a heterologous moiety (e.g., a half-life extending moiety), and the second polypeptide chain comprises a heavy chain of the clotting factor (e.g., FVII).

In some embodiments, the chimeric molecule comprises a formula wherein:

(1) the first polypeptide chain comprises CF_{L}-H or H-CF_{L} and the second polypeptide chain comprises CF_{H}-Ab or Ab-CF_{H};

(2) the first polypeptide chain comprises CF_{L}-Ab or Ab-CF_{L} and the second polypeptide chain comprises CF_{H}-H or H-CF_{H};

(3) the first polypeptide chain comprises CF_{L} and the second polypeptide chain comprises CF_{H}-Ab-H or H-Ab-CF_{H};

(4) the first polypeptide chain comprises CF_{L} and the second polypeptide chain comprises CF_{H}-H-Ab or Ab-H-CF_{H};

(5) the first polypeptide chain comprises CF_{L}-H-Ab or Ab-H-CF_{L} and the second polypeptide chain comprises CF_{H}; or

(6) the first polypeptide chain comprises CF_{L}-Ab-H or H-Ab-CF_{L} and the second polypeptide chain comprises CF_{H};

wherein, CF_{L} is a light chain of a clotting factor (e.g., FVII); CF_{H} is a heavy chain of the clotting factor (e.g., FVII); Ab is an anti-GPIIb/IIIa antibody or antigen-binding molecule thereof and H is a heterologous moiety (e.g., a half-life extending moiety). In some embodiments, the clotting factor is independently selected from the group consisting of FVII, FIX, FX, and any combinations thereof.

This disclosure also provides a chimeric molecule comprising a first polypeptide chain and a second polypeptide chain, which are associated with each other, (1) wherein the first polypeptide chain comprises a light chain of a clotting factor (e.g., FVII, FIX, or FX), and a targeting moiety, which binds to a platelet, and the second polypeptide chain comprises a heavy chain of the clotting factor (e.g., FVII, FIX, or FX) and a heterologous moiety (e.g., a half-life extending moiety); (2) wherein the first polypeptide chain comprises a light chain of a clotting factor (e.g., FVII) and a heterologous moiety (e.g., a half-life extending moiety), and the second polypeptide chain comprises a heavy chain of the clotting factor (e.g., FVII, FIX, or FX) and a targeting moiety, which binds to a platelet; (3) wherein the first polypeptide chain comprises a light chain of a clotting factor (e.g., FVII, FIX, or FX), a heterologous moiety (e.g., a half-life extending moiety), and a targeting moiety, which binds to a platelet, and the second polypeptide comprises a heavy chain of the clotting factor (e.g., FVII, FIX, or FX); or (4) wherein the first polypeptide chain comprises a light chain of a clotting factor (e.g., FVII, FIX, or FX), a targeting moiety, which binds to a platelet, and a heterologous moiety (e.g., a half-life extending moiety) and the second polypeptide chain comprises a heavy chain of the clotting factor (e.g., FVII, FIX, or FX). In some embodiments, the clotting factor is FVII, FIX, or FX.

As used herein, the phrases “which binds to a platelet,” “binding to a platelet,” and variants thereof generally refer to the specific binding of (i) a GPIIb/IIIa antibody or antigen-binding molecule thereof or (ii) a chimeric molecule of the present disclosure to an antigenic site on the surface of the platelet, e.g., an epitope on the extracellular domains of the α and/or β subunits of the GPIIb/IIIa receptor. It is known to a person skilled in the art that GPIIb/IIIa is present in two pools, a plasma membrane pool present in the platelet's resting state and an internal pool of GPIIb/IIIa which is expressed upon platelet activation. See, e.g., Quinn et al., J. Pharmacol. Exp. Ther., 297:496-500 (2001). Accordingly, in some specific embodiments, and particularly for diagnostic uses where the platelet's plasma membrane can be permeabilized, the binding of an anti-GPIIb/IIIa antibody or antigen-binding molecule thereof to platelets, or the binding of a chimeric molecule of the present disclosure to platelets can refer to binding to the plasma membrane pool and/or to the internal pool of GPIIb/IIIa.

In some embodiments, the chimeric molecule comprises a first polypeptide chain and a second polypeptide chain, which are associated with each other, (1) wherein the first polypeptide chain comprises CF_{L}-H or H-CF_{L} and the second polypeptide chain comprises CF_{H}-Ab or Ab-CF_{H}; (2) wherein the first polypeptide chain comprises CF_{L}-Ab or Ab-CF_{L} and the second polypeptide chain comprises CF_{H}-H or H-CF_{H}; (3) wherein the first polypeptide chain comprises CF_{L}-H-Ab or Ab-H-CF_{L} and the second polypeptide chain comprises CF_{H}; or (4) wherein the first polypeptide chain comprises CF_{L}-Ab-H or H-Ab-CF_{L} and the second polypeptide chain comprises CF_{H}; wherein, H is a heterologous moiety (e.g., a half-life extending moiety), CF_{H} is a heavy chain of a clotting factor (e.g., FVII), CF_{L} is a light chain of the clotting factor (e.g., FVII, FIX, or FX), Ab is an anti-GPIIb/IIIa antibody that binds to a platelet, and L is an optional linker.

In some embodiments, the association between the first polypeptide chain and the second polypeptide chain in the chimeric molecule is a covalent bond or a non-covalent bond. Thus, in other embodiments, the association between the first polypeptide chain and the second polypeptide chain in the chimeric molecule is a covalent bond between the heavy chain and the light chain of the clotting factor (e.g., FVII, FIX, or FX). In contrast, in some other embodiments, the covalent bond is a disulfide bond.

The present disclosure also provides a chimeric molecule comprising a single polypeptide chain, which comprises, from N terminus to C terminus, (i) a light chain of a clotting factor (e.g., FVII, FIX, or FX), a heterologous moiety (e.g., a half-life extending moiety), a protease cleavage site, a heavy chain of the clotting factor (e.g., FVII, FIX, or FX), and a targeting moiety (e.g., a GPIIb/IIIa antibody or antigen-binding molecule thereof) which binds to a platelet or (ii) a light chain of a clotting factor (e.g., FVII), a targeting moiety, which binds to a platelet, a protease cleavage site, a heavy chain of the clotting factor (e.g., FVII, FIX, or FX), and a heterologous moiety (e.g., a half-life extending moiety). In some embodiments, the clotting factor is FVII. In other embodiments, the clotting factor is FIX or FX. In yet other embodiments, the clotting factor is FVII, FIX, or FX. In some embodiments, the protease cleavage site is an intracellular processing site. In some embodiments, the intracellular processing site is processed by a proprotein convertase. In some embodiments, the proprotein convertase is selected from the group consisting of PC5, PACE, PC7, and any combinations thereof.

I. Heterologous Moieties

The heterologous moiety or moieties of the chimeric molecules disclosed herein can comprise, consist of, or consist essentially of, for example, prophylactic and/or therapeutic agents (e.g., clotting factors), molecules capable of improving a pharmacokinetic (PK) property (e.g., plasma half-life extending moieties), and detectable moieties (e.g., fluorescent molecules or radionuclides). In some embodiments, the heterologous moiety comprises a clotting factor (e.g., a Factor VII). In some embodiments, a heterologous moiety comprises a molecule that can modify a physicochemical property of a chimeric molecule lacking such heterologous moiety. For example, it can increase the hydrodynamic radius of a chimeric molecule. In other embodiments, the incorporation of a heterologous moiety into a chimeric molecule can improve one or more pharmacokinetic properties without significantly affecting its biological activity or function (e.g., procoagulant activity in chimeric molecules comprising a clotting factor). In other embodiments, a heterologous moiety increases stability of the chimeric molecule of the invention or a fragment thereof.

In some embodiments, the heterologous moiety is a polypeptide comprising, consisting essentially of, or consisting of at least about 10, 100, 200, 300, 400, 500, 600, 700, 800, 900, 1000, 1100, 1200, 1300, 1400, 1500, 1600, 1700, 1800, 1900, 2000, 2500, 3000, or 4000 amino acids. In other embodiments, the heterologous moiety is a polypeptide comprising, consisting essentially of, or consisting of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids.

Non-limiting examples of the heterologous moieties are discussed below.

### 1. Clotting Factors

In some embodiments, the chimeric molecules of this disclosure comprise at least one polypeptide heterologous moiety which is (i) a clotting factor, or (ii) a procoagulant peptide (e.g., a synthetic procoagulant peptide). Blood coagulation is a process that involves a complex interaction of various blood factors that eventually result in a fibrin clot. Generally, the blood factor, which participate in what has been referred to as the coagulation “cascade”, are enzymatically inactive proteins (proenzymes or zymogens) that are converted to proteolytic enzymes by the action of an activator (which itself is an activated clotting factor). Coagulation factors that have undergone such a conversion are generally referred to as “active factors”, and are designated by the addition of the letter “a” to the name of the coagulation factor (e.g. Factor VIIa). In some embodiments, the clotting factor is independently selected from the group consisting of factor FVII (“FVII”), factor IX (“FIX”), or factor X (“FX”), and any combinations thereof. As discussed in detail below, the clotting factor can be, for example, FVII zymogen, activatable FVII, activated FVII (FVIIa), FIX zymogen, activatable FIX, activated FIX (FIXa), FX zymogen, activatable FX, or activated FX (FXa). In some embodiments, the clotting factor can comprise a single polypeptide chain or two polypeptide chains (I the heavy chain and the light chain of FVII). In some embodiments, the chimeric molecule comprises a FVII or activated FVII (FVIIa) clotting factor. In some embodiments, the chimeric molecule of the invention comprises a FIX or activated FIX (FIXa) clotting factor. In other embodiments, the chimeric molecule comprises a FX or activated FX (FXa) clotting factor.

In some embodiments, the chimeric molecule comprises a single clotting factor, which in the chimeric molecule is represented by a formula as H, H1 or H2. In other embodiments, the chimeric molecule comprises two clotting factors. In some embodiments, the two clotting factors are the same, whereas in other embodiments, the two clotting factors are different. In some embodiments, one clotting factor is a fragment of a clotting factor (e.g., a heavy chain of a clotting factor such as FVII) and the second clotting factor is a fragment of the same clotting factor (e.g., a light chain of a clotting factor such as FVIII). In some embodiments, the chimeric molecule comprises more than two clotting factors.

a. Factor VII

In some embodiments, the chimeric molecule comprises a clotting factor which is a mature form of Factor VII or a variant thereof. Factor VII (FVII, F7; also referred to as Factor 7, coagulation factor VII, serum factor VII, serum prothrombin conversion accelerator, SPCA, proconvertin and eptacog alpha) is a serine protease that is part of the coagulation cascade. FVII includes a Gla domain, two EGF domains (EGF-1 and EGF-2), and a serine protease domain (or peptidase Si domain) that is highly conserved among all members of the peptidase Si family of serine proteases, such as for example with chymotrypsin. In some embodiments, the chimeric molecule comprises a Factor VIIa. In certain embodiments, the Factor VIIa is recombinant.

FVII can occur as a single chain zymogen, an activated zymogen-like two-chain polypeptide, or a fully activated two-chain form. The zymogen composed of a single chain polypeptide is converted to a two-chain form connected by disulfide bonds by the action of Factor Xa in the presence of calcium ions and phospholipids, thrombin, or by the action of factor XIIa (without additional cofactors). This hydrolysis of Factor VII is accompanied by an at least 85-fold increase in the Factor VII coagulant activity compared to the single chain form (see, e.g., Radcliffe et al., J. Biol. Chem., 250(2):388-395 (1975) and Handbook of Enzymes, Class 3.4 Hydrolases II: EC3.4.21-3.4.22, Volume 7, coed. By Antje Chang, 2002, (Springer, 2^{nd} edition)). Following vascular damage, blood clotting is triggered when factor VIIa (FVIIa) forms a complex with tissue factor (TF). In hemophilia A and B, the propagation phase of blood coagulation is disrupted due to the lack of factors VIII (FVIII) and IX (FIX), leading to excessive bleeding. However, high doses of recombinant FVIIa (rFVIIa) can bypass the FVIII/FIX deficiency and ameliorate bleeding problems.

The amino acid sequence of the B isoform of FVII zymogen is provided below (the signal sequence (boldened), propeptide sequence (underlined); the peptide bond between R and I (boldened and underlined) is cleaved to activate FVII):

(SEQ ID NO: 128)

| | |
|---|---|
| 1 | MVSQALRLLC LLLGLQGCLA AVFVTQEEAH |
| | GVLHRRRRAN AFLEELRPGS |
| 51 | LERECKEEQC SFEEAREIFK DAERTKLFWI |
| | SYSDGDQCAS SPCQNGGSCK |
| 101 | DQLQSYICFC LPAFEGRNCE THKDDQLICV |
| | NENGGCEQYC SDHTGTKRSC |
| 151 | RCHEGYSLLA DGVSCTPTVE YPCGKIPILE |
| | KRNASKPQGR IVGGKVCPKG |
| 201 | ECPWQVLLLV NGAQLCGGTL INTIWVVSAA |
| | HCFDKIKNWR NLIAVLGEHD |
| 251 | LSEHDGDEQS RRVAQVIIPS TYVPGTTNHD |
| | IALLRLHQPV VLTDHVVPLC |
| 301 | LPERTFSERT LAFVRFSLVS GWGQLLDRGA |
| | TALELMVLNV PRLMTQDCLQ |
| 351 | QSRKVGDSPN ITEYMFCAGY SDGSKDSCKG |
| | DSGGPHATHY RGTWYLTGIV |
| 401 | SWGQGCATVG HFGVYTRVSQ YIEWLQKLMR |
| | SEPRPGVLLR APFP |

It is to be understood the chimeric molecules of this disclosure can include any FVII zymogen (e.g., the A or B isoforms) so long as intended results are achieved (e.g., effectiveness in treatment of a coagulation or hemostatic disorder).

The amino acid sequence of the light chain of FVII is provided below:

(SEQ ID NO: 129)
ANAFLEELRP GSLERECKEE QCSFEEAREI FKDAERTKLF
WISYSDGDQC ASSPCQNGGS CKDQLQSYIC FCLPAFEGRN
CETHKDDQLI CVNENGGCEQ YCSDHIGTKR SCRCHEGYSL
LADGVSCTPT VEYPCGKIPI LEKRNASKPQ GR

The amino acid sequence of the heavy chain of FVII is provided below.

(SEQ ID NO: 130)
IVGGKVCP KGECPWQVLL LVNGAQLCGG TLINTIWVVS
AAHCFDKIKN WRNLIAVLGE HDLSEHDGDE QSRRVAQVII
PSTYVPGTTN HDIALLRLHQ PVVLTDHVVP LCLPERTFSE
RTLAFVRFSL VSGWGQLLDR GATALELMVL NVPRLMTQDC
LQQSRKVGDS PNITEYMFCA GYSDGSKDSC KGDSGGPHAT
HYRGTWYLTG IVSWGQGCAT VGHFGVYTRV SQYIEWLQKL
MRSEPRPGVL LRAPFP

This disclosure also encompasses any allelic variants of FVII.

Other exemplary FVII variants that are encompassed by this disclosure include those with increased specific activity, e.g., mutations that increase the activity of FVII by increasing its enzymatic activity (K_{cat} or Kₘ). Such variants have been described in the art and include, e.g., mutant forms of the molecule as described for example in Persson, Semin Hematol., 41 (1Suppl 1):89-92 (2004); Persson et al., Proc. Natl. Acad Sci. USA 98:13583 (2001); Petrovan and Ruf, J. Biol. Chem. 276:6616 (2001); Persson et al., J. Biol. Chem. 276:29195 (2001); Soejima et al., J. Biol. Chem. 276:17229 (2001); Soejima et al., J. Biol. Chem. 247:49027 (2002); and WO2002/022776.

In one embodiment, a variant form of FVII includes mutations, e.g., V158D-E296V-M298Q. In another embodiment, a variant form of FVII includes a replacement of amino acids 608-619 (LQQSRKVGDSPN (SEQ ID NO:131), corresponding to the 170-loop) from the FVII mature sequence with amino acids EASYPGK (SEQ ID NO:132) from the 170-loop of trypsin. High specific activity variants of FVII are also known in the art. For example, Simioni et al. (N.E. Journal of Medicine 361:1671, 2009) describe an R338L mutation. Chang et al. (J. Biol. Chem. 273:12089, 1988) and Pierri et al. (Human Gene Therapy 20:479, 2009) describe an R338A mutation. Other mutations are known in the art and include those described, e.g., in Zogg and Brandstetter, Structure 17:1669 (2009); Sichler et al., J. Biol. Chem. 278:4121 (2003); and Sturzebecher et al., FEBS Lett. 412:295 (1997). The contents of all of the references above are incorporated herein by reference.

Full activation, which occurs upon conformational change from a zymogen-like form, occurs upon binding to its co-factor, i.e., tissue factor. Also, mutations can be introduced that result in the conformation change in the absence of tissue factor. Hence, reference to FVIIa includes both two-chain forms thereof: the zymogen-like form, and the fully activated two-chain form.

b. Factor IX

In one embodiment, the chimeric molecule comprises a clotting factor which is a mature form of Factor IX or a variant thereof. Factor IX circulates as a 415 amino acid, single chain plasma zymogen. See, Vysotchin et al., J. Biol. Chem. 268:8436 (1993). The amino acid sequence of FIX zymogen is provided below (the signal sequence is underlined (1-28); the propeptide sequence (29-46) is boldened):

(SEQ ID NO: 133)
MQRVNMIMAESPGLITICLLGYLLSAECTVFLDHENANKILNRPKRYNSGK
LEEFVQGNLERECMEEKCSFEEAREVFENTERTTEFWKQYVDGDQCESNPC
LNGGSCKDDINSYECWCPFGFEGKNCELDVTCNIKNGRCEQFCKNSADNKV
VCSCTEGYRLAENQKSCEPAVPFPCGRVSVSQTSKLTRAETVFPDVDYVNS
TEAETILDNITQSTQSFNDFTRVVGGEDAKPGQFPWQVVLNGKVDAFCGGS
IVNEKWIVTAAHCVETGVKITVVAGEHNIEETEHTEQKRNVIRIIPHHNYN
AAINKYNHDIALLELDEPLVLNSYVTPICIADKEYTNIFLKFGSGYVSGWG
RVFHKGRSALVLQYLRVPLVDRATCLRSTKFTIYNNMFCAGFHEGGRDSCQ
GDSGGPHVTEVEGTSFLTGIISWGEECAMKGKYGIYTKVSRYVNWIKEKTK
LT

The zymogen of FIX is activated by FXIa or by the tissue factor/FVIIa complex. Specific cleavages between arginine-alanine 145-146 and arginine-valine 180-181 result in a light chain and a heavy chain linked by a single disulfide bond between cysteine 132 and cysteine 289 (Bajaj et al., Biochemistry 22:4047 (1983)).

The structural organization of FIX is similar to that of the vitamin K-dependent blood clotting proteins FVII, FX and protein C. The approximately 45 amino acids of the amino terminus comprise the gamma-carboxyglutamic acid, or Gla, domain. This is followed by two epidermal growth factor homology domains (EGF), an activation peptide and the catalytic “heavy chain” which is a member of the serine protease family (Vysotchin et al., J. Biol. Chem. 268:8436 (1993); Spitzer et al., Biochemical Journal 265:219 (1990); Brandstetter et al., Proc. Natl. Acad Sci. USA 92:9796 (1995)).

c. Factor X

In one embodiment, the chimeric molecule comprises a clotting factor which is a mature form of Factor X. Factor X is a vitamin-K dependent glycoprotein with a molecular weight of 58.5 kDa, which is secreted from liver cells into the plasma as a zymogen. Initially factor X is produced as a prepropeptide with a signal peptide consisting in total of 488 amino acids. The amino acid sequence of FX zymogen is provided below (the signal sequence (1-23) is underlined and the propeptide (24-40) is boldened):

(SEQ ID NO: 134)
MGRPLHLVLLSASLAGLLLLGESLFIRREQANNILARVTRANSFLEEMKKG
HLERECMEETCSYEEAREVFEDSDKTNEFWNKYKDGDQCETSPCQNQGKCK
DGLGEYTCTCLEGFEGKNCELFTRKLCSLDNGDCDQFCHEEQNSVVCSCAR
GYTLADNGKACIPTGPYPCGKQTLERRKRSVAQATSSSGEAPDSITWKPYD
AADLDPTENPFDLLDFNQTQPERGDNNLTRIVGGQECKDGECPWQALLINE
ENEGFCGGTILSEFYILTAAHCLYQAKRFKVRVGDRNTEQEEGGEAVHEVE
VVIKHNRFTKETYDFDIAVLRLKTPITFRMNVAPACLPERDWAESTLMTQK
TGIVSGFGRTHEKGRQSTRLKMLEVPYVDRNSCKLSSSFIITQNMFCAGYD
TKQEDACQGDSGGPHVTRFKDTYFVTGIVSWGEGCARKGKYGIYTKVTAFL
KWIDRSMKTRGLPKAKSHAPEVITSSPLK

The signal peptide is cleaved off by signal peptidase during export into the endoplasmic reticulum. The propeptide sequence is cleaved off after gamma carboxylation took place at the first 11 glutamic acid residues at the N-terminus of the mature N-terminal chain. A further processing step occurs by cleavage between Arg182 and Ser183. This processing step also leads concomitantly to the deletion of the tripeptide Arg180-Lys181-Arg182. The resulting secreted factor X zymogen consists of an N-terminal light chain of 139 amino acids (M, 16,200) and a C-terminal heavy chain of 306 amino acids (M, 42,000) which are covalently linked via a disulfide bridge between Cys172 and Cys342. Further posttranslational processing steps include the β-hydroxylation of Asp103 as well as N- and O-type glycosylation.

It will be understood that in addition to wild type (WT) versions of these clotting factors or biologically active portions thereof, the heterologous moieties in the chimeric molecules disclosed herein can also comprise precursor truncated forms thereof that have activity, allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the mature form of the clotting factor and which retain the ability to promote clot formation. For example, modified FVII polypeptides and variants thereof which retain at least one activity of FVII, such as TF binding, factor X binding, phospholipid binding, and/or coagulant activity of FVII can be employed. By retaining activity, the activity can be altered, such as reduced or increased, as compared to a wild-type clotting factor so long as the level of activity retained is sufficient to yield a detectable effect.

Exemplary modified polypeptides include, but are not limited to, tissue-specific isoforms and allelic variants thereof, synthetic molecules prepared by translation of nucleic acids, proteins generated by chemical synthesis, such as syntheses that include ligation of shorter polypeptides, through recombinant methods, proteins isolated from human and non-human tissue and cells, chimeric polypeptides and modified forms thereof. The clotting factors can also consist of fragments or portions of WT molecules that are of sufficient length or include appropriate regions to retain at least one activity (upon activation if needed) of a full-length mature polypeptide. Exemplary clotting factor variants are known in the art.

The “Gla domain” refers to the conserved membrane binding motif which is present in vitamin K-dependent proteins, such as prothrombin, coagulation factors VII, IX and X, proteins C, S, and Z. These proteins require vitamin K for the posttranslational synthesis of γ-carboxyglutamic acid, an amino acid clustered in the N-terminal Gla domain of these proteins. All glutamic residues present in the domain are potential carboxylation sites and many of them are therefore modified by carboxylation. In the presence of calcium ions, the Gla domain interacts with phospholipid membranes that include phosphatidylserine. The Gla domain also plays a role in binding to the FVIIa cofactor, tissue factor (TF). Complexed with TF, the Gla domain of FVIIa is loaded with seven Ca²⁺ ions, projects three hydrophobic side chains in the direction of the cell membrane for interaction with phospholipids on the cell surface, and has significant contact with the C-terminal domain of TF.

The Gla domain of factor VII comprises the uncommon amino acid γ-carboxyglutamic acid (Gla), which plays a vital role in the binding of clotting factors to negatively charged phospholipid surfaces. The Gla domain is responsible for the high-affinity binding of calcium ions. It starts at the N-terminal extremity of the mature form of proteins and ends with a conserved aromatic residue. A conserved Gla-x(3)-Gla-x-Cys motif is found in the middle of the domain which seems to be important for substrate recognition by the carboxylase. Using stopped-flow fluorescence kinetic measurements in combination with surface plasmon resonance analysis, the Gla domain has been found to be important in the sequence of events whereby the protease domain of FVIIa initiates contact with sTF (Osterlund et al., Biochem. Biophys. Res. Commun. 337:1276 (2005)). In addition, clearance of clotting factors can be significantly mediated through Gla interactions, e.g., on liver cells and clearance receptors, e.g., EPCR.

In one embodiment, the chimeric molecule comprises a heterologous moiety comprising a clotting factor modified to lack a Gla domain. The Gla domain is responsible for mediating clearance of clotting factors via multiple pathways, such as binding to liver cells, clearance receptors such as EPCR, etc. Thus, eliminating the Gla domain has beneficial effects on half-life of clotting factors. Though Gla domain is also generally required for activity by localizing clotting factors to sites of coagulation, the inclusion of a platelet targeting domain moiety (e.g., a GPIIb/IIIa antibody or antigen-binding molecule thereof) targets the Gla deleted clotting factor to platelets. Accordingly, in one embodiment, the chimeric molecule comprises a targeting moiety (e.g., a GPIIb/IIIa antibody or antigen-binding molecule thereof) and a heterologous moiety comprising a clotting factor that lacks a Gla domain. For example, in the case of Factor VII, the Gla domain is present at the amino terminus of the light chain and consists of amino acids 1-35. The Gla domains of the exemplary clotting factors disclosed herein are known in the art. The Gla domain can be removed using standard molecular biology techniques, replaced with a targeting domain, and the modified light chain incorporated into a construct of the invention. In one embodiment, a cleavage site can be introduced into constructs lacking a Gla domain to facilitate activation of the molecule. For example, in one embodiment, such a cleavage site can be introduced between the amino acids that are cleaved when the clotting factor is activated (e.g., between amino acids 152 and 153 in the case of Factor VII).

In one embodiment, a cleavage site can be introduced into chimeric molecules comprising a clotting factor that lacks a Gla domain to facilitate activation of the molecule. For example, in one embodiment, such a cleavage site can be introduced between the amino acids that are cleaved when the clotting factor is activated (e.g., between amino acids 152 and 153 in the case of Factor VII). Exemplary clotting factors lacking a Gla domain are known in the art. Exemplary clotting factors are those of mammalian, e.g., human, origin.

### 2. Half-life Extending Moieties

In some embodiments, the chimeric molecule comprises at last one heterologous moiety that is a “half-life extending moiety.” Half-life extending moieties, as discussed below in detail, can comprise, for example, (i) XTEN polypeptides; (ii) Fc; (iii) albumin, (iv) albumin binding polypeptide or fatty acid, (v) the C-terminal peptide (CTP) of the 13 subunit of human chorionic gonadotropin, (vi) PAS; (vii) HAP; (viii) transferrin; (ix) polyethylene glycol (PEG); (x) hydroxyethyl starch (HES), (xi) polysialic acids (PSAs); (xii) a clearance receptor or fragment thereof which blocks binding of the chimeric molecule to a clearance receptor; (xiii) low complexity peptides; (xiv) vWF; or (xv) any combinations thereof. In some embodiments, the half-life extending moiety comprises an Fc region. In other embodiments, the half-life extending moiety comprises two Fc regions fused by a linker. Exemplary heterologous moieties also include, e.g., FcRn binding moieties (e.g., complete Fc regions or portions thereof which bind to FcRn), single chain Fc regions (scFc regions, e.g., as described in U.S. Publ. No. 2008-0260738, and Intl. Publ. Nos. WO 2008-012543 and WO 2008-1439545), or processable scFc regions. In some embodiments, a heterologous moiety can include an attachment site for a non-polypeptide moiety such as polyethylene glycol (PEG), hydroxyethyl starch (HES), polysialic acid, or any derivatives, variants, or combinations of these moieties.

In certain embodiments, a chimeric molecule of the disclosure comprises at least one (e.g., one, two, three, four) half-like extending moiety which increases the in vivo half-life of the chimeric molecule compared with the in vivo half-life of the corresponding chimeric molecule lacking such heterologous moiety. In vivo half-life of a chimeric molecule can be determined by any method known to those of skill in the art, e.g., activity assays (chromogenic assay or one stage clotting aPTT assay), ELISA, etc. In some embodiments, the presence of one or more half-life extending moiety results in the half-life of the chimeric molecule to be increased compared to the half-life of the corresponding chimeric molecule lacking such one or more half-life extending moieties. The half-life of the chimeric molecule comprising a half-life extending moiety is at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than the in vivo half-life of the corresponding chimeric molecule lacking such half-life extending moiety.

In one embodiment, the half-life of the chimeric molecule comprising a half-life extending moiety is about 1.5-fold to about 20-fold, about 1.5 fold to about 15 fold, or about 1.5 fold to about 10 fold longer than the in vivo half-life of the corresponding chimeric molecule lacking such half-life extending moiety. In another embodiment, the half-life of chimeric molecule comprising a half-life extending moiety is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to the in vivo half-life of the corresponding chimeric molecule lacking such half-life extending moiety.

(i) XTEN Polypeptides

“XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a chimeric molecule partner, XTENs can serve as a carrier, conferring certain desirable pharmacokinetic, physicochemical and pharmaceutical properties when linked to a clotting factor, a heavy chain of a clotting factor, a light chain or a clotting factor, a targeting moiety, or any other sequences or molecules on the chimeric molecule. Such desirable properties include but are not limited to enhanced pharmacokinetic parameters and solubility characteristics. As used herein, “XTEN” specifically excludes antibodies or antibody fragments such as single-chain antibodies or Fc fragments of a light chain or a heavy chain.

The chimeric molecules of the invention can include a single XTEN polypeptide or two or more (e.g., two, three, four, five) XTEN polypeptides. In one embodiment, a chimeric molecule comprises a FVII, a first XTEN polypeptide, a second XTEN polypeptide, and an anti-GPIIb/IIIa antibody or antigen-binding molecule thereof. The chimeric molecule thus can comprise a formula of FVII-(L1)-X1-(L2)-Ab-(L3)-X2, X2-(L1)-Ab-(L2)-X1-(L3)-FVII, FVII-(L1)-X1-(L2)-X2-(L3)-Ab, or Ab-(L3)-X2-(L2)-X1-(L1)-FVII, wherein FVII comprises FVIIa, X1 is a first XTEN polypeptide, X2 is a second XTEN polypeptide, Ab is an anti-GPIIb/IIIa antibody or antigen-binding molecule thereof as described above, L1 is a first optional linker, L2 is a second optional linker, and L3 is a third optional linker. In another embodiment, a chimeric molecule comprises two polypeptide chains associated with each other, the first polypeptide chain comprising a light chain of FVII and a first XTEN polypeptide the second polypeptide chain comprising a heavy chain of FVII, a second XTEN polypeptide, and a targeting moiety, which binds to a platelet, in any order. In other embodiments, a chimeric molecule comprises two polypeptide chains associated with each other, the first polypeptide chain comprising a light chain of FVII and the first XTEN polypeptide a second polypeptide chain comprising, from N-terminus to C-terminus, a heavy chain of FVII, a second XTEN polypeptide, and a targeting moiety, which binds to a platelet or a heavy chain of FVII, a targeting moiety, which binds to a platelet, and a second XTEN polypeptide.

In some embodiments, the XTEN sequence of the invention is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues.

The XTEN sequence of the invention can comprise one or more sequence motif of 9 to 14 amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids selected from the group consisting of glycine (G), alanine (A), serine (S), threonine (T), glutamate (E) and proline (P). See US 2010-0239554 A1.

In some embodiments, the XTEN comprises non-overlapping sequence motifs in which about 80%, or at least about 85%, or at least about 90%, or about 91%, or about 92%, or about 93%, or about 94%, or about 95%, or about 96%, or about 97%, or about 98%, or about 99% or about 100% of the sequence consists of multiple units of non-overlapping sequences selected from a single motif family selected from TABLE 2, resulting in a family sequence. As used herein, “family” means that the XTEN has motifs selected only from a single motif category from TABLE 2; i.e., AD, AE, AF, AG, AM, AQ, BC, or BD XTEN, and that any other amino acids in the XTEN not from a family motif are selected to achieve a needed property, such as to permit incorporation of a restriction site by the encoding nucleotides, incorporation of a cleavage sequence, or to achieve a better linkage to FVII. In some embodiments of XTEN families, an XTEN sequence comprises multiple units of non-overlapping sequence motifs of the AD motif family, or of the AE motif family, or of the AF motif family, or of the AG motif family, or of the AM motif family, or of the AQ motif family, or of the BC family, or of the BD family, with the resulting XTEN exhibiting the range of homology described above. In other embodiments, the XTEN comprises multiple units of motif sequences from two or more of the motif families of TABLE 2. These sequences can be selected to achieve desired physical/chemical characteristics, including such properties as net charge, hydrophilicity, lack of secondary structure, or lack of repetitiveness that are conferred by the amino acid composition of the motifs, described more fully below. In the embodiments hereinabove described in this paragraph, the motifs incorporated into the XTEN can be selected and assembled using the methods described herein to achieve an XTEN of about 36 to about 3000 amino acid residues. Additional, non-limiting, examples of XTENs linked to FVII are disclosed in U.S. Patent Publication No. 2012/0263701, which is incorporated herein by reference in its entirety.

TABLE 2
XTEN Sequence Motifs of 12 Amino
Acids and Motif Families

| Motif | MOTIF | SEQ |
|---|---|---|
| Family* | SEQUENCE | ID NO: |
| AD | GESPGGSSGSES | 199 |
| AD | GSEGSSGPGESS | 200 |
| AD | GSSESGSSEGGP | 201 |
| AD | GSGGEPSESGSS | 202 |
| AE, AM | GSPAGSPTSTEE | 203 |
| AE, AM, AQ | GSEPATSGSETP | 204 |
| AE, AM, AQ | GTSESATPESGP | 205 |
| AE, AM, AQ | GTSTEPSEGSAP | 206 |
| AF, AM | GSTSESPSGTAP | 207 |
| AF, AM | GTSTPESGSASP | 208 |
| AF, AM | GTSPSGESSTAP | 209 |
| AF, AM | GSTSSTAESPGP | 210 |
| AG, AM | GTPGSGTASSSP | 211 |
| AG, AM | GSSTPSGATGSP | 212 |
| AG, AM | GSSPSASTGTGP | 213 |
| AG, AM | GASPGTSSTGSP | 214 |
| AQ | GEPAGSPTSTSE | 215 |
| AQ | GTGEPSSTPASE | 216 |
| AQ | GSGPSTESAPTE | 217 |
| AQ | GSETPSGPSETA | 218 |
| AQ | GPSETSTSEPGA | 219 |
| AQ | GSPSEPTEGTSA | 220 |
| BC | GSGASEPTSTEP | 221 |
| BC | GSEPATSGTEPS | 222 |
| BC | GTSEPSTSEPGA | 223 |
| BC | GTSTEPSEPGSA | 224 |
| BD | GSTAGSETSTEA | 225 |
| BD | GSETATSGSETA | 226 |
| BD | GTSESATSESGA | 227 |
| BD | GTSTEASEGSAS | 228 |

*Denotes individual motif sequences that, when used together in various permutations, results in a “family sequence”

XTEN can have varying lengths. In one embodiment, the length of the XTEN polypeptide(s) is chosen based on the property or function to be achieved in the fusion protein. Depending on the intended property or function, XTEN can be short or intermediate length sequence or longer sequence that can serve as carriers. In certain embodiments, the XTEN include short segments of about 6 to about 99 amino acid residues, intermediate lengths of about 100 to about 399 amino acid residues, and longer lengths of about 400 to about 1000 and up to about 3000 amino acid residues. Thus, the XTEN linked to FVII (e.g., heavy chain or light chain) or a targeting moiety can have lengths of about 6, about 12, about 36, about 40, about 42, about 72, about 96, about 144, about 288, about 400, about 500, about 576, about 600, about 700, about 800, about 864, about 900, about 1000, about 1500, about 2000, about 2500, or up to about 3000 amino acid residues in length. In other embodiments, the XTEN sequences is about 6 to about 50, about 50 to about 100, about 100 to 150, about 150 to 250, about 250 to 400, about 400 to about 500, about 500 to about 900, about 900 to 1500, about 1500 to 2000, or about 2000 to about 3000 amino acid residues in length. The precise length of an XTEN polypeptide that can be linked to FVII (e.g., light chain or heavy chain) or a targeting moiety (Ab) can vary without adversely affecting the activity of FVII. In one embodiment, one or more of the XTEN used herein has about 42 amino acids, about 72 amino acids, about 108 amino acids, about 144 amino acids, about 180 amino acids, about 216 amino acids, about 252 amino acids, about 288 amino acids, about 324 amino acids, about 360 amino acids, about 396 amino acids, about 432 amino acids, about 468 amino acids, about 504 amino acids, about 540 amino acids, about 576 amino acids, about 612 amino acids, about 624 amino acids, about 648 amino acids, about 684 amino acids, about 720 amino acids, about 756 amino acids, about 792 amino acids, about 828 amino acids, about 836 amino acids, about 864 amino acids, about 875 amino acids, about 912 amino acids, about 923 amino acids, about 948 amino acids, about 1044 amino acids, about 1140 amino acids, about 1236 amino acids, about 1318 amino acids, about 1332 amino acids, about 1428 amino acids, about 1524 amino acids, about 1620 amino acids, about 1716 amino acids, about 1812 amino acids, about 1908 amino acids, or about 2004 amino acids in length and can be selected from one or more of the XTEN family sequences; i.e., AD, AE, AF, AG, AM, AQ, BC, BD, or any combinations thereof.

In some embodiments, the XTEN polypeptide used in the invention is at least 60%, 70%, 80%, 85%, 90%, 91%, 92%, 93%, 94%, 95%, 96%, 97%, 98%, 99%, or 100% identical to a sequence selected from the group consisting of AE42, AG42, AE42_2, AE42_3, AE48, AM48, AE72, AE72_2, AE72_3, AG72, AE108, AG108, AE144, AF144, AE144_2, AE144_3, AG144, AE180, AG180, AE216, AG216, AE252, AG252, AE288, AG288, AE295, AE324, AG324, AE360, AG360, AE396, AG396, AE432, AG432, AE468, AG468, AE504, AG504, AF504, AE540, AG540, AF540, AD576, AE576, AF576, AG576, AE612, AG612, AE624, AE648, AG648, AG684, AE720, AG720, AE756, AG756, AE792, AG792, AE828, AG828, AD836, AE864, AF864, AG864, AE872, AE884, AM875, AE912, AM923, AM1318, BC864, BD864, AE948, AE1044, AE1140, AE1236, AE1332, AE1428, AE1524, AE1620, AE1716, AE1812, AE1908, AE2004A, AG948, AG1044, AG1140, AG1236, AG1332, AG1428, AG1524, AG1620, AG1716, AG1812, AG1908, AG2004, and any combinations thereof. See US 2010-0239554 A1.

In one embodiment, the XTEN sequence is at least 60%, 70%, 80%, 90%, 95%, 96%, 97%, 98%, 99% or 100% identical to an amino acid sequence selected from the group consisting of AE42, AE864, AE576, AE288, AE144, AG864, AG576, AG288, AG144, and any combinations thereof. In another embodiment, the XTEN sequence is selected from the group consisting of AE42, AE864, AE576, AE288, AE144, AG864, AG576, AG288, AG144, and any combinations thereof. In one embodiment, the XTEN sequence is AE144. In a specific embodiment, the XTEN sequence is AE288. The amino acid sequences for certain XTEN sequences of the invention are shown in TABLE 3.

TABLE 3
XTEN Sequences

| XTEN | Amino Acid Sequence |
|---|---|
| AE42 | GAPGSPAGSPTSTEEGTSESATPE |
| SEQ ID NO: 229 | SGPGSEPATSGSETPASS |
| AE42_2 | TGGGSPAGSPTSTEEGTSESATPE |
| SEQ ID NO: 230 | SGPGSEPATSGSETPASS |
| AE42_3 | GTSESATPESGPGSEPATSGSETP |
| SEQ ID NO: 231 | GTSESATPESGPGSEPAT |
| AE72 | GAP TSESATPESG PGSEPATSGS |
| SEQ ID NO: 232 | ETPGTSESAT PESGPGSEPA |
| | TSGSETPGTS ESATPESGPG |
| | TSTEPSEGSA PGASS |
| AE72_2 | GTSESATPESGPGSEPATSGSETPG |
| SEQ ID NO: 233 | TSESATPESGPGSEPATSGSETPGT |
| | SESATPESGPGTSTEPSEGSAP |
| AE72_3 | SPAGSPTSTEEGTSESATPESGPGS |
| SEQ ID NO: 234 | EPATSGSETPGTSESATPESGPGTS |
| | TEPSEGSAPGTSTEPSEGSAPG |
| AE144 | GSEPATSGSETPGTSESATPESGPG |
| SEQ ID NO: 235 | SEPATSGSETPGSPAGSPTSTEEGT |
| | STEPSEGSAPGSEPATSGSETPGSE |
| | PATSGSETPGSEPATSGSETPGTST |
| | EPSEGSAPGTSESAPESGPGSEPAT |
| | SGSETPGTSTEPSEGSAP |
| AE144_2 | GTSESATPESGPGSEPATSGSETPG |
| SEQ ID NO: 236 | TSESATPESGPGSEPATSGSETPGT |
| | SESATPESGPGTSTEPSEGSAPGSP |
| | AGSPTSTEEGTSESATPESGPGSEP |
| | ATSGSETPGTSESATPESGPGSPAG |
| | SPTSTEEGSPAGSPTSTEE |
| AE144_3 | GSPAGSPTSTEEGTSESATPESGPG |
| SEQ ID NO: 237 | TSTEPSEGSAPGSPAGSPTSTEEGT |
| | STEPSEGSAPGTSTEPSEGSAPGTS |
| | ESATPESGPGSEPATSGSETPGSEP |
| | ATSGSETPGSPAGSPTSTEEGTSES |
| | ATPESGPGTSTEPSEGSAP |
| AG 144 | GTPGSGTASSSPGSSTPSGATGSPG |
| SEQ ID NO: 238 | SSPSASTGTGPGSSPSASTGTGPGA |
| | SPGTSSTGSPGASPGTSSTGSPGSS |
| | TPSGATGSPGSSPSASTGTGPGASP |
| | GTSSTGSPGSSPSASTGTGPGTPGS |
| | GTASSSPGSSTPSGATGSP |
| AE288 | GTSESATPESGPGSEPATSGSETPG |
| SEQ ID NO: 239 | TSESATPESGPGSEPATSGSETPGT |
| | SESATPESGPGTSTEPSEGSAPGSP |
| | AGSPTSTEEGTSESATPESGPGSEP |
| | ATSGSETPGTSESATPESGPGSPAG |
| | SPTSTEEGSPAGSPTSTEEGTSTEP |
| | SEGSAPGTSESATPESGPGTSESAT |
| | PESGPGTSESATPESGPGSEPATSG |
| | SETPGSEPATSGSETPGSPAGSPTS |
| | TEEGTSTEPSEGSAPGTSTEPSEGS |
| | APGSEPATSGSETPGTSESATPESG |
| | PGTSTEPSEGSAP |
| AG288 | PGASPGTSSTGSPGASPGTSSTGSP |
| SEQ ID NO: 240 | GTPGSGTASSSPGSSTPSGATGSPG |
| | TPGSGTASSSPGSSTPSGATGSPGT |
| | PGSGTASSSPGSSTPSGATGSPGSS |
| | TPSGATGSPGSSPSASTGTGPGSSP |
| | SASTGTGPGASPGTSSTGSPGTPGS |
| | GTASSSPGSSTPSGATGSPGSSPSA |
| | STGTGPGSSPSASTGTGPGASPGTS |
| | STGSPGASPGTSSTGSPGSSTPSGA |
| | TGSPGSSPSASTGTGPGASPGTSST |
| | GSPGSSPSASTGTGPGTPGSGTASS |
| | SPGSSTPSGATGS |
| AE576 | GSPAGSPTSTEEGTSESATPESGPG |
| SEQ ID NO: 241 | TSTEPSEGSAPGSPAGSPTSTEEGT |
| | STEPSEGSAPGTSTEPSEGSAPGTS |
| | ESATPESGPGSEPATSGSETPGSEP |
| | ATSGSETPGSPAGSPTSTEEGTSES |
| | ATPESGPGTSTEPSEGSAPGTSTEP |
| | SEGSAPGSPAGSPTSTEEGTSTEPS |
| | EGSAPGTSTEPSEGSAPGTSESATP |
| | ESGPGTSTEPSEGSAPGTSESATPE |
| | SGPGSEPATSGSETPGTSTEPSEGS |
| | APGTSTEPSEGSAPGTSESATPESG |
| | PGTSESATPESGPGSPAGSPTSTEE |
| | GTSESATPESGPGSEPATSGSETPG |
| | TSESATPESGPGTSTEPSEGSAPGT |
| | STEPSEGSAPGTSTEPSEGSAPGTS |
| | TEPSEGSAPGTSTEPSEGSAPGTST |
| | EPSEGSAPGSPAGSPTSTEEGTSTE |
| | PSEGSAPGTSESATPESGPGSEPAT |
| | SGSETPGTSESATPESGPGSEPATS |
| | GSETPGTSESATPESGPGTSTEPSE |
| | GSAPGTSESATPESGPGSPAGSPTS |
| | TEEGSPAGSPTSTEEGSPAGSPTST |
| | EEGTSESATPESGPGTSTEPSEGSA |
| | P |
| AG576 | PGTPGSGTASSSPGSSTPSGATGSP |
| SEQ ID NO: 242 | GSSPSASTGTGPGSSPSASTGTGPG |
| | SSTPSGATGSPGSSTPSGATGSPGA |
| | SPGTSSTGSPGASPGTSSTGSPGAS |
| | PGTSSTGSPGTPGSGTASSSPGASP |
| | GTSSTGSPGASPGTSSTGSPGASPG |
| | TSSTGSPGSSPSASTGTGPGTPGSG |
| | TASSSPGASPGTSSTGSPGASPGTS |
| | STGSPGASPGTSSTGSPGSSTPSGA |
| | TGSPGSSTPSGATGSPGASPGTSST |
| | GSPGTPGSGTASSSPGSSTPSGATG |
| | SPGSSTPSGATGSPGSSTPSGATGS |
| | PGSSPSASTGTGPGASPGTSSTGSP |
| | GASPGTSSTGSPGTPGSGTASSSPG |
| | ASPGTSSTGSPGASPGTSSTGSPGA |
| | SPGTSSTGSPGASPGTSSTGSPGTP |
| | GSGTASSSPGSSTPSGATGSPGTPG |
| | SGTASSSPGSSTPSGATGSPGTPGS |
| | GTASSSPGSSTPSGATGSPGSSTPS |
| | GATGSPGSSPSASTGTGPGSSPSAS |
| | TGTGPGASPGTSSTGSPGTPGSGTA |
| | SSSPGSSTPSGATGSPGSSPSASTG |
| | TGPGSSPSASTGTGPGASPGTSSTG |
| | S |
| AE864 | GSPAGSPTSTEEGTSESATPESGPG |
| SEQ ID NO: 243 | TSTEPSEGSAPGSPAGSPTSTEEGT |
| | STEPSEGSAPGTSTEPSEGSAPGTS |
| | ESATPESGPGSEPATSGSETPGSEP |
| | ATSGSETPGSPAGSPTSTEEGTSES |
| | ATPESGPGTSTEPSEGSAPGTSTEP |
| | SEGSAPGSPAGSPTSTEEGTSTEPS |
| | EGSAPGTSTEPSEGSAPGTSESATP |
| | ESGPGTSTEPSEGSAPGTSESATPE |
| | SGPGSEPATSGSETPGTSTEPSEGS |
| | APGTSTEPSEGSAPGTSESATPESG |
| | PGTSESATPESGPGSPAGSPTSTEE |
| | GTSESATPESGPGSEPATSGSETPG |
| | TSESATPESGPGTSTEPSEGSAPGT |
| | STEPSEGSAPGTSTEPSEGSAPGTS |
| | TEPSEGSAPGTSTEPSEGSAPGTST |
| | EPSEGSAPGSPAGSPTSTEEGTSTE |
| | PSEGSAPGTSESATPESGPGSEPAT |
| | SGSETPGTSESATPESGPGSEPATS |
| | GSETPGTSESATPESGPGTSTEPSE |
| | GSAPGTSESATPESGPGSPAGSPTS |
| | TEEGSPAGSPTSTEEGSPAGSPTST |
| | EEGTSESATPESGPGTSTEPSEGSA |
| | PGTSESATPESGPGSEPATSGSETP |
| | GTSESATPESGPGSEPATSGSETPG |
| | TSESATPESGPGTSTEPSEGSAPGS |
| | PAGSPTSTEEGTSESATPESGPGSE |
| | PATSGSETPGTSESATPESGPGSPA |
| | GSPTSTEEGSPAGSPTSTEEGTSTE |
| | PSEGSAPGTSESATPESGPGTSESA |
| | TPESGPGTSESATPESGPGSEPATS |
| | GSETPGSEPATSGSETPGSPAGSPT |
| | STEEGTSTEPSEGSAPGTSTEPSEG |
| | SAPGSEPATSGSETPGTSESATPES |
| | GPGTSTEPSEGSAP |
| AG864 | GASPGTSSTGSPGSSPSASTGTGPG |
| SEQ ID NO: 244 | SSPSASTGTGPGTPGSGTASSSPGS |
| | STPSGATGSPGSSPSASTGTGPGAS |
| | PGTSSTGSPGTPGSGTASSSPGSST |
| | PSGATGSPGTPGSGTASSSPGASPG |
| | TSSTGSPGASPGTSSTGSPGTPGSG |
| | TASSSPGSSTPSGATGSPGASPGTS |
| | STGSPGTPGSGTASSSPGSSTPSGA |
| | TGSPGSSPSASTGTGPGSSPSASTG |
| | TGPGSSTPSGATGSPGSSTPSGATG |
| | SPGASPGTSSTGSPGASPGTSSTGS |
| | PGASPGTSSTGSPGTPGSGTASSSP |
| | GASPGTSSTGSPGASPGTSSTGSPG |
| | ASPGTSSTGSPGSSPSASTGTGPGT |
| | PGSGTASSSPGASPGTSSTGSPGAS |
| | PGTSSTGSPGASPGTSSTGSPGSST |
| | PSGATGSPGSSTPSGATGSPGASPG |
| | TSSTGSPGTPGSGTASSSPGSSTPS |
| | GATGSPGSSTPSGATGSPGSSTPSG |
| | ATGSPGSSPSASTGTGPGASPGTSS |
| | TGSPGASPGTSSTGSPGTPGSGTAS |
| | SSPGASPGTSSTGSPGASPGTSSTG |
| | SPGASPGTSSTGSPGASPGTSSTGS |
| | PGTPGSGTASSSPGSSTPSGATGSP |
| | GTPGSGTASSSPGSSTPSGATGSPG |
| | TPGSGTASSSPGSSTPSGATGSPGS |
| | STPSGATGSPGSSPSASTGTGPGSS |
| | PSASTGTGPGASPGTSSTGSPGTPG |
| | SGTASSSPGSSTPSGATGSPGSSPS |
| | ASTGTGPGSSPSASTGTGPGASPGT |
| | SSTGSPGASPGTSSTGSPGSSTPSG |
| | ATGSPGSSPSASTGTGPGASPGTSS |
| | TGSPGSSPSASTGTGPGTPGSGTAS |
| | SSPGSSTPSGATGSPGSSTPSGATG |
| | SPGASPGTSSTGSP |

In some embodiments wherein the XTEN has less than 100% of its amino acids consisting of 4, 5, or 6 types of amino acid selected from glycine (G), alanine (A), serine (S), threonine (T), glutamate (E) and proline (P), or less than 100% of the sequence consisting of the sequence motifs from Table 2 or the XTEN sequences of Table 3, the other amino acid residues of the XTEN are selected from any of the other 14 natural L-amino acids, but are preferentially selected from hydrophilic amino acids such that the XTEN sequence contains at least about 90%, 91%, 92%, 93%, 94%, 95%, 96%, 97%, 98%, or at least about 99% hydrophilic amino acids. An individual amino acid or a short sequence of amino acids other than glycine (G), alanine (A), serine (S), threonine (T), glutamate (E) and proline (P) may be incorporated into the XTEN to achieve a needed property, such as to permit incorporation of a restriction site by the encoding nucleotides, or to facilitate linking to a payload component, or incorporation of a cleavage sequence. The XTEN amino acids that are not glycine (G), alanine (A), serine (S), threonine (T), glutamate (E) and proline (P) are either interspersed throughout the XTEN sequence, are located within or between the sequence motifs, or are concentrated in one or more short stretches of the XTEN sequence such as at or near the N- or C-terminus. As hydrophobic amino acids impart structure to a polypeptide, the invention provides that the content of hydrophobic amino acids in the XTEN utilized in the conjugation constructs will typically be less than 5%, or less than 2%, or less than 1% hydrophobic amino acid content. Hydrophobic residues that are less favored in construction of XTEN include tryptophan, phenylalanine, tyrosine, leucine, isoleucine, valine, and methionine. Additionally, one can design the XTEN sequences to contain less than 5% or less than 4% or less than 3% or less than 2% or less than 1% or none of the following amino acids: methionine (to avoid oxidation), asparagine and glutamine (to avoid deamidation). In other embodiments, the amino acid content of methionine and tryptophan in the XTEN component used in the conjugation constructs is typically less than 5%, or less than 2%, and most preferably less than 1%. In other embodiments, the XTEN will have a sequence that has less than 10% amino acid residues with a positive charge, or less than about 7%, or less that about 5%, or less than about 2% amino acid residues with a positive charge, the sum of methionine and tryptophan residues will be less than 2%, and the sum of asparagine and glutamine residues will be less than 5% of the total XTEN sequence.

In further embodiments, the XTEN polypeptide used in the invention affects the physical or chemical property, e.g., pharmacokinetics, of the chimeric molecule of the present disclosure. The XTEN sequence used in the present disclosure can exhibit one or more of the following advantageous properties: conformational flexibility, enhanced aqueous solubility, high degree of protease resistance, low immunogenicity, low binding to mammalian receptors, or increased hydrodynamic (or Stokes) radii. In a specific embodiment, the XTEN polypeptide linked to FVII or a targeting moiety (e.g., an anti-GPIIb/IIIa antibody or antigen-binding molecule thereof) in in this invention increases pharmacokinetic properties such as longer terminal half-life or increased area under the curve (AUC), so that the chimeric molecule described herein stays in vivo for an increased period of time compared to wild type clotting factor. In further embodiments, the XTEN polypeptide used in this invention increases pharmacokinetic properties such as longer terminal half-life or increased area under the curve (AUC), so that the clotting factor stays in vivo for an increased period of time compared to wild type FVIIa.

A variety of methods and assays can be employed to determine the physical/chemical properties of proteins comprising the XTEN polypeptide. Such methods include, but are not limited to analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Amau et al., Prot Expr and Purif 48, 1-13 (2006).

Additional examples of XTEN polypeptides that can be used according to the present disclosure and are disclosed in U.S. Pat. Nos. 7,855,279 and 7,846,445, US Patent Publication Nos. 2009/0092582 A1, 2010/0239554 A1, 2010/0323956 A1, 2011/0046060 A1, 2011/0046061 A1, 2011/0077199 A1, 2011/0172146 A1, 2013/0017997 A1, or 2012/0263701 A1, International Patent Publication Nos. WO 2010091122 A1, WO 2010144502 A2, WO 2010144508 A1, WO 2011028228 A1, WO 2011028229 A1, or WO 2011028344 A2; or US 2012/0178691.

(ii) Fc and Single Chain Fc (scFc) Region

In certain embodiments, the chimeric molecule comprises at least one heterologous moiety comprising a Fc region. “Fc” or “Fc region” as used herein means a functional neonatal Fc receptor (FcRn) binding partner comprising an Fc domain, variant, or fragment thereof, unless otherwise specified. An FcRn binding partner is any molecule that can be specifically bound by the FcRn receptor with consequent active transport by the FcRn receptor of the FcRn binding partner. Thus, the term Fc includes any variants of IgG Fc that are functional. The region of the Fc portion of IgG that binds to the FcRn receptor has been described based on X-ray crystallography (Burmeister et al., Nature, 372:379 (1994), incorporated herein by reference in its entirety). The major contact area of the Fc with the FcRn is near the junction of the CH2 and CH3 domains. Fc-FcRn contacts are all within a single Ig heavy chain. FcRn binding partners include, but are not limited to, whole IgG, the Fc fragment of IgG, and other fragments of IgG that include the complete binding region of FcRn. An Fc can comprise the CH2 and CH3 domains of an immunoglobulin with or without the hinge region of the immunoglobulin. Also included are Fc fragments, variants, or derivatives which maintain the desirable properties of an Fc region in a chimeric molecule, e.g., an increase in half-life, e.g., in vivo half-life. Myriad mutants, fragments, variants, and derivatives are described, e.g., in PCT Publication Nos. WO2011/069164, WO2012/006623, WO2012/006635, or WO 2012/006633, all of which are incorporated herein by reference in their entireties. In some embodiments, the chimeric molecule comprises a dotting factor (e.g., a FVII), a targeting moiety (e.g., a GPIIb/IIIa antibody or antigen-binding molecule thereof), and an Fc region.

In one embodiment, the chimeric molecule comprises a heterologous moiety comprising one genetically fused Fc region or a portion thereof within a single polypeptide chain (i.e., a single-chain Fc (scFc) region). An exemplary single-chain human IgG1 Fc amino acid sequence is provided below (the Gly/Ser linker is underlined):

(SEQ ID NO: 135)
DKTHTCPPCPAPELLGGPSVFLFPPKPKDTLMIS
RTPEVTCVVVDVSHEDPEVKFNWYVDGVEVHNAK
TKPREEQYNSTYRVVSVLTVLHQDWLNGKEYKCK
VSNKALPAPIEKTISKAKGQPREPQVYTLPPSRD
ELTKNQVSLTCLVKGFYPSDIAVEWESNGQPENN
YKTTPPVLDSDGSFFLYSKLTVDKSRWQQGNVFS
CSVMHEALHNHYTQKSLSLSPGKGGGGSGGGGSG
GGGSGGGGSDKTHTCPPCPAPELLGGPSVFLFPP
KPKDTLMISRTPEVTCVVVDVSHEDPEVKFNWYV
DGVEVHNAKTKPREEQYNSTYRVVSVLTVLHQDW
LNGKEYKCKVSNKALPAPIEKTISKAKGQPREPQ
VYTLPPSRDELTKNQVSLTCLVKGFYPSDIAVEW
ESNGQPENNYKTTPPVLDSDGSFFLYSKLTVDKS
RWQQGNVFSCSVMHEALHNHYTQKSLSLSPGK

The unprocessed polypeptides comprise at least two immunoglobulin constant regions or portions thereof (e.g., Fc moieties or domains (e.g., 2, 3, 4, 5, 6, or more Fc moieties or domains)) within the same linear polypeptide chain that are capable of folding (e.g., intramolecularly or intermolecularly folding) to form one functional scFc region which is linked by an Fc peptide linker. For example, in one embodiment, a polypeptide of the invention is capable of binding, via its scFc region, to at least one Fc receptor (e.g., an FcRn, an FcγR receptor (e.g., FcγRIII), or a complement protein (e.g., C1q)) in order to improve half-life or trigger an immune effector function (e.g., antibody-dependent cytotoxicity (ADCC), phagocytosis, or complement-dependent cytotoxicity (CDCC) and/or to improve manufacturability). In some embodiments, the chimeric molecule comprises a clotting factor (e.g., a FVII), a targeting moiety (e.g., a GPIIb/IIIa antibody or antigen-binding molecule thereof), and an scFc region.

(iii) Albumins

In certain embodiments, the chimeric molecule comprises a heterologous moiety comprising albumin or a functional fragment thereof. Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ. Nos. US2008/0194481, US2008/0004206, US2008/0161243, US2008/0261877, or US2008/0153751 or PCT Appl. Publ. Nos. WO2008/033413, WO2009/058322, or WO2007/021494, which are incorporated herein by reference in their entireties. An exemplary mature human albumin amino acid sequence is provided below (NCBI Ref. Sequence NP_000468):

(SEQ ID NO: 136)
RGVFRRDAHKSEVAHRFKDLGEENFKALVLIAFAQYLQQCPFEDHVKLVNE
VTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHP
YFYAPELLFFAKRYKAAFTECCQAADKAACLLPKLDELRDEGKASSAKQRL
KCASLQKFGERAFKAWAVARLSQRFPKAEFAEVSKLVTDLTKVHTECCHGD
LLECADDRADLAKYICENQDSISSKLKECCEKPLLEKSHCIAEVENDEMPA
DLPSLAADFVESKDVCKNYAEAKDVFLGMFLYEYARRHPDYSVVLLLRLAK
TYETTLEKCCAAADPHECYAKVFDEFKPLVEEPQNLIKQNCELFEQLGEYK
FQNALLVRYTKKVPQVSTPTLVEVSRNLGKVGSKCCKHPEAKRMPCAEDYL
SVVLNQLCVLHEKTPVSDRVTKCCTESLVNRRPCFSALEVDETYVPKEFNA
ETFTFHADICTLSEKERQIKKQTALVELVKHKPKATKEQLKAVMDDFAAFV
EKCCKADDKETCFAEEGKKLVAASQAALGL

In some embodiments, the chimeric molecule comprises a clotting factor (e.g., a FVII), a targeting moiety (e.g., a GPIIb/IIIa antibody or antigen-binding molecule thereof), and an albumin.

(iv) Albumin Binding Polypeptides and Lipids

In certain embodiments, a heterologous moiety can comprise an albumin binding moiety, which comprises an albumin binding peptide, a bacterial albumin binding domain, an albumin-binding antibody fragment, or any combinations thereof. For example, the albumin binding protein can be a bacterial albumin binding protein, an antibody or an antibody fragment including domain antibodies (see, e.g., U.S. Pat. No. 6,696,245). An albumin binding protein, for example, can be a bacterial albumin binding domain, such as the one of streptococcal protein G (Konig and Skerra (1998) J Immunol. Methods 218, 73-83). Other examples of albumin binding peptides that can be used as conjugation partner are, for instance, those having a Cys-Xaa₁-Xaa₂-Xaa₃-Xaa₄-Cys consensus sequence (SEQ ID NO:137), wherein Xaa₁ is Asp, Asn, Ser, Thr, or Trp; Xaa₂ is Asn, Gln, H is, Ile, Leu, or Lys; Xaa₃ is Ala, Asp, Phe, Trp, or Tyr; and Xaa₄ is Asp, Gly, Leu, Phe, Ser, or Thr as described in U.S. Pub. No. US2003/0069395 or Dennis et al. (2002) J. Biol. Chem. 277, 35035-35043.

Domain 3 from streptococcal protein G, as disclosed by Kraulis et al., FEBS Lett., 378:190-194 (1996) and Linhult et al., Protein Sci., 11:206-213 (2002) is an example of a bacterial albumin-binding domain. Examples of albumin-binding peptides include a series of peptides having the core sequence DICLPRWGCLW (SEQ ID NO:138) such as:

(SEQ ID NO: 139)
RLIEDICLPRWGCLWEDD;
(SEQ ID NO: 140)
QRLMEDICLPRWGCLWEDDF;
(SEQ ID NO: 141)
QGLIGDICLPRWGCLWGDSVK;
and
(SEQ ID NO: 142)
GEWWEDICLPRWGCLWEEED.

See, e.g., Dennis et al., J. Biol. Chem. 2002, 277: 35035-35043 (2002). Examples of albumin-binding antibody fragments are disclosed in Muller and Kontermann, Curr. Opin. Mol. Ther. 9:319-326 (2007); Rooverset al., Cancer Immunol. Immunother. 56:303-317 (2007), and Holt et al., Prot. Eng. Design Sci., 21:283-288 (2008), which are incorporated herein by reference in their entireties. An example of such albumin binding moiety is 2-(3-maleimidopropanamido)-6-(4-(4-iodophenyl)butanamido) hexanoate (“Albu” tag) as disclosed by Trussel et al., Bioconjugate Chem. 20:2286-2292 (2009). Fatty acids, in particular long chain fatty acids (LCFA) and long chain fatty acid-like albumin-binding compounds can be used to extend the in vivo half-life of chimeric molecules of the invention. An example of a LCFA-like albumin-binding compound is 16-(1-(3-(9-(((2,5-dioxopyrrolidin-1-yloxy)carbonyloxy)-methyi)-7-sulfo-9H-fluoren-2-ylamino)-3-oxopropyl)-2,5-dioxopyrrolidin-3-ylthio) hexadecanoic acid (see, e.g., WO 2010/140148).

In some embodiments, the chimeric molecule comprises a dotting factor (e.g., a FVII), a targeting moiety (e.g., a GPIIb/IIIa antibody or antigen-binding molecule thereof), and an albumin binding polypeptide or lipid.

(v) CTP

In certain embodiments, a chimeric molecule disclosed herein comprises at least one heterologous moiety comprising one β subunit of the C-terminal peptide (CTP) of human chorionic gonadotropin or fragment, variant, or derivative thereof. The insertion of one or more CTP peptides into a recombinant protein is known to increase the in vivo half-life of that protein. See, e.g., U.S. Pat. No. 5,712,122, incorporated by reference herein in its entirety.

Exemplary CTP peptides include DPRFQDSSSSKAPPPSLPSPSRLPGPSDTPIL (SEQ ID NO:143) or SSSSKAPPPSLPSPSRLPGPSDTPILPQ (SEQ ID NO:144). See, e.g., U.S. Patent Appl. Publ. No. US 2009/0087411, incorporated by reference. In some embodiments, the chimeric molecule comprises two heterologous moieties that are CTP sequences. In some embodiments, three of the heterologous moieties are CTP sequences. In some embodiments, four of the heterologous moieties are CTP sequences. In some embodiments, five of the heterologous moieties are CTP sequences. In some embodiments, six or more of the heterologous moieties are CTP sequences.

In some embodiments, the chimeric molecule comprises a dotting factor (e.g., a FVII), a targeting moiety (e.g., a GPIIb/IIIa antibody or antigen-binding molecule thereof), and a CTP.

(vi) PAS

In other embodiments, at least one heterologous moiety is a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the chimeric molecule. Yet, the skilled person is aware that an amino acid polymer also can form random coil conformation when residues other than alanine, serine, and proline are added as a minor constituent in the PAS sequence.

The term “minor constituent” as used herein means that amino acids other than alanine, serine, and proline can be added in the PAS sequence to a certain degree, e.g., up to about 12%, i.e., about 12 of 100 amino acids of the PAS sequence, up to about 10%, i.e., about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids.

The amino acids different from alanine, serine and proline can be selected from Arg, Asn, Asp, Cys, Gln, Glu, Gly, His, Ile, Leu, Lys, Met, Phe, Thr, Trp, Tyr, and Val.

Under physiological conditions, the PAS sequence stretch forms a random coil conformation and thereby can mediate an increased in vivo and/or in vitro stability to the chimeric molecule. Since the random coil domain does not adopt a stable structure or function by itself, the biological activity mediated by the activatable clotting factor in the chimeric molecule is essentially preserved. In other embodiments, the PAS sequences that form random coil domain are biologically inert, especially with respect to proteolysis in blood plasma, immunogenicity, isoelectric point/electrostatic behavior, binding to cell surface receptors or internalization, but are still biodegradable, which provides clear advantages over synthetic polymers such as PEG.

Non-limiting examples of the PAS sequences forming random coil conformation comprise an amino acid sequence selected from the group consisting of ASPAAPAPASPAAPAPSAPA (SEQ ID NO:145), AAPASPAPAAPSAPAPAAPS (SEQ ID NO:146), APSSPSPSAPSSPSPASPSS (SEQ ID NO:147), APSSPSPSAPSSPSPASPS (SEQ ID NO:148), SSPSAPSPSSPASPSPSSPA (SEQ ID NO:149), AASPAAPSAPPAAASPAAPSAPPA (SEQ ID NO:150), and ASAAAPAAASAAASAPSAAA (SEQ ID NO:151), or any combinations thereof. Additional examples of PAS sequences are known from, e.g., US Pat. Publ. No. 2010/0292130 and PCT Appl. Publ. No. WO2008/155134 A1.

In some embodiments, the chimeric molecule comprises a dotting factor (e.g., a FVII), a targeting moiety (e.g., a GPIIb/IIIa antibody or antigen-binding molecule thereof), and a PAS.

(vii) HAP

In certain embodiments, at least one heterologous moiety is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)ₙ, (SEQ ID NO:152), (Gly₄Ser)ₙ (SEQ ID NO:153), or Ser(Gly₄Ser)ₙ(SEQ ID NO:154), wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200. See, e.g., Schlapschy M et al., Protein Eng. Design Selection, 20: 273-284 (2007).

In some embodiments, the chimeric molecule comprises a dotting factor (e.g., a FVII), a targeting moiety (e.g., a GPIIb/IIIa antibody or antigen-binding molecule thereof), and a HAP.

(viii) Transferrin

In certain embodiments, at least one heterologous moiety is transferrin or a peptide or fragment, variant, or derivative thereof. Any transferrin can be used to make the chimeric molecules of the invention. As an example, wild-type human TF (TF) is a 679 amino acid protein, of approximately 75 KDa (not accounting for glycosylation), with two main domains, N (about 330 amino acids) and C (about 340 amino acids), which appear to originate from a gene duplication. N domain comprises two subdomains, N1 domain and N2 domain, and C domain comprises two subdomains, C1 domain and C2 domain. See GenBank accession numbers NM001063, XM002793, M12530, XM039845, XM 039847 and 595936 (www.ncbi.nlm.nih.gov), all of which are herein incorporated by reference in their entirety. In one embodiment, the transferrin heterologous moiety includes a transferrin splice variant. In one example, a transferrin splice variant can be a splice variant of human transferrin, e.g., Genbank Accession AAA61140. In another embodiment, the transferrin portion of the chimeric molecule includes one or more domains of the transferrin sequence, e.g., N domain, C domain, N1 domain, N2 domain, C1 domain, C2 domain or any combinations thereof.

Transferrin transports iron through transferrin receptor (TfR)-mediated endocytosis. After the iron is released into an endosomal compartment and Tf-TfR complex is recycled to cell surface, the Tf is released back extracellular space for next cycle of iron transporting. Tf possesses a long half-life that is in excess of 14-17 days (Li et al., Trends Pharmacol. Sci. 23:206-209 (2002)). Transferrin fusion proteins have been studied for half-life extension, targeted deliver for cancer therapies, oral delivery and sustained activation of proinsulin (Brandsma et al., Biotechnol. Adv., 29: 230-238 (2011); Bai et al., Proc. Natl. Acad. Sci. USA 102:7292-7296 (2005); Kim et al., J. Pharmacol. Exp. Ther., 334:682-692 (2010); Wang et al., J. Controlled Release 155:386-392 (2011)).

In some embodiments, the chimeric molecule comprises a clotting factor (e.g., a FVII), a targeting moiety (e.g., a GPIIb/IIIa antibody or antigen-binding molecule thereof), and a transferrin.

(ix) PEG

In some embodiments, at least one heterologous moiety is a soluble polymer known in the art, including, but not limited to, polyethylene glycol, ethylene glycol/propylene glycol copolymers, carboxymethylcellulose, dextran, or polyvinyl alcohol. In some embodiments, the chimeric molecule comprising a PEG heterologous moiety further comprises a heterologous moiety selected from an immunoglobulin constant region or portion thereof (e.g., an Fc region), a PAS sequence, HES, and albumin, fragment, or variant thereof. In still other embodiments, the chimeric molecule comprises an activatable clotting factor or fragment thereof and a PEG heterologous moiety, wherein the chimeric molecule further comprises a heterologous moiety selected from an immunoglobulin constant region or portion thereof (e.g., an Fc moiety), a PAS sequence, HES, and albumin, fragment, or variant thereof. In yet other embodiments, the chimeric molecule comprises a clotting factor or fragment thereof, a second clotting factor or fragment thereof, and a PEG heterologous moiety, wherein the chimeric molecule further comprises a heterologous moiety selected from an immunoglobulin constant region or portion thereof (e.g., an Fc moiety), a PAS sequence, HES, and albumin, fragment, or variant thereof.

In other embodiments, the chimeric molecule comprises a clotting factor or fragment thereof, a synthetic procoagulant polypeptide, and a PEG heterologous moiety, wherein the chimeric molecule further comprises a heterologous moiety selected from an immunoglobulin constant region or portion thereof (e.g., an Fc region), a PAS sequence, HES, and albumin, fragment, or variant thereof. In other embodiments, the chimeric molecule comprises two synthetic procoagulant peptides and a PEG heterologous moiety, wherein the chimeric molecule further comprises a heterologous moiety selected from the group consisting of an immunoglobulin constant region or portion thereof (e.g., an Fc region), a PAS sequence, HES, and albumin, fragment, or variant thereof. In yet another embodiment, the chimeric molecule comprises a clotting factor or fragment thereof, a clotting factor cofactor (e.g., Tissue Factor if the clotting factor is Factor VII), and a PEG heterologous moiety, wherein the chimeric molecule further comprises a heterologous moiety selected from an immunoglobulin constant region or portion thereof (e.g., an Fc region), a PAS sequence, HES, and albumin, fragment, or variant thereof.

The polymer can be of any molecular weight, and can be branched or unbranched. For polyethylene glycol, in one embodiment, the molecular weight is between about 1 kDa and about 100 kDa for ease in handling and manufacturing. Other sizes can be used, depending on the desired profile (e.g., the duration of sustained release desired, the effects, if any on biological activity, the ease in handling, the degree or lack of antigenicity and other known effects of the polyethylene glycol to a protein or analog). For example, the polyethylene glycol can have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

In some embodiments, the polyethylene glycol can have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), each of which is incorporated herein by reference in its entirety.

The number of polyethylene glycol moieties attached to each chimeric molecule of the invention (i.e., the degree of substitution) can also vary. For example, the PEGylated chimeric molecule can be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit Rev. Thera. Drug Carrier Sys. 9:249-304 (1992).

In some embodiments, the chimeric molecule can be PEGylated.

A PEGylated chimeric molecule comprises at least one polyethylene glycol (PEG) molecule. In other embodiments, the polymer can be water-soluble. Non-limiting examples of the polymer can be poly(alkylene oxide), poly(vinyl pyrrolidone), poly(vinyl alcohol), polyoxazoline, or poly(acryloylmorpholine). Additional types of polymer-conjugation to clotting factors are disclosed in U.S. Pat. No. 7,199,223. See also, Singh et al. Curr. Med. Chem. 15:1802-1826 (2008).

There are a number of PEG attachment methods available to those skilled in the art, for example Malik F et al., Exp. Hematol. 20:1028-35 (1992); Francis, Focus on Growth Factors 3(2):4-10 (1992); European Pat. Pub. Nos. EP0401384, EP0154316, and EP0401384; and International Pat. Appl. Pub. Nos. WO92/16221 and WO95/34326.

In some embodiments, the chimeric molecule comprises a clotting factor (e.g., a FVII), a targeting moiety (e.g., a GPIIb/IIIa antibody or antigen-binding molecule thereof), and a PEG.

(x) HES

In certain embodiments, at least one heterologous moiety is a polymer, e.g., hydroxyethyl starch (HES) or a derivative thereof. Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., Krankenhauspharmazie, 8(8), 271-278 (1987); and Weidler et al., Arzneim.-Forschung/Drug Res., 41, 494-498 (1991)).

Amylopectin contains glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. The degree of substitution, denoted as DS, relates to the molar substitution, is known to the skilled people. See Sommermeyer et al., Krankenhauspharmazie, 8(8), 271-278 (1987), as cited above, in particular p. 273.

In one embodiment, hydroxyethyl starch has a mean molecular weight (weight mean) of from 1 to 300 kD, from 2 to 200 kD, from 3 to 100 kD, or from 4 to 70 kD. Hydroxyethyl starch can further exhibit a molar degree of substitution of from 0.1 to 3, preferably 0.1 to 2, more preferred, 0.1 to 0.9, preferably 0.1 to 0.8, and a ratio between C2:C6 substitution in the range of from 2 to 20 with respect to the hydroxyethyl groups. A non-limiting example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7. In a specific embodiment, HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolemia. The characteristics of VOLUVEN® are a mean molecular weight of 130,000+/−20,000 D, a molar substitution of 0.4 and a C2:C6 ratio of about 9:1. In other embodiments, ranges of the mean molecular weight of hydroxyethyl starch are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD. In still other embodiments, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD) or from 12 to 13 kD or from 11 to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD.

In certain embodiments, the heterologous moiety can be a mixture of hydroxyethyl starches having different mean molecular weights and/or different degrees of substitution and/or different ratios of C2:C6 substitution. Therefore, mixtures of hydroxyethyl starches can be employed having different mean molecular weights and different degrees of substitution and different ratios of C2:C6 substitution, or having different mean molecular weights and different degrees of substitution and the same or about the same ratio of C2:C6 substitution, or having different mean molecular weights and the same or about the same degree of substitution and different ratios of C2:C6 substitution, or having the same or about the same mean molecular weight and different degrees of substitution and different ratios of C2:C6 substitution, or having different mean molecular weights and the same or about the same degree of substitution and the same or about the same ratio of C2:C6 substitution, or having the same or about the same mean molecular weights and different degrees of substitution and the same or about the same ratio of C2:C6 substitution, or having the same or about the same mean molecular weight and the same or about the same degree of substitution and different ratios of C2:C6 substitution, or having about the same mean molecular weight and about the same degree of substitution and about the same ratio of C2:C6 substitution.

In some embodiments, the chimeric molecule comprises a clotting factor (e.g., a FVII), a targeting moiety (e.g., a GPIIb/IIIa antibody or antigen-binding molecule thereof), and a HES.

(xi) PSA

In certain embodiments, at least one heterologous moiety is a polymer, e.g., polysialic acids (PSAs) or a derivative thereof. Polysialic acids (PSAs) are naturally occurring unbranched polymers of sialic acid produced by certain bacterial strains and in mammals in certain cells Roth J., et al. (1993) in Polysialic Acid: From Microbes to Man, eds. Roth J., Rutishauser U., Troy F. A. (Birkhauser Verlag, Basel, Switzerland), pp 335-348. They can be produced in various degrees of polymerization from n=about 80 or more sialic acid residues down to n=2 by limited acid hydrolysis or by digestion with neuraminidases, or by fractionation of the natural, bacterially derived forms of the polymer. The composition of different polysialic acids also varies such that there are homopolymeric forms i.e. the alpha-2,8-linked polysialic acid comprising the capsular polysaccharide of E. coli strain K1 and the group-B meningococci, which is also found on the embryonic form of the neuronal cell adhesion molecule (N-CAM). Heteropolymeric forms also exist—such as the alternating alpha-2,8 alpha-2,9 polysialic acid of E. coli strain K92 and group C polysaccharides of N. meningitidis. Sialic acid can also be found in alternating copolymers with monomers other than sialic acid such as group W135 or group Y of N. meningitidis. Polysialic acids have important biological functions including the evasion of the immune and complement systems by pathogenic bacteria and the regulation of glial adhesiveness of immature neurons during fetal development (wherein the polymer has an anti-adhesive function) Cho and Troy, P.N.A.S, USA, 91 (1994) 11427-11431, although there are no known receptors for polysialic acids in mammals. The alpha-2,8-linked polysialic acid of E. coli strain K1 is also known as ‘colominic acid’ and is used (in various lengths) to exemplify the present disclosure. Various methods of attaching or conjugating polysialic acids to a polypeptide have been described (for example, see U.S. Pat. No. 5,846,951; WO-A-0187922, and US 2007/0191597 A1, which are incorporated herein by reference in their entireties.

In some embodiments, the chimeric molecule comprises a clotting factor (e.g., FVII), a targeting moiety (e.g., a GPIIb/IIIa antibody or antigen-binding molecule thereof), and a PSA.

(xii) Clearance Receptors

In certain embodiments, the in vivo half-life of a chimeric molecule of the invention can be extended where the chimeric molecule comprises at least one heterologous molecule comprising a clearance receptor, fragment, variant, or derivative thereof. In specific embodiments wherein the chimeric molecule comprises Factor X, soluble forms of clearance receptors, such as the low density lipoprotein-related protein receptor LRP1, or fragments thereof, can block binding of Factor X to clearance receptors and thereby extend its in vivo half-life.

LRP1 is a 600 kDa integral membrane protein that is implicated in the receptor-mediate clearance of a variety of proteins, such as FVIII or X. See, e.g., Narita et al., Blood 91:555-560 (1998); Lenting et al., Haemophilia 16:6-16 (2010). The amino acid sequence of an exemplary human LRP1 protein is provided below (signal peptide underlined and transmembrane segment boldened; NCBI Reference Sequence: CAA32112):

(SEQ ID NO: 155)
MLTPPLLLLLPLLSALVAAAIDAPKTCSPKQFACRD
QITCISKGWRCDGERDCPDGSDEAPEICPQSKAQR
CQPNEHNCLGTELCVPMSRLCNGVQDCMDGSDEGP
HCRELQGNCSRLGCQHHCVPTLDGPTCYCNSSFQL
QADGKTCKDFDECSVYGTCSQLCTNTDGSSICGCV
EGYLLQPDNRSCKAKNEPVDRPPVLLIANSQNILA
TYLSGAQVSTITPTSTRQTTAMDFSYANETVCWVH
VGDSAAQTQLKCARMPGLKGFVDEHTINISLSLHH
VEQMAIDWLTGNFYFVDDIDDRIFVCNRNGDTCVT
LLDLELYNPKGIALDPAMGKVFFTDYGQIPKVERC
DMDGQNRTKLVDSKIVFPHGITLDLVSRLVYWADA
YLDYIEVVDYEGKGRQTIIQGILIEHLYGLTVFEN
YLYATNSDNANAQQKTSVIRVNRFNSTEYQVVTRV
DKGGALHIYHQRRQPRVRSHACENDQYGKPGGCSD
ICLLANSHKARTCRCRSGFSLGSDGKSCKKPEHEL
FLVYGKGRPGIIRGMDMGAKVPDEHMIPIENLMNP
RALDFHAETGFIYFADTTSYLIGRQKIDGTERETI
LKDGIHNVEGVAVDWMGDNLYWTDDGPKKTISVAR
LEKAAQTRKTLIEGKMTHPRAIVVDPLNGWMYWTD
WEEDPKDSRRGRLERAWMDGSHRDIFVTSKTVLWP
NGLSLDIPAGRLYWVDAFYDRIETILLNGTDRKIV
YEGPELNHAFGLCHHGNYLFWTEYRSGSVYRLERG
VGGAPPTVTLLRSERPPIFEIRMYDAQQQQVGTNK
CRVNNGGCSSLCLATPGSRQCACAEDQVLDADGVT
CLANPSYVPPPQCQPGEFACANSRCIQERWKCDGD
NDCLDNSDEAPALCHQHTCPSDRFKCENNRCIPNR
WLCDGDNDCGNSEDESNATCSARTCPPNQFSCASG
RCIPISWTCDLDDDCGDRSDESASCAYPTCFPLTQ
FTCNNGRCININWRCDNDNDCGDNSDEAGCSHSCS
STQFKCNSGRCIPEHWTCDGDNDCGDYSDETHANC
TNQATRPPGGCHTDEFQCRLDGLCIPLRWRCDGDT
DCMDSSDEKSCEGVTHVCDPSVKFGCKDSARCISK
AWVCDGDNDCEDNSDEENCESLACRPPSHPCANNT
SVCLPPDKLCDGNDDCGDGSDEGELCDQCSLNNGG
CSHNCSVAPGEGIVCSCPLGMELGPDNHTCQIQSY
CAKHLKCSQKCDQNKFSVKCSCYEGWVLEPDGESC
RSLDPFKPFIIFSNRHEIRRIDLHKGDYSVLVPGL
RNTIALDFHLSQSALYWTDVVEDKIYRGKLLDNGA
LTSFEVVIQYGLATPEGLAVDWIAGNIYWVESNLD
QIEVAKLDGTLRTTLLAGDIEHPRAIALDPRDGIL
FWTDWDASLPRIEAASMSGAGRRTVHRETGSGGWP
NGLTVDYLEKRILWIDARSDAIYSARYDGSGHMEV
LRGHEFLSHPFAVTLYGGEVYWTDWRTNTLAKANK
WTGHNVTVVQRTNTQPFDLQVYHPSRQPMAPNPCE
ANGGQGPCSHLCLINYNRTVSCACPHLMKLHKDNT
TCYEFKKFLLYARQMEIRGVDLDAPYYNYIISFTV
PDIDNVTVLDYDAREQRVYWSDVRTQAIKRAFING
TGVETVVSADLPNAHGLAVDWVSRNLFWTSYDTNK
KQINVARLDGSFKNAVVQGLEQPHGLVVHPLRGKL
YWTDGDNISMANMDGSNRTLLFSGQKGPVGLAIDF
PESKLYWISSGNHTINRCNLDGSGLEVIDAMRSQL
GKATALAIMGDKLWWADQVSEKMGTCSKADGSGSV
VLRNSTTLVMHMKVYDESIQLDHKGTNPCSVNNGD
CSQLCLPTSETTRSCMCTAGYSLRSGQQACEGVGS
FLLYSVHEGIRGIPLDPNDKSDALVPVSGTSLAVG
IDFHAENDTIYWVDMGLSTISRAKRDQTWREDVVT
NGIGRVEGIAVDWIAGNIYWTDQGFDVIEVARLNG
SFRYVVISQGLDKPRAITVHPEKGYLFWTEWGQYP
RIERSRLDGTERVVLVNVSISWPNGISVDYQDGKL
YWCDARTDKIERIDLETGENREVVLSSNNMDMFSV
SVFEDFIYWSDRTHANGSIKRGSKDNATDSVPLRT
GIGVQLKDIKVFNRDRQKGTNVCAVANGGCQQLCL
YRGRGQRACACAHGMLAEDGASCREYAGYLLYSER
TILKSIHLSDERNLNAPVQPFEDPEHMKNVIALAF
DYRAGTSPGTPNRIFFSDIHFGNIQQINDDGSRRI
TIVENVGSVEGLAYHRGWDTLYWTSYTTSTITRHT
VDQTRPGAFERETVITMSGDDHPRAFVLDECQNLM
FWTNWNEQHPSIMRAALSGANVLTLIEKDIRTPNG
LAIDHRAEKLYFSDATLDKIERCEYDGSHRYVILK
SEPVHPFGLAVYGEHIFWTDWVRRAVQRANKHVGS
NMKLLRVDIPQQPMGIIAVANDTNSCELSPCRINN
GGCQDLCLLTHQGHVNCSCRGGRILQDDLTCRAVN
SSCRAQDEFECANGECINFSLTCDGVPHCKDKSDE
KPSYCNSRRCKKTFRQCSNGRCVSNMLWCNGADDC
GDGSDEIPCNKTACGVGEFRCRDGTCIGNSSRCNQ
FVDCEDASDEMNCSATDCSSYFRLGVKGVLFQPCE
RTSLCYAPSWVCDGANDCGDYSDERDCPGVKRPRC
PLNYFACPSGRCIPMSWTCDKEDDCEHGEDETHCN
KFCSEAQFECQNHRCISKQWLCDGSDDCGDGSDEA
AHCEGKTCGPSSFSCPGTHVCVPERWLCDGDKDCA
DGADESIAAGCLYNSTCDDREFMCQNRQCIPKHFV
CDHDRDCADGSDESPECEYPTCGPSEFRCANGRCL
SSRQWECDGENDCHDQSDEAPKNPHCTSPEHKCNA
SSQFLCSSGRCVAEALLCNGQDDCGDSSDERGCHI
NECLSRKLSGCSQDCEDLKIGFKCRCRPGFRLKDD
GRTCADVDECSTTFPCSQRCINTHGSYKCLCVEGY
APRGGDPHSCKAVTDEEPFLIFANRYYLRKLNLDG
SNYTLLKQGLNNAVALDFDYREQMIYWTDVTTQGS
MIRRMHLNGSNVQVLHRTGLSNPDGLAVDWVGGNL
YWCDKGRDTIEVSKLNGAYRTVLVSSGLREPRALV
VDVQNGYLYWTDWGDHSLIGRIGMDGSSRSVIVDT
KITWPNGLTLDYVTERIYWADAREDYIEFASLDGS
NRHVVLSQDIPHIFALTLFEDYVYWTDWETKSINR
AHKTTGTNKTLLISTLHRPMDLHVFHALRQPDVPN
HPCKVNNGGCSNLCLLSPGGGHKCACPTNFYLGSD
GRTCVSNCTASQFVCKNDKCIPFWWKCDTEDDCGD
HSDEPPDCPEFKCRPGQFQCSTGICTNPAFICDGD
NDCQDNSDEANCDIHVCLPSQFKCTNTNRCIPGIF
RCNGQDNCGDGEDERDCPEVTCAPNQFQCSITKRC
IPRVWVCDRDNDCVDGSDEPANCTQMTCGVDEFRC
KDSGRCIPARWKCDGEDDCGDGSDEPKEECDERTC
EPYQFRCKNNRCVPGRWQCDYDNDCGDNSDEESCT
PRPCSESEFSCANGRCIAGRWKCDGDHDCADGSDE
KDCTPRCDMDQFQCKSGHCIPLRWRCDADADCMDG
SDEEACGTGVRTCPLDEFQCNNTLCKPLAWKCDGE
DDCGDNSDENPEECARFVCPPNRPFRCKNDRVCLW
IGRQCDGTDNCGDGTDEEDCEPPTAHTTHCKDKKE
FLCRNQRCLSSSLRCNMFDDCGDGSDEEDCSIDPK
LTSCATNASICGDEARCVRTEKAAYCACRSGFHTV
PGQPGCQDINECLRFGTCSQLCNNTKGGHLCSCAR
NFMKTHNTCKAEGSEYQVLYIADDNEIRSLFPGHP
HSAYEQAFQGDESVRIDAMDVHVKAGRVYWTNWHT
GTISYRSLPPAAPPTTSNRHRRQIDRGVTHLNISG
LKMPRGIAIDWVAGNVYWTDSGRDVIEVAQMKGEN
RKTLISGMIDEPHAIVVDPLRGTMYWSDWGNHPKI
ETAAMDGTLRETLVQDNIQWPTGLAVDYHNERLYW
ADAKLSVIGSIRLNGTDPIVAADSKRGLSHPFSID
VFEDYIYGVTYINNRVFKIHKFGHSPLVNLTGGLS
HASDVVLYHQHKQPEVTNPCDRKKCEWLCLLSPSG
PVCTCPNGKRLDNGTCVPVPSPTPPPDAPRPGTCN
LQCFNGGSCFLNARRQPKCRCQPRYTGDKCELDQC
WEHCRNGGTCAASPSGMPTCRCPTGFTGPKCTQQV
CAGYCANNSTCTVNQGNQPQCRCLPGFLGDRCQYR
QCSGYCENFGTCQMAADGSRQCRCTAYFEGSRCEV
NKCSRCLEGACVVNKQSGDVTCNCTDGRVAPSCLT
CVGHCSNGGSCTMNSKMMPECQCPPHMTGPRCEEH
VFSQQQPGHIASILIPLLLLLLLVLVAGVVFWYKR
RVQGAKGFQHQRMTNGAMNVEIGNPTYKMYEGGEP
DDVGGLLDADFALDPDKPTNFTNPVYATLYMGGHG
SRHSLASTDEKRELLGRGPEDEIGDPLA

Other suitable clearance receptors are, e.g., LDLR (low-density lipoprotein receptor), VLDLR (very low-density lipoprotein receptor), and megalin (LRP-2), or fragments thereof. See, e.g., Bovenschen et al., Blood 106:906-912 (2005); Bovenschen, Blood 116:5439-5440 (2010); Martinelli et al., Blood 116:5688-5697 (2010).

In some embodiments, the chimeric molecule comprises a clotting factor (e.g., a FVII), a targeting moiety (e.g., a GPIIb/IIIa antibody or antigen-binding molecule thereof), and a clearance receptor, fragment, variant, or derivative thereof.

II. Linkers

The term “linker” or “linker moiety” (represented as L, L1, or L2 in the formulas disclosed herein) refers to a peptide or polypeptide sequence (e.g., a synthetic peptide or polypeptide sequence), or a non-peptide linker for which its main function is to connect two domains in a linear amino acid sequence of a polypeptide chain, for example, two heterologous moieties in a chimeric molecule of the invention. Accordingly, in some embodiments, linkers are interposed between two heterologous moieties, between a heterologous moiety and a targeting moiety, which binds to a platelet (e.g., an anti-GPIIb/IIIa antibody or antigen-binding molecule thereof disclosed herein), between a clotting factor (either the heavy chain or the light chain) and a targeting moiety, which binds to a platelet (e.g., an anti-GPIIb/IIIa antibody or antigen-binding molecule thereof disclosed herein), or between a clotting factor (either the heavy chain or the light chain) and a heterologous moiety.

When multiple linkers are present in a chimeric molecule of the invention, each of the linkers can be the same or different. Generally, linkers provide flexibility to the chimeric molecule. Linkers are not typically cleaved; however in certain embodiments, such cleavage can be desirable. Accordingly, in some embodiments a linker can comprise one or more protease-cleavable sites, which can be located within the sequence of the linker or flanking the linker at either end of the sequence of the linker.

In some embodiments, the chimeric molecule comprises one or more linkers, wherein one or more of the linkers comprise a peptide linker. In other embodiments, one or more of the linkers comprise a non-peptide linker. In some embodiments, the peptide linker can comprise at least two amino, at least three, at least four, at least five, at least 10, at least 20, at least 30, at least 40, at least 50, at least 60, at least 70, at least 80, at least 90, or at least 100 amino acids. In other embodiments, the peptide linker can comprise at least 200, at least 300, at least 400, at least 500, at least 600, at least 700, at least 800, at least 900, or at least 1,000 amino acids. In some embodiments, the peptide linker can comprise at least about 10, 20, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, or 2000 amino acids. In certain embodiments, the peptide linker can comprise 10, 20, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, or 2000 amino acids.

The peptide linker can comprise 1-5 amino acids, 1-10 amino acids, 1-20 amino acids, 1-30 amino acids, 5-25 amino acids, 5-30 amino acids, 10-30 amino acids, 10-50 amino acids, 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, 900-1000, 1000-1100, 1100-1200, 1200-1300, 1300-1400, 1400-1500, 1500-1600, 1600-1700, 1700-1800, 1800-1900, or 1900-2000 amino acids.

Examples of peptide linkers are well known in the art, for example peptide linkers according to the formula [(Gly)ₓ-Ser_{y}]_{z} where x is from 1 to 4, y is 0 or 1, and z is from 1 to 50 (SEQ ID NO:156). In certain embodiments z is from 1 to 6. In one embodiment, the peptide linker comprises the sequence Gₙ, where n can be an integer from 1 to 100 (SEQ ID NO:250). In a specific embodiment, the specific embodiment, the sequence of the peptide linker is GGGG (SEQ ID NO:157). The peptide linker can comprise the sequence (GA)ₙ (SEQ ID NO:158). The peptide linker can comprise the sequence (GGS)ₙ(SEQ ID NO:159). In other embodiments, the peptide linker comprises the sequence (GGGS)ₙ (SEQ ID NO:160). In still other embodiments, the peptide linker comprises the sequence (GGS)ₙ(GGGGS)ₙ (SEQ ID NO:161). In these instances, n can be an integer from 1-100. In other instances, n can be an integer from 1-20, i.e., 1, 2, 3, 4, 5, 6, 7, 8, 9, 10, 11, 12, 13, 14, 15, 16, 17, 18, 19, or 20. Examples of linkers include, but are not limited to, GGG, SGGSGGS (SEQ ID NO:162), GGSGGSGGSGGSGGG (SEQ ID NO:163), GGSGGSGGGGSGGGGS (SEQ ID NO:164), GGSGGSGGSGGSGGSGGS (SEQ ID NO:165), or GGGGSGGGGSGGGGS (SEQ ID NO:166). In other embodiments, the linker is a poly-G sequence (GGGG)ₙ, where n can be an integer from 1-100 (SEQ ID NO:167).

An exemplary Gly/Ser peptide linker comprises the amino acid sequence (Gly₄Ser)ₙ (SEQ ID NO:251), wherein n is an integer that is the same or higher than 1, 2, 3, 4, 5, 6, 7, 8, 9, 10, 15, 20, 25, 30, 35, 40, 46, 50, 55, 60, 70, 80, 90, or 100. In one embodiment, n=1, i.e., the linker is (Gly₄Ser) (SEQ ID NO:248). In one embodiment, n=2, i.e., the linker is (Gly₄Ser)₂ (SEQ ID NO:168). In another embodiment, n=3, i.e., the linker is (Gly₄Ser)₃ (SEQ ID NO:169). In another embodiment, n=4, i.e., the linker is (Gly₄Ser)₄ (SEQ ID NO:170). In another embodiment, n=5, i.e., the linker is (Gly₄Ser)₅ (SEQ ID NO:171). In yet another embodiment, n=6, i.e., the linker is (Gly₄Ser)₆ (SEQ ID NO:172). In another embodiment, n=7, i.e., the linker is (Gly₄Ser)₇ (SEQ ID NO:173). In yet another embodiment, n=8, i.e., the linker is (Gly₄Ser)₈ (SEQ ID NO:174). In another embodiment, n=9, i.e., the linker is (Gly₄Ser)₉ (SEQ ID NO:175). In yet another embodiment, n=10, i.e., the linker is (Gly₄Ser)₁₀ (SEQ ID NO:176).

Another exemplary Gly/Ser peptide linker comprises the amino acid sequence Ser(Gly₄Ser)ₙ (SEQ ID NO:252), wherein n is an integer that is the same or higher than 1, 2, 3, 4, 5, 6, 7, 8, 9, 10, 15, 20, 25, 30, 35, 40, 46, 50, 55, 60, 70, 80, 90, or 100. In one embodiment, n=1, i.e., the linker is Ser(Gly₄Ser) (SEQ ID NO:177). In one embodiment, n=2, i.e., the linker is Ser(Gly₄Ser)₂ (SEQ ID NO: 178). In another embodiment, n=3, i.e., the linker is Ser(Gly₄Ser)₃ (SEQ ID NO:179). In another embodiment, n=4, i.e., the linker is Ser(Gly₄Ser)₄ (SEQ ID NO:180). In another embodiment, n=5, i.e., the linker is Ser(Gly₄Ser)₅ (SEQ ID NO:181). In yet another embodiment, n=6, i.e., the linker is Ser(Gly₄Ser)₆ (SEQ ID NO:182). In yet another embodiment, n=7, i.e., the linker is Ser(Gly₄Ser)₇ (SEQ ID NO:183). In yet another embodiment, n=8, i.e., the linker is Ser(Gly₄Ser)₈ (SEQ ID NO:184). In yet another embodiment, n=9, i.e., the linker is Ser(Gly₄Ser)₉ (SEQ ID NO:185). In yet another embodiment, n=10, i.e., the linker is Ser(Gly₄Ser)₁₀ (SEQ ID NO:186).

In certain embodiments, said Gly/Ser peptide linker can be inserted between two other sequences of the peptide linker (e.g., any of the peptide linker sequences described herein). In other embodiments, a Gly/Ser peptide linker is attached at one or both ends of another sequence of the peptide linker (e.g., any of the peptide linker sequences described herein). In yet other embodiments, two or more Gly/Ser linkers are incorporated in series in a peptide linker. In one embodiment, a peptide linker of the invention comprises at least a portion of an upper hinge region (e.g., derived from an IgG1, IgG2, IgG3, or IgG4 molecule), at least a portion of a middle hinge region (e.g., derived from an IgG1, IgG2, IgG3, or IgG4 molecule) and a series of Gly/Ser amino acid residues (e.g., a Gly/Ser linker such as (Gly₄Ser)ₙ) (SEQ ID NO:251)).

A particular type of linker which can be present in an heterologous moiety, for example an activatable clotting factor, is herein referred to as a “cleavable linker” which comprises a heterologous protease-cleavage site (e.g., a factor XIa or thrombin cleavage site) that is not naturally occurring in the clotting factor and which can include additional linkers on either the N terminal of C terminal or both sides of the cleavage site. Exemplary locations for such sites include, e.g., placement between a heavy chain of a clotting factor zymogen and a light chain of a clotting factor zymogen.

Peptide linkers can be introduced into polypeptide sequences using techniques known in the art. Modifications can be confirmed by DNA sequence analysis. Plasmid DNA can be used to transform host cells for stable production of the polypeptides produced.

III. Protease Cleavage Site

In some embodiments, a chimeric molecule can comprise a protease cleavage site linking, for example, a light chain of a clotting factor zymogen and a heavy chain of the clotting factor zymogen (e.g., FVII). A protease-cleavage site linking a light chain of a clotting factor zymogen and a heavy chain of the clotting factor zymogen can be selected from any protease-cleavage site known in the art. In one embodiment, the protease-cleavage site is cleaved by a protease selected from the group consisting of factor XIa, factor XIIa, kallikrein, factor VIIa, factor IXa, factor Xa, factor IIa (thrombin), and any combinations thereof. The protease-cleavage sites allow the light chain and the heavy chain of the clotting factor to be cleaved and dissociated from each other at the site of injury. Exemplary FXIa cleavage sites include, e.g., KLTR (SEQ ID NO:187), DFTR (SEQ ID NO:188), TQSFNDFTR (SEQ ID NO:189) and SVSQTSKLTR (SEQ ID NO:190). Exemplary thrombin cleavage sites include, e.g., DFLAEGGGVR (SEQ ID NO:191), TTKIKPR (SEQ ID NO:192), LVPRG (SEQ ID NO:193) and ALRPR (SEQ ID NO:194).

In some embodiments, the protease-cleavage site can be combined with an intracellular processing site for efficient cleavage and activation. For example, an activatable clotting factor in the chimeric molecule can comprise a heterodimer, which comprises a light chain of a clotting factor associated with a heavy chain of the clotting factor by a covalent bond, wherein the N-terminus of the heavy chain of the clotting factor is linked to a protease-cleavage site. The protease-cleavage site can be cleaved off at the site of coagulation, thus activating the clotting factor. Such constructs can be designed by inserting an intracellular processing site between the light chain of the clotting factor zymogen and the protease-cleavage site, which is linked to the heavy chain of the clotting factor zymogen. The intracellular processing site inserted therein can be processed (cleaved) by an intracellular processing enzyme upon expression in a host cell, thereby allowing formation of a zymogen-like heterodimer.

Examples of the intracellular processing enzymes include furin, a yeast Kex2, PCSK1 (also known as PC1/Pc3), PCSK2 (also known as PC2), PCSK3 (also known as furin or PACE), PCSK4 (also known as PC4), PCSK5 (also known as PC5 or PC6), PCSK6 (also known as PACE4), or PCSK7 (also known as PC7/LPC, PC8, or SPC7). Other processing sites are known in the art. In constructs that include more than one processing or cleavage site, it will be understood that such sites can be the same or different.

### E. Exemplary Chimeric Molecules

The chimeric molecule can include a polypeptide that comprises the light chain of a Factor VII (e.g., rFVIIa) associated with the heavy chain of Factor VII (e.g., rFVIIa). Any allelic variant of FVII can also be used in the chimeric molecule. In certain embodiments, the Factor VII in the chimeric polypeptide comprises or consists of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to amino acids 21-444 of the amino acid sequence set forth in SEQ ID NO:128. In some instances, the C-terminus of the light or heavy chain of a FVII is linked directly or via an optional linker to the N-terminus of the variable light or variable heavy chain of any one of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, or BIIB-4-319. The variable light or variable heavy chain of the anti-GPIIb/IIIa antibodies included in the chimeric polypeptide can be at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the variable light or variable heavy chain of any one of BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, or BIIB-4-319. In certain embodiments, if the chimeric polypeptide comprises a variable light chain, the C-terminus of the variable light chain is linked to a CL comprising or consisting of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence set forth in SEQ ID NO:121. In certain embodiments, if the chimeric polypeptide comprises a variable heavy chain, the C-terminus of the variable heavy chain is linked to a CH1 comprising or consisting of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence set forth in SEQ ID NO:122.

An exemplary amino acid sequence of a FVII fused to a linker is shown below (the light chain of FVII is boldened, the heavy chain of FVII italicized, and the linker boldened and underlined; the sequence preceding the light chain of FVII includes the signal sequence and propeptide sequence).

(SEQ ID NO: 195)
M V S Q A L R L L C L L L G L Q G C L A A V F V T Q
E E A H G V L H R R R R A N A F L E E L R P G S L E
R E C K E E Q C S F E E A R E I F K D A E R T K L F
W I S Y S D G D Q C A S S P C Q N G G S C K D Q L Q
S Y I C F C L P A F E G R N C E T H K D D Q L I C V
N E N G G C E Q Y C S D H T G T K R S C R C H E G Y
S L L A D G V S C T P T V E Y P C G K I P I L E K R
N A S K P Q G R I V G G K V C P K G E C P W Q V L L
L V N G A Q L C G G T L I N T I W V V S A A H C F D
K I K N W R N L I A V L G E H D L S E H D G D E Q S
R R V A Q V I I P S T Y V P G T T N H D I A L L R L
H Q P V V L T D H V V P L C L P E R T F S E R T L A
F V R F S L V S G W G Q L L D R G A T A L E L M V L
N V P R L M T Q D C L Q Q S R K V G D S P N I T E Y
M F C A G Y S D G S K D S C K G D S G G P H A T H Y
R G T W Y L T G I V S W G Q G C A T V G H F G V Y T
R V S Q Y I E W L Q K L M R S E P R P G V L L R A P
F P G G G G S G G G G S G G G G S G G G G S G G G G
S G G G G S

An exemplary FVII-linker-BIIB_4_147 VL/CL polypeptide is shown below (the light chain of FVII is boldened, the heavy chain of FVII italicized, the linker boldened and underlined, and the CL region of the Fab light chain is underlined; the sequence preceding the light chain of FVII includes the signal sequence and propeptide sequence):

(SEQ ID NO: 125)
M V S Q A L R L L C L L L G L Q
G C L A A V F V T Q E E A H G
V L H R R R R A N A F L E E L
R P G S L E R E C K E E Q C S
F E E A R E I F K D A E R T K
L F W I S Y S D G D Q C A S S
P C Q N G G S C K D Q L Q S Y
I C F C L P A F E G R N C E T
H K D D Q L I C V N E N G G C
E Q Y C S D H T G T K R S C R
C H E G Y S L L A D G V S C T
P T V E Y P C G K I P I L E K
R N A S K P Q G R I V G G K V
C P K G E C P W Q V L L L V N
G A Q L C G G T L I N T I W V
V S A A H C F D K I K N W R N
L I A V L G E H D L S E H D G
D E Q S R R V A Q V I I P S T
Y V P G T T N H D I A L L R L
H Q P V V L T D H V V P L C L
P E R T F S E R T L A F V R F
S L V S G W G Q L L D R G A T
A L E L M V L N V P R L M T Q
D C L Q Q S R K V G D S P N I
T E Y M F C A G Y S D G S K D
S C K G D S G G P H A T H Y R
G T W Y L T G I V S W G Q G C
A T V G H F G V Y T R V S Q Y
I E W L Q K L M R S E P R P G
V L L R A P F P G G G G S G G
G G S G G G G S G G G G S G G
G G S G G G G S D I V M T Q S
P L S L P V T P G E P A S I S
C R S S Q S L L H S N G Y N Y
L D W Y L Q K P G Q S P Q L L
I Y L G S N R A S G V P D R F
S G S G S G T D F T L K I S R
V E A E D V G V Y Y C M Q A L
R L P R T F G G G T K V E I K
R T V A A P S V F I F P P S D
E Q L K S G T A S V V C L L N
N F Y P R E A K V Q W K V D N
A L Q S G N S Q E S V T E Q D
S K D S T Y S L S S T L T L S
K A D Y E K H K V Y A C E V T
H Q G L S S P V T K S F N R G
E C

An exemplary FVII-linker-BIIB_4_156 VL/CL polypeptide is shown below (the light chain of FVII is boldened, the heavy chain of FVII italicized, the linker boldened and underlined, and the CL region of the Fab light chain is underlined):

(SEQ ID NO: 196)
M V S Q A L R L L C L L L G L Q
G C L A A V F V T Q E E A H G
V L H R R R R A N A F L E E L
R P G S L E R E C K E E Q C S
F E E A R E I F K D A E R T K
L F W I S Y S D G D Q C A S S
P C Q N G G S C K D Q L Q S Y
I C F C L P A F E G R N C E T
H K D D Q L I C V N E N G G C
E Q Y C S D H T G T K R S C R
C H E G Y S L L A D G V S C T
P T V E Y P C G K I P I L E K
R N A S K P Q G R I V G G K V
C P K G E C P W Q V L L L V N
G A Q L C G G T L I N T I W V
V S A A H C F D K I K N W R N
L I A V L G E H D L S E H D G
D E Q S R R V A Q V I I P S T
Y V P G T T N H D I A L L R L
H Q P V V L T D H V V P L C L
P E R T F S E R T L A F V R F
S L V S G W G Q L L D R G A T
A L E L M V L N V P R L M T Q
D C L Q Q S R K V G D S P N I
T E Y M F C A G Y S D G S K D
S C K G D S G G P H A T H Y R
G T W Y L T G I V S W G Q G C
A T V G H F G V Y T R V S Q Y
I E W L Q K L M R S E P R P G
V L L R A P F P G G G G S G G
G G S G G G G S G G G G S G G
G G S G G G G S E I V L T Q S
P A T L S L S P G E R A T L S
C R A S Q S V S S Y L A W Y Q
Q K P G Q A P R L L I Y D A S
N R A T G I P A R F S G S G S
G T D F T L T I S S L E P E D
F A V Y Y C Q Q R S A L P R T
F G G G T K V E I K R T V A A
P S V F I F P P S D E Q L K S
G T A S V V C L L N N F Y P R
E A K V Q W K V D N A L Q S G
N S Q E S V T E Q D S K D S T
Y S L S S T L T L S K A D Y E
K H K V Y A C E V T H Q G L S
S P V T K S F N R G E C

Similarly any of the VL regions having an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the VL domain of any one of BIIB-4-204, BIIB-4-209, BIIB-4-224, BIIB-4-309, BIIB-4-311, BIIB-4-317, BIIB-4-318, or BIIB-4-319 can be introduced between either the C-terminus of the heavy chain of FVII or the C-terminus of the optional linker and the CL domain of the Fab light chain in SEQ ID NOs.: 125 or 196.

In certain embodiments, one or more (e.g., 1, 2, 3, 4) linkers can be introduced between the light and heavy chain of Factor VII. The linker(s) can be a peptide linker.

The Fab light chain of the chimeric molecule can associate with a polypeptide comprising its Fab heavy chain counterpart. For example, the Fab 4_147 light chain of SEQ ID NO:125 can associate with the Fab 4_147 heavy chain (VH/CH1) of SEQ ID NO:127; and the Fab 4_156 light chain of SEQ ID NO:196 can associate with a Fab 4_156 heavy chain (VH/CH1) (e.g., a polypeptide comprising an amino sequence of SEQ ID NO:9 linked to the amino sequence of SEQ ID NO:122).

In one embodiment, the chimeric molecule comprises an XTEN between the heavy chain of the FVII and the Fab light chain. The XTEN may be connected to the Fab light chain via one or more (e.g., 1, 2, 3, 4) linkers. The linkers in the chimeric polypeptide can be peptide linkers. In certain embodiments, the XTEN is AE144. In other embodiments, the XTEN is AE288. In some cases, the heavy chain of FVII is linked to XTEN via a linker. In certain embodiments, this linker has the amino acid sequence:

(SEQ ID NO: 197)
GSPGTSESATPESGPGSEPATSGSETP.

In another embodiment, the chimeric molecule comprises an XTEN directly connected to the Fab light or Fab heavy chain of any of the antibodies disclosed herein. In certain embodiments, the chimeric molecule comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the VL domain or the VH domain of any one of BIIB-4-147, BIIB-4-156, BIIB-4-204, BIIB-4-209, BIIB-4-174, BIIB-4-175, BIIB-4-224, BIIB-4-309, BIIB-4-311, BIIB-4-317, BIIB-4-318, or BIIB-4-319. In some embodiments, these chimeric molecules, when they include a VL domain can also include a CL domain such as the one in SEQ ID NO:125. In some embodiments, these chimeric molecules, when they include a VH domain can also include a CH1 domain such as the one in SEQ ID NO:127. The XTEN of the chimeric molecule can also be connected via one or more (e.g., 1, 2, 3, 4) linkers to the Fab light or Fab heavy chain of the antibodies disclosed herein. The linkers in these chimeric polypeptide can be peptide linkers. In certain embodiments, the XTEN is AE144. In other embodiments, the XTEN is AE288. In some cases, the heavy chain of FVII is linked to XTEN via a linker. In certain embodiments, this linker has the amino acid sequence set forth in SEQ ID NO: 197.

In one embodiment, the chimeric molecule includes the light and heavy chains of Factor VII associated together, a linker having the amino acid sequence set forth in SEQ ID NO: 197 linked to the C-terminus of the heavy chain of FVII, an XTEN termed AE288 (a half-life extending moiety) linked to the C-terminus of SEQ ID NO:197, a GSSS (SEQ ID NO: 198) linker linked to the C-terminus AE288, a (G4S)₆ (SEQ ID NO:172) linker linked to the C-terminus of SEQ ID NO:198, and the N-terminus of an Fab light chain of a GPIIb/IIIa antibody described herein linked to the C-terminus of SEQ ID NO:172. In certain embodiments, one or more of the linkers noted above can be eliminated (e.g., SEQ ID NOs: 197 and/or 198) from the chimeric molecule. In certain embodiments, one or more (e.g., 1, 2, 3, 4) linkers can be introduced between the light and heavy chain of Factor VII. The linker(s) can be a peptide linker. In certain embodiments, the heavy chain of Factor VII can precede the light chain of Factor VII in the chimeric molecule. The Fab light chain of this chimeric molecule can associate with a polypeptide comprising the Fab heavy chain counterpart of the Fab light chain in the chimeric polypeptide. The above-described chimeric molecules can be modified, e.g., to include additional linkers (e.g., between the Factor VII and the half-life extending moiety and between the half-life extending moiety and the anti-GPIIb/IIIa antibody or antigen-binding fragment thereof). In certain instances there can be one or more (e.g., 1, 2, 3, 4) linkers between these components of the chimeric molecule. These chimeric molecules can also be modified to include one or more half-life extending moieties (e.g., AE144, AE288). In addition, instead of an Fab fragment, the chimeric molecules can comprise an scFv, a diabody, sc(Fv)₂, or a whole antibody of any of the anti-GPIIb/IIIa antibodies described herein. In instances where the targeting moiety is an scFv, the chimeric molecule is a two polypeptide chain comprising either (i) the light chain of Factor VII and the heavy chain of Factor VII-scFv or heavy chain of Factor VII-half-life extending moiety-scFv chimeric molecule; or (ii) the heavy chain of Factor VII and the light chain of Factor VII-scFv or light chain of Factor VII-half-life extending moiety-scFv chimeric molecule.

In certain embodiments, the Factor VII of the chimeric molecule is activated. Activation of Factor VII can occur by the cleavage of the Arg190-Ile191 peptide bond of Factor VII (SEQ ID NO: 128) to create a two chain FVII polypeptide. In one embodiment, the Factor VII of the chimeric molecule is activated by concentrating the chimeric polypeptide to about 4 mg/ml at a pH of 8.0 and incubating the polypeptide at 4° C. for several minutes to an hour (e.g., 1, 2, 4, 5, 10, 15, 20, 25, 30, 35, 40, 45, 50, 55 or 60 minutes).

### F. Methods of Preparation

The present disclosure also provides a nucleic acid molecule or a set of nucleic acid molecules encoding (i) a GPIIb/IIIa antibody or antigen-binding molecule thereof disclosed herein, or (ii) any of the chimeric molecules disclosed herein, or (iii) a complement thereof.

In one embodiment, the invention includes a nucleic acid molecule encoding a polypeptide chain, which comprises a light chain of a clotting factor (e.g., FVII, FIX, or FX), a heterologous moiety (e.g., a half-life extending moiety), an intracellular processing site, a heavy chain of the clotting factor (e.g., FVII, FIX, or FX), and a targeting moiety which binds to a platelet (e.g., an anti-GPIIb/IIIa antibody or antigen-binding molecule thereof). In another embodiment, the nucleic acid molecule of the invention encodes a polypeptide chain comprising a light chain of a clotting factor (e.g., FVII, FIX, or FX), a targeting moiety which binds to a platelet (e.g., an anti-GPIIb/IIIa antibody or antigen-binding molecule thereof), an intracellular processing site, a heavy chain of the clotting factor (e.g., FVII, FIX, or FX), and a heterologous moiety (e.g., a half-life extending moiety). In other embodiments, the nucleic acid molecule encodes a polypeptide chain comprising a light chain of a clotting factor (e.g., FVII, FIX, or FX), an intracellular processing site, a heavy chain of the clotting factor (e.g., FVII, FIX, or FX), a heterologous moiety (e.g., a half-life extending moiety), and a targeting moiety which binds to a platelet (e.g., an anti-GPIIb/IIIa antibody or antigen-binding molecule thereof). In some embodiments, the nucleic acid molecule encodes a polypeptide chain comprising a light chain of a clotting factor (e.g., FVII, FIX, or FX), an intracellular processing site, a heavy chain of the clotting factor (e.g., FVII, FIX, or FX), a targeting moiety which binds to a platelet (e.g., an anti-GPIIb/IIIa antibody or antigen-binding molecule thereof), and a heterologous moiety (e.g., a half-life extending moiety). In certain embodiments, the nucleic acid molecule encodes a polypeptide chain comprising a light chain of a clotting factor (e.g., FVII, FIX, or FX), a heavy chain of the clotting factor (e.g., FVII, FIX, or FX), at least one (e.g., one two, three, four) heterologous moiety (e.g., a half-life extending moiety such as the XTEN, AE144 or AE288), and a targeting moiety which binds to a platelet (e.g., an anti-GPIIb/IIIa antibody or antigen-binding molecule thereof such as an scFv, or the light and/or heavy chain of an Fab).

In some embodiments, the nucleic acid molecule comprises a set of nucleotide sequences, a first nucleotide sequence encoding a first polypeptide chain comprising a light chain of a clotting factor (e.g., FVII, FIX, or FX) and a heterologous moiety (e.g., a half-life extending moiety) and a second nucleotide sequence encoding a second polypeptide chain comprising a heavy chain of the clotting factor (e.g., FVII, FIX, or FX) and a targeting moiety which binds to a platelet (e.g., an anti-GPIIb/IIIa antibody or antigen-binding molecule thereof). In other embodiments, the nucleic acid molecule comprises a set of nucleotide sequences, a first nucleotide sequence encoding a first polypeptide chain comprising a light chain of a clotting factor (e.g., FVII, FIX, or FX) and a targeting moiety which binds to a platelet (e.g., an anti-GPIIb/IIIa antibody or antigen-binding molecule thereof) and a second nucleotide sequence encoding a second polypeptide chain comprising a heavy chain of the clotting factor (e.g., FVII, FIX, or FX) and a heterologous moiety (e.g., a half-life extending moiety). In other embodiments, the nucleic acid molecule comprises a set of nucleotide sequences, a first nucleotide sequence encoding a light chain of a clotting factor (e.g., FVII, FIX, or FX) and a second nucleotide sequence encoding a heavy chain of the clotting factor (e.g., FVII, FIX, or FX), a heterologous moiety (e.g., a half-life extending moiety), and a targeting moiety which binds to a platelet (e.g., an anti-GPIIb/IIIa antibody or antigen-binding molecule thereof). In some embodiments, the nucleic acid molecule comprises a set of nucleotide sequences, a first nucleotide sequence encoding a light chain of a clotting factor (e.g., FVII, FIX, or FX) and a second nucleotide sequence encoding a heavy chain of the clotting factor (e.g., FVII, FIX, or FX), a targeting moiety which binds to a platelet (e.g., an anti-GPIIb/IIIa antibody or antigen-binding molecule thereof), and a heterologous moiety (e.g., a half-life extending moiety). In other embodiments, the nucleic acid molecule comprises a set of nucleotide sequences, a first nucleotide sequence encoding a first polypeptide chain comprising a light chain of a clotting factor (e.g., FVII, FIX, or FX), a heavy chain of the clotting factor (e.g., FVII, FIX, or FX), at least one (e.g., one two, three, four) heterologous moiety (e.g., a half-life extending moiety such as the XTEN, AE144 or AE288), and either the light chain or the heavy chain of an Fab of an anti-GPIIb/IIIa antibody described herein; and a second nucleotide sequence encoding the corresponding heavy or light chain of the Fab of the anti-GPIIb/IIIa antibody. It is to be understood that by “heavy chain of the Fab” is meant the VH region attached to CH1 of the heavy chain of the antibody.

Also provided are a vector or a set of vectors comprising such nucleic acid molecule or the set of the nucleic acid molecules or a complement thereof, as well as a host cell comprising the vector.

The instant disclosure also provides a method for producing a GPIIb/IIIa antibody or antigen-binding molecule thereof or chimeric molecule disclosed herein, such method comprising culturing the host cell disclosed herein and recovering the antibody, antigen-binding molecule thereof, or the chimeric molecule from the culture medium.

A variety of methods are available for recombinantly producing a GPIIb/IIIa antibody or antigen-binding molecule thereof disclosed herein, or a chimeric molecule disclosed herein. It will be understood that because of the degeneracy of the code, a variety of nucleic acid sequences will encode the amino acid sequence of the polypeptide. The desired polynucleotide can be produced by de novo solid-phase DNA synthesis or by PCR mutagenesis of an earlier prepared polynucleotide.

In one embodiment a first expression vector comprising a DNA comprising a nucleic acid encoding the amino acid sequence of the chimeric polypeptide set forth in SEQ ID NO:125 is transfected into a host cell (e.g., 293, CHO, COS) and the host cell is cultured under conditions that allow for the expression of the chimeric polypeptide. In another embodiment, a first expression vector comprising a DNA comprising a nucleic acid encoding the amino acid sequence of the chimeric polypeptide set forth in SEQ ID NO:125 except that the VL domain of the Fab light chain is replaced with a VL domain from anyone of BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, or BIIB-4-319 is transfected into a host cell (e.g., 293, CHO, COS) and the host cell is cultured under conditions that allow for the expression of the chimeric polypeptide. The chimeric polypeptide is recovered from the cell or culture medium. A second expression vector comprising a DNA comprising a nucleic acid encoding the amino acid sequence of the heavy chain of the Fab set forth in SEQ ID NO:127 or the counterpart Fab heavy chain (e.g., if the chimeric polypeptide contains the VL of BIIB_4_224, the “counterpart” Fab heavy chain would contain the VH of BIIB_4_224) is transfected into a host cell (e.g., 293, CHO, COS) and the host cell is cultured under conditions that allow for the expression of the heavy chain of the Fab. The heavy chain of the Fab is recovered from the cell or culture medium. The chimeric polypeptide and the heavy chain of the Fab are contacted together to permit the heavy chain of the Fab to associate with the light chain of the Fab in the chimeric polypeptide. In another embodiment, a host cell (e.g., 293, CHO, COS) is co-transfected with the first and second expression vectors described above and the host cell is cultured under conditions that allow for the expression of the chimeric polypeptide and the heavy chain of the Fab. The chimeric polypeptide and the heavy chain are isolated from the cell or culture medium. In certain instances, the heavy chain of the Fab is already associated with the light chain of the Fab in the chimeric polypeptide when the polypeptides are isolated from the cell or culture medium. In other instances, the heavy chain of the Fab is not already associated with the light chain of the Fab in the chimeric polypeptide when the polypeptides are isolated from the cell or culture medium and an additional step is required to facilitate their association. In certain embodiments, the Factor VII of the chimeric molecule is activated. Activation of Factor VII can occur by the cleavage of the Arg190-Ile191 peptide bond of Factor VII (SEQ ID NO:128) to create a two chain FVII polypeptide. In one embodiment, the Factor VII of the chimeric molecule is activated by concentrating the chimeric polypeptide (with or without the heavy chain Fab that associates with the light chain Fab of the chimeric polypeptide) to about 4 mg/ml at a pH of 8.0 and incubating the polypeptide at 4° C. for several minutes to an hour (e.g., 1, 2, 4, 5, 10, 15, 20, 25, 30, 35, 40, 45, 50, 55 or 60 minutes).

Oligonucleotide-mediated mutagenesis is one method for preparing a substitution, in-frame insertion, or alteration (e.g., altered codon) to introduce a codon encoding an amino acid substitution (e.g., into a GPIIb/IIIa antibody variant). For example, the starting polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer. In one embodiment, genetic engineering, e.g., primer-based PCR mutagenesis, is sufficient to incorporate an alteration, as defined herein, for producing a polynucleotide encoding a GPIIb/IIIa antibody or antigen-binding molecule thereof disclosed herein, or any of the chimeric molecules disclosed herein.

For recombinant production, a polynucleotide sequence encoding a polypeptide (e.g., a GPIIb/IIIa antibody or antigen-binding molecule thereof disclosed herein, or any of the chimeric molecules disclosed herein) is inserted into an appropriate expression vehicle, i.e., a vector which contains the necessary elements for the transcription and translation of the inserted coding sequence, or in the case of an RNA viral vector, the necessary elements for replication and translation.

The nucleic acid encoding the polypeptide (e.g., a GPIIb/IIIa antibody or antigen-binding molecule thereof disclosed herein, or any of the chimeric molecules disclosed herein) is inserted into the vector in proper reading frame. The expression vector is then transfected into a suitable target cell which will express the polypeptide. Transfection techniques known in the art include, but are not limited to, calcium phosphate precipitation (Wigler et al. 1978, Cell 14:725) and electroporation (Neumann et al. 1982, EMBO J. 1:841). A variety of host-expression vector systems can be utilized to express the polypeptides described herein (e.g., a GPIIb/IIIa antibody or antigen-binding molecule thereof disclosed herein, or any of the chimeric molecules disclosed herein) in eukaryotic cells. In one embodiment, the eukaryotic cell is an animal cell, including mammalian cells (e.g., 293 cells, PerC6, CHO, BHK, Cos, HeLa cells). When the polypeptide is expressed in a eukaryotic cell, the DNA encoding the polypeptide (e.g., a GPIIb/IIIa antibody or antigen-binding molecule thereof disclosed herein, or any of the chimeric molecules disclosed herein) can also code for a signal sequence that will permit the polypeptide to be secreted. One skilled in the art will understand that while the polypeptide is translated, the signal sequence is cleaved by the cell to form the mature chimeric molecule. Various signal sequences are known in the art, e.g., native FVII signal sequence, native FIX signal sequence, native FX signal sequence, native GPIIb signal sequence, native GPIIIa signal sequence, and the mouse IgK light chain signal sequence. Alternatively, where a signal sequence is not included, the polypeptide (e.g., a GPIIb/IIIa antibody or antigen-binding molecule thereof disclosed herein, or any of the chimeric molecules disclosed herein) can be recovered by lysing the cells.

The GPIIb/IIIa antibody or antigen-binding molecule thereof disclosed herein, or any of the chimeric molecules disclosed herein can be synthesized in a transgenic animal, such as a rodent, goat, sheep, pig, or cow. The term “transgenic animals” refers to non-human animals that have incorporated a foreign gene into their genome. Because this gene is present in germline tissues, it is passed from parent to offspring. Exogenous genes are introduced into single-celled embryos (Brinster et al. 1985, Proc. Natl. Acad. Sci. USA 82:4438). Methods of producing transgenic animals are known in the art including transgenics that produce immunoglobulin molecules (Wagner et al. 1981, Proc. Natl. Acad. Sci. USA 78:6376; McKnight et al. 1983, Cell 34:335; Brinster et al. 1983, Nature 306:332; Ritchie et al. 1984, Nature 312:517; Baldassarre et al. 2003, Theriogenology 59:831; Robl et al. 2003, Theriogenology 59:107; Malassagne et al. 2003, Xenotransplantation 10: 267).

The expression vectors can encode for tags that permit for easy purification or identification of the recombinantly produced polypeptide. Examples include, but are not limited to, vector pUR278 (Ruther et al. 1983, EMBO J. 2:1791) in which the polypeptide (e.g., a GPIIb/IIIa antibody or antigen-binding molecule thereof disclosed herein, or any of the chimeric molecules disclosed herein) coding sequence can be ligated into the vector in frame with the lac z coding region so that a hybrid polypeptide is produced; pGEX vectors can be used to express proteins with a glutathione S-transferase (GST) tag. These proteins are usually soluble and can easily be purified from cells by adsorption to glutathione-agarose beads followed by elution in the presence of free glutathione. The vectors include cleavage sites, e.g., for PreCission Protease (Pharmacia, Peapack, N.J.) for easy removal of the tag after purification.

Numerous expression vector systems can be employed. These expression vectors are typically replicable in the host organisms either as episomes or as an integral part of the host chromosomal DNA. Expression vectors can include expression control sequences including, but not limited to, promoters (e.g., naturally-associated or heterologous promoters), enhancers, signal sequences, splice signals, enhancer elements, and transcription termination sequences. Preferably, the expression control sequences are eukaryotic promoter systems in vectors capable of transforming or transfecting eukaryotic host cells. Expression vectors can also utilize DNA elements which are derived from animal viruses such as bovine papilloma virus, polyoma virus, adenovirus, vaccinia virus, baculovirus, retroviruses (RSV, MMTV or MOMLV), cytomegalovirus (CMV), or SV40 virus. Others involve the use of polycistronic systems with internal ribosome binding sites.

Commonly used expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362). Cells which have integrated the DNA into their chromosomes can be selected by introducing one or more markers which allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation.

An exemplary expression vector is NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in cells, followed by selection in G418 containing medium and methotrexate amplification. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other embodiments, polypeptides of the invention (e.g., a GPIIb/IIIa antibody or antigen-binding molecule thereof disclosed herein, or any of the chimeric molecules disclosed herein) can be expressed using polycistronic constructs. In these expression systems, multiple gene products of interest such as multiple polypeptides of multimer binding protein can be produced from a single polycistronic construct. These systems advantageously use an internal ribosome entry site (IRES) to provide relatively high levels of polypeptides of the invention in eukaryotic host cells. Compatible IRES sequences are disclosed in U.S. Pat. No. 6,193,980 which is also incorporated herein. Those skilled in the art will appreciate that such expression systems can be used to effectively produce the full range of polypeptides disclosed in the instant application.

More generally, once the vector or DNA sequence encoding a polypeptide has been prepared, the expression vector can be introduced into an appropriate host cell. That is, the host cells can be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), flow cytometry, immunohistochemistry, and the like.

As used herein, the term “transformation” refers in a broad sense to the introduction of DNA into a recipient host cell that changes the genotype and consequently results in a change in the recipient cell. Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of polypeptide unless it is clearly specified otherwise. In other words, recovery of polypeptide from the “cells” can mean either from spun down whole cells, or from the cell culture containing both the medium and the suspended cells.

In one embodiment, a host cell endogenously expresses an enzyme (or the enzymes) necessary to cleave a scFc linker (e.g., if such a linker is present and contains intracellular processing site(s)) during processing to form the mature polypeptide. During this processing, the scFc linker can be substantially removed to reduce the presence of extraneous amino acids. In another embodiment of the invention, a host cell is transformed to express one or more enzymes which are exogenous to the cell such that processing of a scFc linker occurs or is improved.

In one embodiment an enzyme which can be endogenously or exogenously expressed by a cell is a member of the furin family of enzymes. Complete cDNA and amino acid sequences of human furin (i.e., PACE) were published in 1990. Van den Ouweland A M et al. (1990) Nucleic Acids Res. 18:664; Erratum in: Nucleic Acids Res. 18:1332 (1990). U.S. Pat. No. 5,460,950, issued to Barr et al., describes recombinant PACE and the coexpression of PACE with a substrate precursor polypeptide of a heterologous protein to improve expression of active, mature heterologous protein. U.S. Pat. No. 5,935,815, likewise describes recombinant human furin (i.e., PACE) and the coexpression of furin with a substrate precursor polypeptide of a heterologous protein to improve expression of active, mature heterologous protein. Possible substrate precursors disclosed in this patent include a precursor of Factor IX. Other family members in the mammalian furin/subtilisin/Kex2p-like proprotein convertase (PC) family in addition to PACE are reported to include PCSK1 (also known as PC1/Pc3), PCSK2 (also known as PC2), PCSK3 (also known as furin or PACE), PCSK4 (also known as PC4), PCSK5 (also known as PC5 or PC6), PCSK6 (also known as PACE4), or PCSK7 (also known as PC7/LPC, PC8, or SPC7). While these various members share certain conserved overall structural features, they differ in their tissue distribution, subcellular localization, cleavage specificities, and preferred substrates. For a review, see Nakayama K (1997) Biochem J. 327:625-35. Similar to PACE, these proprotein convertases generally include, beginning from the amino terminus, a signal peptide, a propeptide (that can be autocatalytically cleaved), a subtilisin-like catalytic domain characterized by Asp, His, Ser, and Asn/Asp residues, and a Homo B domain that is also essential for catalytic activity and characterized by an Arg-Gly-Asp (RGD) sequence. PACE, PACE4, and PC5 also include a Cys-rich domain, the function of which is unknown. In addition, PC5 has isoforms with and without a transmembrane domain; these different isoforms are known as PC5B and PC5A, respectively. Comparison between the amino acid sequence of the catalytic domain of PACE and the amino acid sequences of the catalytic domains of other members of this family of proprotein convertases reveals the following degrees of identity: 70 percent for PC4; 65 percent for PACE4 and PC5; 61 percent for PC1/PC3; 54 percent for PC2; and 51 percent for LPC/PC7/PC8/SPC7. Nakayama K (1997) Biochem J., 327:625-35.

PACE and PACE4 have been reported to have partially overlapping but distinct substrates. In particular, PACE4, in striking contrast to PACE, has been reported to be incapable of processing the precursor polypeptide of FIX. Wasley et al. (1993) J. Biol. Chem. 268:8458-65; Rehemtulla et al. (1993) Biochemistry. 32:11586-90. U.S. Pat. No. 5,840,529, discloses nucleotide and amino acid sequences for human PC7 and the notable ability of PC7, as compared to other PC family members, to cleave HIV gp160 to gp120 and gp41.

Nucleotide and amino acid sequences of rodent PC5 were first described as PC5 by Lusson et al. (1993) Proc Natl Acad Sci USA 90:6691-5 and as PC6 by Nakagawa et al. (1993) J Biochem (Tokyo) 113:132-5. U.S. Pat. No. 6,380,171 discloses nucleotide and amino acid sequences for human PCSA, the isoform without the transmembrane domain. The sequences of these enzymes and method of cloning them are known in the art.

Genes encoding the polypeptides of the invention (e.g., a GPIIb/IIIa antibody or antigen-binding molecule thereof disclosed herein, or any of the chimeric molecules disclosed herein) can also be expressed in non-mammalian cells such as bacteria or yeast or plant cells. In this regard it will be appreciated that various unicellular non-mammalian microorganisms such as bacteria can also be transformed; i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules.

In addition to prokaryates, eukaryotic microbes can also be used. Saccharomyces cerevisiae, or common baker's yeast, is the most commonly used among eukaryotic microorganisms although a number of other strains are commonly available.

For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

Other yeast hosts such Pichia can also be employed. Yeast expression vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Alternatively, polypeptide-coding nucleotide sequences can be incorporated in transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for polypeptides in operable linkage with a promoter and enhancer from a mammary gland specific gene, such as casein or beta lactoglobulin.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-) affinity chromatography, e.g., after preferential biosynthesis of a synthetic hinge region polypeptide or prior to or subsequent to the HIC chromatography step described herein. An affinity tag sequence (e.g. a His(6) tag (SEQ ID NO: 253) can optionally be attached or included within the polypeptide sequence to facilitate downstream purification.

Once expressed, the chimeric molecules can be purified according to standard procedures of the art, including ammonium sulfate precipitation, affinity column chromatography, HPLC purification, gel electrophoresis and the like (see generally Scopes, Protein Purification (Springer-Verlag, N.Y., (1982)) and see specifically the methods used in the instant Examples. Substantially pure proteins of at least about 90 to 95% homogeneity are preferred, and 98 to 99% or more homogeneity most preferred, for pharmaceutical uses.

### G. Pharmaceutical Compositions

The present disclosure also provides pharmaceutical compositions comprising one or more of:
(i) a GPIIb/IIIa antibody or antigen-binding molecule thereof disclosed herein;
(ii) a chimeric molecule disclosed herein;
(iii) a nucleic acid molecule or the set of nucleic acid molecules disclosed herein; or
(iv) a vector or set of vectors disclosed herein,
   and a pharmaceutically acceptable carrier.

In some embodiments, administering (i) a chimeric molecule disclosed herein, (ii) a nucleic acid molecule or a set of nucleic acid molecules disclosed herein, (iii) a vector or a set of vectors disclosed herein, or (iii) a pharmaceutical composition disclosed herein, can be used, for example, to reduce the frequency or degree of a bleeding episode in a subject in need, and/or reducing or preventing an occurrence of a bleeding episode in a subject in need thereof. In such instances the antibody used will be a Class I or Class I antibody. In some embodiments, the subject has developed or has a tendency to develop an inhibitor against treatment with FVIII, FIX, or both. In some embodiments, the inhibitor against FVIII or FIX is a neutralizing antibody against FVIII, FIX, or both. In some embodiments, the bleeding episode can be caused by a blood coagulation disorder, for example, hemophilia A or hemophilia B. In other embodiments, the bleeding episode can be the result of hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, or any combinations thereof. In certain embodiments, the subject is a human subject.

A pharmaceutical composition comprising a Class III antibody or antigen-binding fragment can be used to reduce or prevent platelet aggregation or thrombosis in a human subject in need thereof.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

In one embodiment, the pharmaceutical composition (e.g., a composition comprising the polypeptide(s) or nucleic acid molecule(s) encoding the polypeptide(s)) is one in which the clotting factor is present in activatable form when administered to a subject. Such an activatable molecule can be activated in vivo at the site of clotting after administration to a subject.

### H. Methods of Treatment

The antibodies, antigen-binding fragments thereof and chimeric molecules of the disclosure can be useful in methods of treating a subject with a disease or condition. For example, the antibodies, antigen-binding fragments thereof and chimeric molecules based on Class I or Class II antibodies described herein can be used to treat, prevent, or ameliorate a disease or condition that includes, but is not limited to, hemostatic or coagulation disorders. In certain embodiments, the Class I or Class II antibodies or antigen-binding fragments thereof, and chimeric molecules based on Class I or Class II antibodies described herein can be used to treat, prevent, or ameliorate bleeding episodes and in the pen-operative management of patients with congenital hemophilia A and B with inhibitors, acquired hemophilia, congenital FVII deficiency, and Glanzmann's thrombasthenia. In other embodiments, these agents can be used to treat, prevent, or ameliorate hemophilia A and B, or trauma in a subject in need thereof. In certain embodiments, the antibodies, antigen-binding fragments thereof and chimeric molecules based on Class III antibodies described herein can be used to treat, prevent, or ameliorate a disease or condition that involves platelet aggregation or platelet thrombus formation.

This disclosure provides a method of treating, ameliorating, or preventing a hemostatic disorder to a subject comprising administering a therapeutically effective amount of a chimeric molecule of the disclosure (that includes an antibody or antigen-binding fragment of Class I or Class II anti-GPIIb/IIIa antibodies disclosed herein) which comprises a clotting factor. The treatment, amelioration, and prevention by the chimeric molecule can be a bypass therapy. The subject in the bypass therapy can have already developed an inhibitor to a clotting factor, e.g., FVIII or FIX, or is subject to developing a clotting factor inhibitor. In one embodiment, a chimeric molecule composition of the invention is administered in combination with at least one other agent that promotes hemostasis. As an example, but not as a limitation, hemostatic agent can include a FV, FVII, FVIII, FIX, FX, FXI, FXII, FXIII, prothrombin, or fibrinogen or activated forms of any of the preceding. The clotting factor or hemostatic agent can also include anti-fibrinolytic drugs, e.g., epsilon-amino-caproic acid, tranexamic acid.

The chimeric molecules of the disclosure treat or prevent a hemostatic disorder by promoting the formation of a fibrin clot. The chimeric molecule of the invention can activate any member of a coagulation cascade. The clotting factor can be a participant in the extrinsic pathway, the intrinsic pathway or both. A chimeric molecule of the invention (that includes an antibody or antigen-binding fragment of Class I or Class II anti-GPIIb/IIIa antibodies disclosed herein) can be used to treat hemostatic disorders, e.g., those known to be treatable with the particular clotting factor present in the chimeric molecule. The hemostatic disorders that can be treated by administration of the chimeric molecule of the invention include, but are not limited to, hemophilia A, hemophilia B, von Willebrand's disease, Factor XI deficiency (PTA deficiency), Factor XII deficiency, as well as deficiencies or structural abnormalities in fibrinogen, prothrombin, Factor V, Factor VII, Factor X, or Factor XIII.

In one embodiment, the hemostatic disorder is an inherited disorder. In one embodiment, the subject has hemophilia A, and the chimeric molecule comprises activated or protease-activatable FVII linked to or associated with a GPIIb/IIIa antibody or antigen-binding molecule thereof and a half-life extending heterologous moiety. In another embodiment, the subject has hemophilia A and the chimeric molecule comprises activated or protease-activatable FVII linked to or associated with an Fab or scFv of an GPIIb/IIIa antibody and a half-life extending heterologous moiety. In other embodiments, the subject has hemophilia B and the chimeric molecule comprises activated or protease-activatable FVII or FX linked to or associated with a GPIIb/IIIa antibody or antigen-binding molecule thereof (of Class I or Class II) and a half-life extending heterologous moiety. In some embodiments, the subject has inhibitory antibodies to FVIII or FVIIIa and the chimeric molecule comprises activated or protease-activatable FVII linked to or associated with a GPIIb/IIIa antibody or antigen-binding molecule thereof (of Class I or Class II) and a half-life extending heterologous moiety. In yet other embodiments, the subject has inhibitory antibodies against FIX or FIXa and the chimeric molecule comprises activated or protease-activatable FVII linked to or associated with a GPIIb/IIIa antibody or antigen-binding molecule thereof (of Class I or Class II) and a half-life extending heterologous moiety. In still other embodiments, the subject has inhibitory antibodies to FVIII or FVIIIa and the chimeric molecule comprises activated or protease-activatable FX linked to or associated with a GPIIb/IIIa antibody or antigen-binding molecule thereof (of Class I or Class II) and a half-life extending heterologous moiety. In certain embodiments, the subject has inhibitory antibodies against FIX or FIXa and the chimeric molecule comprises activated or protease-activatable FX linked to or associated with a GPIIb/IIIa antibody or antigen-binding molecule thereof (of Class I or Class II) and a half-life extending heterologous moiety.

Chimeric molecules of the disclosure comprising a clotting factor (e.g., FVII) can be used to prophylactically treat a subject with a hemostatic or coagulation disorder. Chimeric molecules of the invention comprising a clotting factor (e.g., FVII) can be used to treat an acute bleeding episode in a subject with a hemostatic disorder.

In one embodiment, the hemostatic disorder is the result of a deficiency in a clotting factor, e.g., FVII, FIX, or FVIII. In another embodiment, the hemostatic disorder can be the result of a defective clotting factor. In another embodiment, the hemostatic disorder can be an acquired disorder. The acquired disorder can result from an underlying secondary disease or condition. The unrelated condition can be, as an example, but not as a limitation, cancer, an autoimmune disease, or pregnancy. The acquired disorder can result from old age or from medication to treat an underlying secondary disorder (e.g. cancer chemotherapy).

The disclosure thus relates to a method of treating a subject in need of a general hemostatic agent comprising administering a therapeutically effective amount of at least one chimeric molecule of the invention (that includes an antibody or antigen-binding fragment of Class I or Class II anti-GPIIb/IIIa antibodies disclosed herein). For example, in one embodiment, the subject in need of a general hemostatic agent is undergoing, or is about to undergo, surgery. The chimeric molecule of the invention can be administered prior to or after surgery as a prophylactic. The chimeric molecule of the invention can be administered during or after surgery to control an acute bleeding episode. The surgery can include, but is not limited to, liver transplantation, liver resection, or stem cell transplantation. In another embodiment, the chimeric molecule of the invention can be used to treat a subject having an acute bleeding episode who does not have a hemostatic disorder. The acute bleeding episode can result from severe trauma, e.g., surgery, an automobile accident, wound, laceration gun shot, or any other traumatic event resulting in uncontrolled bleeding.

The disclosure also relates to methods of reducing or preventing platelet aggregation. The method involves administering a subject (e.g, a human) in need thereof a therapeutically effective amount of a Class III antibody or antigen-binding fragment thereof. In certain embodiments the Class III antibody or antigen-binding fragment thereof may include a heterologous moiety such as a half-life extending moiety (e.g., AE144, AE288).

The disclosure further relates to methods of reducing or preventing platelet thrombus formation (e.g., intracoronary atherothrombosis). The method involves administering a subject (e.g, a human) in need thereof a therapeutically effective amount of a Class III antibody or antigen-binding fragment thereof. In certain embodiments the Class III antibody or antigen-binding fragment thereof may include a heterologous moiety such as a half-life extending moiety (e.g., AE144, AE288).

The disclosure also relates to methods of treating a human subject undergoing high-risk percutaneous transluminal coronary angioplasty (PTCA), or having, or at risk of developing acute coronary syndrome (ACS) or unstable angina (UA). The method involves administering the subject in need thereof a therapeutically effective amount of a Class III antibody or antigen-binding fragment thereof. In certain embodiments the Class III antibody or antigen-binding fragment thereof may include a heterologous moiety such as a half-life extending moiety (e.g., AE144, AE288).

### I. Administration

The antibodies, antigen-binding fragments thereof, chimeric molecules, or nucleic acids encoding same of the disclosure can be administered intravenously, subcutaneously, intramuscularly, or via any mucosal surface, e.g., orally, sublingually, buccally, sublingually, nasally, rectally, vaginally or via pulmonary route. The chimeric molecule can be implanted within or linked to a biopolymer solid support that allows for the slow release of the chimeric molecule to the desired site. The route and/or mode of administration of the antibody or antigen-binding fragment thereof can also be tailored for the individual case, e.g., by monitoring the subject,

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle. For buccal and sublingual administration the composition can take the form of tablets, lozenges or fast dissolving films according to conventional protocols. For administration by inhalation, the chimeric molecules for use according to the present disclosure are conveniently delivered in the form of an aerosol spray from a pressurized pack or nebulizer (e.g., in PBS), with a suitable propellant.

In one embodiment, the route of administration of the polypeptides of the invention is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the invention, a form for administration would be a solution for injection, in particular for intravenous or intraarterial injection or drip. Usually, a suitable pharmaceutical composition for injection can comprise a buffer (e.g., acetate, phosphate or citrate buffer), a surfactant (e.g. polysorbate), optionally a stabilizer agent (e.g., human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture will preferably have labels or package inserts indicating that the associated compositions are useful for treating a subject suffering from, or predisposed to clotting disorders.

The pharmaceutical composition can also be formulated for rectal administration as a suppository or retention enema, e.g., containing conventional suppository bases such as cocoa butter or other glycerides.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

In one embodiment, the dose of a biologically active moiety (e.g., comprising FVII), can range from about 90 to 270 μg/kg or 0.090 to 0.270 mg/kg. In another embodiment, the dose of a biologically active moiety (e.g., comprising FX), can range from about 1 μg/kg to 400 mg/kg.

Dosages can range from 1000 μg/kg to 0.1 ng/kg body weight. In one embodiment, the dosing range is 1 ug/kg to 100 μg/kg. The protein can be administered continuously or at specific timed intervals. In vitro assays can be employed to determine optimal dose ranges and/or schedules for administration. In vitro assays that measure clotting factor activity are known in the art, e.g., STA-CLOT Vlla-rTF clotting assay. Additionally, effective doses can be extrapolated from dose-response curves obtained from animal models, e g., a hemophiliac dog (Mount et al. 2002, Blood 99: 2670).

Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. In some methods, two or more polypeptides can be administered simultaneously, in which case the dosage of each polypeptide administered falls within the ranges indicated.

Polypeptides of the invention can be administered on multiple occasions. Intervals between single dosages can be daily, weekly, monthly or yearly. Intervals can also be irregular as indicated by measuring blood levels of modified polypeptide or antigen in the patient. Alternatively, polypeptides can be administered as a sustained release formulation, in which case less frequent administration is required. Dosage and frequency vary depending on the half-life of the polypeptide in the patient.

The dosage and frequency of administration can vary depending on whether the treatment is prophylactic or therapeutic. In prophylactic applications, compositions containing the polypeptides of the invention or a cocktail thereof are administered to a patient not already in the disease state to enhance the patient's resistance or minimize effects of disease. Such an amount is defined to be a “prophylactic effective dose.” A relatively low dosage is administered at relatively infrequent intervals over a long period of time. Some patients continue to receive treatment for the rest of their lives.

Polypeptides of the invention can optionally be administered in combination with other agents that are effective in treating the disorder or condition in need of treatment (e.g., prophylactic or therapeutic).

As used herein, the administration of polypeptides of the invention in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the polypeptides of the instant invention can be used in conjunction or combination with an agent or agents (e.g., to provide a combined therapeutic regimen). Exemplary agents with which a polypeptide of the invention can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the polypeptides of the instant invention can vary by subject or can be administered according to what is known in the art. See for example, Bruce A Chabner et al., Antineoplastic Agents, in Goodman & Gilman's The Pharmacological Basis of Therapeutics 1233-1287 ((Hardman et al., eds., 9th ed. 1996). In another embodiment, an amount of such an agent consistent with the standard of care is administered.

As previously discussed, the polypeptides of the present disclosure, can be administered in a pharmaceutically effective amount for the in vivo treatment of clotting disorders. In this regard, it will be appreciated that the polypeptides of the invention can be formulated to facilitate administration and promote stability of the active agent. Preferably, pharmaceutical compositions in accordance with the present disclosure comprise a pharmaceutically acceptable, non-toxic, sterile carrier such as physiological saline, non-toxic buffers, preservatives and the like. Of course, the pharmaceutical compositions of the present disclosure can be administered in single or multiple doses to provide for a pharmaceutically effective amount of the polypeptide.

In one embodiment, a chimeric molecule of the invention is administered as a nucleic acid molecule. Nucleic acid molecules can be administered using techniques known in the art, including via vector, plasmid, liposome, DNA injection, electroporation, gene gun, intravenously injection or hepatic artery infusion. Vectors for use in gene therapy embodiments are known in the art.

In keeping with the scope of the present disclosure, the chimeric molecule of the invention can be administered to a human or other animal in accordance with the aforementioned methods of treatment in an amount sufficient to produce a therapeutic or prophylactic effect.

### J. Other Methods of Use

The instant disclosure also provides a method to target or deliver a therapeutic or prophylactic agent (e.g., a clotting factor such as FVII) to the surface of platelets, wherein the method comprises fusing the agent to one of the GPIIb/IIIa antibodies or antigen-binding fragments thereof disclosed herein.

In addition, the disclosure provides a method to increase the activity of a therapeutic or prophylactic agent (e.g., a clotting factor such as FVII) comprising fusing the agent to a GPIIb/IIIa antibody or antigen-binding molecule thereof disclosed herein (e.g., a Class I or Class II antibody or antigen-binding fragment).

Further, the disclosure provides a method to improve the pharmacokinetic properties of a clotting factor comprising fusing the clotting factor to the GPIIb/IIIa antibody or antigen-binding molecule thereof disclosed herein (e.g., a Class I or Class II antibody or antigen-binding fragment).

In some embodiments, these methods further comprise fusing or conjugating a clotting factor and/or the GPIIb/IIIa antibody or antigen-binding molecule thereof disclosed herein to a half-life extending moiety. In some embodiments, the therapeutic or prophylactic agent is a FVII, a FIX, or a FX.

The present disclosure also provides a method of measuring the level of platelets in a body fluid sample (e.g., plasma) of a subject in need thereof comprising contacting the GPIIb/IIIa antibody or antigen binding molecule thereof disclosed herein with the sample from the subject and measuring the level of platelets in the body fluid. This method can further comprise fusing or conjugating the clotting factor and/or the GPIIb/IIIa antibody or antigen-binding molecule thereof disclosed herein to a detectable heterologous moiety, for example, a fluorescent molecule or a radionuclide.

This disclosure also provides a method of isolating or separating platelets from other cells in a sample (e.g., a blood sample). The method comprises contacting the sample with an GPIIb/IIIa antibody or antigen binding molecule thereof disclosed herein and separating the cells that have bound to the GPIIb/IIIa antibody or antigen binding molecule thereof from the unbound fraction.

In addition, the disclosure also provides a method of detecting platelets in a sample (e.g., blood sample) of a subject comprising contacting the sample with a detectably labeled GPIIb/IIIa antibody or antigen binding molecule. The detectable label can be, for example, a fluorescent molecule or a radionuclide.

Furthermore, the disclosure includes methods of isolating or enriching activated platelets from a sample. This method involves contacting the sample with an antibody or antigen-binding fragment of a Class I antibody and isolating the bound fraction of cells. The bound fraction predominantly contains the activated platelets.

Also, the disclosure encompasses the use of Class III antibodies or antigen-binding fragments thereof as diagnostic tools for evaluating fibrinogen blocking. For example, the Class III antibodies or antigen-binding fragments thereof can be used as a surrogate for fibrinogen, to block the ligand binding site in assays. The Class III antibodies or antigen-binding fragments thereof can also be used as probes (e.g., linked to a detectable label) to identify a sample that is capable of binding fibrinogen. In one embodiment, the disclosure provides a method involving, contacting a sample with a Class III antibody or antigen-binding fragment thereof disclosed herein linked to or conjugated with a detectable label and identifying cells to which the Class III antibody or antigen-binding fragment thereof are bound as a sample that is capable of binding to fibrinogen when compared to those cells in the sample that are not bound by the antibody or antigen-binding fragment thereof.

The following examples are included for purposes of illustration only and are not intended to be limiting of the invention. All patents and publications referred to herein are expressly incorporated by reference in their entireties.

### EXAMPLES

### Example 1: Design of Antibody Selections and Antibody Production

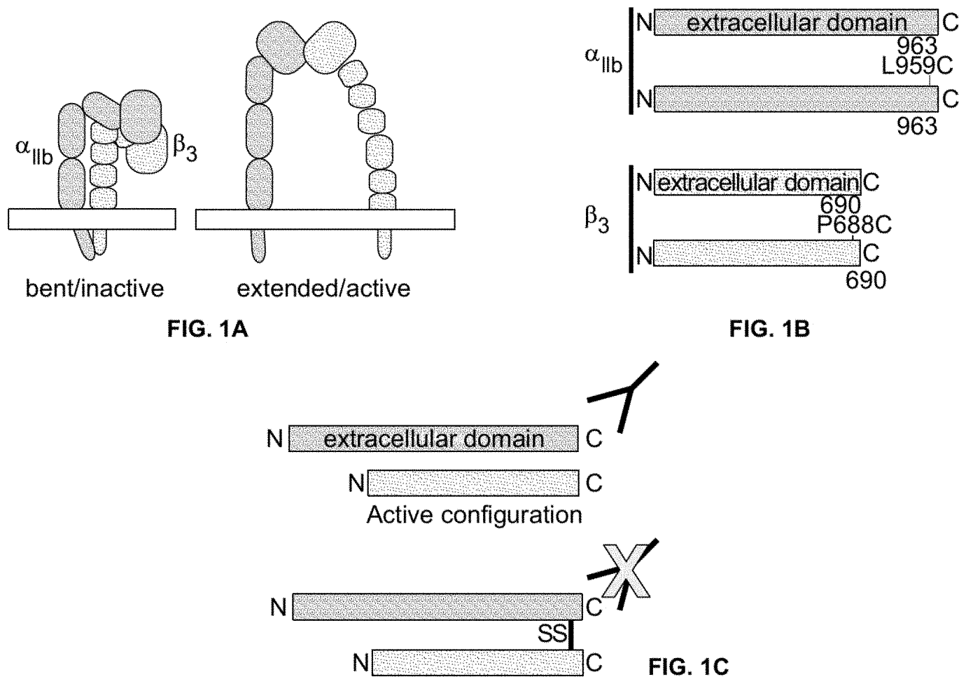

Glycoprotein IIb/IIIa (GP2b3a, also known as integrin α_{IIb}β₃) is a platelet-resident receptor. It can exist in two major conformational states: in the bent/inactive form, it is incapable of binding ligand, such as fibrinogen; however, in the extended/active formation, which can be triggered by platelet activation in the clotting cascade, it is capable of binding to fibrinogen and propagating platelet aggregation (FIG. 1A). GPIIb/IIIa bearing a non-native disulfide bond (αIIb L959C (SEQ ID NO:2), β3 P688C (SEQ ID NO:4) linking the alpha and beta chains has been demonstrated to lock the integrin in an inactive conformation (Zhu et al., Mol Cell, 32(6):849-61 (2008)) (FIG. 1B). In this same study, the wild type (WT) GPIIb/IIIa ectodomain (αIIb (SEQ ID NO:1) and β3 (SEQ ID NO:3)) was shown to exist in a conformational equilibrium between active and inactive conformations.

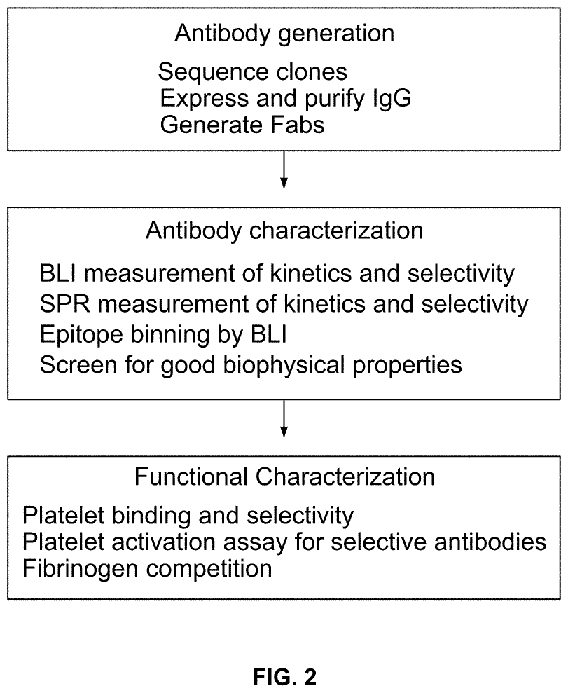

Both forms of GPIIb/IIIa were recombinantly expressed and purified according to methods known in the art. This disclosure describes antibodies against GPIIb/IIIa that are capable of targeting the inactive platelet integrin as well as antibodies that display preference for binding to active GPIIb/IIIa in recombinant form and on platelets. To generate these classes of antibodies, Adimab expression libraries were screened in accordance with the methods disclosed in US Patent Publications 20100056386 and 20090181855. After iterative rounds of selective pressure towards the targeted antigen GPIIb/IIIa (SEQ ID NOs: 1 and 3) and efforts to diminish binding to undesired antigen, GPIIb/IIIa (SEQ ID NOs: 2 and 4) (FIG. 1C), colonies were sequenced to identify unique clones, using techniques known in the art. Following two campaigns, 564 antibodies were expressed and purified on protein A resin from yeast, followed by standard Fab generation using methods known in the art. A general outline of the triage of GPIIb/IIIa-specific antibodies is depicted in FIG. 2. This analysis led to the identification of twelve GPIIb/IIIa-specific antibodies: BIIB-4-147, BIIB-4-156, BIIB-4-174, BIIB-4-175, BIIB-4-204, BIIB-4-209, BIIB-4-224, BIIB-4-309, BIIB-4-311, BIIB-4-317, BIIB-4-318, BIIB-4-319. (FIGS. 3 and 4). The amino acid and nucleic acid sequences of the variable regions of these antibodies are provided below.

### Sequences of the Heavy Chain Variable Domain (VH) of the Identified Antibodies (CDRs are Underlined):

BIIB-4-147_VH Amino Acid Sequence
(SEQ ID NO: 5)
QVQLVQSGAEVKKPGASVKVSCKASGYTFTSYGISWVRQAPGQGLEWMGWISAYNGNTNYAQKLQGRVTMTTDTS
TSTAYMELRSLRSDDTAVYYCARDLEYYDSSGYAYGYFDLWGRGTLVTVSS
BIIB-4-147_VH Nucleic Acid Sequence
(SEQ ID NO: 6)
CAGGTTCAGCTGGTGCAGTCTGGAGCTGAGGTGAAGAAGCCTGGGGCCTCAGTGAAGGTCTCCTGCAAGGCTTCT
GGTTACACCTTTACCAGCTATGGTATCAGCTGGGTGCGACAGGCCCCTGGACAAGGGCTTGAGTGGATGGGATGG
ATCAGCGCTTACAATGGTAACACAAACTATGCACAGAAGCTCCAGGGCAGAGTCACCATGACCACAGACACATCC
ACGAGCACAGCCTACATGGAGCTGAGGAGCCTGAGATCTGACGACACGGCGGTGTACTACTGCGCCAGAGACTTG
GAATACTACGACAGCAGCGGATACGCCTATGGCTACTTCGACCTATGGGGGAGAGGTACCTTGGTCACCGTCTCC
TCA
BIIB-4-156_VH Amino Acid Sequence
(SEQ ID NO: 9)
QVQLVQSGAEVKKPGSSVKVSCKASGGTFSSYAISWVRQAPGQGLEWMGGIIPIFGTANYAQKFQGRVTITADES
TSTAYMELSSLRSEDTAVYYCARDTGYYGASLYFDYWGQGTLVTVSS
BIIB-4-156_VH Nucleic Acid Sequence
(SEQ ID NO: 10)
CAGGTGCAGCTGGTGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACGAATCC
ACGAGCACAGCCTACATGGAGCTGAGCAGCCTGAGATCTGAGGACACGGCGGTGTACTACTGCGCCAGAGACACG
GGATACTACGGTGCTAGCTTATATTTCGACTATTGGGGACAGGGTACATTGGTCACCGTCTCCTCA
BIIB-4-174_VH Amino Acid Sequence
(SEQ ID NO: 13)
QVQLVQSGAEVKKPGSSVKVSCKASGGTFSSYAISWVRQAPGQGLEWMGGIIPIFGTANYAQKFQGRVTITADES
TSTAYMELSSLRSEDTAVYYCARGPPSAYGDYVWDIWGQGTMVTVSS
BIIB-4-174_VH Nucleic Acid Sequence
(SEQ ID NO: 14)
CAGGTGCAGCTGGTGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACGAATCC
ACGAGCACAGCCTACATGGAGCTGAGCAGCCTGAGATCTGAGGACACGGCGGTGTACTACTGCGCCAGAGGACCG
CCTAGCGCCTACGGAGACTACGTCTGGGACATATGGGGTCAGGGTACAATGGTCACCGTCTCCTCA
BIIB-4-175_VH Amino Acid Sequence
(SEQ ID NO: 17)
EVQLVESGGGLVQPGGSLRLSCAASGFTFSDHHMDWVRQAPGKGLEWVGRTRNKANSYTTEYAASVKGRFTISRD
PSKNSLYLQMNSLKTEDTAVYYCARGPPYYADLGMGVWGQGTTVTVSS
BIIB-4-175_VH Nucleic Acid Sequence
(SEQ ID NO: 18)
GAGGTGCAGCTGGTGGAGTCTGGGGGAGGCTTGGTCCAGCCTGGAGGGTCCCTGAGACTCTCCTGTGCAGCCTCT
GGATTCACCTTCAGTGACCACCACATGGACTGGGTCCGCCAGGCTCCAGGGAAGGGGCTGGAGTGGGTTGGCCGT
ACTAGAAACAAAGCTAACAGTTACACCACAGAATACGCCGCGTCTGTGAAAGGCAGATTCACCATCTCAAGAGAT
GATTCAAAGAACTCACTGTATCTGCAAATGAACAGCCTGAAAACCGAGGACACGGCGGTGTACTACTGCGCCAGA
GGACCGCCTTACTACGCAGACCTCGGAATGGGCGTATGGGGCCAGGGAACAACTGTCACCGTCTCCTCA
BIIB-4-204_VH Amino Acid Sequence
(SEQ ID NO: 21)
QVQLVQSGAEVKKPGASVKVSCKASGYTFTSYSMHWVRQAPGQGLEWMGIINPSGGSTSYAQKFQGRVTMTRDTS
TSTVYMELSSLRSEDTAVYYCARSYDIGYFDLWGRGTLVTVSS
BIIB-4-204_VH Nucleic Acid Sequence
(SEQ ID NO: 22)
CAGGTGCAGCTGGTGCAGTCTGGGGCTGAGGTGAAGAAGCCTGGGGCCTCAGTGAAGGTTTCCTGCAAGGCATCT
GGATACACCTTCACCAGCTACAGCATGCACTGGGTGCGACAGGCCCCTGGACAAGGGCTTGAGTGGATGGGAATA
ATCAACCCTAGTGGTGGTAGCACAAGCTACGCACAGAAGTTCCAGGGCAGAGTCACCATGACCAGGGACACGTCC
ACGAGCACAGTCTACATGGAGCTGAGCAGCCTGAGATCTGAGGACACGGCGGTGTACTACTGCGCCAGATCTTAC
GACATAGGCTACTTCGACCTATGGGGGAGAGGTACCTTGGTCACCGTCTCCTCA
BIIB-4-209_VH Amino Acid Sequence
(SEQ ID NO: 25)
QVQLVQSGAEVKKPGASVKVSCKASGYTFTSYGISWVRQAPGQGLEWMGWISAYNGNTNYAQKLQGRVTMTTDTS
TSTAYMELRSLRSDDTAVYYCARGRPYDHYFDYWGQGTLVTVSS
BIIB-4-209_VH Nucleic Acid Sequence
(SEQ ID NO: 26)
CAGGTTCAGCTGGTGCAGTCTGGAGCTGAGGTGAAGAAGCCTGGGGCCTCAGTGAAGGTCTCCTGCAAGGCTTCT
GGTTACACCTTTACCAGCTATGGTATCAGCTGGGTGCGACAGGCCCCTGGACAAGGGCTTGAGTGGATGGGATGG
ATCAGCGCTTACAATGGTAACACAAACTATGCACAGAAGCTCCAGGGCAGAGTCACCATGACCACAGACACATCC
ACGAGCACAGCCTACATGGAGCTGAGGAGCCTGAGATCTGACGACACGGCGGTGTACTACTGCGCCAGAGGAAGG
CCTTACGACCACTACTTTGACTACTGGGGACAGGGTACATTGGTCACCGTCTCCTCA
BIIB-4-224_VH Amino Acid Sequence
(SEQ ID NO: 29)
QLQLQESGPGLVKPSETLSLTCTVSGGSISSSSYYWGWIRQPPGKGLEWIGSIYYSGSTYYNPSLKSRVTISVDT
SKNQFSLKLSSVTAADTAVYYCARDFYSSVYGMDVWGQGTTVTVSS
BIIB-4-224_VH Nucleic Acid Sequence
(SEQ ID NO: 30)
CAGCTGCAGCTGCAGGAGTCGGGCCCAGGACTGGTGAAGCCTTCGGAGACCCTGTCCCTCACCTGCACTGTCTCT
GGTGGCTCCATCAGCAGTAGTAGTTACTACTGGGGCTGGATCCGCCAGCCCCCAGGGAAGGGGCTGGAGTGGATT
GGGAGTATCTATTATAGTGGGAGCACCTACTACAACCCGTCCCTCAAGAGTCGAGTCACCATATCCGTAGACACG
TCCAAGAACCAGTTCTCCCTGAAGCTGAGTTCTGTGACCGCCGCAGACACGGCGGTGTACTACTGCGCCAGAGAC
TTCTACAGCAGTGTATACGGTATGGACGTTTGGGGCCAGGGAACAACTGTCACCGTCTCCTCA
BIIB-4-309_VH Amino Acid Sequence
(SEQ ID NO: 33)
QVQLVQSGAEVKKPGASVKVSCKASGYTFTSYGISWVRQAPGQGLEWMGWISAYNGNTNYAQKLQGRVTMTTDTS
TSTAYMELRSLRSDDTAVYYCARDGLGSSPWSAFDIWGQGTMVTVSS
BIIB-4-309_VH Nucleic Acid Sequence
(SEQ ID NO: 34)
CAGGTTCAGCTGGTGCAGTCTGGAGCTGAGGTGAAGAAGCCTGGGGCCTCAGTGAAGGTCTCCTGCAAGGCTTCT
GGTTACACCTTTACCAGCTATGGTATCAGCTGGGTGCGACAGGCCCCTGGACAAGGGCTTGAGTGGATGGGATGG
ATCAGCGCTTACAATGGTAACACAAACTATGCACAGAAGCTCCAGGGCAGAGTCACCATGACCACAGACACATCC
ACGAGCACAGCCTACATGGAGCTGAGGAGCCTGAGATCTGACGACACGGCGGTGTACTACTGCGCCAGAGACGGA
CTGGGATCCAGCCCATGGTCAGCTTTCGACATATGGGGTCAGGGTACAATGGTCACCGTCTCCTCA
BIIB-4-311_VH Amino Acid Sequence
(SEQ ID NO: 37)
QVQLVQSGAEVKKPGASVKVSCKASGYTFTSYYMHWVRQAPGQGLEWMGVINPSGGSTSYAQKFQGRVTMTRDTS
TSTVYMELSSLRSEDTAVYYCARLMSGSSGSWGQGTLVTVSS
BIIB-4-311_VH Nucleic Acid Sequence
(SEQ ID NO: 38)
CAGGTGCAGCTGGTGCAGTCTGGGGCTGAGGTGAAGAAGCCTGGGGCCTCAGTGAAGGTTTCCTGCAAGGCATCT
GGATACACCTTCACCAGCTACTATATGCACTGGGTGCGACAGGCCCCTGGACAAGGGCTTGAGTGGATGGGAGTC
ATCAACCCTAGTGGTGGTAGCACAAGCTACGCACAGAAGTTCCAGGGCAGAGTCACCATGACCAGGGACACGTCC
ACGAGCACAGTCTACATGGAGCTGAGCAGCCTGAGATCTGAGGACACGGCGGTGTACTACTGCGCCAGATTGATG
AGCGGATCGTCCGGAAGTTGGGGACAGGGTACATTGGTCACCGTCTCCTCA
BIIB-4-317_VH Amino Acid Sequence
(SEQ ID NO: 41)
QVQLVQSGAEVKKPGASVKVSCKASGYTFTGYYMHWVRQAPGQGLEWMGSINPNSGGTNYAQKFQGRVTMTRDTS
ISTAYMELSRLRSDDTAVYYCARDSSWKHDYWGQGTLVTVSS
BIIB-4-317_VH Nucleic Acid Sequence
(SEQ ID NO: 42)
CAGGTGCAGCTGGTGCAGTCTGGGGCTGAGGTGAAGAAGCCTGGGGCCTCAGTGAAGGTCTCCTGCAAGGCTTCT
GGATACACCTTCACCGGCTACTATATGCACTGGGTGCGACAGGCCCCTGGACAAGGGCTTGAGTGGATGGGAAGC
ATCAACCCTAACAGTGGTGGCACAAACTATGCACAGAAGTTTCAGGGCAGGGTCACCATGACCAGGGACACGTCC
ATCAGCACAGCCTACATGGAGCTGAGCAGGCTGAGATCTGACGACACGGCGGTGTACTACTGCGCCAGAGACAGC
AGCTGGAAACACGATTACTGGGGACAGGGTACATTGGTCACCGTCTCCTCA
BIIB-4-318_VH Amino Acid Sequence
(SEQ ID NO: 45)
QVQLQESGPGLVKPSETLSLTCAVSGYSISSGYYWGWIRQPPGKGLEWIGSIYHSGSTNYNPSLKSRVTISVDTS
KNQFSLKLSSVTAADTAVYYCARSPRWRSTYANWFNPWGQGTIVTVSS
BIIB-4-318_VH Nucleic Acid Sequence
(SEQ ID NO: 46)
CAGGTGCAGCTGCAGGAGTCGGGCCCAGGACTGGTGAAGCCTTCGGAGACCCTGTCCCTCACCTGCGCTGTCTCT
GGTTACTCCATCAGCAGTGGTTACTACTGGGGCTGGATCCGGCAGCCCCCAGGGAAGGGGCTGGAGTGGATTGGG
AGTATCTATCATAGTGGGAGCACCAACTACAACCCGTCCCTCAAGAGTCGAGTCACCATATCAGTAGACACGTCC
AAGAACCAGTTCTCCCTGAAGCTGAGTTCTGTGACCGCCGCAGACACGGCGGTGTACTACTGCGCCAGATCACCT
AGATGGAGATCCACCTACGCCAACTGGTTCAATCCCTGGGGACAGGGTACATTGGTCACCGTCTCCTCA
BIIB-4-319_VH Amino Acid Sequence
(SEQ ID NO: 49)
QVQLQESGPGLVKPSETLSLTCAVSGYSISSGYYWAWIRQPPGKGLEWIGSIYHSGSTYYNPSLKSRVTISVDTS
KNQFSLKLSSVTAADTAVYYCAREHSSSGQWNVWGQGTMVTVSS
BIIB-4-319_VH Nucleic Acid Sequence
(SEQ ID NO: 50)
CAGGTGCAGCTGCAGGAGTCGGGCCCAGGACTGGTGAAGCCTTCGGAGACCCTGTCCCTCACCTGCGCTGTCTCT
GGTTACTCCATCAGCAGTGGTTACTACTGGGCTTGGATCCGGCAGCCCCCAGGGAAGGGGCTGGAGTGGATTGGG
AGTATCTATCATAGTGGGAGCACCTACTACAACCCGTCCCTCAAGAGTCGAGTCACCATATCAGTAGACACGTCC
AAGAACCAGTTCTCCCTGAAGCTGAGTTCTGTGACCGCCGCAGACACGGCGGTGTACTACTGCGCCAGAGAGCAT
AGCAGCAGCGGCCAATGGAACGTATGGGGTCAGGGTACAATGGTCACCGTCTCCTCA

### Sequences of the Light Chain Variable Domain (VL) of the Identified Antibodies (CDRs are Underlined):

BIIB-4-147_VL Amino Acid Sequence
(SEQ ID NO: 7)
DIVMTQSPLSLPVTPGEPASISCRSSQSLLHSNGYNYLDWYLQKPGQSPQLLIYLGSNRASGVPDRFSGSGSGTD
FTLKISRVEAEDVGVYYCMQALRLPRTFGGGTKVEIK
BIIB-4-147_VL Nucleic Acid Sequence
(SEQ ID NO: 8)
GATATTGTGATGACTCAGTCTCCACTCTCCCTGCCCGTCACCCCTGGAGAGCCGGCCTCCATCTCCTGCAGGTCT
AGTCAGAGCCTCCTGCATAGTAATGGATACAACTATTTGGATTGGTACCTGCAGAAGCCAGGGCAGTCTCCACAG
CTCCTGATCTATTTGGGTTCTAATCGGGCCTCCGGGGTCCCTGACAGGTTCAGTGGCAGTGGATCAGGCACAGAT
TTTACACTGAAAATCAGCAGAGTGGAGGCTGAGGATGTTGGGGTTTATTACTGCATGCAGGCACTCCGCCTCCCT
AGGACTTTTGGCGGAGGGACCAAGGTTGAGATCAAA
BIIB-4-156_VL Amino Acid Sequence
(SEQ ID NO: 11)
EIVLTQSPATLSLSPGERATLSCRASQSVSSYLAWYQQKPGQAPRLLIYDASNRATGIPARFSGSGSGTDFTLTI
SSLEPEDFAVYYCQQRSALPRTFGGGTKVEIK
BIIB-4-156_VL Nucleic Acid Sequence
(SEQ ID NO: 12)
GAAATTGTGTTGACACAGTCTCCAGCCACCCTGTCTTTGTCTCCAGGGGAAAGAGCCACCCTCTCCTGCAGGGCC
AGTCAGAGTGTTAGCAGCTACTTAGCCTGGTACCAACAGAAACCTGGCCAGGCTCCCAGGCTCCTCATCTATGAT
GCATCCAACAGGGCCACTGGCATCCCAGCCAGGTTCAGTGGCAGTGGGTCTGGGACAGACTTCACTCTCACCATC
AGCAGCCTAGAGCCTGAAGATTTTGCAGTTTATTACTGTCAGCAGAGAAGTGCCCTCCCTAGGACTTTTGGCGGA
GGGACCAAGGTTGAGATCAAA
BIIB-4-174_VL Amino Acid Sequence
(SEQ ID NO: 15)
EIVLTQSPATLSLSPGERATLSCRASQSVSSYLAWYQQKPGQAPRLLIYDSSNRATGIPARFSGSGSGTDFTLTI
SSLEPEDFAVYYCQQRSHLPPTFGGGTKVEIK
BIIB-4-174_VL Nucleic Acid Sequence
(SEQ ID NO: 16)
GAAATTGTGTTGACACAGTCTCCAGCCACCCTGTCTTTGTCTCCAGGGGAAAGAGCCACCCTCTCCTGCAGGGCC
AGTCAGAGTGTTAGCAGCTACTTAGCCTGGTACCAACAGAAACCTGGCCAGGCTCCCAGGCTCCTCATCTATGAT
TCATCCAACAGGGCCACTGGCATCCCAGCCAGGTTCAGTGGCAGTGGGTCTGGGACAGACTTCACTCTCACCATC
AGCAGCCTAGAGCCTGAAGATTTTGCAGTTTATTACTGTCAGCAGAGAAGTCACCTGCCTCCTACTTTTGGCGGA
GGGACCAAGGTTGAGATCAAA
BIIB-4-175_VL Amino Acid Sequence
(SEQ ID NO: 19)
EIVMTQSPATLSVSPGERATLSCRASQSVSSNLAWYQQKPGQAPRLLIYGASTRATGIPARFSGSGSGTEFTLTI
SSLQSEDFAVYYCQQFNLYPYTFGGGTKVEIK
BIIB-4-175_VL Nucleic Acid Sequence
(SEQ ID NO: 20)
GAAATAGTGATGACGCAGTCTCCAGCCACCCTGTCTGTGTCTCCAGGGGAAAGAGCCACCCTCTCCTGCAGGGCC
AGTCAGAGTGTTAGCAGCAACTTAGCCTGGTACCAGCAGAAACCTGGCCAGGCTCCCAGGCTCCTCATCTATGGT
GCATCCACCAGGGCCACTGGTATCCCAGCCAGGTTCAGTGGCAGTGGGTCTGGGACAGAGTTCACTCTCACCATC
AGCAGCCTGCAGTCTGAAGATTTTGCAGTTTATTACTGTCAGCAGTTCAATCTCTACCCTTACACTTTTGGCGGA
GGGACCAAGGTTGAGATCAAA
BIIB-4-204_VL Amino Acid Sequence
(SEQ ID NO: 23)
EIVLTQSPATLSLSPGERATLSCRASQSVSSYLAWYQQKPGQAPRLLIYDASKRATGIPARFSGSGSGTDFTLTI
SSLEPEDFAVYYCQQDSFLPFTFGGGTKVEIK
BIIB-4-204_VL Nucleic Acid Sequence
(SEQ ID NO: 24)
GAAATTGTGTTGACACAGTCTCCAGCCACCCTGTCTTTGTCTCCAGGGGAAAGAGCCACCCTCTCCTGCAGGGCC
AGTCAGAGTGTTAGCAGCTACTTAGCCTGGTACCAACAGAAACCTGGCCAGGCTCCCAGGCTCCTCATCTATGAT
GCATCCAAAAGGGCCACTGGCATCCCAGCCAGGTTCAGTGGCAGTGGGTCTGGGACAGACTTCACTCTCACCATC
AGCAGCCTAGAGCCTGAAGATTTTGCAGTTTATTACTGTCAGCAGGACAGTTTCCTCCCTTTCACTTTTGGCGGA
GGGACCAAGGTTGAGATCAAA
BIIB-4-209_VL Amino Acid Sequence
(SEQ ID NO: 27)
EIVLTQSPATLSLSPGERATLSCRASQSVSSYLAWYQQKPGQAPRLLIYDASNRATGIPARFSGSGSGTDFTLTI
SSLEPEDFAVYYCQQAYNYPFTFGGGTKVEIK
BIIB-4-209_VL Nucleic Acid Sequence
(SEQ ID NO: 28)
GAAATTGTGTTGACACAGTCTCCAGCCACCCTGTCTTTGTCTCCAGGGGAAAGAGCCACCCTCTCCTGCAGGGCC
AGTCAGAGTGTTAGCAGCTACTTAGCCTGGTACCAACAGAAACCTGGCCAGGCTCCCAGGCTCCTCATCTATGAT
GCATCCAACAGGGCCACTGGCATCCCAGCCAGGTTCAGTGGCAGTGGGTCTGGGACAGACTTCACTCTCACCATC
AGCAGCCTAGAGCCTGAAGATTTTGCAGTTTATTACTGTCAGCAGGCCTATAATTACCCTTTCACTTTTGGCGGA
GGGACCAAGGTTGAGATCAAA
BIIB-4-224_VL Amino Acid Sequence
(SEQ ID NO: 31)
DIQLTQSPSSLSASVGDRVTITCRASQSISSFLNWYQQKPGKAPKLLIYAASSLQSGVPSRFSGSGSGTDFTLTI
SSLQPEDFATYYCQQSYVHPLTFGGGTKVEIK
BIIB-4-224_VL Nucleic Acid Sequence
(SEQ ID NO: 32)
GACATCCAGTTGACCCAGTCTCCATCCTCCCTGTCTGCATCTGTAGGAGACAGAGTCACCATCACTTGCCGGGCA
AGTCAGAGCATTAGCAGCTTTTTAAATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAGCAAAGCTACGTCCACCCTCTCACTTTTGGCGGA
GGGACCAAGGTTGAGATCAAA
BIIB-4-309_VL Amino Acid Sequence
(SEQ ID NO: 35)
DIVMTQSPLSLPVTPGEPASISCRSSQSLLHSNGYNYLDWYLQKPGQSPQLLIYLGSNRASGVPDRFSGSGSGTD
FTLKISRVEAEDVGVYYCMQARRSPLTFGGGTKVEIK
BIIB-4-309_VL Nucleic Acid Sequence
(SEQ ID NO: 36)
GATATTGTGATGACTCAGTCTCCACTCTCCCTGCCCGTCACCCCTGGAGAGCCGGCCTCCATCTCCTGCAGGTCT
AGTCAGAGCCTCCTGCATAGTAATGGATACAACTATTTGGATTGGTACCTGCAGAAGCCAGGGCAGTCTCCACAG
CTCCTGATCTATTTGGGTTCTAATCGGGCCTCCGGGGTCCCTGACAGGTTCAGTGGCAGTGGATCAGGCACAGAT
TTTACACTGAAAATCAGCAGAGTGGAGGCTGAGGATGTTGGGGTTTATTACTGCATGCAGGCAAGACGAAGCCCT
CTCACTTTTGGCGGAGGGACCAAGGTTGAGATCAAA
BIIB-4-311_VL Amino Acid Sequence
(SEQ ID NO: 39)
EIVLTQSPGTLSLSPGERATLSCRASQSVSSSYLAWYQQKPGQAPRLLIYGASSRATGIPDRFSGSGSGTDFTLT
ISRLEPEDFAVYYCQQYGGFPLTFGGGTKVEIK
BIIB-4-311_VL Nucleic Acid Sequence
(SEQ ID NO: 40)
GAAATTGTGTTGACGCAGTCTCCAGGCACCCTGTCTTTGTCTCCAGGGGAAAGAGCCACCCTCTCCTGCAGGGCC
AGTCAGAGTGTTAGCAGCAGCTACTTAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGGGTCTGGGACAGACTTCACTCTCACC
ATCAGCAGACTGGAGCCTGAAGATTTTGCAGTGTATTACTGTCAGCAGTACGGAGGCTTCCCTCTCACTTTTGGC
GGAGGGACCAAGGTTGAGATCAAA
BIIB-4-317_VL Amino Acid Sequence
(SEQ ID NO: 43)
EIVLTQSPATLSLSPGERATLSCRASQSVSSYLAWFQQKPGQAPRLLIYDASNRATGIPARFSGSGSGTDFTLTI
SSLEPEDFAVYYCQQYSFYPLTFGGGTKVEIK
BIIB-4-317_VL Nucleic Acid Sequence
(SEQ ID NO: 44)
GAAATTGTGTTGACACAGTCTCCAGCCACCCTGTCTTTGTCTCCAGGGGAAAGAGCCACCCTCTCCTGCAGGGCC
AGTCAGAGTGTTAGCAGCTACTTAGCCTGGTTCCAACAGAAACCTGGCCAGGCTCCCAGGCTCCTCATCTATGAT
GCATCCAACAGGGCCACTGGCATCCCAGCCAGGTTCAGTGGCAGTGGGTCTGGGACAGACTTCACTCTCACCATC
AGCAGCCTAGAGCCTGAAGATTTTGCAGTTTATTACTGTCAGCAGTACAGTTTCTACCCTCTCACTTTTGGCGGA
GGGACCAAGGTTGAGATCAAA
BIIB-4-318_VL Amino Acid Sequence
(SEQ ID NO: 47)
DIQLTQSPSSVSASVGDRVTITCRASQGISSWLAWYQQKPGKAPKLLIYGASSLQSGVPSRFSGSGSGTDFTLTI
SSLQPEDFATYYCQQAAPFPLTFGGGTKVEIK
BIIB-4-318_VL Nucleic Acid Sequence
(SEQ ID NO: 48)
GACATCCAGTTGACCCAGTCTCCATCTTCCGTGTCTGCATCTGTAGGAGACAGAGTCACCATCACTTGTCGGGCG
AGTCAGGGTATTAGCAGCTGGTTAGCCTGGTATCAGCAGAAACCAGGGAAAGCCCCTAAGCTCCTGATCTATGGT
GCATCCAGTTTGCAAAGTGGGGTCCCATCAAGGTTCAGCGGCAGTGGATCTGGGACAGATTTCACTCTCACCATC
AGCAGCCTGCAGCCTGAAGATTTTGCAACTTACTACTGTCAGCAGGCAGCCCCCTTCCCTCTCACTTTTGGCGGA
GGGACCAAGGTTGAGATCAAA
BIIB-4-319_VL Amino Acid Sequence
(SEQ ID NO: 51)
EIVLTQSPATLSLSPGERATLSCRASQSVSSYLAWYQQKPGQAPRLLIYDASNRATGIPARFSGSGSGTDFTLTI
SSLEPEDFAVYYCQQRSFYFTFGGGTKVEIK
BIIB-4-319_VL Nucleic Acid Sequence
(SEQ ID NO: 52)
GAAATTGTGTTGACACAGTCTCCAGCCACCCTGTCTTTGTCTCCAGGGGAAAGAGCCACCCTCTCCTGCAGGGCC
AGTCAGAGTGTTAGCAGCTACTTAGCCTGGTACCAACAGAAACCTGGCCAGGCTCCCAGGCTCCTCATCTATGAT
GCATCCAACAGGGCCACTGGCATCCCAGCCAGGTTCAGTGGCAGTGGGTCTGGGACAGACTTCACTCTCACCATC
AGCAGCCTAGAGCCTGAAGATTTTGCAGTTTATTACTGTCAGCAGAGAAGTTTTTACTTCACTTTTGGCGGAGGG
ACCAAGGTTGAGATCAAA

### Example 2: Determination of Binding Kinetics and Epitope Binning

Antibodies were initially screened to identify clones that bound preferentially to GPIIb/IIIa in the extended conformation, with diminished binding for the inactive or bent conformation of GPIIb/IIIa. 564 antibodies were screened for binding to target antigen using Bio-Layer Interferometry (BLI). BLI was performed on the OctetRed94 instrument manufactured by ForteBio according to standard procedures. The top 188 antibodies were classified based on binding kinetics and selectivity for active recombinant human GPIIb/IIIa protein (preference for active target (SEQ ID NOS:1 and 3) vs. no preference for active target (SEQ ID NOS:2 and 4)) in a monovalent assay format.

Examples of observed binding kinetics for non-selective GPIIb/IIIa antibodies are shown in FIGS. 6A-F. Examples of antibodies that displayed preference for the active conformation of GPIIb/IIIa are depicted in FIGS. 7A-D. BIIB-4-156, BIIB-4-224, BIIB-4-309, and BIIB-4-311 were identified as antibodies that demonstrated preference for active GPIIb/IIIa, with weaker binding observed for bent/inactive GPIIb/IIIa (FIG. 8). Surface Plasmon Resonance (SPR) confirmed the differences in binding kinetics observed for BIIB-4-156, BIIB-4-224, BIIB-4-309, and BIIB-4-311 to active vs. inactive GPIIb/IIIa, where monovalent affinities were also compared to those of the BLI measurements (FIG. 9 and FIG. 10).

A selection of antibodies from the two campaigns was then subjected to cross-blocking/epitope binning on the OctetRed94 to determine common epitope groupings. The target antigen (SEQ ID NOS:1 and 3) was collected on the Octet sensor and then incubated in the presence of the first antibody. Next, the antibody:antigen complex was incubated in the presence of a second antibody. If the binding signal was observed to increase upon incubation with the second antibody, it was concluded that the two antibodies do not share a common epitope group. Examples of antibodies in the epitope binning assay and their assigned cross-blocking bin are highlighted in FIG. 17.

### Example 3: Screening for Biophysical Behavior

188 antibodies were screened by self-interaction nanoparticle spectroscopy to determine which clones had inferior biophysical properties according to the methods described within Liu et al. 2014 (Liu et al., MAbs, 6(2): 483-92 (2014)). Following incubation on the surface of nanoparticles, absorbance across a spectrum of wavelengths were measured, with higher wavelengths of maximum absorbance indicative of reduced inter-particle distances resulting from antibody self-association. This experiment was useful in identifying antibodies displaying a propensity to self-interact (FIG. 11).

### Example 4: Platelet Binding, Platelet Activation, and Fibrinogen Competition

The antibodies were then subjected to a series of analyses on human platelets to: (i) confirm target binding on platelets, (ii) confirm binding preferences between active/extended and inactive/bent GPIIb/IIIa, for those that displayed selectivity in BLI experiments, by analyzing binding to active or resting platelets, (iii) to determine if antibody binding is capable of activating platelets, and (iv) to determine if the antibody binding is disruptive to fibrinogen association with GPIIb/IIIa, which is critical for platelet aggregation and clot formation. These analyses helped identify antibodies that can either associate with all conformations of GPIIb/IIIa or selectively bind to active/extended GPIIb/IIIa, that do not activate platelets, and that do not prohibit fibrinogen binding to GPIIb/IIIa.

Selected antibodies from the analyses described in the Examples above were tested for binding to active or resting gel-purified human platelets by flow cytometry. Platelet activation was achieved by the addition of 1 μM ADP and 5 mg/ml thrombin receptor activating peptide-SFLLRN (SEQ ID NO: 254) (Anaspec Inc. Cat. #2419). Antibody binding in the format of a Fab was detected by flow cytometry techniques known in the art. Examples of antibodies that display preference for active versus resting platelets are shown in FIGS. 12 A-C. FIG. 12D summarizes the selectivity of the 12 disclosed antibodies for active versus resting platelets. These results correlate with the affinity measurements in BLI and SPR conducted with purified recombinant GPIIb/IIIa.

To differentiate the conformation-selective antibodies from previously identified antibodies for active-specific integrin conformations, the propensity for antibody:platelet association to stimulate platelet activation was assessed. Resting gel-purified human platelets were incubated with BIIB-4-156, BIIB-4-224, BIIB-4-309, or BIIB-4-311 and subsequently P-selectin surface expression and PAC-1 binding to platelets were assessed by flow cytometry. P-selectin (CD62p) is expressed on the surface of human platelets upon activation. PAC-1 is an ligand-mimetic IgM that recognizes active/extended GPIIb/IIIa on the surface of activated platelets. Binding of P-selectin antibodies and PAC-1 to platelets pre-incubated with conformation-selective Fab was compared to platelets activated by incubation with 1 μM ADP and 5 mg/ml SFLLRN (SEQ ID NO: 254). None of the four conformation-selective antibodies were capable of stimulating platelet activation (FIG. 13).

Fibrinogen is the natural ligand of the integrin GPIIb/IIIa on the surface of platelets and this binding is critical for platelet aggregation and downstream clotting events. Therefore, antibodies were screened for the ability to prohibit binding of fibrinogen to GPIIb/IIIa on platelets. Activated gel-purified platelets were prepared by incubation with 1 μM ADP and 5 mg/ml SFLLRN (SEQ ID NO: 254) and incubated with GPIIb/IIIa antibodies. The binding of fluorescently-labeled fibrinogen (Life Technologies Cat. No. F-13191) was assessed by flow cytometry. An example of this analysis is shown in FIG. 14A of BIIB-4-156 (a conformation-selective antibody that does not activate platelets), which does not disrupt fibrinogen association when compared to a control antibody (Santa Cruz Cat. No. SC-7310). Of the tested antibodies, BIIB-4-174 and BIIB-4-175 were found to strongly prohibit fibrinogen association with platelets (FIG. 14B). FIG. 15 provides a list of the antibodies that interfere and those that did not interfere with the binding of fibrinogen to GPIIb/IIIa.

### Example 5: Platelet-Targeted Chimeric Proteins

Antibodies against GPIIb/IIIa (SEQ ID NOs.: 1 and 3) were used to target recombinant FVIIa (rFVIIa) clotting factor to the surface of human platelets. The disclosed antibodies were generated as fusion proteins in HEK293 cells by recombinantly fusing the C-terminus of the FVIIa heavy chain via a linker with the N-terminus of the Fab of the VL of BIIB_4_147 antibody by molecular biology techniques known in the art. The nucleic acid sequence encoding this chimeric BIIB_4_147 FVIIa polypeptide is provided below (the sequence encoding the linker is underlined):

(SEQ ID NO: 124)
ATGGTCTCCCAGGCCCTCAGGCTCCTCTGCCTTCTGCTTGGGCTTCAGGGC
TGCCTGGCTGCAGTCTTCGTAACCCAGGAGGAAGCCCACGGCGTCCTGCAC
CGGCGCCGGCGCGCCAACGCGTTCCTGGAGGAGCTGCGGCCGGGCTCCCTG
GAGAGGGAGTGCAAGGAGGAGCAGTGCTCCTTCGAGGAGGCCCGGGAGATC
TTCAAGGACGCGGAGAGGACGAAGCTGTTCTGGATTTCTTACAGTGATGGG
GACCAGTGTGCCTCAAGTCCATGCCAGAATGGGGGCTCCTGCAAGGACCAG
CTCCAGTCCTATATCTGCTTCTGCCTCCCTGCCTTCGAGGGCCGGAACTGT
GAGACGCACAAGGATGACCAGCTGATCTGTGTGAACGAGAACGGCGGCTGT
GAGCAGTACTGCAGTGACCACACGGGCACCAAGCGCTCCTGTCGGTGCCAC
GAGGGGTACTCTCTGCTGGCAGACGGGGTGTCCTGCACACCCACAGTTGAA
TATCCATGTGGAAAAATACCTATTCTAGAAAAAAGAAATGCCAGCAAACCC
CAAGGCCGAATTGTGGGGGGCAAGGTGTGCCCCAAAGGGGAGTGTCCATGG
CAGGTCCTGTTGTTGGTGAATGGAGCTCAGTTGTGTGGGGGGACCCTGATC
AACACCATCTGGGTGGTCTCCGCGGCCCACTGTTTCGACAAAATCAAGAAC
TGGAGGAACCTGATCGCGGTGCTGGGCGAGCACGACCTCAGCGAGCACGAC
GGGGATGAGCAGAGCCGGCGGGTGGCGCAGGTCATCATCCCCAGCACGTAC
GTCCCGGGCACCACCAACCACGACATCGCGCTGCTCCGCCTGCACCAGCCC
GTGGTCCTCACTGACCATGTGGTGCCCCTCTGCCTGCCCGAACGGACGTTC
TCTGAGAGGACGCTGGCCTTCGTGCGCTTCTCATTGGTCAGCGGCTGGGGC
CAGCTGCTGGACCGTGGCGCCACGGCCCTGGAGCTCATGGTCCTCAACGTG
CCCCGGCTGATGACCCAGGACTGCCTGCAGCAGTCACGGAAGGTGGGAGAC
TCCCCAAATATCACGGAGTACATGTTCTGTGCCGGCTACTCGGATGGCAGC
AAGGACTCCTGCAAGGGGGACAGTGGAGGCCCACATGCCACCCACTACCGG
GGCACGTGGTACCTGACGGGCATCGTCAGCTGGGGCCAGGGCTGCGCAACC
GTGGGCCACTTTGGGGTGTACACCAGGGTCTCCCAGTACATCGAGTGGCTG
CAAAAGCTCATGCGCTCAGAGCCACGCCCAGGAGTCCTCCTGCGAGCCCCA
TTTCCCGGTGGCGGTGGCTCCGGCGGAGGTGGGTCCGGTGGCGGCGGATCA
GGTGGGGGTGGATCAGGCGGTGGAGGTTCCGGTGGCGGGGGATCCGATATT
GTGATGACTCAGTCTCCACTCTCCCTGCCCGTCACCCCTGGAGAGCCGGCC
TCCATCTCCTGCAGGTCTAGTCAGAGCCTCCTGCATAGTAATGGATACAAC
TATTTGGATTGGTACCTGCAGAAGCCAGGGCAGTCTCCACAGCTCCTGATC
TATTTGGGTTCTAATCGGGCCTCCGGGGTCCCTGACAGGTTCAGTGGCAGT
GGATCAGGCACAGATTTTACACTGAAAATCAGCAGAGTGGAGGCTGAGGAT
GTTGGGGTTTATTACTGCATGCAGGCACTCCGCCTCCCTAGGACTTTTGGC
GGAGGGACCAAGGTTGAGATCAAACGGACCGTGGCTGCACCATCTGTCTTC
ATCTTCCCGCCATCTGATGAGCAGTTGAAATCTGGAACTGCCTCTGTTGTG
TGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGAC
AGCAAGGACAGCACCTACAGCCTCAGCAGCACCCTGACGCTGAGCAAAGCA
GACTACGAGAAACACAAAGTCTACGCCTGCGAAGTCACCCATCAGGGCCTG
AGCTCGCCCGTCACAAAGAGCTTCAACAGGGGAGAGTGTTAG

The amino acid sequence of this BIIB_4_147VL/CL FVIIA chimeric polypeptide is provided below (heavy chain of FVII boldened; light chain of FVII italicized; linker underlined):

(SEQ ID NO: 125)
M V S Q A L R L L C L L L G L Q
G C L A A V F V T Q E E A H G
V L H R R R R A N A F L E E L
R P G S L E R E C K E E Q C S
F E E A R E I F K D A E R T K
L F W I S Y S D G D Q C A S S
P C Q N G G S C K D Q L Q S Y
I C F C L P A F E G R N C E T
H K D D Q L I C V N E N G G C
E Q Y C S D H T G T K R S C R
C H E G Y S L L A D G V S C T
P T V E Y P C G K I P I L E K
R N A S K P Q G R I V G G K V
C P K G E C P W Q V L L L V N
G A Q L C G G T L I N T I W V
V S A A H C F D K I K N W R N
L I A V L G E H D L S E H D G
D E Q S R R V A Q V I I P S T
Y V P G T T N H D I A L L R L
H Q P V V L T D H V V P L C L
P E R T F S E R T L A F V R F
S L V S G W G Q L L D R G A T
A L E L M V L N V P R L M T Q
D C L Q Q S R K V G D S P N I
T E Y M F C A G Y S D G S K D
S C K G D S G G P H A T H Y R
G T W Y L T G I V S W G Q G C
A T V G H F G V Y T R V S Q Y
I E W L Q K L M R S E P R P G
V L L R A P F P G G G G S G G
G G S G G G G S G G G G S G G
G G S G G G G S D I V M T Q S
P L S L P V T P G E P A S I S
C R S S Q S L L H S N G Y N Y
L D W Y L Q K P G Q S P Q L L
I Y L G S N R A S G V P D R F
S G S G S G T D F T L K I S R
V E A E D V G V Y Y C M Q A L
R L P R T F G G G T K V E I K
R T V A A P S V F I F P P S D
E Q L K S G T A S V V C L L N
N F Y P R E A K V Q W K V D N
A L Q S G N S Q E S V T E Q D
S K D S T Y S L S S T L T L S
K A D Y E K H K V Y A C E V T
H Q G L S S P V T K S F N R G
E C

The nucleic acid sequence encoding the BIIB_4_147 VH/CH1 polypeptide that associates with the Fab light chain of the chimeric polypeptide described above is provided below (the nucleic acid sequence encoding the signal sequence is omitted):

(SEQ ID NO: 126)
CAGGTTCAGCTGGTGCAGTCTGGAGCTGAGGTGAAGAAGCCTGGGGCCTCA
GTGAAGGTCTCCTGCAAGGCTTCTGGTTACACCTTTACCAGCTATGGTATC
AGCTGGGTGCGACAGGCCCCTGGACAAGGGCTTGAGTGGATGGGATGGATC
AGCGCTTACAATGGTAACACAAACTATGCACAGAAGCTCCAGGGCAGAGTC
ACCATGACCACAGACACATCCACGAGCACAGCCTACATGGAGCTGAGGAGC
CTGAGATCTGACGACACGGCGGTGTACTACTGCGCCAGAGACTTGGAATAC
TACGACAGCAGCGGATACGCCTATGGCTACTTCGACCTATGGGGGAGAGGT
ACCTTGGTCACCGTCTCCTCAGCTAGCACGAAGGGGCCCAGCGTGTTCCCC
CTGGCCCCCAGCAGCAAGAGCACCAGCGGCGGCACCGCCGCCCTGGGCTGC
CTGGTGAAGGACTACTTCCCCGAACCGGTGACGGTGTCGTGGAACTCAGGC
GCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACC
CAGACCTACATCTGCAACGTGAATCACAAGCCCAGCAACACCAAGGTGGAC
AAGAAAGTTGAGCCCAAATCTTGTTGA

The amino acid sequence of the BIIB_4_147 VH/CH1 polypeptide is provided below:

(SEQ ID NO: 127)
Q V Q L V Q S G A E V K K P G A S V K V S C K A S G
Y T F T S Y G I S W V R Q A P G Q G L E W M G W I S
A Y N G N T N Y A Q K L Q G R V T M T T D T S T S T
A Y M E L R S L R S D D T A V Y Y C A R D L E Y Y D
S S G Y A Y G Y F D L W G R G T L V T V S S A S T K
G P S V F P L A P S S K S T S G G T A A L G C L V K
D Y F P E P V T V S W N S G A L T S G V H T F P A V
L Q S S G L Y S L S S V V T V P S S S L G T Q T Y I
C N V N H K P S N T K V D K K V E P K S C

The procoagulant activity of the platelet-targeting chimeric proteins was assessed by rotational thromboelastometry (ROTEM) in blood from human hemophilia A donors. BIIB-4-147 fused with FVIIa displayed a 12-fold increase in clotting time when compared to the addition of rFVIIa alone (FIG. 16).

### Example 6: Integrin Specificity

The antibodies described herein were selected to target the integrin GPIIb/IIIa (SEQ ID NOs:1 and 3). The only known productive assembly of alpha and beta subunits as functional integrin heterodimers for the alpha IIb subunit is with the beta III subunit. However, the beta III subunit is capable of functional pairing with the related alpha V subunit (Hynes R O, Cell, 110(6):673-87 (2002)). The amino sequence of the human alpha V protein ectodomain is shown below:

(SEQ ID NO: 245)
MAFPPRRRLRLGPRGLPLLLSGLLLPLCRAFNLDVDSPAEYSGPEGSYFGF
AVDFFVPSASSRMFLLVGAPKANTTQPGIVEGGQVLKCDWSSTRRCQPIEF
DATGNRDYAKDDPLEFKSHQWFGASVRSKQDKILACAPLYHWRTEMKQERE
PVGTCFLQDGTKTVEYAPCRSQDIDADGQGFCQGGFSIDFTKADRVLLGGP
GSFYWQGQLISDQVAEIVSKYDPNVYSIKYNNQLATRTAQAIFDDSYLGYS
VAVGDFNGDGIDDFVSGVPRAARTLGMVYIYDGKNMSSLYNFTGEQMAAYF
GFSVAATDINGDDYADVFIGAPLFMDRGSDGKLQEVGQVSVSLQRASGDFQ
TTKLNGFEVFARFGSAIAPLGDLDQDGFNDIAIAAPYGGEDKKGIVYIFNG
RSTGLNAVPSQILEGQWAARSMPPSFGYSMKGATDIDKNGYPDLIVGAFGV
DRAILYRARPVITVNAGLEVYPSILNQDNKTCSLPGTALKVSCFNVRFCLK
ADGKGVLPRKLNFQVELLLDKLKQKGAIRRALFLYSRSPSHSKNMTISRGG
LMQCEELIAYLRDESEFRDKLTPITIFMEYRLDYRTAADTTGLQPILNQFT
PANISRQAHILLDCGEDNVCKPKLEVSVDSDQKKIYIGDDNPLTLIVKAQN
QGEGAYEAELIVSIPLQADFIGVVRNNEALARLSCAFKTENQTRQVVCDLG
NPMKAGTQLLAGLRFSVHQQSEMDTSVKFDLQIQSSNLFDKVSPVVSHKVD
LAVLAAVEIRGVSSPDHVFLPIPNWEHKENPETEEDVGPVVQHIYELRNNG
PSSFSKAMLHLQWPYKYNNNTLLYILHYDIDGPMNCTSDMEINPLRIKISS
LQTTEKNDTVAGQGERDHLITKRDLALSEGDIHTLGCGVAQCLKIVCQVGR
LDRGKSAILYVKSLLWTETFMNKENQNHSYSLKSSASFNVIEFPYKNLPIE
DITNSTLVTTNVTWGIQPAPMPVP

To determine the integrin specificity of the antibodies discovered in our selections, the association of antibody with purified recombinant ectodomain of GPIIb/IIIa (SEQ ID NOs:1 and 3) and integrin alpha v beta III (SEQ ID NOs:245 and 3) was assessed using BLI in a monovalent assay format. BLI was performed on the OctetRed94 instrument, manufactured by ForteBio, according to standard procedures. The present disclosure describes the integrin binding specificity for antibodies BIIB-4-147 (SEQ ID NOs:5 and 7), BIIB-4-156 (SEQ ID NOs:9 and 11), BIIB-4-174 (SEQ ID NOs:13 and 15), BIIB-4-175 (SEQ ID NOs:17 and 19), BIIB-4-204 (SEQ ID NOs:21 and 23), BIIB-4-209 (SEQ ID NOs:25 and 27), BIIB-4-224 (SEQ ID NOs:29 and 31), BIIB-4-309 (SEQ ID NOs:33 and 35), BIIB-4-311 (SEQ ID NOs:37 and 39), BIIB-4-317 (SEQ ID NOs:41 and 43), BIIB-4-318 (SEQ ID NOs:45 and 47), and BIIB-4-319 (SEQ ID NOs:49 and 51). Examples of individual BLI binding profiles are disclosed herein (FIG. 20 A-D). A table listing the integrin binding specificity of the twelve disclosed antibodies, as determined by BLI in the monovalent format, is depicted in FIG. 20E. These studies indicate that BIIB-4-147, BIIB-4-174, BIIB-4-175, BIIB-4-224, BIIB-4-309, BIIB-4-311, BIIB-4-318 are highly specific for GPIIb/IIIa.

### Example 7: Generation of BIIB-4-309-FVIIa

To determine if the specificity of the Fabs described above for the active conformation of GPIIb/IIIa was maintained when fused to FVIIa, a Fab BIIB_4_309-FVIIa fusion was generated.

In this fusion, shown below, the N-terminus of the heavy chain of the Fab BIIB_4_309 was recombinantly fused to the C-terminus of the heavy chain FVIIa-XTEN via a linker (Gly₄Ser)₆ (SEQ ID NO:172).

```
(SEQ ID NO: 246)
    1 ANAFLEELRP GSLERECKEE QCSFEEAREI FKDAERTKLF
        WISYSDGDQC
  51 ASSPCQNGGS CKDQLQSYIC FCLPAFEGRN CETHKDDQLI
        CVNENGGCEQ
101 YCSDHTGTKR SCRCHEGYSL LADGVSCTPT VEYPCGKIPI
        LEKRNASKPQ
151 GRTVGGKVCP KGECPWQVLL LVNGAQLCGG TLINTIWVVS
        AAHCFDKIKN
201 WRNLIAVLGE HDLSEHDGDE QSRRVAQVII PSTYVPGTTN
        HDIALLRLHQ
251 PVVLTDHVVP LCLPERTFSE RTLAFVRFSL VSGWGQLLDR
        GATAIELMVL
301 NVPRLMTQDC LQQSRKVGDS PNITEYMFCA GYSDGSKDSC
        KGDSGGPHAT
351 HYRGTWYLTG IVSWGQGCAT VGHFGVYTRV SQYIEWLQKL
        MRSEPRPGVL
401 LRAPFPGSPG TSESATPESG PGSEPATSGS ETPGTSESAT
        PESGPGSEPA
451 TSGSETPGTS ESATPESGPG TSTEPSEGSA PGSPAGSPTS
        TEEGTSESAT
501 PESGPGSEPA TSGSETPGTS ESATPESGPG SPAGSPTSTE
        EGSPAGSPTS
551 TEEGTSTEPS EGSAPGTSES ATPESGPGTS ESATPESGPG
        TSESATPESG
601 PGSEPATSGS ETPGSEPATS GSETPGSPAG SPTSTEEGTS
        TEPSEGSAPG
651 TSTEPSEGSA PGSEPATSGS ETPGTSESAT PESGPGTSTE
        PSEGSAPGGG
701 GSGGGGSGGG GSGGGGSGGG GSGGGGSQVQ LVQSGAEVKK
        PGASVKVSCK
751 ASGYTFTSYG ISWVRQAPGQ GLEWMGWISA YNGNTNYAQK
        LQGRVTMTTD
801 TSTSTAYMEL RSLRSDDTAV YYCARDGLGS SPWSAFDIWG
        QGTMVTVSSA
851 STKGPSVFPL APSSKSTSGG TAALGCLVED YFPEPVTVSW
        NSGALTSGVH
901 TFPAVLQSSG LYSLSSVVTV PSSSLGTQTY ICNVNHKPSN
        TKVDKKVEPK
951 SC*
```

The amino acid sequence of FVIIa (bold) is followed by a linker GSPGTSESATPESGPGSEPATSGSETP (SEQ ID NO:197) followed by an XTEN sequence, AE288 (SEQ ID NO:239) (underlined), which is followed by the linker (Gly₄Ser)₆ (SEQ ID NO:172) (double underlined), which is followed by the Fab BIIB_4_309 heavy chain VH/CH1 (bold, underlined). The light chain of FVIIa associates with the heavy chain FVIIa-XTEN while the heavy chain component of the Fab associates with the light chain component of the Fab. The amino acid sequence of the Fab BIIB_4_309 light chain (VL/CL) is shown below:

(SEQ ID NO: 247)

| | |
|---|---|
| 1 | DIVMTQSPLS LPVTPGEPAS ISCRSSQSLL HSNGYNYLDW |
| | YLQKPGQSPQ |
| 51 | LLIYLGSNRA SGVPDRFSGS GSGTDFTLKI SRVEAEDVGV |
| | YYCMQARRSP |
| 101 | LTFGGGTKVE IKRTVAAPSV FIFPPSDEQL KSGTASVVCL |
| | LNNFYPREAK |
| 151 | VQWKVDNALQ SGNSQESVTE QDSKDSTYSL SSTLTLSKAD |
| | YEKHKVYACE |
| 201 | VTHQGLSSPV TKSFNRGEC* |

The DNA encoding these proteins was generated using molecular biology methods known in the art. The constructs were transiently expressed in HEK 293 cell and purified by standard methods.

### Example 8: Binding of Fab BIIB_4_309-FVIIa to the Active Conformation of GPIIb/IIIa

To determine the GPIIb/IIIa binding specificity of BIIB_4_309-FVIIa, binding assays were performed using surface plasmon resonance (SPR) technology. For this purpose biotinylated human GPIIb/IIIa ectodomain in the active and inactive conformations were generated as described above in Example 1. The GPIIb/IIIa ectodomain protein was immobilized on an SPR chip coated with streptavidin (GE Healthcare). Next, the association and dissociation of Fab binding to GPIIb/IIIa at sequentially increasing concentrations of the Fab were measured following methods known in the art.

As shown in FIG. 21, the SPR experiment demonstrates binding specificity of BIIB_4_309-FVIIa for the active conformation of GPIIb/IIIa.

These results indicate that the specificity of Fab BIIB_4_309 for the active conformation of GPIIb/IIIa is maintained when fused to FVIIa.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody or antigen-binding fragment thereof that specifically binds to Glycoprotein IIb/IIIa (GPIIb/IIIa), wherein the antibody or antigen-binding fragment thereof:
preferentially binds to GPIIb/IIIa on activated platelets compared to resting platelets; and
(ii) does not activate platelets.

2. The antibody or antigen-binding fragment thereof of claim 1, wherein the antibody or antigen-binding fragment thereof does not inhibit the association of fibrinogen with GPIIb/IIIa.

3. The antibody or antigen-binding fragment thereof of claim 1, wherein the antibody or antigen-binding fragment thereof comprises:
(i) the complementarity determining regions (CDRs) of the heavy chain variable domain (VH) amino acid sequence set forth in SEQ ID NOs. 9, 29, 33, or 37;
(ii) an amino acid sequence that is at least 85% identical to the VH amino acid sequence set forth in SEQ ID NOs. 9, 29, 33, or 37;
(iii) the complementarity determining regions of the light chain variable domain (VL) amino acid sequence set forth in SEQ ID NOs. 11, 31, 35, or 39; or
(iv) an amino acid sequence that is at least 85% identical to the VL amino acid sequence set forth in SEQ ID NOs. 11, 31, 35, or 39.

4.-8. (canceled)

9. An antibody or antigen-binding fragment thereof that specifically binds to Glycoprotein IIb/IIIa (GPIIb/IIIa), wherein the antibody or antigen-binding fragment thereof:
(i) binds to GPIIb/IIIa on both activated platelets and resting platelets; and
(ii) does not activate platelets.

10. The antibody or antigen-binding fragment thereof of claim 9, wherein the antibody or antigen-binding fragment thereof comprises:
(i) the complementarity determining regions of VH amino acid sequence set forth in SEQ ID NOs. 5, 13, 17, 21, 25, 41, 45, or 49;
(ii) a VH amino acid sequence that is at least 85% identical to the amino acid sequence set forth in SEQ ID NOs. 5, 13, 17, 21, 25, 41, 45, or 49;
(iii) the complementarity determining regions of the VL amino acid sequence set forth in SEQ ID NOs. 7, 15, 19, 23, 27, 43, 47, or 51; or
(iv) a VL amino acid sequence that is at least 85% identical to the amino acid sequence set forth in SEQ ID NOs. 7, 15, 19, 23, 27, 43, 47, or 51.

11.-22. (canceled)

23. An antibody or antigen-binding fragment thereof that specifically binds to Glycoprotein IIb/IIIa (GPIIb/IIIa), wherein the antibody or antigen-binding fragment thereof:
(a) specifically binds to GPIIb/IIIa at the same epitope as an antibody comprising the heavy chain variable domain (VH) and the light chain variable domain (VL) amino acid sequences set forth in:
(i) SEQ ID NOs. 5 and 7;
(ii) SEQ ID NOs. 9 and 11;
(iii) SEQ ID NOs. 13 and 15;
(iv) SEQ ID NOs. 17 and 19;
(v) SEQ ID NOs. 21 and 23;
(vi) SEQ ID NOs. 25 and 27;
(vii) SEQ ID NOs. 29 and 31;
(viii) SEQ ID NOs. 33 and 35;
(ix) SEQ ID NOs. 37 and 39;
(x) SEQ ID NOs. 41 and 43;
(xi) SEQ ID NOs. 45 and 47; or
(xii) SEQ ID NOs. 49 and 51; or
(b) competitively inhibits GPIIb/IIIa binding by an antibody comprising the heavy chain variable domain (VH) and the light chain variable domain (VL) amino acid sequences set forth in:
(i) SEQ ID NOs. 5 and 7;
(ii) SEQ ID NOs. 9 and 11;
(iii) SEQ ID NOs. 13 and 15;
(iv) SEQ ID NOs. 17 and 19;
(v) SEQ ID NOs. 21 and 23;
(vi) SEQ ID NOs. 25 and 27;
(vii) SEQ ID NOs. 29 and 31;
(viii) SEQ ID NOs. 33 and 35;
(ix) SEQ ID NOs. 37 and 39;
(x) SEQ ID NOs. 41 and 43;
(xi) SEQ ID NOs. 45 and 47; or
(xii) SEQ ID NOs. 49 and 51.

24.-25. (canceled)

26. An antibody or antigen-binding fragment thereof that specifically binds to Glycoprotein IIb/IIIa (GPIIb/IIIa), comprising a VH-CDR1, VH-CDR2, VH-CDR3, VL-CDR1, VL-CDR2, and VL-CDR3, wherein
(i) the VH-CDR1 sequence comprises YTFTSYGIS (SEQ ID NO:53), the VH-CDR2 sequence comprises WISAYNGNTNYAQKLQG (SEQ ID NO:54), the VH-CDR3 sequence comprises ARDLEYYDSSGYAYGYFDL (SEQ ID NO:55), the VL-CDR1 sequence comprises RSSQSLLHSNGYNYLD (SEQ ID NO:83), the VL-CDR2 sequence comprises LGSNRAS (SEQ ID NO:84), and the VL-CDR3 sequence comprises MQALRLPRT (SEQ ID NO:85);
(ii) the VH-CDR1 sequence comprises GTFSSYAIS (SEQ ID NO:56), the VH-CDR2 sequence comprises GIIPIFGTANYAQKFQG (SEQ ID NO:57), the VH-CDR3 sequence comprises ARDTGYYGASLYFDY (SEQ ID NO:58), the VL-CDR1 sequence comprises RASQSVSSYLA (SEQ ID NO:86), the VL-CDR2 sequence comprises DASNRAT (SEQ ID NO:87), and the VL-CDR3 sequence comprises QQRSALPRT (SEQ ID NO:88);
(iii) the VH-CDR1 sequence comprises GTFSSYAIS (SEQ ID NO:56), the VH-CDR2 sequence comprises GIIPIFGTANYAQKFQG (SEQ ID NO:57), the VH-CDR3 sequence comprises ARGPPSAYGDYVWDI (SEQ ID NO:59), the VL-CDR1 sequence comprises RASQSVSSYLA (SEQ ID NO:86), the VL-CDR2 sequence comprises DSSNRAT (SEQ ID NO:89), and the VL-CDR3 sequence comprises QQRSHLPPT (SEQ ID NO:90);
(iv) the VH-CDR1 sequence comprises FTFSDHHMD (SEQ ID NO:60), the VH-CDR2 sequence comprises RTRNKANSYTTEYAASVKG (SEQ ID NO:61), the VH-CDR3 sequence comprises ARGPPYYADLGMGV (SEQ ID NO:62), the VL-CDR1 sequence comprises RASQSVSSNLA (SEQ ID NO:91), the VL-CDR2 sequence comprises GASTRAT (SEQ ID NO:92), and the VL-CDR3 sequence comprises QQFNLYPYT (SEQ ID NO:93);
(v) the VH-CDR1 sequence comprises YTFTSYSMH (SEQ ID NO:63), the VH-CDR2 sequence comprises IINPSGGSTSYAQKFQG (SEQ ID NO:64), the VH-CDR3 sequence comprises ARSYDIGYFDL (SEQ ID NO:65), the VL-CDR1 sequence comprises RASQSVSSYLA (SEQ ID NO:86), the VL-CDR2sequence comprises DASKRAT (SEQ ID NO:94), and the VL-CDR3 sequence comprises QQDSFLPFT (SEQ ID NO:95);
(vi) the VH-CDR1 sequence comprises YTFTSYGIS (SEQ ID NO:53), the VH-CDR2 sequence comprises WISAYNGNTNYAQKLQG (SEQ ID NO:54), the VH-CDR3 sequence comprises ARGRPYDHYFDY (SEQ ID NO:66), the VL-CDR1 sequence comprises RASQSVSSYLA (SEQ ID NO:86), the VL-CDR2sequence comprises DASNRAT (SEQ ID NO:87), and the VL-CDR3 sequence comprises QQAYNYPFT (SEQ ID NO:96);
(vii) the VH-CDR1 sequence comprises GSISSSSYYWG (SEQ ID NO:67), the VH-CDR2 sequence comprises SIYYSGSTYYNPSLKS (SEQ ID NO:68), the VH-CDR3 sequence comprises ARDFYSSVYGMDV (SEQ ID NO:69), the VL-CDR1 sequence comprises RASQSISSFLN (SEQ ID NO:97), the VL-CDR2sequence comprises AASSLQS (SEQ ID NO:98), and the VL-CDR3 sequence comprises QQSYVHPLT (SEQ ID NO:99);
(viii) the VH-CDR1 sequence comprises YTFTSYGIS (SEQ ID NO:53), the VH-CDR2 sequence comprises WISAYNGNTNYAQKLQG (SEQ ID NO:54), the VH-CDR3 sequence comprises ARDGLGSSPWSAFDI (SEQ ID NO:70), the VL-CDR1 sequence comprises RSSQSLLHSNGYNYLD (SEQ ID NO:100), the VL-CDR2 sequence comprises LGSNRAS (SEQ ID NO:101), and the VL-CDR3 sequence comprises MQARRSPLT (SEQ ID NO:102);
(ix) the VH-CDR1 sequence comprises YTFTSYYMH (SEQ ID NO:71), the VH-CDR2 sequence comprises VINPSGGSTSYAQKFQG (SEQ ID NO:72), the VH-CDR3 sequence comprises ARLMSGSSGS (SEQ ID NO:73), the VL-CDR1 sequence comprises RASQSVSSSYLA (SEQ ID NO:103), the VL-CDR2 sequence comprises GASSRAT (SEQ ID NO:104), and the VL-CDR3 sequence comprises QQYGGFPLT (SEQ ID NO:105);
(x) the VH-CDR1 sequence comprises YTFTGYYMH (SEQ ID NO:74), the VH-CDR2 sequence comprises SINPNSGGTNYAQKFQG (SEQ ID NO:75), the VH-CDR3 sequence comprises ARDSSWKHDY (SEQ ID NO:76), the VL-CDR1 sequence comprises RASQSVSSYLA (SEQ ID NO:86), the VL-CDR2 sequence comprises DASNRAT (SEQ ID NO:87), and the VL-CDR3 sequence comprises QQYSFYPLT (SEQ ID NO:106);
(xi) the VH-CDR1 sequence comprises YSISSGYYWG (SEQ ID NO:77), the VH-CDR2 sequence comprises SIYHSGSTNYNPSLKS (SEQ ID NO:78), the VH-CDR3 sequence comprises ARSPRWRSTYANWFNP (SEQ ID NO:79), the VL-CDR1 sequence comprises RASQGISSWLA (SEQ ID NO:107), the VL-CDR2sequence comprises GASSLQS (SEQ ID NO:108), and the VL-CDR3 sequence comprises QQAAPFPLT (SEQ ID NO:109); or
(xii) the VH-CDR1 sequence comprises YSISSGYYWA (SEQ ID NO:80), the VH-CDR2 sequence comprises SIYHSGSTYYNPSLKS (SEQ ID NO:81), the VH-CDR3 sequence comprises AREHSSSGQWNV (SEQ ID NO: 82), the VL-CDR1 sequence comprises RASQSVSSYLA (SEQ ID NO:86), the VL-CDR2sequence comprises DASNRAT (SEQ ID NO:87), and the VL-CDR3 sequence comprises QQRSFYFT (SEQ ID NO:110).

27.-32. (canceled)

33. The antibody or antigen binding fragment thereof of claim 26, wherein the antibody or antigen binding fragment thereof is a whole antibody, a Fab, a Fab′, a F(ab)2, an scFv, an sc(Fv)2, or a diabody.

34. A chimeric molecule comprising (i) the antibody or antigen-binding fragment thereof of claim 26, and (ii) a heterologous moiety.

35. The chimeric molecule of claim 34, wherein the heterologous moiety comprises a clotting factor.

36.-42. (canceled)

43. The chimeric molecule of claim 34, further comprising a second heterologous moiety.

44. The chimeric molecule according to claim 43, wherein the second heterologous moiety comprises a half-life extending moiety.

45.-46. (canceled)

47. A chimeric molecule comprising (i) the antibody or antigen-binding fragment thereof of claim 26, (ii) a recombinant Factor VIIa comprising a heavy chain and a light chain, and (iii) a half-life extending moiety.

48.-52. (canceled)

53. A pharmaceutical composition comprising the antibody or antigen-binding fragment thereof of claim 26, and a pharmaceutically acceptable carrier.

54. A method of reducing the frequency or degree of a bleeding episode in a human subject in need thereof, comprising administering to the subject an effective amount of the antibody or antigen-binding fragment thereof of claim 26.

55.-57. (canceled)

58. A method of treating a blood coagulation disorder in a human subject in need thereof, comprising administering to the subject an effective amount of the antibody or antigen-binding fragment thereof of claim 26.

59.-60. (canceled)

61. A method of detecting platelets, comprising:
contacting a human blood preparation with the antibody or antigen-binding fragment thereof of claim 26; and
detecting cells in the blood preparation to which the antibody or antigen-binding fragment thereof binds.

62. A method for enriching platelets, comprising:
contacting a human blood preparation with the antibody or antigen-binding fragment thereof of claim 26; and
enriching cells to which the antibody or antigen-binding fragment thereof are bound as compared to those cells in the blood preparation that are not bound by the antibody or antigen-binding fragment thereof.

63. An isolated nucleic acid comprising a nucleotide sequence that is at least 80% identical to a nucleotide sequence selected from the group consisting of SEQ ID NOs: 6, 8, 10, 12, 14, 16, 18, 20, 22, 24, 26, 28, 30, 32, 34, 36, 38, 40, 42, 44, 46, 48, 50, and 52.

64.-67. (canceled)

68. A recombinant vector comprising the nucleic acid of claim 63.

69. A host cell comprising the recombinant vector of claim 68.

70.-72. (canceled)


SEQUENCE LISTING (254 sequences)

SEQ ID NO: 1 - protein, 994 aa, Homo sapiens
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERA

SEQ ID NO: 2 - protein, 994 aa, Homo sapiens
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRACEERA

SEQ ID NO: 3 - protein, 716 aa, Homo sapiens
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKG

SEQ ID NO: 4 - protein, 716 aa, Homo sapiens
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECCKG

SEQ ID NO: 5 - protein, 126 aa, Artificial Sequence
  source: /note=“Description of Artificial Sequence Synthetic polypeptide”
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDLEYYDSSGYAYGYFDLWGRGTLVTVSS

SEQ ID NO: 6 - DNA, 378 bp, Artificial Sequence
  source: /note=“Description of Artificial Sequence Synthetic polynucleotide”
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggcggtgtactactgcgccagagacttggaatactacgacagcagcggatacgcctatggctacttcgacctatgggggagaggtaccttggtcaccgtctcctca

SEQ ID NO: 7 - protein, 112 aa, Artificial Sequence
  source: /note=“Description of Artificial Sequence Synthetic polypeptide”
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALRLPRTFGGGTKVEIK

SEQ ID NO: 8 - DNA, 336 bp, Artificial Sequence
  source: /note=“Description of Artificial Sequence Synthetic polynucleotide”
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaggcactccgcctccctaggacttttggcggagggaccaaggttgagatcaaa

SEQ ID NO: 9 - protein, 122 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDTGYYGASLYFDYWGQGTLVTVSS

SEQ ID NO: 10 - DNA, 366 bp, Artificial Sequence
  source: /note=“Description of Artificial Sequence Synthetic polynucleotide”
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagagacacgggatactacggtgctagcttatatttcgactattggggacagggtacattggtcaccgtctcctca

SEQ ID NO: 11 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSALPRTFGGGTKVEIK

SEQ ID NO: 12 - DNA, 321 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagagaagtgccctccctaggacttttggcggagggaccaaggttgagatcaaa

SEQ ID NO: 13 - protein, 122 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGPPSAYGDYVWDIWGQGTMVTVSS

SEQ ID NO: 14 - DNA, 366 bp, Artificial Sequence
  source: /note=“Description of Artificial Sequence Synthetic polynucleotide”
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagaggaccgcctagcgcctacggagactacgtctgggacatatggggtcagggtacaatggtcaccgtctcctca

SEQ ID NO: 15 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDSSNRATGIPARFSGSGSGTDFTLTISSLEPEDFAVYYCQQRSHLPPTFGGGTKVEIK

SEQ ID NO: 16 - DNA, 321 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgattcatccaacagggccactggcatcccagccaggttcagtggcagtgggtctgggacagacttcactctcaccatcagcagcctagagcctgaagattttgcagtttattactgtcagcagagaagtcacctgcctcctacttttggcggagggaccaaggttgagatcaaa

SEQ ID NO: 17 - protein, 123 aa, Artificial Sequence
  source: /note=“Description of Artificial Sequence Synthetic polypeptide”
EVQLVESGGGLVQPGGSLRLSCAASGFTFSDHHMDWVRQAPGKGLEWVGRTRNKANSYTTEYAASVKGRFTISRDDSKNSLYLQMNSLKTEDTAVYYCARGPPYYADLGMGVWGQGTTVTVSS

SEQ ID NO: 18 - DNA, 369 bp, Artificial Sequence
  source: /note=“Description of Artificial Sequence Synthetic polynucleotide”
gaggtgcagctggtggagtctgggggaggcttggtccagcctggagggtccctgagactctcctgtgcagcctctggattcaccttcagtgaccaccacatggactgggtccgccaggctccagggaaggggctggagtgggttggccgtactagaaacaaagctaacagttacaccacagaatacgccgcgtctgtgaaaggcagattcaccatctcaagagatgattcaaagaactcactgtatctgcaaatgaacagcctgaaaaccgaggacacggcggtgtactactgcgccagaggaccgccttactacgcagacctcggaatgggcgtatggggccagggaacaactgtcaccgtctcctca

SEQ ID NO: 19 - protein, 107 aa, Artificial Sequence
  source: /note=“Description of Artificial Sequence Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQQFNLYPYTFGGGTKVEIK

SEQ ID NO: 20 - DNA, 321 bp, Artificial Sequence
  source: /note=“Description of Artificial Sequence Synthetic polynucleotide”
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagttcaatctctacccttacacttttggcggagggaccaaggttgagatcaaa

SEQ ID NO: 21 - protein, 118 aa, Artificial Sequence
  source: /note=“Description of Artificial Sequence Synthetic polypeptide”
QVQLVQSGAEVKKPGASVKVSCKASGYTFTSYSMHWVRQAPGQGLEWMGIINPSGGSTSYAQKFQGRVTMTRDTSTSTVYMELSSLRSEDTAVYYCARSYDIGYFDLWGRGTLVTVSS

SEQ ID NO: 22 - DNA, 354 bp, Artificial Sequence
  source: /note=“Description of Artificial Sequence Synthetic polynucleotide”
caggtgcagctggtgcagtctggggctgaggtgaagaagcctggggcctcagtgaaggtttcctgcaaggcatctggatacaccttcaccagctacagcatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggcggtgtactactgcgccagatcttacgacataggctacttcgacctatgggggagaggtaccttggtcaccgtctcctca

SEQ ID NO: 23 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASKRATGIPARFSGSGSGTDFTLTISSLEPEDFAVYYCQQDSFLPFTFGGGTKVEIK

SEQ ID NO: 24 - DNA, 321 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaaaagggccactggcatcccagccaggttcagtggcagtgggtctgggacagacttcactctcaccatcagcagcctagagcctgaagattttgcagtttattactgtcagcaggacagtttcctccctttcacttttggcggagggaccaaggttgagatcaaa

SEQ ID NO: 25 - protein, 119 aa, Artificial Sequence
  source: /note=“Description of Artificial Sequence Synthetic polypeptide”
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGRPYDHYFDYWGQGTLVTVSS

SEQ ID NO: 26 - DNA, 357 bp, Artificial Sequence
  source: /note=“Description of Artificial Sequence Synthetic polynucleotide”
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggcggtgtactactgcgccagaggaaggccttacgaccactactttgactactggggacagggtacattggtcaccgtctcctca

SEQ ID NO: 27 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQAYNYPFTFGGGTKVEIK

SEQ ID NO: 28 - DNA, 321 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaggcctataattaccctttcacttttggcggagggaccaaggttgagatcaaa

SEQ ID NO: 29 - protein, 121 aa, Artificial Sequence
  source: /note=“Description of Artificial Sequence Synthetic polypeptide”
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDFYSSVYGMDVWGQGTTVTVSS

SEQ ID NO: 30 - DNA, 363 bp, Artificial Sequence
  source: /note=“Description of Artificial Sequence Synthetic polynucleotide”
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagttctgtgaccgccgcagacacggcggtgtactactgcgccagagacttctacagcagtgtatacggtatggacgtttggggccagggaacaactgtcaccgtctcctca

SEQ ID NO: 31 - protein, 107 aa, Artificial Sequence
  source: /note=“Description of Artificial Sequence Synthetic polypeptide”
DIQLTQSPSSLSASVGDRVTITCRASQSISSFLNWYQQKPGKAPKLLIYAASSLQSGVPSRFSGSGSGTDFTLTISSLQPEDFATYYCQQSYVHPLTFGGGTKVEIK

SEQ ID NO: 32 - DNA, 321 bp, Artificial Sequence
  source: /note=“Description of Artificial Sequence Synthetic polynucleotide”
gacatccagttgacccagtctccatcctccctgtctgcatctgtaggagacagagtcaccatcacttgccgggcaagtcagagcattagcagctttttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagctacgtccaccctctcacttttggcggagggaccaaggttgagatcaaa

SEQ ID NO: 33 - protein, 122 aa, Artificial Sequence
  source: /note=“Description of Artificial Sequence Synthetic polypeptide”
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDGLGSSPWSAFDIWGQGTMVTVSS

SEQ ID NO: 34 - DNA, 366 bp, Artificial Sequence
  source: /note=“Description of Artificial Sequence Synthetic polynucleotide”
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggcggtgtactactgcgccagagacggactgggatccagcccatggtcagctttcgacatatggggtcagggtacaatggtcaccgtctcctca

SEQ ID NO: 35 - protein, 112 aa, Artificial Sequence
  source: /note=“Description of Artificial Sequence Synthetic polypeptide”
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRSPLTFGGGTKVEIK

SEQ ID NO: 36 - DNA, 336 bp, Artificial Sequence
  source: /note=“Description of Artificial Sequence Synthetic polynucleotide”
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaggcaagacgaagccctctcacttttggcggagggaccaaggttgagatcaaa

SEQ ID NO: 37 - protein, 117 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGVINPSGGSTSYAQKFQGRVTMTRDTSTSTVYMELSSLRSEDTAVYYCARLMSGSSGSWGQGTLVTVSS

SEQ ID NO: 38 - DNA, 351 bp, Artificial Sequence
  source: /note=“Description of Artificial Sequence Synthetic polynucleotide”
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggagtcatcaaccctagtggtggtagcacaagctacgcacagaagttccagggcagagtcaccatgaccagggacacgtccacgagcacagtctacatggagctgagcagcctgagatctgaggacacggcggtgtactactgcgccagattgatgagcggatcgtccggaagttggggacagggtacattggtcaccgtctcctca

SEQ ID NO: 39 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYGGFPLTFGGGTKVEIK

SEQ ID NO: 40 - DNA, 324 bp, Artificial Sequence
  source: /note=“Description of Artificial Sequence Synthetic polynucleotide”
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtacggaggcttccctctcacttttggcggagggaccaaggttgagatcaaa

SEQ ID NO: 41 - protein, 117 aa, Artificial Sequence
  source: /note=“Description of Artificial Sequence Synthetic polypeptide”
QVQLVQSGAEVKKPGASVKVSCKASGYTFTGYYMHWVRQAPGQGLEWMGSINPNSGGTNYAQKFQGRVTMTRDTSISTAYMELSRLRSDDTAVYYCARDSSWKHDYWGQGTLVTVSS

SEQ ID NO: 42 - DNA, 351 bp, Artificial Sequence
  source: /note=“Description of Artificial Sequence Synthetic polynucleotide”
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggaagcatcaaccctaacagtggtggcacaaactatgcacagaagtttcagggcagggtcaccatgaccagggacacgtccatcagcacagcctacatggagctgagcaggctgagatctgacgacacggcggtgtactactgcgccagagacagcagctggaaacacgattactggggacagggtacattggtcaccgtctcctca

SEQ ID NO: 43 - protein, 107 aa, Artificial Sequence
  source: /note=“Description of Artificial Sequence Synthetic polypeptide”
EIVLTQSPATLSLSPGERATLSCRASQSVSSYLAWFQQKPGQAPRLLIYDASNRATGIPARFSGSGSGTDFTLTISSLEPEDFAVYYCQQYSFYPLTFGGGTKVEIK

SEQ ID NO: 44 - DNA, 321 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagcagctacttagcctggttccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagtacagtttctaccctctcacttttggcggagggaccaaggttgagatcaaa

SEQ ID NO: 45 - protein, 123 aa, Artificial Sequence
  source: /note=“Description of Artificial Sequence Synthetic polypeptide”
QVQLQESGPGLVKPSETLSLTCAVSGYSISSGYYWGWIRQPPGKGLEWIGSIYHSGSTNYNPSLKSRVTISVDTSKNQFSLKLSSVTAADTAVYYCARSPRWRSTYANWFNPWGQGTLVTVSS

SEQ ID NO: 46 - DNA, 369 bp, Artificial Sequence
  source: /note=“Description of Artificial Sequence Synthetic polynucleotide”
caggtgcagctgcaggagtcgggcccaggactggtgaagccttcggagaccctgtccctcacctgcgctgtctctggttactccatcagcagtggttactactggggctggatccggcagcccccagggaaggggctggagtggattgggagtatctatcatagtgggagcaccaactacaacccgtccctcaagagtcgagtcaccatatcagtagacacgtccaagaaccagttctccctgaagctgagttctgtgaccgccgcagacacggcggtgtactactgcgccagatcacctagatggagatccacctacgccaactggttcaatccctggggacagggtacattggtcaccgtctcctca

SEQ ID NO: 47 - protein, 107 aa, Artificial Sequence
  source: /note=“Description of Artificial Sequence Synthetic polypeptide”
DIQLTQSPSSVSASVGDRVTITCRASQGISSWLAWYQQKPGKAPKLLIYGASSLQSGVPSRFSGSGSGTDFTLTISSLQPEDFATYYCQQAAPFPLTFGGGTKVEIK

SEQ ID NO: 48 - DNA, 321 bp, Artificial Sequence
  source: /note=“Description of Artificial Sequence Synthetic polynucleotide”
gacatccagttgacccagtctccatcttccgtgtctgcatctgtaggagacagagtcaccatcacttgtcgggcgagtcagggtattagcagctggttagcctggtatcagcagaaaccagggaaagcccctaagctcctgatctatggtgcatccagtttgcaaagtggggtcccatcaaggttcagcggcagtggatctgggacagatttcactctcaccatcagcagcctgcagcctgaagattttgcaacttactactgtcagcaggcagcccccttccctctcacttttggcggagggaccaaggttgagatcaaa

SEQ ID NO: 49 - protein, 119 aa, Artificial Sequence
  source: /note=“Description of Artificial Sequence Synthetic polypeptide”
QVQLQESGPGLVKPSETLSLTCAVSGYSISSGYYWAWIRQPPGKGLEWIGSIYHSGSTYYNPSLKSRVTISVDTSKNQFSLKLSSVTAADTAVYYCAREHSSSGQWNVWGQGTMVTVSS

SEQ ID NO: 50 - DNA, 357 bp, Artificial Sequence
  source: /note=“Description of Artificial Sequence Synthetic polynucleotide”
caggtgcagctgcaggagtcgggcccaggactggtgaagccttcggagaccctgtccctcacctgcgctgtctctggttactccatcagcagtggttactactgggcttggatccggcagcccccagggaaggggctggagtggattgggagtatctatcatagtgggagcacctactacaacccgtccctcaagagtcgagtcaccatatcagtagacacgtccaagaaccagttctccctgaagctgagttctgtgaccgccgcagacacggcggtgtactactgcgccagagagcatagcagcagcggccaatggaacgtatggggtcagggtacaatggtcaccgtctcctca

SEQ ID NO: 51 - protein, 106 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSFYFTFGGGTKVEIK

SEQ ID NO: 52 - DNA, 318 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagagaagtttttacttcacttttggcggagggaccaaggttgagatcaaa

SEQ ID NO: 53 - protein, 9 aa, Artificial Sequence
  source: /note=“Description of Artificial Sequence Synthetic peptide”
YTFTSYGIS

SEQ ID NO: 54 - protein, 17 aa, Artificial Sequence
  source: /note=“Description of Artificial Sequence Synthetic peptide”
WISAYNGNTNYAQKLQG

SEQ ID NO: 55 - protein, 19 aa, Artificial Sequence
  source: /note=“Description of Artificial Sequence Synthetic peptide”
ARDLEYYDSSGYAYGYFDL

SEQ ID NO: 56 - protein, 9 aa, Artificial Sequence
  source: /note=“Description of Artificial Sequence Synthetic peptide”
GTFSSYAIS

SEQ ID NO: 57 - protein, 17 aa, Artificial Sequence
  source: /note=“Description of Artificial Sequence Synthetic peptide”
GIIPIFGTANYAQKFQG

SEQ ID NO: 58 - protein, 15 aa, Artificial Sequence
  source: /note=“Description of Artificial Sequence Synthetic peptide”
ARDTGYYGASLYFDY

SEQ ID NO: 59 - protein, 15 aa, Artificial Sequence
  source: /note=“Description of Artificial Sequence Synthetic peptide”
ARGPPSAYGDYVWDI

SEQ ID NO: 60 - protein, 9 aa, Artificial Sequence
  source: /note=“Description of Artificial Sequence Synthetic peptide”
FTFSDHHMD

SEQ ID NO: 61 - protein, 19 aa, Artificial Sequence
  source: /note=“Description of Artificial Sequence Synthetic peptide”
RTRNKANSYTTEYAASVKG

SEQ ID NO: 62 - protein, 14 aa, Artificial Sequence
  source: /note=“Description of Artificial Sequence Synthetic peptide”
ARGPPYYADLGMGV

SEQ ID NO: 63 - protein, 9 aa, Artificial Sequence
  source: /note=“Description of Artificial Sequence Synthetic peptide”
YTFTSYSMH

SEQ ID NO: 64 - protein, 17 aa, Artificial Sequence
  source: /note=“Description of Artificial Sequence Synthetic peptide”
IINPSGGSTSYAQKFQG

SEQ ID NO: 65 - protein, 11 aa, Artificial Sequence
  source: /note=“Description of Artificial Sequence Synthetic peptide”
ARSYDIGYFDL

SEQ ID NO: 66 - protein, 12 aa, Artificial Sequence
  source: /note=“Description of Artificial Sequence Synthetic peptide”
ARGRPYDHYFDY

SEQ ID NO: 67 - protein, 11 aa, Artificial Sequence
  source: /note=“Description of Artificial Sequence Synthetic peptide”
GSISSSSYYWG

SEQ ID NO: 68 - protein, 16 aa, Artificial Sequence
  source: /note=“Description of Artificial Sequence Synthetic peptide”
SIYYSGSTYYNPSLKS

SEQ ID NO: 69 - protein, 13 aa, Artificial Sequence
  source: /note=“Description of Artificial Sequence Synthetic peptide”
ARDFYSSVYGMDV

SEQ ID NO: 70 - protein, 15 aa, Artificial Sequence
  source: /note=“Description of Artificial Sequence Synthetic peptide”
ARDGLGSSPWSAFDI

SEQ ID NO: 71 - protein, 9 aa, Artificial Sequence
  source: /note=“Description of Artificial Sequence Synthetic peptide”
YTFTSYYMH

SEQ ID NO: 72 - protein, 17 aa, Artificial Sequence
  source: /note=“Description of Artificial Sequence Synthetic peptide”
VINPSGGSTSYAQKFQG

SEQ ID NO: 73 - protein, 10 aa, Artificial Sequence
  source: /note=“Description of Artificial Sequence Synthetic peptide”
ARLMSGSSGS

SEQ ID NO: 74 - protein, 9 aa, Artificial Sequence
  source: /note=“Description of Artificial Sequence Synthetic peptide”
YTFTGYYMH

SEQ ID NO: 75 - protein, 17 aa, Artificial Sequence
  source: /note=“Description of Artificial Sequence Synthetic peptide”
SINPNSGGTNYAQKFQG

SEQ ID NO: 76 - protein, 10 aa, Artificial Sequence
  source: /note=“Description of Artificial Sequence Synthetic peptide”
ARDSSWKHDY

SEQ ID NO: 77 - protein, 10 aa, Artificial Sequence
  source: /note=“Description of Artificial Sequence Synthetic peptide”
YSISSGYYWG

SEQ ID NO: 78 - protein, 16 aa, Artificial Sequence
  source: /note=“Description of Artificial Sequence Synthetic peptide”
SIYHSGSTNYNPSLKS

SEQ ID NO: 79 - protein, 16 aa, Artificial Sequence
  source: /note=“Description of Artificial Sequence Synthetic peptide”
ARSPRWRSTYANWFNP

SEQ ID NO: 80 - protein, 10 aa, Artificial Sequence
  source: /note=“Description of Artificial Sequence Synthetic peptide”
YSISSGYYWA

SEQ ID NO: 81 - protein, 16 aa, Artificial Sequence
  source: /note=“Description of Artificial Sequence Synthetic peptide”
SIYHSGSTYYNPSLKS

SEQ ID NO: 82 - protein, 12 aa, Artificial Sequence
  source: /note=“Description of Artificial Sequence Synthetic peptide”
AREHSSSGQWNV

SEQ ID NO: 83 - protein, 16 aa, Artificial Sequence
  source: /note=“Description of Artificial Sequence Synthetic peptide”
RSSQSLLHSNGYNYLD

SEQ ID NO: 84 - protein, 7 aa, Artificial Sequence
  source: /note=“Description of Artificial Sequence Synthetic peptide”
LGSNRAS

SEQ ID NO: 85 - protein, 9 aa, Artificial Sequence
  source: /note=“Description of Artificial Sequence Synthetic peptide”
MQALRLPRT

SEQ ID NO: 86 - protein, 11 aa, Artificial Sequence
  source: /note=“Description of Artificial Sequence Synthetic peptide”
RASQSVSSYLA

SEQ ID NO: 87 - protein, 7 aa, Artificial Sequence
  source: /note=“Description of Artificial Sequence Synthetic peptide”
DASNRAT

SEQ ID NO: 88 - protein, 9 aa, Artificial Sequence
  source: /note=“Description of Artificial Sequence Synthetic peptide”
QQRSALPRT

SEQ ID NO: 89 - protein, 7 aa, Artificial Sequence
  source: /note=“Description of Artificial Sequence Synthetic peptide”
DSSNRAT

SEQ ID NO: 90 - protein, 9 aa, Artificial Sequence
  source: /note=“Description of Artificial Sequence Synthetic peptide”
QQRSHLPPT

SEQ ID NO: 91 - protein, 11 aa, Artificial Sequence
  source: /note=“Description of Artificial Sequence Synthetic peptide”
RASQSVSSNLA

SEQ ID NO: 92 - protein, 7 aa, Artificial Sequence
  source: /note=“Description of Artificial Sequence Synthetic peptide”
GASTRAT

SEQ ID NO: 93 - protein, 9 aa, Artificial Sequence
  source: /note=“Description of Artificial Sequence Synthetic peptide”
QQFNLYPYT

SEQ ID NO: 94 - protein, 7 aa, Artificial Sequence
  source: /note=“Description of Artificial Sequence Synthetic peptide”
DASKRAT

SEQ ID NO: 95 - protein, 9 aa, Artificial Sequence
  source: /note=“Description of Artificial Sequence Synthetic peptide”
QQDSFLPFT

SEQ ID NO: 96 - protein, 9 aa, Artificial Sequence
  source: /note=“Description of Artificial Sequence Synthetic peptide”
QQAYNYPFT

SEQ ID NO: 97 - protein, 11 aa, Artificial Sequence
  source: /note=“Description of Artificial Sequence Synthetic peptide”
RASQSISSFLN

SEQ ID NO: 98 - protein, 7 aa, Artificial Sequence
  source: /note=“Description of Artificial Sequence Synthetic peptide”
AASSLQS

SEQ ID NO: 99 - protein, 9 aa, Artificial Sequence
  source: /note=“Description of Artificial Sequence Synthetic peptide”
QQSYVHPLT

SEQ ID NO: 100 - protein, 16 aa, Artificial Sequence
  source: /note=“Description of Artificial Sequence Synthetic peptide”
RSSQSLLHSNGYNYLD

SEQ ID NO: 101 - protein, 7 aa, Artificial Sequence
  source: /note=“Description of Artificial Sequence Synthetic peptide”
LGSNRAS

SEQ ID NO: 102 - protein, 9 aa, Artificial Sequence
  source: /note=“Description of Artificial Sequence Synthetic peptide”
MQARRSPLT

SEQ ID NO: 103 - protein, 12 aa, Artificial Sequence
  source: /note=“Description of Artificial Sequence Synthetic peptide”
RASQSVSSSYLA

SEQ ID NO: 104 - protein, 7 aa, Artificial Sequence
  source: /note=“Description of Artificial Sequence Synthetic peptide”
GASSRAT

SEQ ID NO: 105 - protein, 9 aa, Artificial Sequence
  source: /note=“Description of Artificial Sequence Synthetic peptide”
QQYGGFPLT

SEQ ID NO: 106 - protein, 9 aa, Artificial Sequence
  source: /note=“Description of Artificial Sequence Synthetic peptide”
QQYSFYPLT

SEQ ID NO: 107 - protein, 11 aa, Artificial Sequence
  source: /note=“Description of Artificial Sequence Synthetic peptide”
RASQGISSWLA

SEQ ID NO: 108 - protein, 7 aa, Artificial Sequence
  source: /note=“Description of Artificial Sequence Synthetic peptide”
GASSLQS

SEQ ID NO: 109 - protein, 9 aa, Artificial Sequence
  source: /note=“Description of Artificial Sequence Synthetic peptide”
QQAAPFPLT

SEQ ID NO: 110 - protein, 8 aa, Artificial Sequence
  source: /note=“Description of Artificial Sequence Synthetic peptide”
QQRSFYFT

SEQ ID NO: 111 - protein, 9 aa, Artificial Sequence
  source: /note=“Description of Artificial Sequence Synthetic peptide”
  VARIANT (1)..(1): /replace=“Gly”
  VARIANT (4)..(4): /replace=“Ser”
  VARIANT (5)..(5): /replace=“Gly”
  VARIANT (7)..(7): /replace=“Ala” or “Ser” or “Tyr”
  VARIANT (8)..(8): /replace=“Met” or “His”
  VARIANT (9)..(9): /replace=“His”
  MISC_FEATURE (1)..(9): /note=“Variant residues given in the sequence have no preference with respect to those in the annotations for variant positions”
YTFTSYGIS

SEQ ID NO: 112 - protein, 9 aa, Artificial Sequence
  source: /note=“Description of Artificial Sequence Synthetic peptide”
  VARIANT (1)..(1): /replace=“Gly”
  VARIANT (4)..(4): /replace=“Ser”
  VARIANT (5)..(5): /replace=“Gly”
  VARIANT (7)..(7): /replace=“Ala”
  MISC_FEATURE (1)..(9): /note=“Variant residues given in the sequence have no preference with respect to those in the annotations for variant positions”
YTFTSYGIS

SEQ ID NO: 113 - protein, 17 aa, Artificial Sequence
  source: /note=“Description of Artificial Sequence Synthetic peptide”
  VARIANT (1)..(1): /replace=“Val” or “Ser”
  VARIANT (5)..(5): /replace=“Asn”
  VARIANT (6)..(6): /replace=“Ser”
  VARIANT (8)..(8): /replace=“Gly”
  VARIANT (10)..(10): /replace=“Asn”
  MISC_FEATURE (1)..(17): /note=“Variant residues given in the sequence have no preference with respect to those in the annotations for variant positions”
IINPSGGSTSYAQKFQG

SEQ ID NO: 114 - protein, 17 aa, Artificial Sequence
  source: /note=“Description of Artificial Sequence Synthetic peptide”
  VARIANT (1)..(1): /replace=“Val”
  MISC_FEATURE (1)..(17): /note=“Variant residues given in the sequence have no preference with respect to those in the annotations for variant positions”
IINPSGGSTSYAQKFQG

SEQ ID NO: 115 - protein, 10 aa, Artificial Sequence
  source: /note=“Description of Artificial Sequence Synthetic peptide”
  VARIANT (1)..(1): /replace=“Gly”
  VARIANT (10)..(10): /replace=“Ala”
  MISC_FEATURE (1)..(10): /note=“Variant residues given in the sequence have no preference with respect to those in the annotations for variant positions”
YSISSGYYWG

SEQ ID NO: 116 - protein, 11 aa, Artificial Sequence
  source: /note=“Description of Artificial Sequence Synthetic peptide”
  VARIANT (1)..(1): /replace=“Gly”
  VARIANT (6)..(6): /replace=“Ser”
  VARIANT (7)..(7): /replace=“ ”
  MISC_FEATURE (1)..(11): /note=“Variant residues given in the sequence have no preference with respect to those in the annotations for variant positions”
YSISSGSYYWG

SEQ ID NO: 117 - protein, 16 aa, Artificial Sequence
  source: /note=“Description of Artificial Sequence Synthetic peptide”
  VARIANT (9)..(9): /replace=“Tyr”
  MISC_FEATURE (1)..(16): /note=“Variant residues given in the sequence have no preference with respect to those in the annotations for variant positions”
SIYHSGSTNYNPSLKS

SEQ ID NO: 118 - protein, 12 aa, Artificial Sequence
  source: /note=“Description of Artificial Sequence Synthetic peptide”
  VARIANT (5)..(5): /replace=“Gly”
  VARIANT (6)..(6): /replace=“Ile”
  VARIANT (9)..(9): /replace=“ ”
  VARIANT (10)..(10): /replace=“Asn” or “Phe” or “Trp”
  VARIANT (12)..(12): /replace=“Asn”
  MISC_FEATURE (1)..(12): /note=“Variant residues given in the sequence have no preference with respect to those in the annotations for variant positions”
RASQSVSSSYLA

SEQ ID NO: 119 - protein, 7 aa, Artificial Sequence
  source: /note=“Description of Artificial Sequence Synthetic peptide”
  VARIANT (1)..(1): /replace=“Gly” or “Leu”
  VARIANT (2)..(2): /replace=“Ser” or “Gly”
  VARIANT (4)..(4): /replace=“Thr” or “Ser” or “Lys”
  VARIANT (7)..(7): /replace=“Ser”
  MISC_FEATURE (1)..(7): /note=“Variant residues given in the sequence have no preference with respect to those in the annotations for variant positions”
DASNRAT

SEQ ID NO: 120 - protein, 9 aa, Artificial Sequence
  source: /note=“Description of Artificial Sequence Synthetic peptide”
  VARIANT (1)..(1): /replace=“Met”
  VARIANT (3)..(3): /replace=“Ser” or “Asp” or “Tyr” or “Phe” or “Arg”
  VARIANT (4)..(4): /replace=“Tyr” or “Ser” or “Leu” or “Arg” or “Gly” or “Asn”
  VARIANT (5)..(5): /replace=“Val” or “Phe” or “Arg” or “Gly” or “Leu” or “Asn” or “Ala” or “His”
  VARIANT (6)..(6): /replace=“His” or “Tyr” or “Leu” or “Ser”
  VARIANT (8)..(8): /replace=“Phe” or “Arg” or “Tyr” or “Pro”
  MISC_FEATURE (1)..(9): /note=“Variant residues given in the sequence have no preference with respect to those in the annotations for variant positions”
QQAAPFPLT

SEQ ID NO: 121 - protein, 107 aa, Artificial Sequence [sequence omitted]

SEQ ID NO: 122 - protein, 103 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 123 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - DNA, 2082 bp, Artificial Sequence
  source: /note=“Description of Artificial Sequence Synthetic polynucleotide”
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggtggcggtggctccggcggaggtgggtccggtggcggcggatcaggtgggggtggatcaggcggtggaggttccggtggcgggggatccgatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaggcactccgcctccctaggacttttggcggagggaccaaggttgagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 125 - protein, 693 aa, Artificial Sequence
  source: /note=“Description of Artificial Sequence Synthetic polypeptide”
MVSQALRLLCLLLGLQGCLAAVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFPGGGGSGGGGSGGGGSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALRLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - DNA, 690 bp, Artificial Sequence
  source: /note=“Description of Artificial Sequence Synthetic polynucleotide”
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggcggtgtactactgcgccagagacttggaatactacgacagcagcggatacgcctatggctacttcgacctatgggggagaggtaccttggtcaccgtctcctcagctagcacgaaggggcccagcgtgttccccctggcccccagcagcaagagcaccagcggcggcaccgccgccctgggctgcctggtgaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttga

SEQ ID NO: 127 - protein, 229 aa, Artificial Sequence
  source: /note=“Description of Artificial Sequence Synthetic polypeptide”
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDLEYYDSSGYAYGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 128 - protein, 444 aa, Artificial Sequence
  source: /note=“Description of Artificial Sequence Synthetic polypeptide”
MVSQALRLLCLLLGLQGCLAAVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 129 - protein, 152 aa, Artificial Sequence [sequence omitted]

SEQ ID NO: 130 - protein, 254 aa, Artificial Sequence [sequence omitted]

SEQ ID NO: 131 - protein, 12 aa, Artificial Sequence
  source: /note=“Description of Artificial Sequence Synthetic peptide”
LQQSRKVGDSPN

SEQ ID NO: 132 - protein, 7 aa, Artificial Sequence
  source: /note=“Description of Artificial Sequence Synthetic peptide”
EASYPGK

SEQ ID NO: 133 - protein, 461 aa, Artificial Sequence
  source: /note=“Description of Artificial Sequence Synthetic polypeptide”
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 134 - protein, 488 aa, Artificial Sequence
  source: /note=“Description of Artificial Sequence Synthetic polypeptide”
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 135 - protein, 474 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 591 aa, Homo sapiens
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 137 - protein, 6 aa, Artificial Sequence
  source: /note=“Description of Artificial Sequence Synthetic peptide”
  VARIANT (2)..(2): /replace=“Asn” or “Ser” or “Thr” or “Trp”
  VARIANT (3)..(3): /replace=“Gln” or “His” or “Ile” or “Leu” or “Lys”
  VARIANT (4)..(4): /replace=“Asp” or “Phe” or “Trp” or “Tyr”
  VARIANT (5)..(5): /replace=“Gly” or “Leu” or “Phe” or “Ser” or “Thr”
  MISC_FEATURE (1)..(6): /note=“Variant residues given in the sequence have no preference with respect to those in the annotations for variant positions”
CDNADC

SEQ ID NO: 138 - protein, 11 aa, Unknown
  source: /note=“Description of Unknown Albumin-binding peptide”
DICLPRWGCLW

SEQ ID NO: 139 - protein, 18 aa, Unknown
  source: /note=“Description of Unknown Albumin-binding peptide”
RLIEDICLPRWGCLWEDD

SEQ ID NO: 140 - protein, 20 aa, Unknown
  source: /note=“Description of Unknown Albumin-binding peptide”
QRLMEDICLPRWGCLWEDDF

SEQ ID NO: 141 - protein, 21 aa, Unknown
  source: /note=“Description of Unknown Albumin-binding peptide”
QGLIGDICLPRWGCLWGDSVK

SEQ ID NO: 142 - protein, 20 aa, Unknown
  source: /note=“Description of Unknown Albumin-binding peptide”
GEWWEDICLPRWGCLWEEED

SEQ ID NO: 143 - protein, 32 aa, Homo sapiens
DPRFQDSSSSKAPPPSLPSPSRLPGPSDTPIL

SEQ ID NO: 144 - protein, 28 aa, Homo sapiens
SSSSKAPPPSLPSPSRLPGPSDTPILPQ

SEQ ID NO: 145 - protein, 20 aa, Artificial Sequence
  source: /note=“Description of Artificial Sequence Synthetic peptide”
ASPAAPAPASPAAPAPSAPA

SEQ ID NO: 146 - protein, 20 aa, Artificial Sequence
  source: /note=“Description of Artificial Sequence Synthetic peptide”
AAPASPAPAAPSAPAPAAPS

SEQ ID NO: 147 - protein, 20 aa, Artificial Sequence
  source: /note=“Description of Artificial Sequence Synthetic peptide”
APSSPSPSAPSSPSPASPSS

SEQ ID NO: 148 - protein, 19 aa, Artificial Sequence
  source: /note=“Description of Artificial Sequence Synthetic peptide”
APSSPSPSAPSSPSPASPS

SEQ ID NO: 149 - protein, 20 aa, Artificial Sequence
  source: /note=“Description of Artificial Sequence Synthetic peptide”
SSPSAPSPSSPASPSPSSPA

SEQ ID NO: 150 - protein, 24 aa, Artificial Sequence
  source: /note=“Description of Artificial Sequence Synthetic peptide”
AASPAAPSAPPAAASPAAPSAPPA

SEQ ID NO: 151 - protein, 20 aa, Artificial Sequence
  source: /note=“Description of Artificial Sequence Synthetic peptide”
ASAAAPAAASAAASAPSAAA

SEQ ID NO: 152 - protein, 20 aa, Artificial Sequence
  source: /note=“Description of Artificial Sequence Synthetic peptide”
  VARIANT (2)..(20): /replace=“ ”
  MISC_FEATURE (1)..(20): /note=“This sequence may encompass 1-20 ”Gly“ repeating units”
  MISC_FEATURE (1)..(20): /note=“Variant residues given in the sequence have no preference with respect to those in the annotations for variant positions”
GGGGGGGGGGGGGGGGGGGG

SEQ ID NO: 153 - protein, 100 aa, Artificial Sequence
  source: /note=“Description of Artificial Sequence Synthetic polypeptide”
  VARIANT (6)..(100): /replace=“ ”
  MISC_FEATURE (1)..(100): /note=“This sequence may encompass 1-20 ”Gly Gly Gly Gly Ser“ repeating units”
  MISC_FEATURE (1)..(100): /note=“Variant residues given in the sequence have no preference with respect to those in the annotations for variant positions”
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 154 - protein, 101 aa, Artificial Sequence
  source: /note=“Description of Artificial Sequence Synthetic polypeptide”
  VARIANT (7)..(101): /replace=“ ”
  MISC_FEATURE (2)..(101): /note=“This region may encompass 1-20 ”Gly Gly Gly Gly Ser“ repeating units”
  MISC_FEATURE (1)..(101): /note=“Variant residues given in the sequence have no preference with respect to those in the annotations for variant positions”
SGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 155 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 156 - protein, 250 aa, Artificial Sequence
  source: /note=“Description of Artificial Sequence Synthetic polypeptide”
  VARIANT (2)..(4): /replace=“ ”
  VARIANT (5)..(5): /replace=“ ”
  VARIANT (7)..(9): /replace=“ ”
  VARIANT (10)..(10): /replace=“ ”
  VARIANT (12)..(14): /replace=“ ”
  VARIANT (15)..(15): /replace=“ ”
  VARIANT (17)..(19): /replace=“ ”
  VARIANT (20)..(20): /replace=“ ”
  VARIANT (22)..(24): /replace=“ ”
  VARIANT (25)..(25): /replace=“ ”
  VARIANT (27)..(29): /replace=“ ”
  VARIANT (30)..(30): /replace=“ ”
  VARIANT (32)..(34): /replace=“ ”
  VARIANT (35)..(35): /replace=“ ”
  VARIANT (37)..(39): /replace=“ ”
  VARIANT (40)..(40): /replace=“ ”
  VARIANT (42)..(44): /replace=“ ”
  VARIANT (45)..(45): /replace=“ ”
  VARIANT (47)..(49): /replace=“ ”
  VARIANT (50)..(50): /replace=“ ”
  VARIANT (52)..(54): /replace=“ ”
  VARIANT (55)..(55): /replace=“ ”
  VARIANT (57)..(59): /replace=“ ”
  VARIANT (60)..(60): /replace=“ ”
  VARIANT (62)..(64): /replace=“ ”
  VARIANT (65)..(65): /replace=“ ”
  VARIANT (67)..(69): /replace=“ ”
  VARIANT (70)..(70): /replace=“ ”
  VARIANT (72)..(74): /replace=“ ”
  VARIANT (75)..(75): /replace=“ ”
  VARIANT (77)..(79): /replace=“ ”
  VARIANT (80)..(80): /replace=“ ”
  VARIANT (82)..(84): /replace=“ ”
  VARIANT (85)..(85): /replace=“ ”
  VARIANT (87)..(89): /replace=“ ”
  VARIANT (90)..(90): /replace=“ ”
  VARIANT (92)..(94): /replace=“ ”
  VARIANT (95)..(95): /replace=“ ”
  VARIANT (97)..(99): /replace=“ ”
  VARIANT (100)..(100): /replace=“ ”
  VARIANT (102)..(104): /replace=“ ”
  VARIANT (105)..(105): /replace=“ ”
  VARIANT (107)..(109): /replace=“ ”
  VARIANT (110)..(110): /replace=“ ”
  VARIANT (112)..(114): /replace=“ ”
  VARIANT (115)..(115): /replace=“ ”
  VARIANT (117)..(119): /replace=“ ”
  VARIANT (120)..(120): /replace=“ ”
  VARIANT (122)..(124): /replace=“ ”
  VARIANT (125)..(125): /replace=“ ”
  VARIANT (127)..(129): /replace=“ ”
  VARIANT (130)..(130): /replace=“ ”
  VARIANT (132)..(134): /replace=“ ”
  VARIANT (135)..(135): /replace=“ ”
  VARIANT (137)..(139): /replace=“ ”
  VARIANT (140)..(140): /replace=“ ”
  VARIANT (142)..(144): /replace=“ ”
  VARIANT (145)..(145): /replace=“ ”
  VARIANT (147)..(149): /replace=“ ”
  VARIANT (150)..(150): /replace=“ ”
  VARIANT (152)..(154): /replace=“ ”
  VARIANT (155)..(155): /replace=“ ”
  VARIANT (157)..(159): /replace=“ ”
  VARIANT (160)..(160): /replace=“ ”
  VARIANT (162)..(164): /replace=“ ”
  VARIANT (165)..(165): /replace=“ ”
  VARIANT (167)..(169): /replace=“ ”
  VARIANT (170)..(170): /replace=“ ”
  VARIANT (172)..(174): /replace=“ ”
  VARIANT (175)..(175): /replace=“ ”
  VARIANT (177)..(179): /replace=“ ”
  VARIANT (180)..(180): /replace=“ ”
  VARIANT (182)..(184): /replace=“ ”
  VARIANT (185)..(185): /replace=“ ”
  VARIANT (187)..(189): /replace=“ ”
  VARIANT (190)..(190): /replace=“ ”
  VARIANT (192)..(194): /replace=“ ”
  VARIANT (195)..(195): /replace=“ ”
  VARIANT (197)..(199): /replace=“ ”
  VARIANT (200)..(200): /replace=“ ”
  VARIANT (202)..(204): /replace=“ ”
  VARIANT (205)..(205): /replace=“ ”
  VARIANT (207)..(209): /replace=“ ”
  VARIANT (210)..(210): /replace=“ ”
  VARIANT (212)..(214): /replace=“ ”
  VARIANT (215)..(215): /replace=“ ”
  VARIANT (217)..(219): /replace=“ ”
  VARIANT (220)..(220): /replace=“ ”
  VARIANT (222)..(224): /replace=“ ”
  VARIANT (225)..(225): /replace=“ ”
  VARIANT (227)..(229): /replace=“ ”
  VARIANT (230)..(230): /replace=“ ”
  VARIANT (232)..(234): /replace=“ ”
  VARIANT (235)..(235): /replace=“ ”
  VARIANT (237)..(239): /replace=“ ”
  VARIANT (240)..(240): /replace=“ ”
  VARIANT (242)..(244): /replace=“ ”
  VARIANT (245)..(245): /replace=“ ”
  VARIANT (247)..(249): /replace=“ ”
  VARIANT (250)..(250): /replace=“ ”
  MISC_FEATURE (1)..(250): /note=“This sequence may encompass 1-50 ”(Gly) x-(Ser)y“ repeating units, wherein x is 1-4 and y is 0-1; See specification as filed for detailed description of substitutions and preferred embodiments”
  MISC_FEATURE (1)..(250): /note=“Variant residues given in the sequence have no preference with respect to those in the annotations for variant positions”
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 157 - protein, 4 aa, Artificial Sequence
  source: /note=“Description of Artificial Sequence Synthetic peptide”
GGGG

SEQ ID NO: 158 - protein, 200 aa, Artificial Sequence
  source: /note=“Description of Artificial Sequence Synthetic polypeptide”
  VARIANT (3)..(200): /replace=“ ”
  MISC_FEATURE (1)..(200): /note=“This region may encompass 1-100 ”Gly Ala“ repeating units”
  MISC_FEATURE (1)..(200): /note=“Variant residues given in the sequence have no preference with respect to those in the annotations for variant positions”
GAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGA

SEQ ID NO: 159 - protein, 300 aa, Artificial Sequence
  source: /note=“Description of Artificial Sequence Synthetic polypeptide”
  VARIANT (4)..(300): /replace=“ ”
  MISC_FEATURE (1)..(300): /note=“This region may encompass 1-100 ”Gly Gly Ser“ repeating units”
  MISC_FEATURE (1)..(300): /note=“Variant residues given in the sequence have no preference with respect to those in the annotations for variant positions”
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 160 - protein, 400 aa, Artificial Sequence
  source: /note=“Description of Artificial Sequence Synthetic polypeptide”
  VARIANT (5)..(400): /replace=“ ”
  MISC_FEATURE (1)..(400): /note=“This region may encompass 1-100 ”Gly Gly Gly Ser“ repeating units”
  MISC_FEATURE (1)..(400): /note=“Variant residues given in the sequence have no preference with respect to those in the annotations for variant positions”
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 161 - protein, 800 aa, Artificial Sequence
  source: /note=“Description of Artificial Sequence Synthetic polypeptide”
  VARIANT (4)..(300): /replace=“ ”
  MISC_FEATURE (1)..(300): /note=“This region may encompass 1-100 ”Gly Gly Ser“ repeating units”
  VARIANT (306)..(800): /replace=“ ”
  MISC_FEATURE (301)..(800): /note=“This region may encompass 1-100 ”Gly Gly Gly Gly Ser“ repeating units”
  MISC_FEATURE (1)..(800): /note=“Variant residues given in the sequence have no preference with respect to those in the annotations for variant positions”
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 162 - protein, 7 aa, Artificial Sequence
  source: /note=“Description of Artificial Sequence Synthetic peptide”
SGGSGGS

SEQ ID NO: 163 - protein, 15 aa, Artificial Sequence
  source: /note=“Description of Artificial Sequence Synthetic peptide”
GGSGGSGGSGGSGGG

SEQ ID NO: 164 - protein, 16 aa, Artificial Sequence
  source: /note=“Description of Artificial Sequence Synthetic peptide”
GGSGGSGGGGSGGGGS

SEQ ID NO: 165 - protein, 18 aa, Artificial Sequence
  source: /note=“Description of Artificial Sequence Synthetic peptide”
GGSGGSGGSGGSGGSGGS

SEQ ID NO: 166 - protein, 15 aa, Artificial Sequence
  source: /note=“Description of Artificial Sequence Synthetic peptide”
GGGGSGGGGSGGGGS

SEQ ID NO: 167 - protein, 400 aa, Artificial Sequence
  source: /note=“Description of Artificial Sequence Synthetic polypeptide”
  VARIANT (5)..(400): /replace=“ ”
  MISC_FEATURE (1)..(400): /note=“This sequence may encompass 1-100 ”Gly Gly Gly Gly“ repeating units”
  MISC_FEATURE (1)..(400): /note=“Variant residues given in the sequence have no preference with respect to those in the annotations for variant positions”
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 168 - protein, 10 aa, Artificial Sequence
  source: /note=“Description of Artificial Sequence Synthetic peptide”
GGGGSGGGGS

SEQ ID NO: 169 - protein, 15 aa, Artificial Sequence
  source: /note=“Description of Artificial Sequence Synthetic peptide”
GGGGSGGGGSGGGGS

SEQ ID NO: 170 - protein, 20 aa, Artificial Sequence
  source: /note=“Description of Artificial Sequence Synthetic peptide”
GGGGSGGGGSGGGGSGGGGS

SEQ ID NO: 171 - protein, 25 aa, Artificial Sequence
  source: /note=“Description of Artificial Sequence Synthetic peptide”
GGGGSGGGGSGGGGSGGGGSGGGGS

SEQ ID NO: 172 - protein, 30 aa, Artificial Sequence
  source: /note=“Description of Artificial Sequence Synthetic polypeptide”
GGGGSGGGGSGGGGSGGGGSGGGGSGGGGS

SEQ ID NO: 173 - protein, 35 aa, Artificial Sequence
  source: /note=“Description of Artificial Sequence Synthetic polypeptide”
GGGGSGGGGSGGGGSGGGGSGGGGSGGGGSGGGGS

SEQ ID NO: 174 - protein, 40 aa, Artificial Sequence
  source: /note=“Description of Artificial Sequence Synthetic polypeptide”
GGGGSGGGGSGGGGSGGGGSGGGGSGGGGSGGGGSGGGGS

SEQ ID NO: 175 - protein, 45 aa, Artificial Sequence
  source: /note=“Description of Artificial Sequence Synthetic polypeptide”
GGGGSGGGGSGGGGSGGGGSGGGGSGGGGSGGGGSGGGGSGGGGS

SEQ ID NO: 176 - protein, 50 aa, Artificial Sequence
  source: /note=“Description of Artificial Sequence Synthetic polypeptide”
GGGGSGGGGSGGGGSGGGGSGGGGSGGGGSGGGGSGGGGSGGGGSGGGGS

SEQ ID NO: 177 - protein, 6 aa, Artificial Sequence
  source: /note=“Description of Artificial Sequence Synthetic peptide”
SGGGGS

SEQ ID NO: 178 - protein, 11 aa, Artificial Sequence
  source: /note=“Description of Artificial Sequence Synthetic peptide”
SGGGGSGGGGS

SEQ ID NO: 179 - protein, 16 aa, Artificial Sequence
  source: /note=“Description of Artificial Sequence Synthetic peptide”
SGGGGSGGGGSGGGGS

SEQ ID NO: 180 - protein, 21 aa, Artificial Sequence
  source: /note=“Description of Artificial Sequence Synthetic peptide”
SGGGGSGGGGSGGGGSGGGGS

SEQ ID NO: 181 - protein, 26 aa, Artificial Sequence
  source: /note=“Description of Artificial Sequence Synthetic peptide”
SGGGGSGGGGSGGGGSGGGGSGGGGS

SEQ ID NO: 182 - protein, 31 aa, Artificial Sequence
  source: /note=“Description of Artificial Sequence Synthetic polypeptide”
SGGGGSGGGGSGGGGSGGGGSGGGGSGGGGS

SEQ ID NO: 183 - protein, 36 aa, Artificial Sequence
  source: /note=“Description of Artificial Sequence Synthetic polypeptide”
SGGGGSGGGGSGGGGSGGGGSGGGGSGGGGSGGGGS

SEQ ID NO: 184 - protein, 41 aa, Artificial Sequence
  source: /note=“Description of Artificial Sequence Synthetic polypeptide”
SGGGGSGGGGSGGGGSGGGGSGGGGSGGGGSGGGGSGGGGS

SEQ ID NO: 185 - protein, 46 aa, Artificial Sequence
  source: /note=“Description of Artificial Sequence Synthetic polypeptide”
SGGGGSGGGGSGGGGSGGGGSGGGGSGGGGSGGGGSGGGGSGGGGS

SEQ ID NO: 186 - protein, 51 aa, Artificial Sequence
  source: /note=“Description of Artificial Sequence Synthetic polypeptide”
SGGGGSGGGGSGGGGSGGGGSGGGGSGGGGSGGGGSGGGGSGGGGSGGGGS

SEQ ID NO: 187 - protein, 4 aa, Unknown
  source: /note=“Description of Unknown FXIa cleavage site peptide”
KLTR

SEQ ID NO: 188 - protein, 4 aa, Unknown
  source: /note=“Description of Unknown FXIa cleavage site peptide”
DFTR

SEQ ID NO: 189 - protein, 9 aa, Unknown
  source: /note=“Description of Unknown FXIa cleavage site peptide”
TQSFNDFTR

SEQ ID NO: 190 - protein, 10 aa, Unknown
  source: /note=“Description of Unknown FXIa cleavage site peptide”
SVSQTSKLTR

SEQ ID NO: 191 - protein, 10 aa, Unknown
  source: /note=“Description of Unknown Thrombin cleavage site peptide”
DFLAEGGGVR

SEQ ID NO: 192 - protein, 7 aa, Unknown
  source: /note=“Description of Unknown Thrombin cleavage site peptide”
TTKIKPR

SEQ ID NO: 193 - protein, 5 aa, Unknown
  source: /note=“Description of Unknown Thrombin cleavage site peptide”
LVPRG

SEQ ID NO: 194 - protein, 5 aa, Unknown
  source: /note=“Description of Unknown Thrombin cleavage site peptide”
ALRPR

SEQ ID NO: 195 - protein, 474 aa, Artificial Sequence
  source: /note=“Description of Artificial Sequence Synthetic polypeptide”
MVSQALRLLCLLLGLQGCLAAVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFPGGGGSGGGGSGGGGSGGGGSGGGGSGGGGS

SEQ ID NO: 196 - protein, 688 aa, Artificial Sequence
  source: /note=“Description of Artificial Sequence Synthetic polypeptide”
MVSQALRLLCLLLGLQGCLAAVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFPGGGGSGGGGSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSALPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 27 aa, Artificial Sequence
  source: /note=“Description of Artificial Sequence Synthetic peptide”
GSPGTSESATPESGPGSEPATSGSETP

SEQ ID NO: 198 - protein, 4 aa, Artificial Sequence
  source: /note=“Description of Artificial Sequence Synthetic peptide”
GSSS

SEQ ID NO: 199 - protein, 12 aa, Artificial Sequence
  source: /note=“Description of Artificial Sequence Synthetic peptide”
GESPGGSSGSES

SEQ ID NO: 200 - protein, 12 aa, Artificial Sequence
  source: /note=“Description of Artificial Sequence Synthetic peptide”
GSEGSSGPGESS

SEQ ID NO: 201 - protein, 12 aa, Artificial Sequence
  source: /note=“Description of Artificial Sequence Synthetic peptide”
GSSESGSSEGGP

SEQ ID NO: 202 - protein, 12 aa, Artificial Sequence
  source: /note=“Description of Artificial Sequence Synthetic peptide”
GSGGEPSESGSS

SEQ ID NO: 203 - protein, 12 aa, Artificial Sequence
  source: /note=“Description of Artificial Sequence Synthetic peptide”
GSPAGSPTSTEE

SEQ ID NO: 204 - protein, 12 aa, Artificial Sequence
  source: /note=“Description of Artificial Sequence Synthetic peptide”
GSEPATSGSETP

SEQ ID NO: 205 - protein, 12 aa, Artificial Sequence
  source: /note=“Description of Artificial Sequence Synthetic peptide”
GTSESATPESGP

SEQ ID NO: 206 - protein, 12 aa, Artificial Sequence
  source: /note=“Description of Artificial Sequence Synthetic peptide”
GTSTEPSEGSAP

SEQ ID NO: 207 - protein, 12 aa, Artificial Sequence
  source: /note=“Description of Artificial Sequence Synthetic peptide”
GSTSESPSGTAP

SEQ ID NO: 208 - protein, 12 aa, Artificial Sequence
  source: /note=“Description of Artificial Sequence Synthetic peptide”
GTSTPESGSASP

SEQ ID NO: 209 - protein, 12 aa, Artificial Sequence
  source: /note=“Description of Artificial Sequence Synthetic peptide”
GTSPSGESSTAP

SEQ ID NO: 210 - protein, 12 aa, Artificial Sequence
  source: /note=“Description of Artificial Sequence Synthetic peptide”
GSTSSTAESPGP

SEQ ID NO: 211 - protein, 12 aa, Artificial Sequence
  source: /note=“Description of Artificial Sequence Synthetic peptide”
GTPGSGTASSSP

SEQ ID NO: 212 - protein, 12 aa, Artificial Sequence
  source: /note=“Description of Artificial Sequence Synthetic peptide”
GSSTPSGATGSP

SEQ ID NO: 213 - protein, 12 aa, Artificial Sequence
  source: /note=“Description of Artificial Sequence Synthetic peptide”
GSSPSASTGTGP

SEQ ID NO: 214 - protein, 12 aa, Artificial Sequence
  source: /note=“Description of Artificial Sequence Synthetic peptide”
GASPGTSSTGSP

SEQ ID NO: 215 - protein, 12 aa, Artificial Sequence
  source: /note=“Description of Artificial Sequence Synthetic peptide”
GEPAGSPTSTSE

SEQ ID NO: 216 - protein, 12 aa, Artificial Sequence
  source: /note=“Description of Artificial Sequence Synthetic peptide”
GTGEPSSTPASE

SEQ ID NO: 217 - protein, 12 aa, Artificial Sequence
  source: /note=“Description of Artificial Sequence Synthetic peptide”
GSGPSTESAPTE

SEQ ID NO: 218 - protein, 12 aa, Artificial Sequence
  source: /note=“Description of Artificial Sequence Synthetic peptide”
GSETPSGPSETA

SEQ ID NO: 219 - protein, 12 aa, Artificial Sequence
  source: /note=“Description of Artificial Sequence Synthetic peptide”
GPSETSTSEPGA

SEQ ID NO: 220 - protein, 12 aa, Artificial Sequence
  source: /note=“Description of Artificial Sequence Synthetic peptide”
GSPSEPTEGTSA

SEQ ID NO: 221 - protein, 12 aa, Artificial Sequence
  source: /note=“Description of Artificial Sequence Synthetic peptide”
GSGASEPTSTEP

SEQ ID NO: 222 - protein, 12 aa, Artificial Sequence
  source: /note=“Description of Artificial Sequence Synthetic peptide”
GSEPATSGTEPS

SEQ ID NO: 223 - protein, 12 aa, Artificial Sequence
  source: /note=“Description of Artificial Sequence Synthetic peptide”
GTSEPSTSEPGA

SEQ ID NO: 224 - protein, 12 aa, Artificial Sequence
  source: /note=“Description of Artificial Sequence Synthetic peptide”
GTSTEPSEPGSA

SEQ ID NO: 225 - protein, 12 aa, Artificial Sequence
  source: /note=“Description of Artificial Sequence Synthetic peptide”
GSTAGSETSTEA

SEQ ID NO: 226 - protein, 12 aa, Artificial Sequence
  source: /note=“Description of Artificial Sequence Synthetic peptide”
GSETATSGSETA

SEQ ID NO: 227 - protein, 12 aa, Artificial Sequence
  source: /note=“Description of Artificial Sequence Synthetic peptide”
GTSESATSESGA

SEQ ID NO: 228 - protein, 12 aa, Artificial Sequence
  source: /note=“Description of Artificial Sequence Synthetic peptide”
GTSTEASEGSAS

SEQ ID NO: 229 - protein, 42 aa, Artificial Sequence
  source: /note=“Description of Artificial Sequence Synthetic polypeptide”
GAPGSPAGSPTSTEEGTSESATPESGPGSEPATSGSETPASS

SEQ ID NO: 230 - protein, 42 aa, Artificial Sequence
  source: /note=“Description of Artificial Sequence Synthetic polypeptide”
TGGGSPAGSPTSTEEGTSESATPESGPGSEPATSGSETPASS

SEQ ID NO: 231 - protein, 42 aa, Artificial Sequence
  source: /note=“Description of Artificial Sequence Synthetic polypeptide”
GTSESATPESGPGSEPATSGSETPGTSESATPESGPGSEPAT

SEQ ID NO: 232 - protein, 78 aa, Artificial Sequence
  source: /note=“Description of Artificial Sequence Synthetic polypeptide”
GAPTSESATPESGPGSEPATSGSETPGTSESATPESGPGSEPATSGSETPGTSESATPESGPGTSTEPSEGSAPGASS

SEQ ID NO: 233 - protein, 72 aa, Artificial Sequence
  source: /note=“Description of Artificial Sequence Synthetic polypeptide”
GTSESATPESGPGSEPATSGSETPGTSESATPESGPGSEPATSGSETPGTSESATPESGPGTSTEPSEGSAP

SEQ ID NO: 234 - protein, 72 aa, Artificial Sequence
  source: /note=“Description of Artificial Sequence Synthetic polypeptide”
SPAGSPTSTEEGTSESATPESGPGSEPATSGSETPGTSESATPESGPGTSTEPSEGSAPGTSTEPSEGSAPG

SEQ ID NO: 235 - protein, 143 aa, Artificial Sequence
  source: /note=“Description of Artificial Sequence Synthetic polypeptide”
GSEPATSGSETPGTSESATPESGPGSEPATSGSETPGSPAGSPTSTEEGTSTEPSEGSAPGSEPATSGSETPGSEPATSGSETPGSEPATSGSETPGTSTEPSEGSAPGTSESAPESGPGSEPATSGSETPGTSTEPSEGSAP

SEQ ID NO: 236 - protein, 144 aa, Artificial Sequence
  source: /note=“Description of Artificial Sequence Synthetic polypeptide”
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEE

SEQ ID NO: 237 - protein, 144 aa, Artificial Sequence
  source: /note=“Description of Artificial Sequence Synthetic polypeptide”
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAP

SEQ ID NO: 238 - protein, 144 aa, Artificial Sequence
  source: /note=“Description of Artificial Sequence Synthetic polypeptide”
GTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSP

SEQ ID NO: 239 - protein, 288 aa, Artificial Sequence
  source: /note=“Description of Artificial Sequence Synthetic polypeptide”
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 240 - protein, 288 aa, Artificial Sequence
  source: /note=“Description of Artificial Sequence Synthetic polypeptide”
PGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGS

SEQ ID NO: 241 - protein, 576 aa, Artificial Sequence
  source: /note=“Description of Artificial Sequence Synthetic polypeptide”
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 242 - protein, 576 aa, Artificial Sequence
  source: /note=“Description of Artificial Sequence Synthetic polypeptide”
PGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGS

SEQ ID NO: 243 - protein, 864 aa, Artificial Sequence
  source: /note=“Description of Artificial Sequence Synthetic polypeptide”
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 244 - protein, 864 aa, Artificial Sequence
  source: /note=“Description of Artificial Sequence Synthetic polypeptide”
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 245 - protein, 993 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVP

SEQ ID NO: 246 - protein, 952 aa, Artificial Sequence
  source: /note=“Description of Artificial Sequence Synthetic polypeptide”
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFPGSPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGGGGSGGGGSGGGGSGGGGSGGGGSGGGGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDGLGSSPWSAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 247 - protein, 219 aa, Artificial Sequence
  source: /note=“Description of Artificial Sequence Synthetic polypeptide”
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 5 aa, Artificial Sequence
  source: /note=“Description of Artificial Sequence Synthetic peptide”
GGGGS

SEQ ID NO: 249 - protein, 30 aa, Artificial Sequence; identical to SEQ ID NO: 172
  source: /note=“Description of Artificial Sequence Synthetic polypeptide”
  VARIANT (2)..(4): /replace=“ ”
  VARIANT (5)..(5): /replace=“ ”
  VARIANT (7)..(9): /replace=“ ”
  VARIANT (10)..(10): /replace=“ ”
  VARIANT (12)..(14): /replace=“ ”
  VARIANT (15)..(15): /replace=“ ”
  VARIANT (17)..(19): /replace=“ ”
  VARIANT (20)..(20): /replace=“ ”
  VARIANT (22)..(24): /replace=“ ”
  VARIANT (25)..(25): /replace=“ ”
  VARIANT (27)..(29): /replace=“ ”
  VARIANT (30)..(30): /replace=“ ”
  MISC_FEATURE (1)..(30): /note=“This sequence may encompass 1-6 ” (Gly)x-(Ser)y“ repeating units, wherein x is 1-4 and y is 0-1; See specification as filed for detailed description of substitutions and preferred embodiments”
  MISC_FEATURE (1)..(30): /note=“Variant residues given in the sequence have no preference with respect to those in the annotations for variant positions”

SEQ ID NO: 250 - protein, 100 aa, Artificial Sequence
  source: /note=“Description of Artificial Sequence Synthetic polypeptide”
  VARIANT (2)..(100): /replace=“ ”
  MISC_FEATURE (1)..(100): /note=“This region may encompass 1-100 ”Gly“ repeating units”
  MISC_FEATURE (1)..(100): /note=“Variant residues given in the sequence have no preference with respect to those in the annotations for variant positions”
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 251 - protein, 500 aa, Artificial Sequence
  source: /note=“Description of Artificial Sequence Synthetic polypeptide”
  VARIANT (6)..(500): /replace=“ ”
  MISC_FEATURE (1)..(500): /note=“This region may encompass 1-100 ”Gly Gly Gly Gly Ser“ repeating units”
  MISC_FEATURE (1)..(500): /note=“Variant residues given in the sequence have no preference with respect to those in the annotations for variant positions”
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 252 - protein, 501 aa, Artificial Sequence
  source: /note=“Description of Artificial Sequence Synthetic polypeptide”
  VARIANT (7)..(501): /replace=“ ”
  MISC_FEATURE (2)..(501): /note=“This region may encompass 1-100 ”Gly Gly Gly Gly Ser“ repeating units”
  MISC_FEATURE (1)..(501): /note=“Variant residues given in the sequence have no preference with respect to those in the annotations for variant positions”
SGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 253 - protein, 6 aa, Artificial Sequence
  source: /note=“Description of Artificial Sequence Synthetic 6xHis tag”
HHHHHH

SEQ ID NO: 254 - protein, 6 aa, Artificial Sequence
  source: /note=“Description of Artificial Sequence Synthetic peptide”
SFLLRN